(19) United States
(12) Patent Application Publication
(10) Pub. No.: US 2007/0098738 A1
(43) Pub. Date: May 3, 2007

(54) MSP-3-like family of genes

(76) Inventors: Pierre Druilhe, Paris (FR); Subhash Singh, Mambai (IN); Daw Soe Soe, Paris (FR); Pedro Mejia, Montrouge (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/409,174

(22) Filed: Apr. 24, 2006

Related U.S. Application Data
(63) Continuation of application No. PCT/EP04/12909, filed on Oct. 22, 2004.

(30) Foreign Application Priority Data
Oct. 24, 2003 (EP) 03292673.5

(51) Int. Cl. A61K 39/002; C07H 21/04; C12N 1/10; C12N 15/74; C07K 14/445

(52) U.S. Cl. 424/191.1; 435/69.3; 435/471; 435/258.1; 530/350; 536/23.7

(57) ABSTRACT

The present invention relates to the protection against malaria. More particularly, the invention pertains to a novel family of genes encompassing the already known MSP-3 gene, and showing exceptional redundancy of exposed epitopes, hence suggesting that this family of genes plays an important part in the immunogenicity of the parasite. The characterization of this gene family enables the definition of novel immunogenic and vaccine compositions against P. falciparum. The invention also relates to an antigenic polypeptidic composition, comprising at least one MSP-3-b-like motif and at least one MSP-3-c/d-like motif.

The present invention relates to the protection against malaria. More particularly, the invention pertains to a novel family of genes encompassing the already known MSP-3 gene (now designated MSP3-1 as shown on FIG. 1), and showing exceptional redundancy of exposed epitopes, hence suggesting that this family of genes plays an important part in the immunogenicity of the parasite. The characterization of this gene family and as a consequence, of the family of corresponding gene products, enables the definition of novel immunogenic and vaccine compositions against P. falciparum.

The parasites responsible for malaria in human, including especially Plasmodium falciparum, exhibit different morphologies in the human host and express different antigens as a function of their localization in the organism of the infected host. The morphological and antigenic differences of these parasites during their life cycle in man enable at least four distinct stages of development to be defined.

The very first stage of development of the parasite in man corresponds to the sporozoite form introduced into the blood of the host by bites of insect vectors of the parasite. The second stage corresponds to the passage of the parasite into the liver and to the infection of the hepatic cells in which the parasites develop to form the hepatic schizonts which, when they are mature (for example, in the case of P. falciparum on the 6^{th} day after penetration of the sporozoites) release hepatic merozoites by bursting. The third stage is characterized by the infection of the blood erythrocytes by the asexual forms (merozoites) of the parasite; this erythrocytic stage of development corresponds to the pathogenic phase of the disease. The fourth stage corresponds to the formation of the forms with sexual potential (or gametocytes) which will become extracellular sexual forms or gametes in the mosquito.

Antibodies have been repeatedly shown to play an important part in the development of clinical immunity to Plasmodium falciparum malaria.

Numerous immunological studies now suggest that human antibodies of the cytophilic subclasses (IgG1 and IgG3) are particularly critical to the state of premunition. This anti-parasite immunity is a strain-independent, non-sterilizing type of immunity which is acquired after lengthy exposure (15-20 years) to the parasite. It is commonly observed in Africa and in Papua-New Guinea but it has only recently been documented in S-E Asia (Soe, Khin Saw et al. 2001). Although antibodies can act directly upon merozoite invasion of red blood cells, the most efficient in vivo mechanism for antibody-mediated parasite control in endemic areas requires the participation of monocytes (Khusmith and Druilhe 1983); (Lunel and Druilhe 1989). The antibody-dependent cellular inhibition (ADCI) assay mimics this cooperation between monocytes and cytophilic parasite-specific antibodies and appears today as the best in vitro surrogate marker of acquired immunity against P. falciparum blood stages.

Two molecules have so far been identified as targets of ADCI-effective human antibodies, namely the 48-kDa Merozoite surface-protein 3,—hereafter designated as MSP-3—(Oeuvray, Bouharoun-Tayoun et al. 1994) and the 220-kDa Glutamate-rich protein, —hereafter designated as GLURP—(Theisen, Soe et al. 1998). It has also been shown that GLURP and MSP-3 can inhibit parasite growth in vivo by passive transfer in P. falciparum—humanized SCID mice (Badell, Oeuvray et al. 2000). The association of human antibodies against MSP-3 with clinical protection is also indicated by a number of immuno-epidemiological studies, which demonstrate that the levels of MSP-3 specific cytophilic antibodies (IgG1 and IgG3) are significantly associated with a reduced risk of malaria attacks (Roussillon 1999). These studies have further shown that cytophilic IgG3 antibodies play a major part in protection against malaria, hence bringing epidemiological support to the concept that antibodies against MSP-3 can actively control parasite multiplication in vivo by cooperation with cells bearing Fcγ II receptors (Bouharoun-Tayoun, Oeuvray et al. 1995). These receptors display higher affinity for the IgG3 subclass than for the IgG1 subclass (Pleass and Woof 2001). The major B-cell epitopes recognized by these human IgG antibodies have been localized to conserved sequences in the MSP-3₂₁₂₋₂₅₇ region (Oeuvray, Bouharoun-Tayoun et al. 1994; Theisen, Soe et al. 2000; Theisen, Dodoo et al. 2001). Nucleotide-sequencing have demonstrated that these important epitopes are highly conserved among a number of P. falciparum laboratory lines and field isolates from Africa and Asia (Huber, Felger et al. 1997); (McColl and Anders 1997).

The inventors have now characterized a series of 9 P. falciparum genes, all clustered at the 3′ terminus of chromosome 10, which encode proteins and epitopes within, which are all targets for naturally occuring antibodies in malaria exposed individuals, mediating P. falciparum erythrocytic stage killing by cooperation with blood monocytes, and which exhibit an unusual degree of sequence conservation among various P. falciparum isolates.

The present invention hence pertains to a family of isolated genes, called the MSP-3-like family, the products of which having common structural and immunological features, as well as to some of these genes and the corresponding proteins, taken individually.

Antigenic polypeptides comprising epitopes from said novel proteins, as well as antigenic polypeptidic compositions comprising at least two of said epitopes and/or epitopes derived from any of the MSP3-like proteins, are also part of the invention.

Other important aspects of the invention are immunogenic compositions and vaccines against malaria, comprising as an immunogen a recombinant protein, a polypeptide or a polypeptidic composition as mentioned above.

Recombinant antibodies and part thereof, which cross-react with several products of the MSP-3-like gene family, also constitute an object of the present invention, either taken as such or in a medicament for passive immunotherapy or in a kit for the in vitro diagnosis of malaria.

The invention also concerns methods for the in vitro diagnosis of malaria in an individual, either by using an antigenic polypeptide, or by using an antibody as defined above, as well as kits comprising at least part of the necessary reagents (polypeptides, antibodies . . . ) for performing these methods.

Of course, nucleotide sequences encoding at least one of the novel P. falciparum antigens according to the invention, and their use in a medicament or a nucleic acid vaccine against P. falciparum, are also part of the invention.

Throughout the present text, a number of terms are used, that should be understood according to the following definitions:

In what follows, the term “gene” is synonymous to either a “naturally occurring sequence” including a coding sequence, or to a recombinant or synthetic sequence including a coding sequence. In the present text, a “gene” does also not necessarily contain regulatory elements, contrarily to the acceptation of this word which is often used in the scientific literature. Accordingly the gene according to the invention is any nucleotide sequence which comprises the Open Reading Frame of the naturally occurring sequence of Plasmodium or which comprises the same and further contains all or part of the regulatory sequences for expression of said naturally occurring sequence. According to the present definition, the gene is an isolated nucleic acid molecule, i.e, a nucleotide sequence which is not in its natural environment. Such a nucleotide sequence is also described as a purified.

In the present text, the expression “family of genes” has the same meaning as in the scientific literature, i.e., it designates a group of several genes which have a number of features or characteristics (structural or functional) in common.

According to the present invention, a “MSP-3-c/d-like motif” is an amino acids sequence of 20 amino acids, which is identical to any of the sequences of SEQ ID Nos: 25 to 30, or which is obtained by shuffling of at least two of these sequences. For example, a sequence having the amino acids 1 to 5 of SEQ ID No:25, followed by the amino acids 6 to 12 of SEQ ID No:29 and the amino acids 13 to 20 of SEQ ID No:27, is a MSP-3-c/d-like motif. In other words, a “MSP-3-c/d-like motif” is an amino acids sequence of 20 amino acids, wherein the amino acids are chosen among the following:

TABLE 1

| a.a. | | | | | | | | | | | | | | | | | | | | |
|---|---|---|---|---|---|---|---|---|---|---|---|---|---|---|---|---|---|---|---|---|
| position | 1 | 2 | 3 | 4 | 5 | 6 | 7 | 8 | 9 | 10 | 11 | 12 | 13 | 14 | 15 | 16 | 17 | 18 | 19 | 20 |
| a.a. | L | E | L | I | K | L | T | S | K | D | E | E | D | I | I | K | H | N | E | D |
| | | S | H | V | N | I | S | L | W | | K | N | N | | V | D | E | S | D | Q |
| | | | S | L | Y | V | P | | S | | R | | Q | | S | N | | | | |
| | | | Q | | P | | | | | | I | | | | P | | | | | |
| | | | | | | | | | | | | | | | A | | | | | |

Several of these aminoacids have a similar charge and will unlikely change the overall structure of the molecule or the recognition by antibodies, e.g., valine, isoleucine, leucine.

Any amino acids sequence of 20 amino acids, which comprises the most conserved amino acids indicated above (i.e., amino acids at positions 1, 2, 8, 10, 12, 14 and 17 to 20), and wherein the amino acid residues at other positions are different from the above one and which is recognized by an antibody directed against any of the MSP-3-c/d motifs of SEQ ID Nos: 25 to 30, will also be considered as a “MSP-3-c/d-like motif”, according to the present invention. This latter functional property can be tested by any immunoassay such as those known by the skilled artisan and/or described below.

In the present text, a “MSP-3-b-like motif” designates an amino acids sequence of 11 to 14 amino acids, which is identical to any of the sequences of SEQ ID Nos: 17 to 24, or which is obtained by shuffling of at least two of these sequences. For example, a sequence having the amino acids 1 to 5 of SEQ ID No:17, followed by the amino acids 6 to 11 of SEQ ID No:22, is a MSP-3-b-like motif. In other words, a “MSP-3-b-like motif” is an amino acids sequence of 11 to 14 amino acids, wherein the amino acids are chosen among the following, wherein “−” means “no amino acid”:

TABLE 2

| | | | | | | | | | | | | | | |
|---|---|---|---|---|---|---|---|---|---|---|---|---|---|---|
| a.a. position | 1 | 2 | 3 | 4 | 5 | 6 | 7 | 8 | 9 | 10 | 11 | 12 | 13 | 14 |

| | | | | | | | | | | | | | | |
|---|---|---|---|---|---|---|---|---|---|---|---|---|---|---|
| a.a. | I | L | E | R | G | W | E | F | G | G | G | V | P | E |
| | Y | F | D | D | A | G | L | I | S | | S | A | Y | F |
| | — | — | P | — | L | S | A | G | A | | A | L | L | — |
| | | | — | | — | I | | L | | | | I | | |
| | | | | | | E | | S | | | | | S | |

Several of these aminoacids have a similar charge and will unlikely change the overall structure of the molecule or the recognition by antibodies, e.g., valine, isoleucine, leucine.

A subgroup of MSP-3-b-like motifs corresponds to the sequences of SEQ ID Nos:17, 18 and 22 and their combination, i.e., the sequences of 11 amino-acids selected as follows:

TABLE 3

| | | | | | | | | | | | |
|---|---|---|---|---|---|---|---|---|---|---|---|
| a.a. position | 1 | 2 | 3 | 4 | 5 | 6 | 7 | 8 | 9 | 10 | 11 |

| | | | | | | | | | | | |
|---|---|---|---|---|---|---|---|---|---|---|---|
| a.a. | I | L | G | W | E | F | G | G | G | V | P |
| | Y | F | A | | | I | | | | A | |

Any amino acids sequence of 11 to 14 amino acids, which comprises the most conserved amino acids indicated above (i.e., the amino acids indicated in table 3, which correspond to particular amino acids at positions 1, 2, and 5 to 13 of Table 2), and wherein the amino acid residues at other positions are different from the above one, and which is recognized by an antibody directed against any of the MSP-3-b motifs of SEQ ID Nos: 17 to 24, will also be considered as a “MSP-3-b-like motif”, according to the present invention. This latter functional property can be tested by any immunoassay such as those known by the skilled artisan and/or described below.

In what follows, reference is sometimes made to a gene or a protein which is an “homologue” of a particular gene or protein the sequence of which is disclosed. This word herein designates close sequences in different Plasmodium strains (in particular, P. falciparum strains), i.e., sequences exhibiting at least 70%, and preferably at least 90% of sequence identity, with the sequence of reference.

A “conservative substitution” means, in an amino acid sequence, a substitution of one amino acid residue by another one which has similar properties having regard to hydrophobicity and/or steric hindrance, so that the tertiary structure of the polypeptide is not dramatically changed. For example, replacing a guanine by an alanine or vice-versa, is a conservative substitution. Valine, leucine and isoleucine are also amino acids that can be conservatively substituted by each other. Other groups of conservative substitution are, without being limitative, (D, E), (K, R), (N, Q), and (F, W, Y). A variant of a polypeptide, obtained by conservative substitution of at least one amino acid of said polypeptide, will be designated here as a “conservative variant” of said polypeptide.

The expression “derived from” applied to sequences of either nucleotides or amino-acid residues indicates that the concerned sequence is designed starting from the knowledge of the structure and/or properties identified for the family of sequences according to the invention. However, the concerned sequences can be prepared by any appropriate technical process, including by recombinant technology or by synthesis. Hence the sequences are not restricted to those obtained from naturally occurring genes or proteins. They can even be chimeric sequences.

Further definitions are provided in the following text, when necessary.

The inventors herein describe a group of 9 genes, 6 of which have never been described, and which are all clustered in the same region of chromosome 10. This chromosome indeed contains a series of 9 open reading frames, separated by non coding regions, and comprises in a row (5′-3′) genes encoding proteins denominated first GLURP, followed at 1300 base-pairs by MSP-3 (now denominated MSP-3-1) followed by 7 other genes denominated MSP-3-2 (also designated sometimes MSP6), MSP-3-3, MSP-3-4, MSP-3-5, MSP-3-6, MSP-3-7, MSP-3-8. This organisation is shown in FIG. 1.

Besides being clustered in the same chromosomal region, those 9 genes have outstanding features, that indicate that they are privileged products for vaccine development against P. falciparum blood stage infection:

It was shown that all 9 genes were expressed simultaneously in all parasites studied, i.e., that the corresponding proteins could be detected in P. falciparum erythrocytic stages and are all located on the merozoite surface. This was previously shown for GLURP, MSP-3-1 and MSP-3-2 (designated “MSP6” by (Trucco, Fernandez-Reyes et al. 2001) and has been further demonstrated for the remaining by the construction of particular sequences in the N-terminus of those genes which are unique for each of them, which do not share cross-reactive epitopes, and which corresponding antibodies all react with the merozoite surface. Moreover, transcription was demonstrated by RT-PCR with unique primers specific of each.

Moreover, the 8 MSP-3-like genes share the same general gene organisation, which is illustrated in FIG. 1, with an initial N-terminus “signature” of 4 aminoacids (indicated “s” in FIG. 1) identical in each of them and identical to similar MSP-3 homologous proteins described in Plasmodium vivax and Plasmodium Knowlesi.

A first object of the present invention is hence a family (or group) of isolated or purified genes which have the following properties:
they are located on chromosome 10 of Plasmodium falciparum;
they are highly conserved in Plasmodium falciparum strains;
they are expressed in Plasmodium falciparum at the erythrocytic stages;
they encode proteins which have a NLRN or NLRK signature at their N-terminal extremity and which are located at the merozoite surface,

wherein said family comprises at least 3 genes.

The invention also relates to a family of fragments of said family of genes. A particular family has at least 3 polynucleotide fragements of said genes. The invention also relates to the polynucleotide fragments contained in the C-terminal sequence of genes of the family.

Particular polynucleotide fragments of the family of genes of the invention, or particular families of such polynucleotide fragments are derived from said genes, and encode the C-terminal part of said genes. Said C-terminal part is described hereafter including in the examples and in FIG. 10. The invention relates also to combinations of said fragements, including combinations having multiple polynucleotides encoding the C-terminal part of said genes, especially recombinant sequences.

Other polynucleotide fragments of said genes or of said family of genes, including recombined fragments, are fragments of the sequence encoding the C-terminal part of said genes.

Polynucleotides of the invention have 30 to 1500, especially 30 to 500 nucleotides, especially 30 up to 250, or to 240, or to 210, or to 180, or to 150, or to 120 or to 90 nucleotides.

The NLRN or NLRK signature is most often followed by A or G, in the proteins of the MSP-3 family according to the invention.

The genes of the family preferably also share the same general organization, as shown in FIG. 1.

An example of such a family is the whole, MSP-3-like family, comprising the genes of sequences SEQ ID Nos: 1, 3, 5, 7, 9, 11, 13, and 15 or fragments thereof as defined above. Any group of at least 3 genes or fragments thereof as defined above, selected amongst these genes is also considered as a gene family according to the invention.

Except from the N-terminal signature mentioned above, the remaining of the N-terminus part is highly variable from one gene product to the other, whereas, in contrast, the C-terminus is identical in its organisation for all genes, except 2 (MSP-3-5 and MSP-3-6) including the “b” epitope-like stretch (“b”), the “c/d” epitope-like (“c/d”), the Glutamic-rich region, and at the extreme C-term a leucine zipper.

Based on the organisation of said genes in the C-terminus part of the gene products, the inventors provide a particular family of genes, within the cluster of 9 genes cited hereabove and within the 8MSP3-like genes disclosed above. This particular family encompasses MSP3-1, MSP3-2, MSP3-3, MSP3-4, MSP3-7 and MSP3-8 genes among the 8MSP3-like genes of Plasmodium strains especially those genes in P. falciparum strains.

Said particular family of genes encodes for a corresponding particular family of proteins (also designated polypeptides) encompassing MSP3-1, MSP3-2, MSP3-3, MSP3-4, MSP3-7 and MSP3-8 polypeptides.

Said particular family of genes and particular family of corresponding proteins is also characterized by the fact that said genes further have a conserved C-terminal sequence which encodes epitopes, especially T-epitopes which are conserve among the genes of the family and wherein said terminal sequence further comprises divergences in codons in regions outside of the epitopes (encompassing a MSP-3-b-like motif and a MSP-3-c/d-like motif) which divergences are conserved among the genes of the family.

Particular families of genes according to the invention are hence families as described above, wherein said genes further have the following property: they encode proteins which have a MSP-3-b-like motif and/or a MSP-3-c/d-like motif. An example of such a family is the family encompassing the genes of sequences SEQ ID Nos: 1, 3, 5, 7, 13, and 15, or any group of at least 3 genes selected amongst these sequences.

All 7 proteins (GLURP+the 6 homologous MSP-3-like molecules) elicit antibodies in individual exposed to malaria.

For those gene products in which it has been investigated, particularly GLURP, MSP-3-1 and MSP-3-2, the immune responses elicited are associated with clinical protection against malaria attacks under field conditions. This association is highly statistically significant, particularly with antibodies made of the IgG3 isotype, and was confirmed in three settings, Dielmo and Ndiop in africa, Oo-do I Burma. For reasons of homology described below, it is extremely likely that the same finding will be made for the remaining 5 genes.

In the case of GLURP, MSP-3-1 and MSP-3-2, the regions targeted by antibodies associated with protection are the non repeat region R0 of GLURP and the C-terminus non repeated region of MSP-3-1 and MSP-3-2. The various peptides derived from MSP-3-1 are shown in FIG. 2. Protection was associated with antibodies to peptides MSP-3-b, c and d.

Antibodies to the 7 gene products are all effective at mediating P. falciparum blood stage killing, in the monocyte-dependent, antibody-mediated ADCI mechanism, under in vitro conditions. These results, described in Example 1, show that antibodies to each of those regions are equally effective at achieving P. falciparum erythrocytic stage growth inhibition under in vitro conditions.

Preferred family of genes according to the invention therefore further have the following property: antibodies to the products of said genes mediate Plasmodium falciparum blood stage killing, in the monocyte-dependent, antibody-mediated ADCI mechanism, under in vitro conditions.

According to another preferred embodiment, a family of genes according to the invention therefore further has the following property: antibodies to the products of said genes mediate Plasmodium falciparum growth inhibition in mice infected by P. falciparum (confer e.g. the asay disclosed in Examples 1 and 8).

The inventors have also demonstrated that there is a very unusual high degree of sequence conservation of each of the 7 genes, among various P. falciparum isolates. This had been previously shown for GLURP and led to choose the R0 non-repetitive region which has the highest conservation among various isolates, yet has some aminoacid substitutions. This was also shown for MSP-3-1 which sequence was found to be outstandingly conserved among 111 isolates for the region covering peptides MSP-3-a, b, c and d, i.e., the region used for immunisation of volunteers, where no single aminoacid substitution and therefore no aminoacid change was found whatsoever. This was recently further confirmed for the remaining of the C-terminus of MSP-3-1 and the whole C-term conserved region of MSP-3-2, MSP-3-3, MSP-3-4, MSP-3-7, and MSP-3-8 (FIGS. 9 and 10). This remarkable degree of sequence conservation of this gene family is in marked contrast with the relatively large polymorphism observed for most of the other vaccine candidates currently studied, and is obviously an important criterium that strengthens the potential of this gene family for vaccine development.

The invention especially points out that the feature characterizing the particular family of 6 genes (and the corresponding particular family of 6 proteins) MSP3-1, MSP3-2, MSP3-3, MSP3-4, MSP3-7 and MSP3-8, lies both (i) in the conversation of nucleotides or amino-acids in the regions comprising or defining epitopes (for example epitopes contained in the b- or in the c/d- like motifs defined above) and (ii) in the conservation of divergent nucleotides (and encoded amino-acids) comprised within the c-terminal part especially in regions located outside of the epitopes contained in the particular motifs (including, b-, c/d-, motifs).

It is noted that the two types of opposite conservations, i.e. (i) conservation of nucleotides (amino acids) shared by determined regions of the C-terminal sequence of the 6MSP3-like genes (or gene products) and (ii) the conservation of divergent nucleotides and encoding amino acids in other regions of said C-terminal sequence of the 6MSP3-like genes (or gene products) is of interest for the definition of means capable of eliciting or improving an immunological response and preferably a protective immunological response against infection by Plasmodium strains, especially P. falciparum strains.

Gene families as described above, comprising at least 3 genes selected amongst the genes of sequences SEQ ID Nos: 1, 3, 5, 7, 9, 11, 13, and 15, and in particular amongst the genes of sequences SEQ ID Nos 1, 3, 5, 7, 13 and 15) or their homologues in Plasmodium, particularly Plasmodium falciparum strains are therefore also preferred gene families of the invention.

A particular family especially consists of the 6MSP3-like genes, i.e. MSP3-1, MSP3-2, MSP3-3, MSP3-4, MSP3-7 and MSP3-8 or another family comprises at least 3 genes including MSP3-1 and MSP3-2 genes.

Another aspect of the present invention is an isolated or purified Plasmodium falciparum gene which has the sequence of SEQ ID No:5, 7, 13 or 15, or an isolated gene corresponding to an homologue of a Plasmodium falciparum gene of sequence of SEQ ID No:5, 7, 9, 11, 13 or 15 in particular of SEQ ID Nos: 5, 7, 13 or 15 in a Plasmodium strain.

These genes, which are non described MSP-3-like genes, can be very useful for the skilled artisan in a number of applications in the research, diagnostic and vaccinations fields, for the reasons described above and hereafter. In particular, they can be used to produce recombinant MSP-3-like proteins. Accordingly, recombinant proteins of SEQ ID Nos: 6, 8, 10, 12, 14 and 16 in particular proteins SEQ ID Nos: 6, 8, 14 and 16, are also part of the present invention, as well as any recombinant protein having the sequence of a protein which is an homologue of a protein of SEQ ID Nos: 6, 8, 10, 12, 14 or 16 in particular of a protein of SEQ ID Nos: 6, 8, 14 or 16, in a Plasmodium strain different from the 3D7 strain.

The invention also concerns genes or polynucleotide fragments thereof, which are variants of the above defined genes or fragments thereof, and which hybridize in stringent conditions with said genes or fragments of genes. The variants have especially the same length as, or alternatively are shorter than the gene or gene fragments to which they hybridize in stringent conditions.

“Stringent hybridization conditions” are defined herein as conditions that allow specific hybridization of two nucleic acid especially two DNA molecules at about 65° C., for example in a solution of 6×SSC, 0.5% SDS, 5× Denhardt's solution and 100 μg/ml of denatured non specific DNA or any solution with an equivalent ionic strength, and after a washing step carried out at 65° C., for example in a solution of at most 0.2×SSC and 0.1% SDS or any solution with an equivalent ionic strength. However, the stringency of the conditions can be adapted by the skilled person as a function of the size of the sequence to be hybridized, its GC nucleotide content, and any other parameter, for example following protocols described by Sambrook et al, 2001 (Molecular Cloning: A Laboratory Manual, 3^{rd} Edition, Laboratory Press, Cold Spring Harbor, N.Y.).

The comparison of sequences between genes of the MSP-3 family shows a very unusual conservation of the epitopes and also a conservation of the divergent amino-acid residues located between the epitopes, said conservations occurring between members of the family, especially of those targeted by biologically active antibodies, which is critical for protection, especially for those members which genes are represented as SEQ ID Nos 1, 3, 5, 7, 13 and 15. The comparison of the sequences are summarised in FIG. 11. Hence the isolation and characterization of the various genes of the MSP3-like family has been significant for the comprehension of immunological response and for the design of means having improved interest for the preparation of immunogenic compositions or protective compositions, against Plasmodium, especially against Plasmodium falciparum.

The inventors have identified 2 regions which are very similar, if not totally identical, between members of the family and concern one critical region in the MSP-3-b peptide and one in the MSP-3-c and d peptides (region that is covered by both peptides MSP-3-c and MSP-3-d). The small differences between these very conserved epitopes among the various genes is summarised in FIGS. 12 and 13. It is noteworthy and highly significant that the most conserved regions across the various genes are those two that are the target of biologically active antibodies in the ADCI assay in vitro and by passive transfer in SCID mice (see above).

The inventors have also demonstrated that there exists immunological cross-reactivity between the different proteins of the MSP-3 family, as a consequence of those structural homologies between members of the gene family (examples 5 to 8).

The practical consequence at immunological and vaccine development level is that immunisation by any of the members of the gene family will induce antibodies reactive to the same and to all of the remaining gene products.

Therefore, the present invention constitutes a very particular type of multi-gene family where, instead of epitope polymorphism, which is usually the feature of multi-gene families described to-date, epitope conservation is the main characteristic and where, in case of deletion, mutation in one given gene, another or all other members of the family can take over the antigenic function. In addition, all genes are simultaneously expressed by one given parasite.

The invention thus also concerns a protein which is encoded by a gene among those disclosed here above. In a particular embodiment, the protein is a recombinant protein.

An antigenic polypeptide comprising a fragment of at least 5, or at least 10, preferably at least 15, consecutive amino acids from a protein according to the invention is therefore part of the invention. In particular embodiments of the invention, such an antigenic polypeptide has 80 or less, especially up to 70, or up to 60, or up to 50 or up to 40 and possibly up to 30 amino-acid residues.

Polypeptides limited to fragments of MSP3-1(HERAKNAYQKANQAVLKAKEASSY,AKEASSYDYILGWEFGGGVPEHKKEEN, PEHKKEENMLSHLYVSSKDKENISKENE) disclosed in Oeuvray, Bouharoun-Tayoun et al 1994 or fragments of MSP-3-2 (ILGWEFGGG-[AV]-P) disclosed in Trucco, Fernandez-Reyes et al 2001, are not as such within the invention considered here.

Any fragment derived from MSP3-1 or MSP3-2 complying with the above definitions is nevertheless within the scope of the invention when it is included in antigenic compositions disclosed in the present application.

As described above for the family of MSP3-like genes, especially the family of MSP3-1, MSP3-2, MSP3-3, MSP3-4, MSP3-7 and MSP3-8 and the corresponding families of polypeptides, the invention also relates to compositions of antigenic polypeptides comprising at least two antigenic polypeptides derived from the family of MSP3-like proteins, which polypeptides are capable of eliciting or improving an immunological response representative of the response obtained against the native polypetides in a human host, in particular representative of a protective response against Plasmodium strains, especially against P. falciparum. Said compositions are thus antigenic polypeptidic composition and advantageously are immunogenic compositions.

Preferred antigenic polypeptides according to the invention are those that comprise at least one MSP3-b-like and/or at least one MSP3-c/d-like motifs, as defined above.

For example, any antigenic polypeptide according to the above definitions, comprising at least one motif selected amongst the sequences SEQ ID Nos: 19 to 24 (b-like motifs) and 27 to 30 (c/d-like motifs), or consisting of any of such motifs, is part of the invention, as well as any antigenic polypeptide comprising at least one motif selected amongst the variants obtained by conservative substitution of at least one amino-acid in the sequences SEQ ID Nos: 19 to 24 and 27 to 30, provided said antigenic polypeptide is not limited to a fragment of MSP-3-1 or MSP-3-2 as disclosed in the prior art cited above. Alternatively, corresponding b- or c/d-like motifs or antigenic polypeptide comprising said motifs which comply with the above definitions of the antigenic polypeptides and which can be derived from other strains of Plasmodium, especially from Plasmodium falciparum are within the scope of the invention. Such fragments can be derived from the sequences illustrated on FIG. 10.

When combined in a composition with antigenic polypeptides of the invention, especially designed starting from SEQ ID Nos: 19 to 24 or 27 to 30 or variants thereof as described above, antigenic polypeptides originating from MSP3-1 or MSP3-2 as disclosed in SEQ ID Nos: 17, 18, 25 or 26, or variants thereof having conservative substitutions or having sequences derived from other Plasmodium especially other Plasmodium falciparum strains are also within the scope of the invention.

Another aspect of the present invention is an antigenic polypeptidic composition comprising at least two different MSP-3-b-like motifs, and/or at least two different MSP-3-c/d-like motifs. By “polypeptidic composition” is meant a composition comprising polypeptidic components, i.e., polypeptides or molecules comprising a polypeptidic moiety, such as lipopolypeptides, conjugates consisting of polypeptides bound to a support, etc. The polypeptidic compositions according to the invention can be solutions, caplets, etc.

In a particular embodiment of the antigenic polypeptidic composition according to the invention, the at least two different MSP-3-b-like motifs are selected amongst the sequences of SEQ ID Nos: 17 to 24 and conservative variants thereof, and/or the at least two different MSP-3-c/d-like motifs are selected amongst the sequences of SEQ ID Nos: 25 to 30 and conservative variants thereof.

Antigenic polypeptides of the invention or antigenic polypeptidic compositions of the invention as disclosed above advantageously comprise or consist of polypeptidic components which have 10 to 80 amino acids for each polypetidic components, in particular, 10 (or 12, or 15, or 20) to 70, or 10 (or 12, or 15, or 20) to 60, or 10 (or 12, or 15, or 20) to 50, or 10 (or 12, or 15, or 20) to 40, or 10 (or 12, or 15, or 20) to 30 amino acids for each polypeptidic component.

The antigenic polypeptidic compositions of the invention are advantageously immunogenic compositions, capable of eliciting or improving the production of antibodies in a host, especially in a human host.

Therefore, each polypeptide or polipeptidic component is, according to the above characterization of the genes and polypeptides family, characterized in that, in addition to the above features relating to the presence of one or several motifs among defined motifs b-, c/d-, and possibly a-, e- and f-like motifs, they are derived from the C-terminal polypeptides of MSP3-1, MSP3-2, MSP3-3, MSP3-4, MSP3-7 and MSP3-8 proteins.

The particular C-terminal polypeptidic sequences of these MSP3-like proteins are described in the examples which follow and in the figures (FIG. 10) for various strains of Plasmodium falciparum, for MSP3-1, MSP3-2, MSP3-3, MSP3-4, MSP3-7 and MSP3-8 proteins. All the sequences which are described in FIG. 10, taken individually or as combination of at least one, preferably at least two of these sequences, especially combinations of sequences from the different MSP3-like proteins are within the scope of the invention.

The invention also relates to homologues sequences of these particular polypeptides, derived from other strains of Plasmodium especially from P. falciparum. These homologue sequences (including chimeric sequences) can be used to derive the polypeptidic components of the invention.

In the antigenic polypeptidic composition of the invention, the at least two different MSP-3-b-like motifs, and/or at least two different MSP-3-c/d-like motifs can be carried by distinct molecules (i.e., the composition can comprise a diversity of molecules each containing only one motif); alternatively, each polypeptidic component of these composition can carry at least two motifs. An antigenic composition as described above, which contains molecules that comprise at least two different MSP-3-b-like motifs, and/or at least two different MSP-3-c/d-like motifs, is hence an object of the present invention. These molecules can be complex molecules, in which the at least two motifs are part of distinct peptides covalently linked to a common carrier; preferably, their polypeptidic moiety is constituted by a unique polypeptide comprising said motifs. Fusion proteins, comprising several parts coming from different MSP-3 proteins, can be included in these compositions.

Particular polypeptidic compositions of the invention comprise or consist of fusion polypeptides, such as, for example:
A fusion polypeptide comprising or consisting of the C-terminal sequence of all or of at least 3 MSP3-like proteins selected among MSP3-1, MSP3-2, MSP3-3, MSP3-4, MSP3-7 and MSP3-8; preferred fusion polypeptides will at least comprise said C-Terminal sequence of MSP3-1 and/or MSP3-2 proteins;
Fusion polypeptides of polypeptidic components shorter than said C-terminal sequences (i.e. shorter that 80 amino acids) wherein said polypeptidic components comprise or consist of: at least one motif comprising an epitope, selected among motifs designated as b and c/d motifs as described in the present application, said motif being derived from one MSP3-like protein among MSP3-1, MSP3-2, MSP3-3, MSP3-4, MSP3-7 and MSP3-8 being associated with the same motif or with a different motif characteristic of another of these MSP3-like proteins, or being associated with the same motif of several (2, 3, 4, 5,) of the MSP3-like proteins or with different motifs of several (2, 3, 4, 5) of the MSP3-like proteins.

The polypeptidic components of the compositions of the invention can be prepared by any appropriate preparation processes, including by processes involving recombinant expression or by chemical synthesis. The same applies to the molecules derived from the association of said polypeptidic components, including to prepare fusion polypeptides.

In view of the conservation of the epitopes, the inventors have investigated whether cytophilic antibodies against GLURP and MSP-3 are involved in the development of immunity to clinical malaria in an Asian population of Myanmar, as they have been reported to be in Africa, i.e., in a different human and parasite genetic background. Results, disclosed in Example 7 below, show that levels of cytophilic IgG3 antibodies against conserved regions of MSP-3-1 and GLURP are significantly correlated with clinical protection against P. falciparum malaria. In contrast, levels of non-cytophilic IgG4 antibodies against GLURP increased with the number of malaria attacks. Most importantly, there was a complementary effect of the MSP-3-1- and GLURP-specific IgG3 antibodies in malaria protection. In those individuals not responding to one of the antigens, a strong response to the other was consistently detected and associated with protection, suggesting that the induction of antibodies against both MSP3 and GLURP could be important for the development of protective immunity.

According to another embodiment of the invention, the antigenic polypeptidic composition hence further comprises an antigenic polypeptidic molecule comprising at least 10 consecutive amino acid residues from the R0 region of GLURP (SEQ ID No: 34).

As mentioned above, an antigenic polypeptidic composition according to the invention can comprise a limited number of molecules each comprising a variety of epitopes, or a variety of molecules each comprising a limited number of epitopes. According to a particular embodiment, the composition of antigenic polypeptides comprises from 2, preferably from 3 to less than 9 especially from 2 to 6 polypeptides encoded by the genes of the invention.

As said above the epitopes can be corresponding epitopes originating from different MSP3-like proteins among MSP3-1, MSP3-2, MSP3-3, MSP3-4, MSP3-7 and MSP3-8 or different epitopes, e.g. b and c/d-like motifs of the same or of different of these proteins.

Various embodiments of the invention, are illustrated based on the above defined features.

The invention relates to an antigenic polypeptidic composition, wherein the MSP3-b-like motifs are comprised in polypeptidic components having from 10 to 80 amino acid residues.

Alternatively or in addition, the invention relates to an antigenic polypeptidic composition, wherein the MSP3-c/d-like motifs are comprised in polypeptididic components having from 20 to 80 amino acid residues.

According to a further embodiment, the invention relates to an antigenic polypeptidic composition, wherein MSP3-b-like motif(s) and the MSP3-c/d-like motif(s) are comprised in a unique polypeptidic component.

An antigenic polypeptidic composition of the invention can be characterized, in that the MSP3-b-like motifs and/or the MSP3-c/d-like motifs are separated in the polypeptidic component, by the aminoacid sequence naturally contained between them in the MSP3-like protein from which they derive.

In another particular embodiment of the invention, the antigenic polypeptidic composition, is characterized in that the polypeptidic components, or each of the polypeptidic component comprise or consist of an amino-acid sequence derived from one or several MSP3-like proteins, said amino-acid sequence consisting of all or part of the C-terminal sequence of one or several MSP3-like proteins selected among MSP3-1, MSP3-2, MSP3-3, MSP3-4, MSP3-7 and MSP3-8 proteins of Plasmodium, especially of Plasmodium falciparum.

An antigenic polypeptidic composition of the invention can also be characterized in that the polypeptidic component(s) consists of the C-terminal sequences of MSP3-like proteins including at least MSP3-1 and MSP3-2 or fragments of said C-terminal sequences comprising or consisting of the MSP3-1-b, MSP3-1 c/d, MSP3-2-b and MSP3-2 c/d motifs.

In such an antigenic polypeptidic composition, the polypeptidic component(s) further comprise amino-acid sequences consisting of the C-terminal sequences of MSP3-like proteins selected among MSP3-3, MSP3-4, MSP3-7 and MSP3-8 or fragments of said C-terminal sequences comprising or consisting of the MSP3-b-like and the MSP3- c/d-like motifs.

In another embodiment, the antigenic polypeptidic composition is characterized in that the polypeptidic components are several fusion polypeptides wherein each fusion polypeptide comprises or consists of a polypeptide having the sequence consisting of:
(i) the C-terminal sequence of at least two MSP3-like proteins selected among MSP3-1, MSP3-2, MSP3-3, MSP3-4, MSP3-7 and MSP3-8 or;
(ii) several, especially at least 2 peptide fragments of the C-terminal sequence of at least two MSP3-like proteins selected among MSP3-1, MSP3-2, MSP3-3, MSP3-4, MSP3-7 and MSP3-8, wherein each peptide fragment comprises or consists of a least one MSP3-b-like motif or at least one MSP3-c/d-like motif.

Another antigenic polypeptidic composition of the invention comprises or consists of a fusion polypeptide comprising or consisting of:
(i) the C-terminal sequence of each of the MSP3-like proteins selected among MSP3-1, MSP3-2, MSP3-3, MSP3-4, MSP3-7 and MSP3-8 or;
(ii) one or several, especially at least 2, peptide fragments of the C-terminal sequence of each of the MSP3-like proteins selected among MSP3-1, MSP3-2, MSP3-3, MSP3-4, MSP3-7 and MSP3-8, wherein each peptide fragment comprises or consists of a least one MSP3-b-like motif or at least one MSP3-c/d-like motif, wherein the fragments of the C-terminal sequences of the various MSP3-like proteins form a unique amino-acid sequence.

In a further antigenic polypeptidic composition, the peptide fragments of the C-terminal sequence of at least two MSP3-like proteins is selected among MSP3-1, MSP3-2, MSP3-3, MSP3-4, MSP3-7 and MSP3-8 and contain at least one MSP3-b-like motif and one or several further motif selected among the MSP3-a, -c/d, -e and -f-like motifs and said motifs are contiguous or not in said peptide fragments.

In another particular embodiment of the invention as disclosed above, antigenic polypeptidic composition, wherein the C-terminal sequence of the MSP3-like proteins are the following sequences:
(i) for MSP3-1, any sequence of FIG. 10A, and especially the sequence of strain 3D7;
(ii) for MSP3-2, any sequence of FIGS. 10-B-D and especially the sequence of strain 3D7; or a fragment of any of said sequences starting at amino-acid residue 161 (or 165 for sequences MSP3.2FL D4) and ending at amino acid residue 371 (or 376 for sequence MSP3.2FL D4),
(iii) for MSP3-3, any sequence of FIGS. 10D-E, and especially the sequence of strain 3D7;
(iv) for MSP3-4, any sequence of FIGS. 10E-F, and especially the sequence of strain 3D7;
(v) for MSP3-7, any sequence of FIGS. 10F-H, and especially the sequence of strain 3D7;
(vi) for MSP3-8, any sequence of FIG. 10I, and especially the sequence of strain 3D7.

A further example of antigenic polypeptidic composition according to the above described possibilities is a combination, especially a mixotope, comprising a variety, especially at least two of synthetic peptides comprising the sequence:

(SEQ ID No:31)

X1-X2-X3-X4-X5-X6-X7-X8-X9-G-X9-X10-X11-X12,

wherein:

X1=I, Y or none;

X2=L, F or none;

X3=E, D, P or none;

X4=R, D or none;

X5=G, A, L or none;

X6=W, G, S, I or E;

X7=E, L or A;

X8=F, I, G, L or S;

X9=G, S or A;

X10=V, A, L, I or S;

X11=P, Y or L;

X12=E, F or none.

A “mixotope” is a combinatorial library of peptides which can be obtained in a single synthesis, as described by (Gras-Masse, Georges et al. 1999).

Another mixture or especially a mixotope derived from MSP-3-b and which can be included in an antigenic polypeptidic composition according to the invention is combination, especially a mixotope, comprising a variety, especially at least two of synthetic peptides comprising the sequence

(SEQ ID No:32)

X₁-X₂-X₃-W-E-X₄-G-G-G-X₅-P,

wherein:

X₁=I or Y:

X₂=L or F;

X₃=G or A;

X₄=F or I; and

X₅=V or A.

Similarly, another antigenic polypeptidic composition according to the invention is a combination, especially a mixotope, comprising a variety, especially at least two of synthetic peptides comprising the sequence

(SEQ ID No:33)

L-X1-X2-X3-X4-X3-X5-X6-X7-D-X8-X9-X10-I-X11-X12-
X13-X14-X15-X16,

wherein:

X1=E, or S;

X2=L, H, S or Q;

X3=I, V or L;

X4=K, N, Y or P;

X5=T, S or P;

X6=S or L;

X7=K, W or S;

X8=E, K, R or I;

X9=E or N;

X10=D, N or Q;

X11=I, V, S, P or A;

X12=K, D or N;

X13=H or E;

X14=N or S;

X15=E or D;

X16=D or Q.

The combination of several polypeptides of each of the above three groups, or of polypeptides of several of the above three groups can form either a mixture of peptides or polypeptides derived from the C-terminal region of the MSP3-like proteins comprising said peptides or can form fusion polypeptides or can form a mixture of various fusion polypeptides.

The above antigenic mixotope compositions can be in particular a mix of at least 50, at least 100, or at least 500 peptides of different sequences. The can also comprise a combinatorial library of synthetic peptides corresponding to each of the observed and potential substitutions. An antigenic composition, comprising a mix of the two above-described combinations or mixotopes, is also included in the present invention.

In any of the above-described antigenic polypeptides or antigenic polypeptidic compositions a lipidic molecule can be linked to at least part of the polypeptidic molecules. An example of lipidic molecule that can be used therefore is a C-terminal palmitoylysylamide residue.

As already mentioned above, at least part of the polypeptides or polypeptidic molecules in the antigenic polypeptide according to the invention, can be bound to a support, thereby constituting conjugates. Preferred supports in this embodiment of the invention are viral particles, nitrocellulose or polystyrene beads, and biodegradable polymers such as lipophosphoglycanes or poly-L lactic acid.

Another aspect of the present invention concerns an immunogenic composition comprising as an immunogen a protein or a polypeptide or a polypeptidic composition especially prepared by recombination as any of those described above.

As discussed in Example 5, the gene family described herein presents a remarkable characteristic, which is the epitope conservation between the various members of the family, which leads to immunogenic cross-reactivity between the various products of the gene family. The vaccination potential of MSP-3-1 and its fragments, illustrated in Examples 2 and 3, together with the epitope conservation and the cross-reactivity mentioned above, are remarkable features that make this gene family and the polypeptidic compositions derived therefrom particularly interesting candidates for vaccination against malaria. Another aspect of the present invention is hence the use of a recombinant protein or a polypeptide or a polypeptidic composition as described above, for the preparation of a vaccine against malaria, as well as such a vaccine, comprising as an immunogen said recombinant protein or polypeptide or polypeptidic composition, in association with a suitable pharmaceutical vehicle.

An immunogenic composition and a vaccine according to the invention can further comprise at least one antigen selected amongst LSA-1 (Guerin-Marchand, Druilhe et al. 1987), LSA-3 (Daubersies, Thomas et al. 2000), LSA-5, SALSA (Bottius, BenMohamed et al. 1996), STARP (Fidock, Bottius et al. 1994), TRAP (Robson, Hall et al. 1988), PfEXP1 (Simmons, Woollett et al. 1987), CS (Dame, Williams et al. 1984), MSP1 (Miller, Roberts et al. 1993), MSP2 (Thomas, Carr et al. 1990), MSP4 (Marshall, Tieqiao et al. 1998), MSP5 (Marshall, Tieqiao et al. 1998), AMA-1 (Peterson, Marshall et al. 1989; Escalante, Grebert et al. 2001), SERP (Knapp, Hundt et al. 1989) and GLURP (supra). GLURP, and/or LSA-3 and/or SERP proteins are especially of interest, for use in an immunogenic composition of the invention.

A particular immunogenic composition among the above described one comprise in particular antigens selected among LSA-3, SERP and GLURP or their combinations or immunogenic functional fragments thereof.

According to one particular embodiment of the invention, the immunogenic composition or the vaccine is formulated for intradermal or intramuscular injection. In that case, said immunogenic composition or vaccine preferably comprises between 1 and 100 μg of immunogen per injection dose, more preferably between 2 and 50 μg. Alternatively, the immunogenic composition or the vaccine can be formulated for oral administration, as described by (BenMohamed, Belkaid et al. 2002).

The immunogenic composition or vaccine of the invention can also further comprise SBAS2 and/or Alum and/or Montanide as an adjuvant.

Other aspects of the present invention relate to antibodies, especially purified antibodies, and fragments of antibodies directed against the antigens disclosed herein. As described above and in Example 5, the epitope conservation in the MSP-3 family leads to cross-reactivity of the antibodies obtained against one antigen. For example, a synthetic or recombinant antibody which cross-reacts with several proteins of the MSP-3 family, especially with MSP-3-3 and/or MSP-3-4 and/or MSP-3-7 and/or MSP-3-8, and which mediates Plasmodium falciparum blood stage growth inhibition or killing, in the monocyte-dependent, antibody-mediated ADCI mechanism, under in vitro conditions, is a particularly interesting antibody according to the invention.

A pool of antibodies and/or fragments of antibodies directed against several proteins selected amongst the MSP-3 family, particularly amongst the proteins of SEQ ID Nos: 6, 8, 14 and 16, and/or polypeptides according to the invention, is also part of the invention.

Another pool of antibodies and/or fragments of antibodies according to the invention is directed against a polypeptidic composition as described above.

Preferred antibodies (or fragments) according to the invention are human or humanized antibodies. These antibodies or fragments of antibodies can be produced for example in Lemna, as well as in maize, tobacco, CHO cells, and the like. When produced in CHO cells, they can be obtained for example by using the method described in WO 03/016354.

The present invention also pertains to the use of a composition comprising an antibody or a pool of antibodies or fragments thereof as described above, for the preparation of a medicament against malaria. Of course, a medicament for passive immunotherapy of malaria, comprising such an antibody or a pool of antibodies, is also considered as part of the invention. Such medicament can further comprise antibodies directed against at least one antigen selected amongst LSA-1, LSA-3, LSA-5, SALSA, STARP, TRAP, PfEXP1, CS, MSP1, MSP2, MSP4, MSP5, AMA-1, SERP and GLURP.

Methods for the prophylaxis, the attenuation or the treatment of malaria, by administering to a patient in need thereof, an immunogenic composition, a vaccine, or a medicament comprising antibodies, as described above, are also enclosed in the invention.

The invention also concerns a method for the in vitro diagnosis of malaria in an individual likely to be infected by P. falciparum, which comprises the bringing of a biological sample from said individual into contact with a protein or an antigenic polypeptide of the invention, under conditions enabling the formation of antigen/antibody complexes between said antigenic peptide or polypeptide and the antibodies possibly present in the biological sample, and the in vitro detection of the antigen/antibody complexes possibly formed. In this method, the in vitro diagnosis can be performed by an ELISA assay. It is also possible to bring the biological sample into contact with one or several antigenic peptides originating from other antigens selected amongst LSA-1, LSA-3, LSA-5, SALSA, STARP, TRAP, PfEXP1, CS, MSP-3-1, MSP-3-2, MSP-3-5, MSP-3-6, MSP1, MSP2, MSP4, MSP5, AMA-1, SERP and GLURP, in particular from LSA-3, SERP and GLURP as an additional step of the method.

An alternative method for the in vitro diagnosis of malaria in an individual likely to be infected by P. falciparum comprises the bringing of a biological sample from said individual into contact with antibodies according to the invention, under conditions enabling the formation of antigen/antibody complexes between said antibodies and the antigens specific for P. falciparum possibly present in the biological sample, and the in vitro detection of the antigen/antibody complexes possibly formed.

Kits for the in vitro diagnosis of malaria, based on the particular features of the MSP-3 family, are also contemplated. For example, they can comprise at least one peptide or polypeptide according to the invention, possibly bound to a support. Such a can further comprise reagents for enabling the formation of antigen/antibody complexes between said antigenic peptide or polypeptide and the antibodies possibly present in a biological sample, and reagents enabling the in vitro detection of the antigen/antibody complexes possibly formed.

Another kit for the in vitro diagnosis of malaria, according to the invention, comprises antibodies as described above, and, if necessary, reagents for enabling the formation of antigen/antibody complexes between said antibodies and antigens from the proteins of the MSP-3 family possibly present in a biological sample, and reagents enabling the in vitro detection of the antigen/antibody complexes possibly formed.

Also part of the present invention is a recombinant nucleotide sequence comprising a sequence coding for a protein or an antigenic polypeptide according to the invention. Particular sequences according to the invention are nucleotide sequences comprising a sequence encoding at least two MSP-3-b-like and/or MSP-3-c/d-like motifs, wherein at least one of said motifs is selected amongst the motifs of SEQ ID Nos: 19 to 24 and 27 to 30, or their conservative variants. A first example of such a recombinant nucleotide sequence comprises a sequence encoding a fusion protein comprising several MSP-3-b-like motifs, wherein at least two of said motifs are selected amongst the motifs of SEQ ID Nos: 17 to 24 and their conservative variants. A second example is a recombinant nucleotide sequence comprising a sequence encoding a fusion protein comprising several MSP-3-b-like motifs, wherein at least two of said motifs are selected amongst the motifs of SEQ ID Nos: 25 to 30 and their conservative variants.

Another example is a sequence encoding at least two MSP-3-b-like and/or MSP-3-c/d-like motifs, wherein at least one of said motifs is selected amongst the motifs of SEQ ID Nos: 19 to 24 and 27 to 30, or their conservative variants and comprising a recombinant nucleotide sequence comprising a sequence encoding a fusion protein comprising several MSP-3-b-like motifs, wherein at least two of said motifs are selected amongst the motifs of SEQ ID Nos: 25 to 30.

The invention also pertains to a recombinant cloning and/or expression vector, comprising a nucleotide sequence as described above, which can be, for example, under the control of a promoter and regulatory elements homologous or heterologous vis-à-vis a host cell, for expression in the host cell.

An expression vector as described in the above paragraph can advantageously be used for the preparation of a medicament for genetic immunisation against Plasmodium falciparum.

The invention also pertains to a nucleic acid vaccine (e.g. polynucleotide vaccine) comprising a nucleotide sequence of the invention.

A recombinant host cell, for example a bacterium, a yeast, an insect cell, or a mammalian cell, which is transformed by an expression vector as described above, is also part of the present invention.

Several aspects and advantages of the present invention are illustrated in the following figures and experimental data.

### LEGENDS TO THE FIGURES

FIG. 1: Organisation of nine genes clustered in the same region of chromosome 10. Nine open reading frames are separated by non coding regions, and encode in a row (5′-3′) genes encoding proteins denominated first GLURP, followed at 1300 base-pairs by MSP-3 (now denominated MSP-3-1) followed by 7 other genes denominated MSP-3-2, MSP-3-3, MSP-3-4, MSP-3-5, MSP-3-6, MSP-3-7, MSP-3-8.

FIG. 2 : Various peptides derived from MSP-3-1. Protection was associated with antibodies to peptides MSP-3b, c and d.

FIGS. 3, 4 and 5: In vivo studies. Passive transfer experiments of specific antibodies into P. falciparum-infected, human RBCs-grafted, immunocompromised mice.

Antibodies to MSP 3-b peptide, MSP-3-d peptide and to GLURP-R0 region were all found able, under passive transfer conditions in vivo, to clear a P. falciparum parasitemia established in immunocompromised SCID mice.

FIG. 6: In vivo studies. Confirmation results.

A human recombinant antibody directed to the MSP-3-b epitope, cross-reactive with MSP-3-2 recombinant protein which, upon passive transfer, can clear the parasitemia in P. falciparum SCID mice.

FIG. 7: Results obtained using antibodies elicited by artificial immunisation of human volunteers using a Long Synthetic Peptide covering the region MSP-3-b, c, d peptides.

The same effect is observed, both under in vitro conditions and under in vivo conditions, in the P. falciparum SCID mouse model.

FIG. 8: Comparison between the biological effect of total African IgG with purified anti-MSP-3-b antibodies adjusted at the same concentration as in the total African IgG.

A stronger and more complete effect of the anti-MSP-3-b antibodies alone is observed, which stresses their vaccine potential.

FIG. 9: Alignement ClustalW séquences nucleotidiques famille MSP-3.

FIG. 10: Alignement ClustalW séquences peptidiques famille MSP-3.

FIG. 11: Comparison of sequences between genes MSP3 family. This comparison shows a very unusual conservation of the epitopes between members of the family, those targeted by biologically active antibodies, which is critical for protection.

FIG. 12: MSP-3-b -like motifs

FIG. 13: MSP-3-c-d-like motifs

FIG. 14: A. Pattern of IgG3 antibody responses against each of the antigens in the 30 protected individuals of OoDo (means and standard errors of the ratios of IgG3-specific responses). B. Pattern of IgG3 responses in 7 protected OoDo inhabitants with low IgG3 anti-MSP3 response (low IgG3 cut off values were defined as those under the lower 95% confidence interval limits of the mean, ie.anti-MSP3b IgG3 ratios <2.30). C Pattern of IgG3 responses in 15 protected OoDo inhabitants with low IgG3 anti-R0 response (low IgG3 cut off values were defined as those under the lower 95% confidence interval limits of the mean IgG3 ratios, ie. IgG3 ratios of anti-GLURP R0<1.38). D. Changes at 5 years interval in 7 protected individuals with high IgG3 MSP3 responses in 1998. E. Changes at 5 years interval in 6 protected individuals with high IgG3 anti-GLURP R0 responses in 1998.

FIG. 15: ADCI activity of antibodies affinity purified on various constructs derived from the MSP-3 gene family. The results are expressed a the mean SGI (specific growth inihibitory index) as compared to a positive control, the pool of the immune African immunoglobulins (PIAG) which has been used for passive transfer into Thai children. The sequences used for affinity purification correspond to the C-terminus region, which is the most homologous part between the genes and the only one very well conserved and are indicated by a line below the C-term region in FIG. 18.

Results show that all antibodies specific to each region to each of the 6 genes are strongly active in the ADCI mechanism as much as the pool of African immunoglobulins shown to be effective at clearing P. falciparum by passive transfer in infected individuals.

FIG. 16: pattern of cross-reactivity, of antibodies affinity purified on the C-terminus region of each of the members of the MSP-3 family, with other members of the MSP-3 family. GLURP, 571 and BSA serve as negative controls.

Results show that antibodies affinity purified on a given C-terminus region of one member of the family cross react, to various extent, to all other members of the MSP-3 family. The strongest cross-reactive pattern is obtained with MSP-3-4 which shows a strong positive signal with all other members followed by MSP-3-8. However, this dot-blot merely shows cross-reactive epitopes in each of the member of the MSP-3 family.

FIG. 17: patterns of cross reactivity, of antibodies affinity purified on the C-terminues region of each fo the members of the MSP-3 family, with peptides derived from the MSP-3-1 and the MSP-3-2 members of the MSP-3 family. The peptides are peptides a, b, c, d, and f, from MSP-3-1 and from MSP-3-2. The recombinant MSP-3 C-term and BSA serve as positive and negative controls respectively.

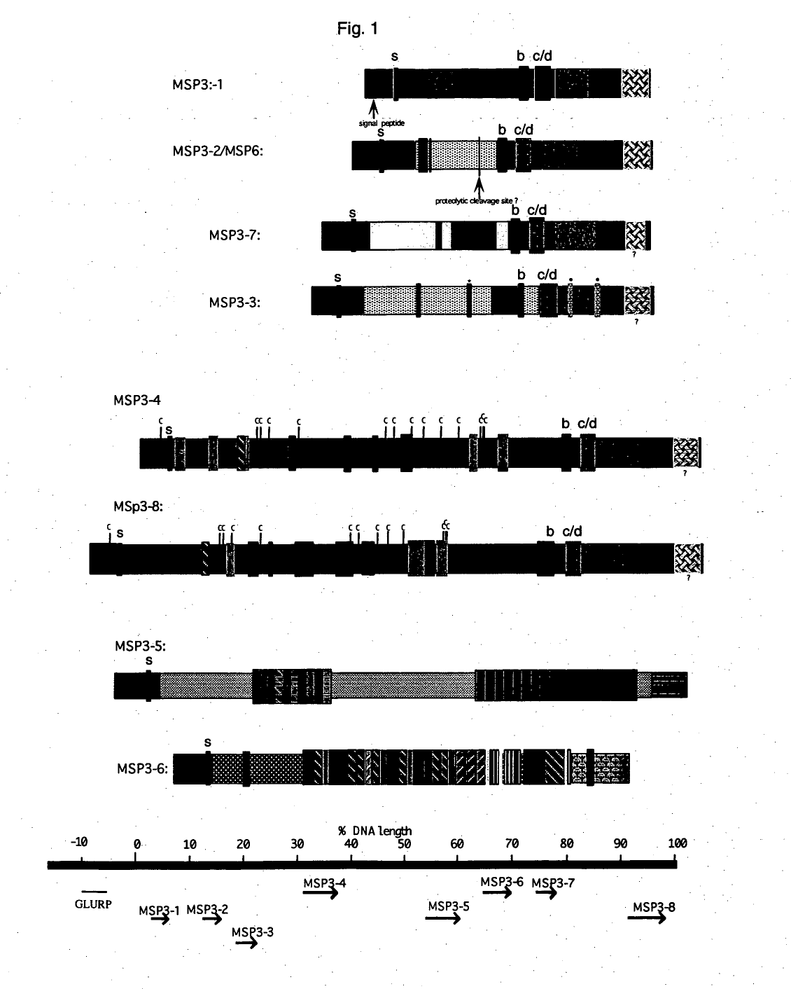

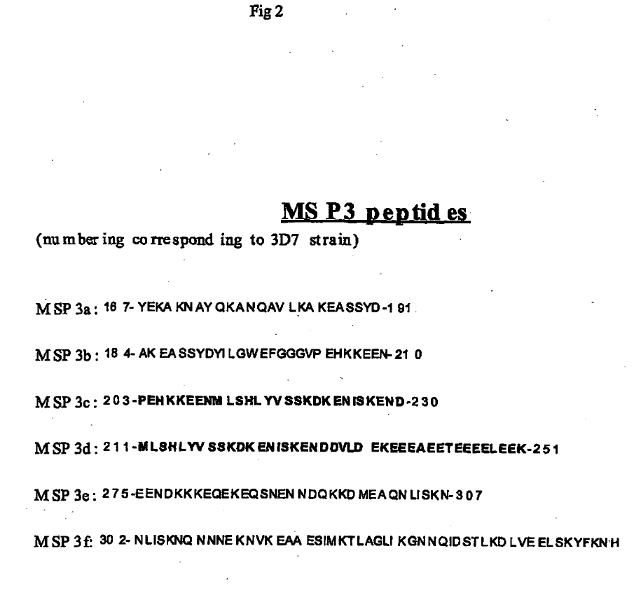

Results show that antibodies to the C-terminus regions of the various members of the family react, to various extents, with various regions of the C-terminus of MSP-3-1, particularly MSP-3b and c, and the strongest response being obtained on MSP-3-f. The cross reactivity with various peptides of MSP3-2 is not as strong as that obtained with MSP-3-1. Finally, the very strong cross-reactivity obtained with MSP-3-1 CT, the C terminus recombinant, also suggests a cross-reactivity with an epitope not defined by any of the individual peptides but most likely a conformational epitope generated by the longer C-term recombinant. In this case, the extent of cross-reactivity of any given affinity purified antibody to any given member of the family demonstrate the structural homology of the various members of that family and the existence of cross-reactive epitopes, including those generated by 3-dimensional conformation. The same holds true for MSP-3-2.

FIG. 18: A schematic representation of the various members of the MSP-3 family. Underlined is the C-terminus region which was used to build up recombinant antigens which were used in the immunoassays.

FIG. 19: Schematic presentation of P. falciparum MSP3 protein and the design of MSP3 recombinant proteins (MSP3-NTHis and MSP3-CTHis), and peptides (MSP3a, MSP3b, MSP3c, MSP3d, MSP3e and MSP3f). The representation of the N-terminal part of MSP3 is compressed here (indicated by dotted line). DG210 represents the λgt11 expression clone originally identified as the target of protective antibodies [Bouharoun-Tayoun H, Druilhe P. 1992]. The numbers show amino acid positions for each region based on the sequence derived from 3D7 strain.

FIG. 20: Total IgG response against different regions of MSP3 in hyperimmune sera (n=30) from Ivory Coast, used to prepare protective IgG for passive transfer experiment in humans [Sabchareon A, Burnouf T, Ouattara D, et al. 1991]. Antibody reactivity was considered to be positive if the ratio of the mean O.D. of the test sera to the mean O.D. of control sera+3× standard deviation of the control sera, was ≧1. The figure represents the mean antibody titer (expressed as ratio) of positive sera against each region. The table shows percent prevalence of positive sera reactive to different regions of MSP3 in terms of total IgG.

FIG. 21: Prevalence and mean titer of antibodies against different regions of MSP3 in sera (n=48) from the village of Dielmo. Antibody reactivity was considered to be positive if the ratio of the mean O.D. of test sera to the mean O.D. of control sera+3× standard deviation of the control sera, was ≧1. The figure represents antibody titers (expressed in ratio) of the positive sera against each region. The table shows percent prevalence of positive sera reactive to different regions of MSP3 in terms of IgG isotype.

FIG. 22: Effect of affinity-purified human anti-MSP3 antibodies on parasite growth in ADCI assay. The histograms represent mean values of % SGI (as explained in the text) from two independent experiments±standard error; values of >30% are significant. PIAG, positive control IgG from the pool of Ivory Coast adult sera used for passive transfer in humans [Sabchareon A, Burnouf T, Ouattara D, et al. 1991].

FIG. 23: In vivo, transfer of affinity purified human anti-MSP3 antibodies together with human peripheral blood monocytes in P.f.-HuRBC-BXN mice. The curves show the course of parasitemia as determined by microscopic examination of thin blood smears for mice injected with anti-MSP3b antibodies (grey diamonds) and with anti-MSP3d antibodies (white circles). The arrows indicate the days at which injections were made, first of human monocytes (HuMn) and then followed by monocytes together with anti-MSP3 antibodies (200 μl, IFA 1:200).

FIG. 24: MSP3a, MSP3b, MSP3c.

FIG. 25: MSP3d, MSP3e, MSP3f.

FIG. 26: Panel (A): Schematic presentation of a 32 kb contig located on P. falciparum Chromosome 10 (1404403 to 1436403 bp of the 3D7 strain) indicating the relative positions of the MSP3-like ORFs. MSP3.1 and MSP3.2 are genes known to encode merozoite surface proteins MSP3 (Oeuvray, et al., 1994) and MSP6 (Trucco, et al., 2001), respectively. Panel (B): ClustalW alignment and Boxshade representation of the amino acid sequences of the MSP3-family of proteins with related C-terminal sequence organization. MSP3.5 and MSP3.6 do not share the C-terminal sequence similarities with other members. White letters on black backgrounds indicates identical residues, whereas similar residues are indicated by black letters on a gray background. Dashes represent gaps to optimize alignments. The patterns shared by MSP3-family members are: the N-terminal signal-peptide (dotted line box); the signature sequence of the MSP3 family of proteins [1]; the glutamic rich region [2]; and the leucine-zipper domain [3]. Sequences highlighted in black are related to regions identified as targets of protective antibodies identified in MSP3.1. Panel (C): A cladogram showing sequence analogy between different MSP3-like ORFs derived by comparing the encoded protein sequences.

FIG. 27: Schematic presentation of the protein sequences encoded by MSP3-like ORFs. The N-terminal region of each molecule has ( )—signal peptide and ( )—signature motif of 4-6 a. a. The C-terminal part has sequence organization similar to MSP3.1 in all members of the MSP3 family except MSP3.5 and MSP3.6. ( )—and ( ) represent the regions sharing sequence relatedness to targets of protective antibodies, identified in MSP3.1. ( )—and ( ) represents glutamic acid rich region and putative leucine-zipper domain respectively. ( )—represents regions sharing similarities with MSP3.1. Other features observed in different members are: ( )—the heptad repeats in MSP3.1; regions in other ORFs with sequence discordance with MSP3.1: ( ), ( ) and ( )—in MSP3.2, MSP3.7 and MSP3.3 respectively. ( )—represents regions in MSP3.4 and MSP3.8 with similarities to DBL domains in var and ebl-family of proteins together with the position of cysteine residues. ( ) and ( )—are the MSP3.1 unrelated regions of MSP3.4 and MSP3.8 respectively, which are similar to each other. ( ) and ( )—represent regions in MSP3.5 and MSP3.6 respectively, together with other shaded repeat regions, which do not have similarity with other MSP3-like ORFs. The bold lines represent ( )—recombinant proteins covering the unique regions identified in each member, which do not share sequence similarities with other P. falciparum proteins and ( )—recombinant proteins covering the related C-terminal region present in all members except MSP3.5 and MSP3.6.

FIG. 28: Specificity of antibodies affinity-purified against recombinant proteins covering the unique-regions' identified in each member of the MSP3-like ORFs. 2 μg of the purified His-tag recombinant protein (MSP3.1u, . . . MSP3.8u) was dot blotted on nitrocellulose strips. Antibodies affinity-purified against each unique region recombinant protein, from hyperimmune sera (anti-MSP3.1u, anti-MSP3.8u), were tested against a panel of all unique region recombinant proteins, as shown in the figure above. The pattern of antibody reactivity shows high specificity of the affinity-purified antibodies towards the recombinant proteins against which they were affinity-purified.

FIG. 29: Expression analysis of MSP3-like ORFs. Panel A: Transcript analysis by RT-PCR.

Arrowheads indicate the size of the cDNA amplification obtained using primer sets specific for each ORF. Note that the transcript for MSP3.5 was less abundant as compared to other members of the family. Panel B: Detection of protein encoded by different MSP3-like ORFs in P. falciparum 3D7 blood stage extract, by Western blot analysis, using antibodies affinity-purified against unique non cross-reactive region identified in each ORF. Arrowheads indicate the size of the P. falciparum protein detected by denaturing SDS-PAGE. The numbers represent positions of the molecular weight markers. Antibodies affinity-purified against unique regions of MSP3.5 and MSP3.6 did not detect specific protein products in the parasite extract. Panel C: IFA analysis of acetone-fixed thin smear of the blood stage parasites, using the same antibodies used for Western blot analysis, shows merozoite surface staining. The size-bar drawn in the lower right-hand corner of each microscopic field represents 5 μm. Antibodies affinity-purified against the unique region of MSP3.5 did not react to parasite proteins in IFA.

FIG. 30: Pattern of antibody subclass reactivity observed against different members of the MSP3-family of proteins in a pool of hyperimmune sera from malaria endemic village Dielmo, Senegal. The histograms represent mean O.D.450 values obtained after subtracting the reactivity against BSA. [NH4 SCN].

FIG. 31: Graphical presentation of antibody binding avidity against members of the MSP3-family of proteins under increasing concentrations of NH4SCN. Shown here are two examples of affinity-purified antibodies panel A: reactivity of anti-MSP3.4 antibodies against MSP3.1 and panel B: reactivity of anti-MSP3.7 antibodies towards itself. The measure of antigen-antibody reactivity in absence of NH4SCN was considered to be 100%, and the reactivity obtained in presence of increasing concentrations of NH4SCN was expressed as fractions of that 100%. Since the antibody binding did not display a linear relationship with increasing concentrations of the chaotropic salt, antibody binding avidity was determined by calculating the ‘% area covered by each curve’, as represented by the shaded area in the figure.

FIG. 32: Cross-reactivity displayed by antibodies generated by artificial immunization in mice. Groups of 5 Balb/C mice each were immunized with the related C-terminal recombinant proteins from MSP3.1 and MSP3.2, with montanide as adjuvant. The histograms show mean O.D.450 values obtained for the reactivity of anti-MSP3.1 and anti-MSP3.2 mice sera against different members of the MSP3-family of proteins. The error-bars represent s.d. values.

FIG. 33: Effect of human antibodies affinity-purified against the related C-terminal region of the MSP3 family of proteins on parasite growth in ADCI assay. The histograms represent mean values of % SGI (as explained in the text) from two independent experiments±standard error; values of >30% are significant. PIAG, positive control IgG from the pool of Ivory Coast adult sera used for passive transfer in humans (Sabchareon, et al., 1991).

FIG. 34: (A) Schematic presentation of P. falciparum MSP6 protein and the design of MSP6 Cterm recombinant protein and peptides (MSP6a, MSP6b, MSP6c, MSP6d, MSP6e and MSP6f). The representation of the N-terminal part of the molecule is compressed here (indicated by dotted line). The numbers show amino acid positions for each region based on the sequence derived from 3D7 strain. (B) The homologous alignment of different MSP6 peptide regions with their corresponding regions from MSP3. The solid circles represent identity while the vertical lines show similarity of the amino acid residues shared between the two related molecules (using Wilbur-Lipman algorithm for pair-wise alignment, PAM250).

FIG. 35: Prevalence and titer of antibodies against different regions of MSP6 in hyperimmune sera (n=30) from Ivory Coast. Antibody reactivity was considered to be positive if the ratio of the mean O.D. of the test sera to the mean O.D. of the control sera+3×standard deviation of the control sera, was >or =1. The figure represents antibody titers, expressed as ratio for each serum and the dotted line represents the base line of ratio equal to one. The table shows percent prevalence of the sera with positive IgG reactivity to different regions of MSP6.

FIG. 36: Effect of human affinity-purified anti-MSP6 antibodies on parasite growth in ADCI assays. The histograms represent mean values of % SGI (as explained in the text) from three independent experiments±standard error; values >30% were considered significant. PIAG, positive control IgG from the pool of Ivory Coast adult sera used for passive transfer in humans. The level of parasite inhibition obtained affinity-purified antibodies was adjusted in proportion to the effect observed by PIAG, which was considered to be 100%. NIgG, negative control IgG from pool of French donors, never exposed to malaria. Anti-RESA antibodies were affinity-purified from a pool of hyperimmune sera (Ivory Coast) against a synthetic peptide (sequence H-[EENVEHDA]₂-[EENV]₂—OH).

### EXAMPLES

### Example 1

### In vitro Blood Stage Killing of P. falciparum by Antibodies to the Gene Products, by the ADCI Mechanism

2.A. Materials and Methods: the ADCI assay

2.A.1. Introduction

The Antibody Dependent Cellular Inhibition (ADCI) assay is designed to assess the capability of antibodies to inhibit the in vitro growth of Plasmodium falciparum in the presence of monocytes. Studies have shown that antibodies that proved protective against P. falciparum blood stages by passive transfer in humans are unable to inhibit the parasite in vitro unless they are able to cooperate with blood monocytes. It was also shown that antibodies that were not protective in vivo had no effect on P. falciparum growth in the ADCI assay. The ADCI is therefore an in vitro assay the results of which reflect the protective effect of anti-malarial antibodies observed under in vivo conditions in humans.

The antibodies able to cooperate with monocytes should be obviously cytophilic: IgG1 and IgG3 isotypes are efficient in ADCI while IgG2, IgG4 and IgM are not efficient. This is consistent with the findings that in sera from protected individuals, cytophilic anti-P.falciparum antibodies are predominant, while in non-protected patients the antibodies produced against the parasite are mostly non-cytophilic.

The results suggest that ADCI likely involves the following succession of events: at the time of schizonts rupture, the contact between some merozoite surface component and cytophilic antibodies bound to monocytes via their Fc fragment triggers the release of soluble mediators which diffuse in the culture medium and block the division of surrounding intra-erythrocytic parasites.

The major steps involved in the ADCI protocol are:
(i). Serum IgG preparation using ion exchange chromatography
(ii). Monocyte isolation from a healthy blood donor
(iii). Preparation of P. falciparum parasites including synchronization and schizont enrichment.
(iv). Parasite culture, for 96 hrs, in the presence of antibodies and monocytes.
(v). Inhibition effect assessed by microscopic observation and parasite counting.
   2.A.2. Materials

### IgG Preparation

1. Tris buffer: 0.025 M Tris-HCl, 0.035 M NaCl, pH 8.8.
2. Phosphate Buffer Saline (PBS), pH 7.4.
3. GF-05-Trisacryl filtration column (IBF, Biothecnics, Villeneuve La Garenne, France).
4. DEAE-Trisacryl ion exchange chromatography column (IBF).
5. G25 Filtration column.
6. Amicon filters and tubes for protein concentration (Mol. Wt. cut off: 50,000 Da).
7. Sterile Millex filters, 0.22 μm pore size (Millipore Continental Water Systems, Bedford Mass.).
8. Spectrophotometer equipped with Ultra Violet lamp.

### Monocyte Preparation

1. Heparinized blood collected from a healthy donor, 20-40 mL volume.
2. Ficoll-Hypaque density gradient (Pharmacia LKB Uppsala, Sweden).
3. Hank's solution supplemented with NaHCO₃, pH 7.0.
4. RPMI 1640 culture medium supplemented with 35 mM Hepes and 23 mM NaHCO₃,; prepare with mineral water; store at 4° C.
5. Reagents for non-specific esterase (NSE) staining: fixing solution, nitrite, dye, buffer and substrate
6. 96-well sterile plastic plates (TPP, Switzerland).
7. Refrigerated centrifuge.
8. CO₂ incubator.
9. Inverted microscope.

### Parasite Preparation

1. RPMI 1640 culture medium (see above).
2. 10% Albumax stock solution; store at 4° C. for up to 1 month.
3. 5% Sorbitol for parasite synchronization.
4. Plasmagel for schizont enrichment.
5. Reagents for fixing and staining of thin smears: methanol, eosine, methylene blue.
   2.A.3. Methods

### IgG preparation

IgGs are extracted from human sera (see Note 1) as follows:
1. Dilute the serum at a ratio of 1 to 3 in Tris buffer.
2. Filter the diluted serum through a GF-05 Trisacryl gel filtration column previously equilibrated in the Tris buffer. Ensure that the ratio of serum to filtration gel is 1 volume of undiluted serum to 4 volumes of GF-05 gel.
3. Pool the protein-containing fractions
4. Load over a DEAE-Trisacryl ion exchange chromatography column previously equilibrated with Tris buffer. Ensure that the ratio of serum to filtration gel is 1 volume of undiluted serum to 4 volumes of DEAE gel.
5. Collect fractions of 1 mL volume.
6. Measure the optical density (OD) of each fraction using a 280 nm filter.
7. Calculate the IgG concentration as follows:
   IgG Concentration (mg/mL)=OD 280 nm/1.4
8. Pool the fractions containing IgGs.
9. Concentrate the IgG solution using Amicon filters. Amicon filters are first soaked in distilled water for 1 hour and than adapted to special tubes in which the IgG solution is added.
10. Centrifuge the tubes at 876 g for 2 hr at 4° C. This usually leads to a 25-fold concentration.
11. Perform a final step of gel filtration using a G25 column previously equilibrated in RPMI culture medium.
12. Collect the IgG fractions in RPMI.
13. Measure the optical density (OD) of each fraction using a 280 nm filter.
14. Calculate the IgG concentration.
15. Pool the fractions containing IgGs.
16. Sterilize the IgG fractions by filtration through 0.22 μm pore size filters.
17. Store the sterile IgG solution at 4° C. for up to 1 month (or add Albumax for longer storage—but not recommended—).

### Monocyte Preparation

The procedure for monocyte preparation is based on that described by Boyum (Scand. J. Clin. Lab. Invest. 1968, 21, 77-89) and includes the following steps:
1. Dilute the heparinized blood 3-fold in Hank's solution.
2. Carefully layer two volumes of diluted blood onto 1 volume of Ficoll-Hypaque (maximum volume of 20 mL of diluted blood per tube).
3. Centrifuge at 560 g for 20 min at 20° C.
4. Remove the mononuclear cell layer at the Ficoll/plasma interface.
5. Add 45 mL of Hank's solution to the mononuclear cell suspension.
6. Centrifuge at 1000 g for 15 min at 20° C.
7. Carefully resuspend the pelleted cells in 45 mL of Hank's solution.
8. Centrifuge again at 1000 g for 15 min at 20° C. Repeat this washing step twice more.
9. Finally, centrifuge at 180 g for 6 min at 20° C., to remove any platelets that remains in the supernatant.
10. Resuspend the mononoclear cells in 2 mL of RPMI.
11. Calculate the mononuclear cell concentration (i.e. lymphocytes plus monocytes) in the cell suspension: dilute a 20 μL aliquot of the cell suspension 3-fold in RPMI and count cell numbers using a hemocytometer (Malassez type for example).
12. Determine the number of monocytes using the Non Specific Esterase (NSE) staining technique:
   (i). In microtube A, add 40 μL of mononuclear cell suspension to 40 μL of fixing solution.
   (ii). In microtube B, mix the NSE staining reagents in the following order: 60 μL of nitrite, 60 μL of dye,180 μL of buffer, and 30 μL of substrate
   (iii). Add the mixture in microtube B to the cells in microtube A.
   (iv). Take a 20 μL sample of the stained cells and measure the proportion of monocytes: lymphocytes: monocytes will be colored in brown whereas the lymphocytes will be uncolored. Usually the proportion of monocytes is 10-20% of the total mononuclear cells.
13.Adjust the cell suspension to a concentration of 2×10⁵ monocytes per 100 μL, with RPMI.
14.Aliquot the cell suspension in a 96-well plate at 100 μL/well.
15. Incubate for 90 min at 37° C., 5% CO₂. During this incubation, monocytes will adhere to the plastic.
16. Remove the non-adherent cells and wash the monocytes by adding, and thoroughly removing, 200 μL of RPMI in each well.
17. Repeat this washing procedure 3 times in order to remove all the non-adherent cells.
18. At least 95% of the recovered cells will be monocytes. Control for the cell appearance and the relative homogeneity of cell distribution in the different wells by observation using an inverted microscope (see Notes 2, 3, and 4)

### Parasite Preparation

P. falciparum strains are cultivated in RPMI 1640 supplemented with 0.5% Albumax.

Parasites are synchronized by Sorbitol treatments as follows:
1. Dilute the sorbitol stock to 5% in mineral water.
2. Centrifuge the asynchronous parasite culture suspension at 1200 rpm for 10 min at 20° C.
3. Resuspend the pellet in the 5% sorbitol solution. This will lead to the selective lysis of schizont infected RBC without any effect on the rings and young trophozoites.

When required, schizonts are enriched by flotation on plasmagel as follows:
1. Centrifuge cultures containing asynchronous parasites at 250 g for 10 min at 20° C.
2. Resuspend the pellet at a final concentration of 20% red blood cells (RBC), 30% RPMI, 50% plasmagel.
3. Incubate at 37° C. for 30 min. Schizont-infected RBC will remain in the supernatant, whereas young trophozoite-infected and uninfected RBC will sediment.
4. Collect carefully the supernatant, by centrifugation at 250 g for 10 min at 20° C.
5. Prepare a thin smear from the pelleted cells, stain, and determine the parasitemia by microscopic examination.
6. Usually, using this method, synchronous schizont infected RBC are recovered at ˜70% parasitemia.
   For the ADCI assay, synchronized early schizont parasites are used. Usually the parasitemia is 0.5-1.0% and the hematocrit 4%.

### The ADCI Assay

1. After the last washing step, add in each monocyte containing well:
   (i). 40 μL of RPMI supplemented with 0.5% Albumax (culture medium).
   (ii). 10 μL of the antibody solution to be tested. Usually the IgGs are used at 10% of their original concentration in the serum (˜20 mg/mL for adults from hyperendemic areas, and ˜12 mg/mL for children from endemic area and primary attack patients). (see Note 5).
   (iii). 50 μL of parasite culture, at 0.5% parasitemia and 4% hematocrit.

2. Control Wells consist of the following;
(i). Monocytes (MN) and parasites with normal IgG (N IgG) prepared from the serum of a donor with no history of malaria.
(ii). Parasite culture with IgG to be tested without MN.
3. Maintain the culture at 37° C. for 96 hrs in a candle-jar (or a low O₂, 5% CO₂ incubator).
4. Add 50 μL of culture medium to each well after 48 and 72 hrs.
5. Remove the supernatant after 96 hrs. Prepare thin smears from each well, stain, and determine the parasitemia by microscopic examination. In order to ensure a relative precision in the parasite counting, a minimum of 50,000 red blood cells (RBC) should be counted and the percentage of infected RBC calculated (see Notes 6 and 7).
6. Calculate the specific Growth Inhibitory Index (SGI), taking into account the possible inhibition induced by monocytes or antibodies alone:
   SGI=100×(1−[Percent parasitemia with MN and Abs/Percent parasitemia with Abs]/[Percent parasitemia with MN+ N IgG/Percent parasitemia with N IgG])
   2.A.4. Notes
1. IgG preparation from sera to be tested is an essential step because a non-antibody dependent inhibition of parasite growth has frequently been observed when unfractionated sera were used, probably due to oxidized lipids.
2. Monocyte (MN) function in ADCI is dependent upon several factors such as water used to prepare RPMI 1640. Highly purified water, such as Millipore water, although adequate for parasite culturing, leads to a poor yield in the number of MN recovered after adherence to the plastic wells. On the other hand, water which contains traces of minerals, such as commercially available Volvic water, or glass-distilled water, provide consistently a good monocyte function.
3. Improved monocyte adherence can be obtained by coating the culture wells with fibronectin i.e. coating with autologous plasma from the MN donor, followed by washing with RPMI 1640, prior to incubation with mononuclear cells.
4. MN from subjects with a viral infection (e.g. influenza) are frequently able to induce a non IgG dependent inhibition of parasite growth. This non-specific inhibition effect could prevent the observation of the IgG-dependent inhibition in ADCI. Therefore, MN donors suspected of having a viral infection, or who have had fever in the past 8 days, should be avoided. The results from ADCI are not reliable when the direct effect of MN alone is greater than 50% inhibition. The preparation of MN in medium containing heterologous serum, such as FCS, results in the differenciation of MN, their progressive transformation into macrophages which have lost their ADCI promoting effect.
5. If required, murine IgG can be tested in ADCI with Human MN. The IgG2a isotype is able to bind to the human Fc γ receptor II present on monocytes shown to be involved in the ADCI mechanism.
6. A possible variation of the ADCI assay is the assessment of a competition effect between protective cytophilic antibodies (adults from hyperendemic area) directed to the merozoite surface antigens, and non-protective antibodies (children from endemic area and primary attack patients) which recognize the same antigens but are not able to trigger the monocyte activation because they do not bind to Fc gamma receptors. Therefore non-cytophilc Ig directed to the “critical” antigens may block the ADCI effect of protective antibodies. Each IgG fraction should be used at 10% of its original concentration in the serum.
7. The ADCI assay protocol can be modified and performed as a two-step ADCI with short-term activation of monocytes according to the following procedure:
   (i). Incubate MN for 12-18 hrs with test Ig and synchronous mature schizonts infected RBC, at 5-10% parasitemia. During this first culture time, infected RBC rupture occurs and merozoites are released.
   (ii). Collect supernatants from each well and centrifuge them at 700 g.
   (iii). Distribute the supernatants in a 96-well plate, at 100 μL/well
   (iv). Add to each well 100 μL of P. falciparum asynchronous culture containing fresh medium, at 0.5-1% parasitemia, 5% hematocrit (particular care is taken to reduce to a minimum the leucocyte contamination of the RBC preparation used for this second culture).
   (v). At 36 hr of culture, add 1 mCi of ³H hypoxanthine to each well.
   (vi). At 48 hr of culture, harvest cells and estimate ³H uptake by counting in a liquid scintillation counter.

2.B. Results

TABLE 1

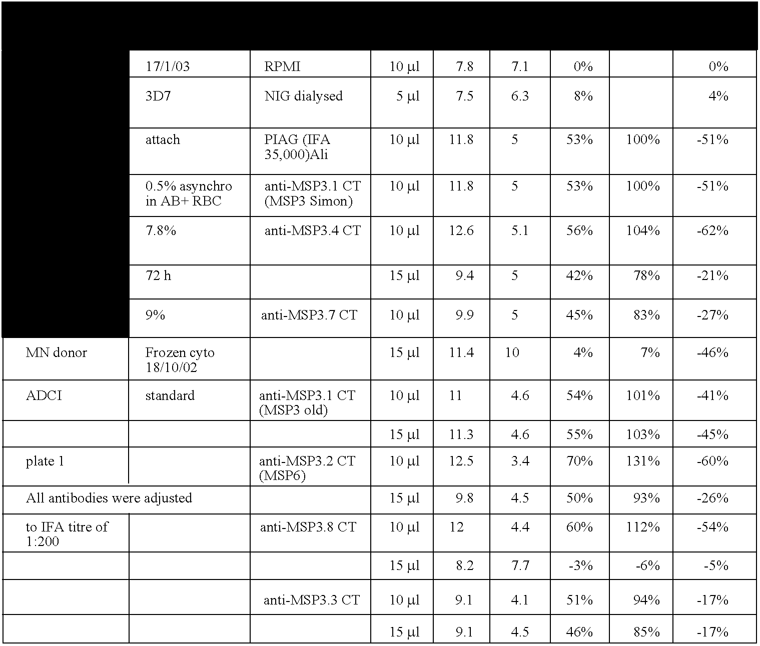

The C-terminal regions used to produce the antibodies are indicated in FIG. 18, and correspond to the horizontal lines below each of the proteins. They have been cloned in E.coli using the PTCR-His vector.

### Example 2

### In vivo Assays by Passive Transfer of Antibodies in Mice

The in vitro results shown in example 1 were confirmed under in vivo conditions by passive transfer experiments of specific antibodies into P. falciparum-infected, human RBCs-grafted, immunocompromised mice.

The materials and methods used to perform the experiments described in the present example are described in (Brahimi, Perignon et al. 1993; Badell, Oeuvray et al. 2000). In particular, the methods to obtain the antibodies have been described by Brahimi et al.

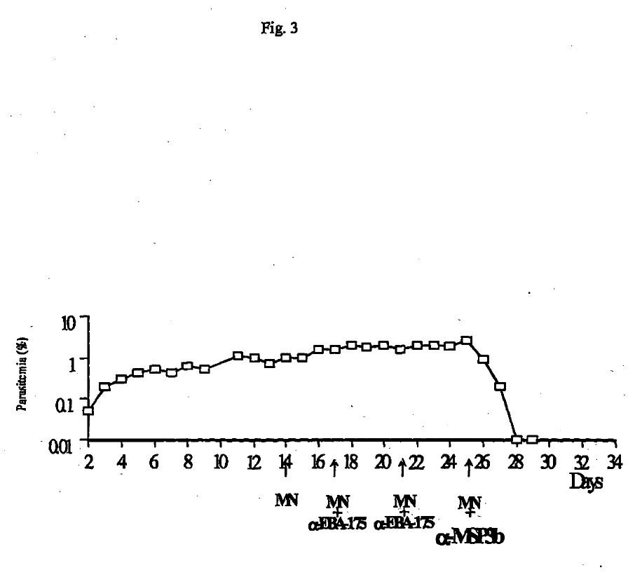

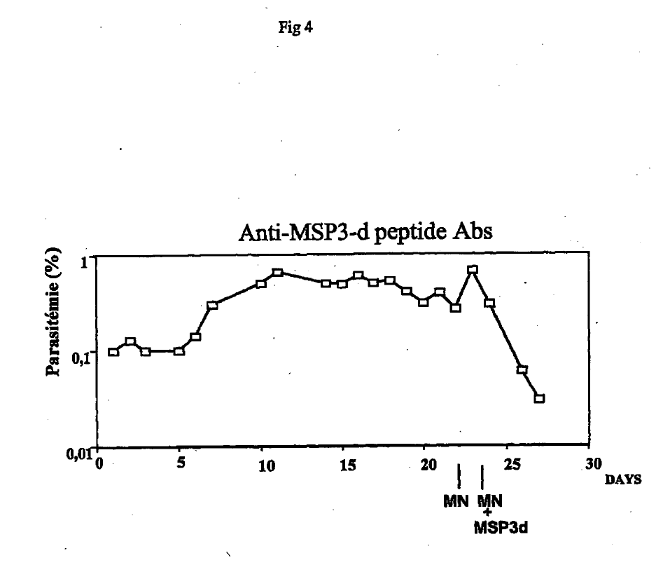

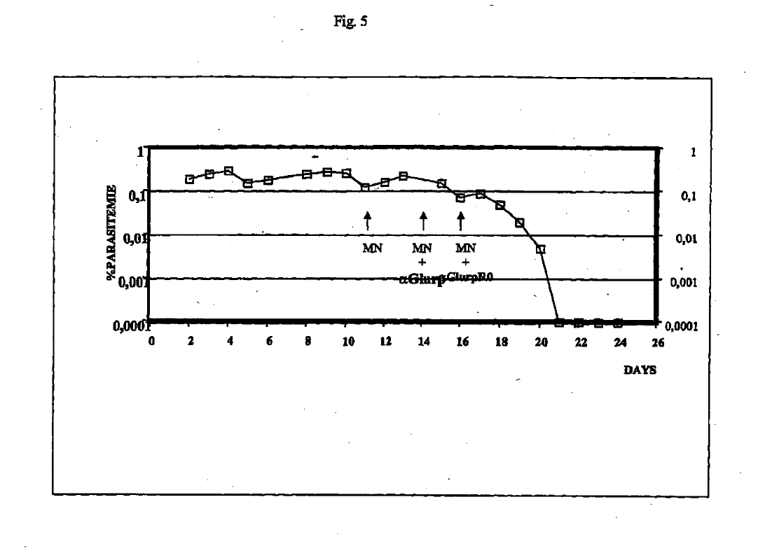

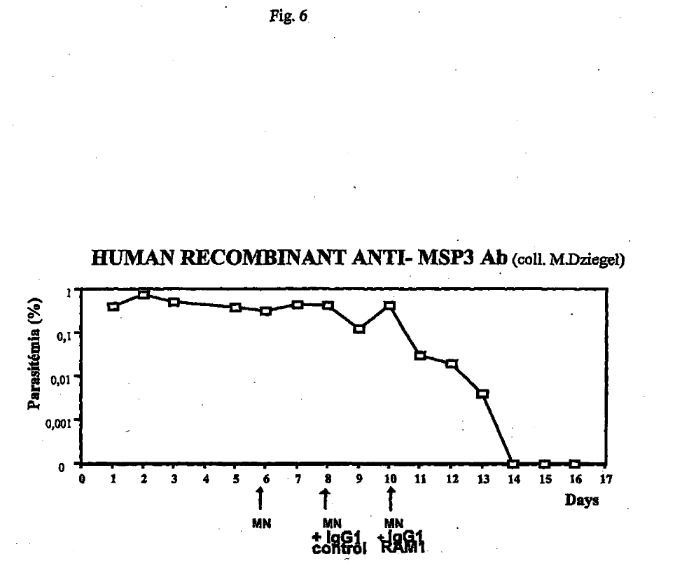

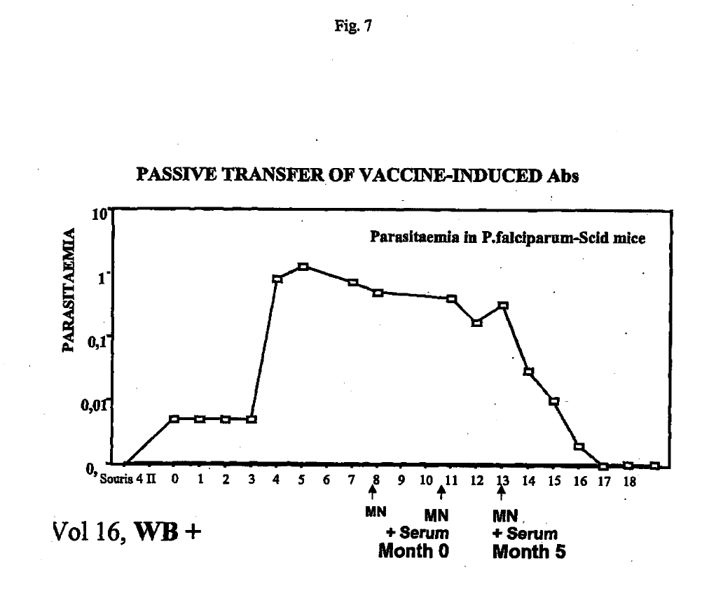

Due to the complexity of the handling of this model, all antibodies could not be tested so far but antibodies to MSP-3-b peptide, MSP-3-d peptide and to GLURP-R0 region were all found able, under passive transfer conditions in vivo, to clear a P. falciparum parasitemia established in immunocompromised SCID mice (FIGS. 3, 4 and 5). It can be seen that the clearance effect of anti-MSP-3-b and MSP-3-d antibodies is extremely strong and, conversely that the clearance induced by anti-GLURP antibodies, adjusted to the same antibody concentration, is less effective: the time to clearance of parasites following transfer is about twice as long with anti-GLURP as that obtained with anti-MSP-3 antibodies. Again here, for reasons described herein, the cross-reactivity network between the 6 genes described in detail implies that antibodies directed to the other genes will most likely have the same biological effect if transferred in P. falciparum infected mice. Finally, this in vivo effect was further confirmed by using a human recombinant antibody directed to the MSP-3-b epitope (FIG. 6), cross-reactive with MSP-3-2 recombinant protein and which, upon passive transfer, can clear the parasitemia in P. falciparum SCID mice. Essentially similar results were also obtained using antibodies elicited by artificial immunisation of human volunteers using a Long Synthetic Peptide covering the region MSP-3-b, c, d peptides which showed the same effect, both under in vitro conditions and under in vivo conditions, in the P. falciparum SCID mouse model (FIG. 7).

### Example 3

### Immunization Experiments in Monkeys

The protective data gathered under in vitro and in vivo conditions was further confirmed independently by showing that aotus monkeys immunised by MSP-3-1 in recombinant form adjuvated by Freund complete adjuvant, produced antibodies effective in the ADCI mechanism and that the monkeys, when challenged by a virulent P. falciparum blood stage inoculation, were able to control and to clear their P. falciparum parasitemia, whereas control monkeys did not.

### Example 4

### Comparison of the Biological Effects Obtained with Total African IqG and with Purified anti-MSP-3-b Antibodies

The comparison of the biological effect obtained with total African IgG and with purified anti-MSP-3-b antibodies adjusted at the same concentration as in the total African IgG shows a stronger and more complete effect of the anti-MSP-3-b antibodies alone, which stresses their vaccine potential. In the course of previous and present studies, the inventors observed that affinity-purified antibodies to MSP-3-b peptide had apparently a faster and stronger effect than total African IgG, from which they were extracted. This observation was extremely intriguing, since P. falciparum being made of nearly 6000 different proteins, and peptide MSP-3-b being only a small region of one of them, one can compute that anti-MSP-3-b antibodies would correspond to less than 1/10,000 of the total antibodies raised by exposure to the parasite.

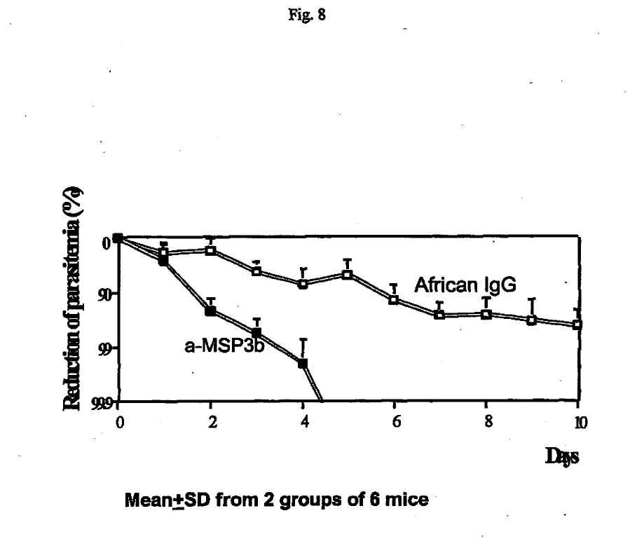

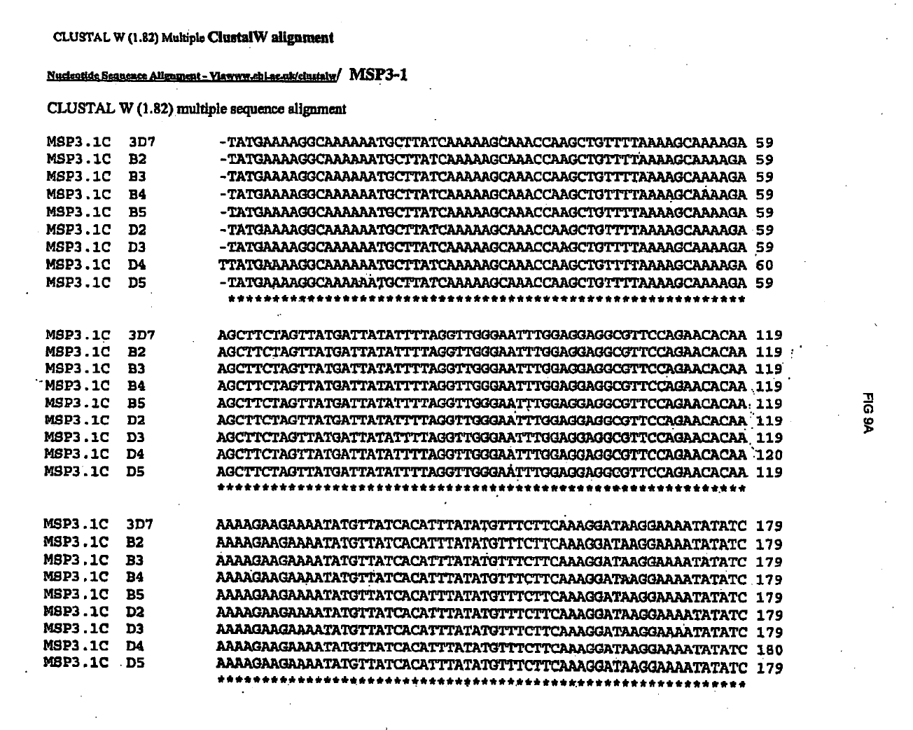

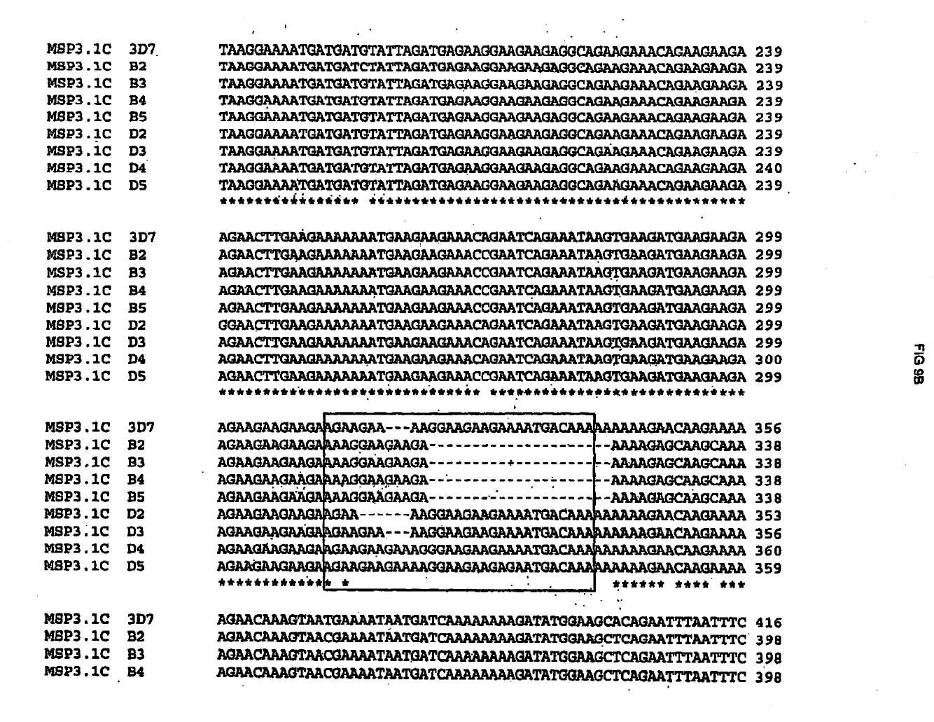

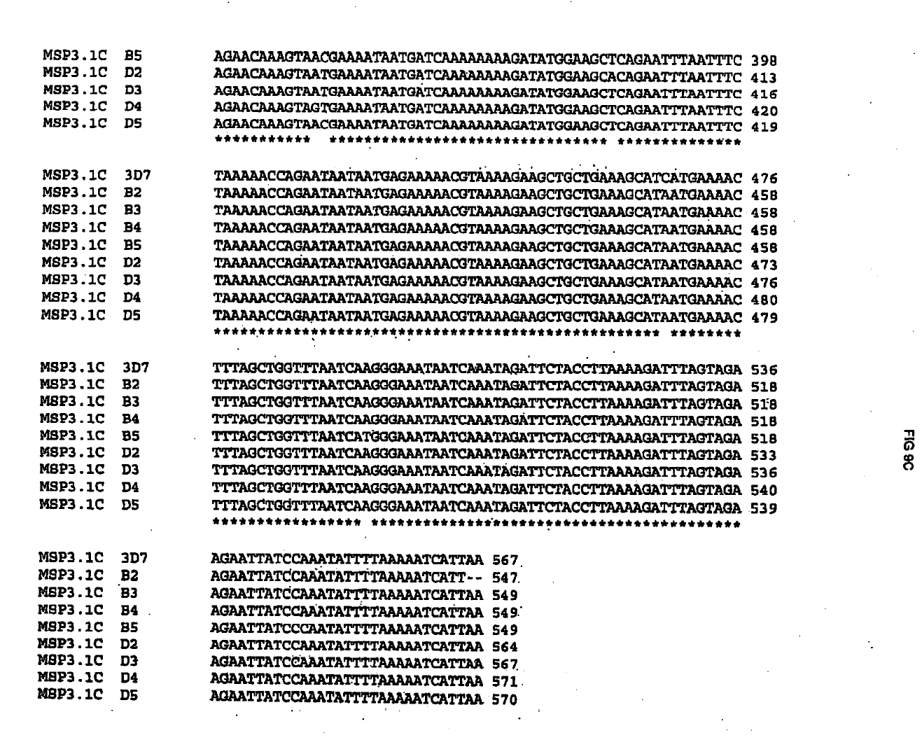

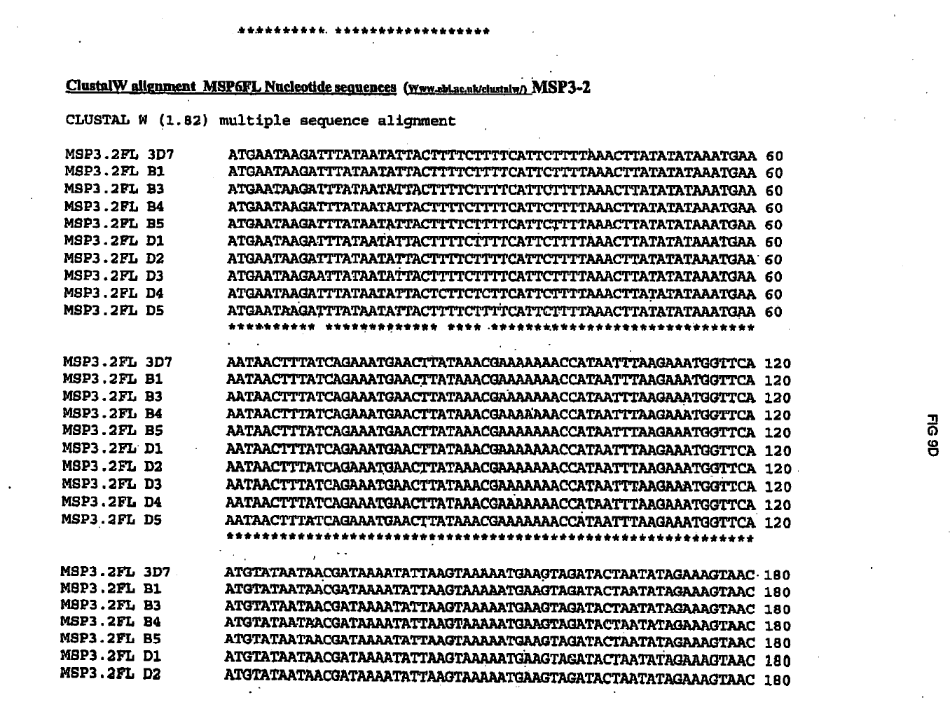

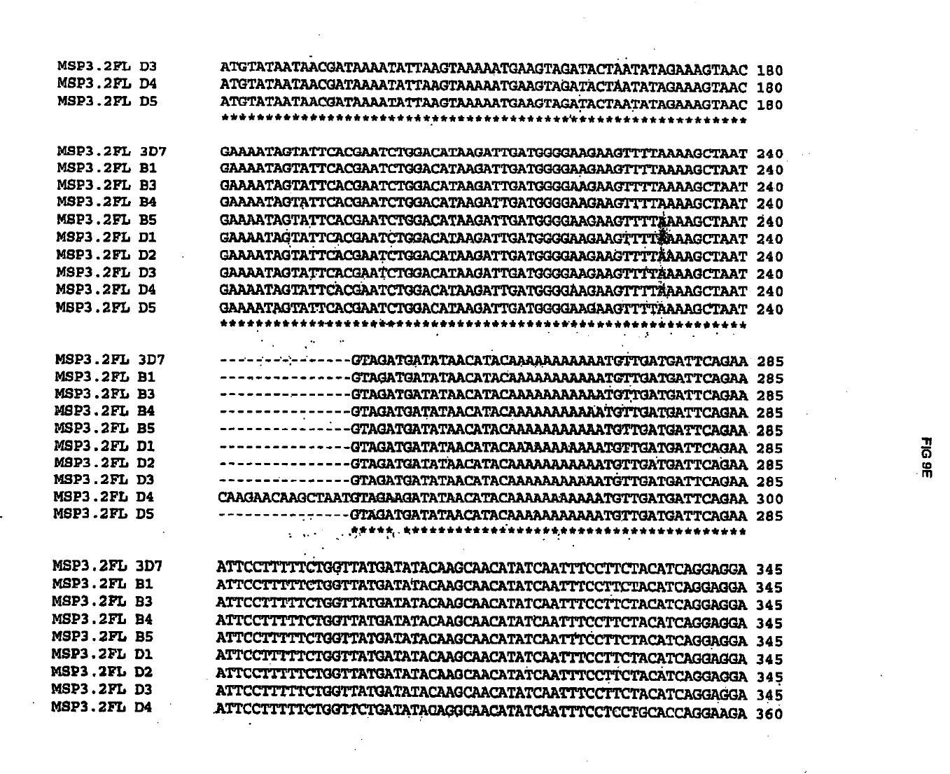

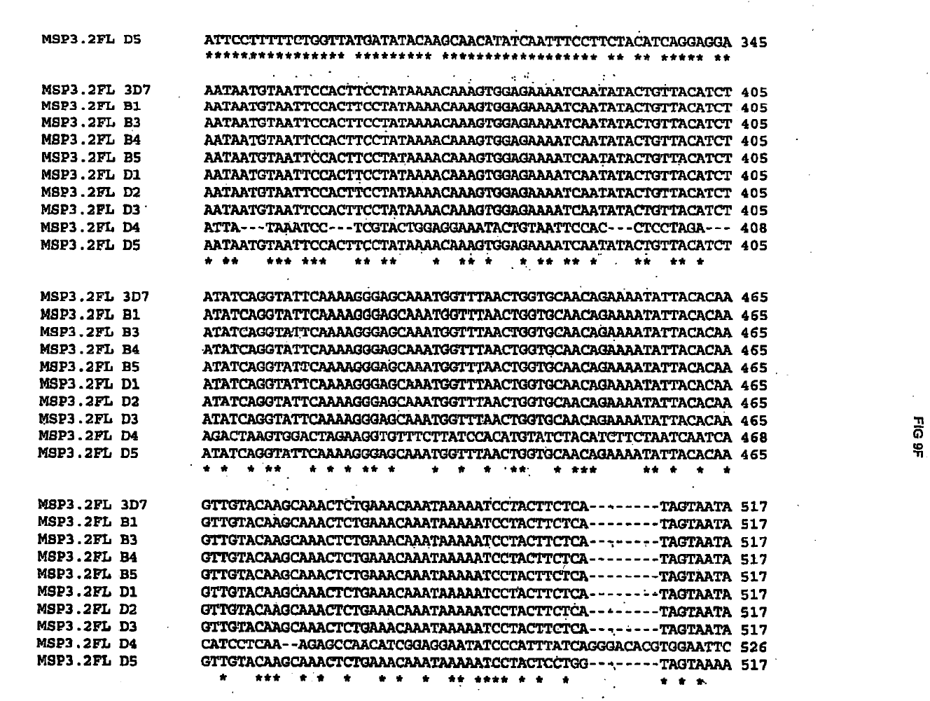

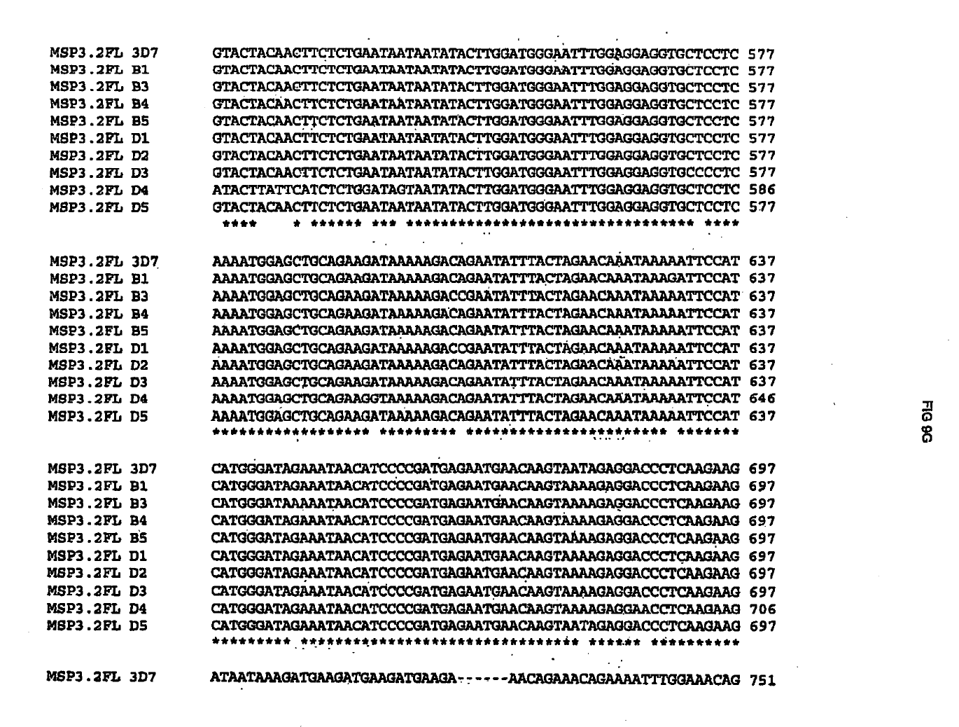

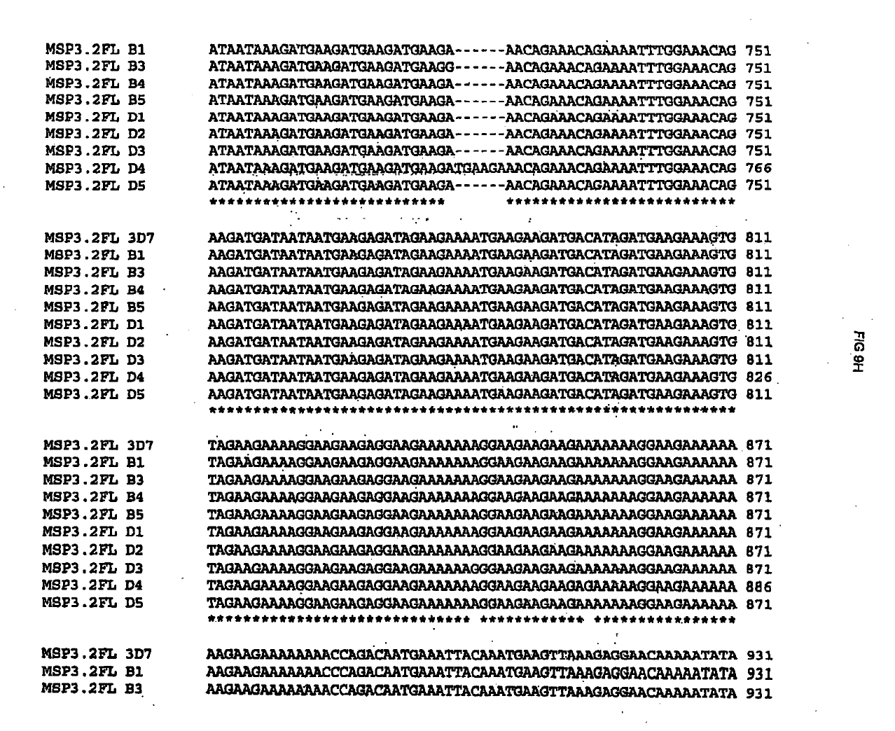

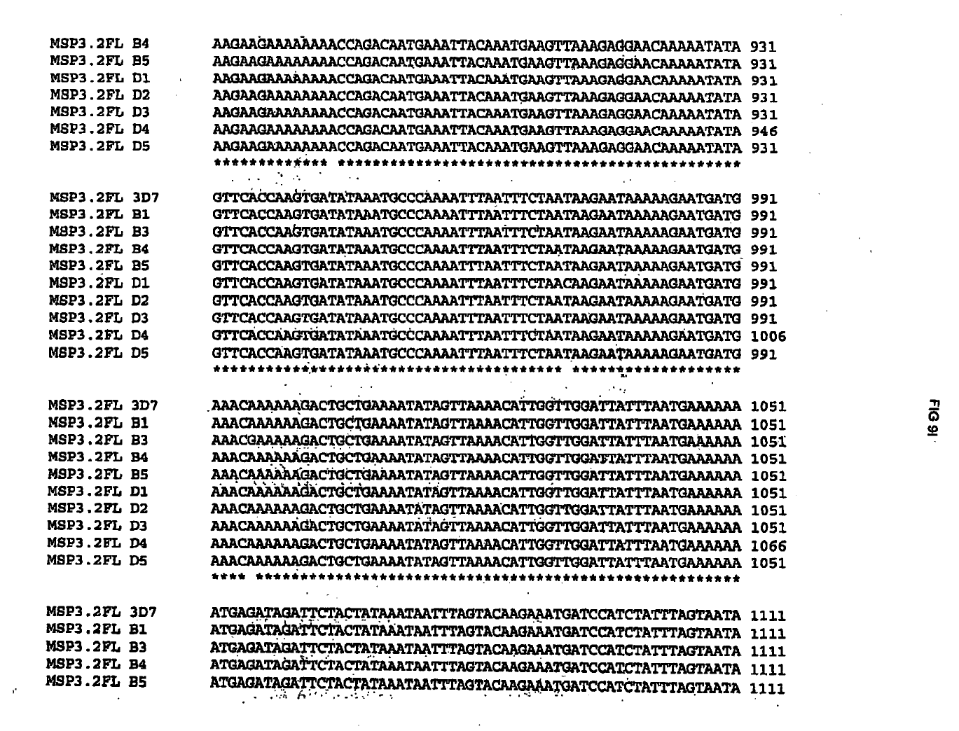

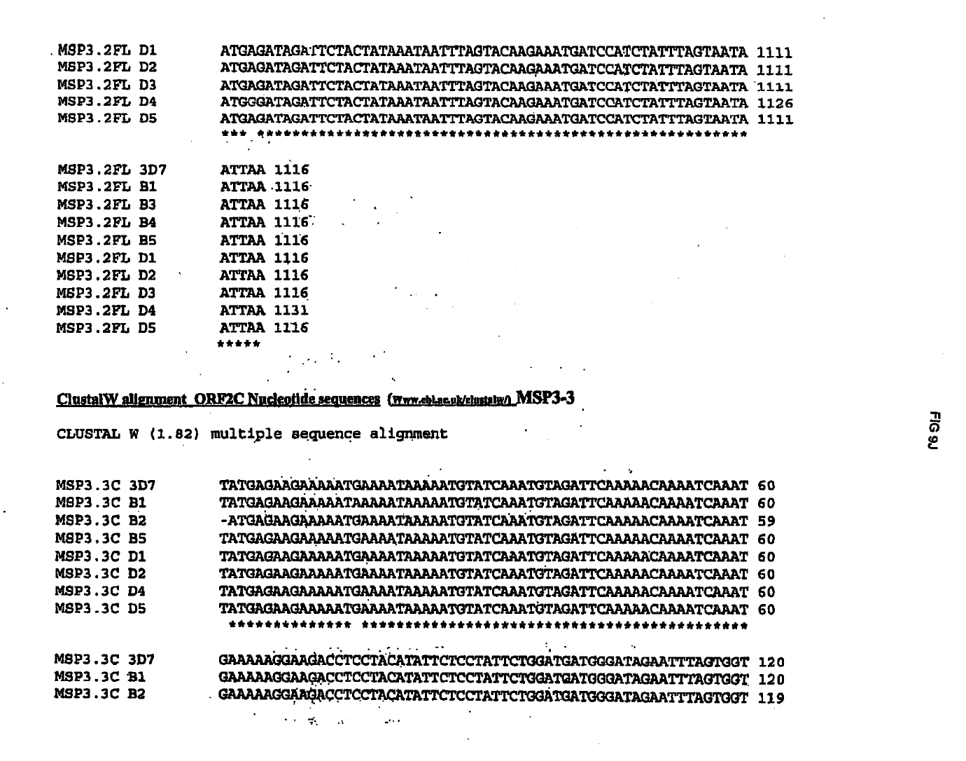

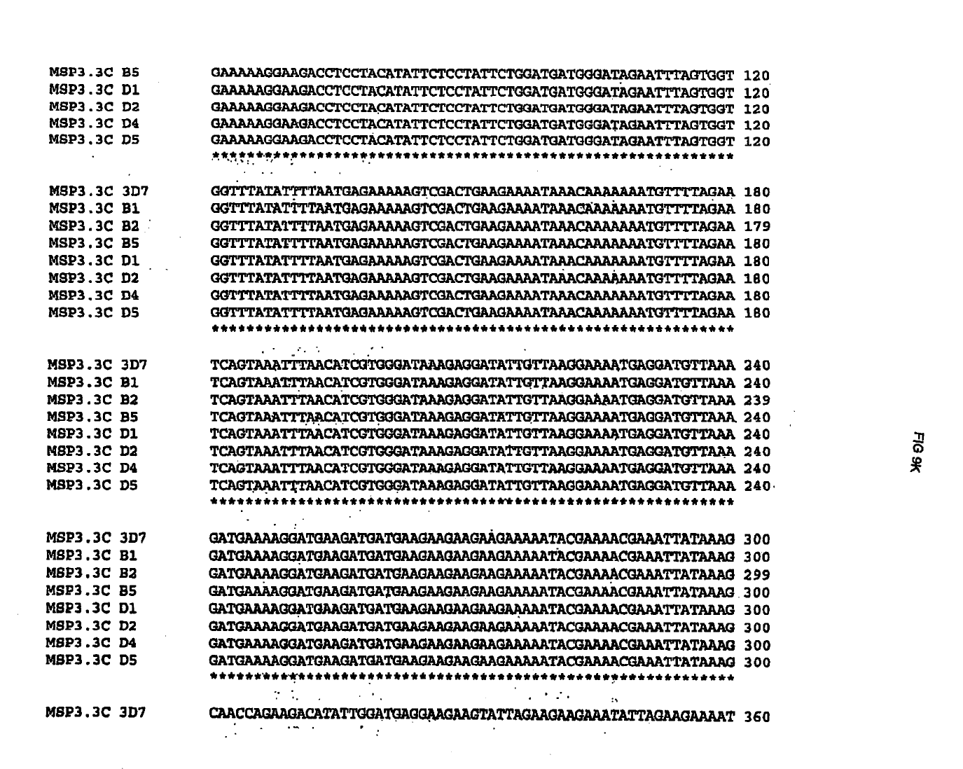

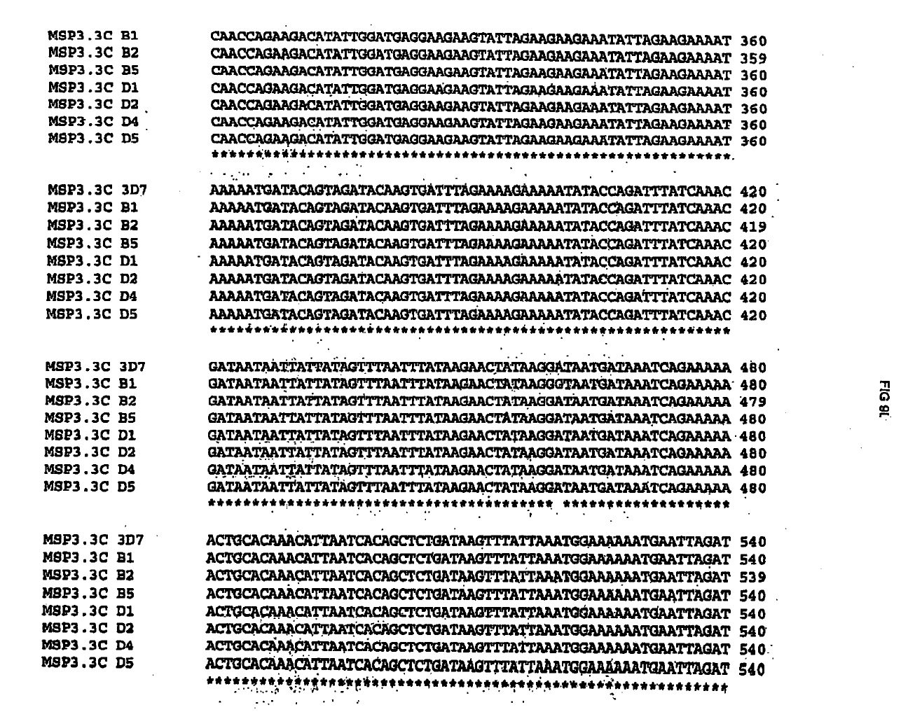

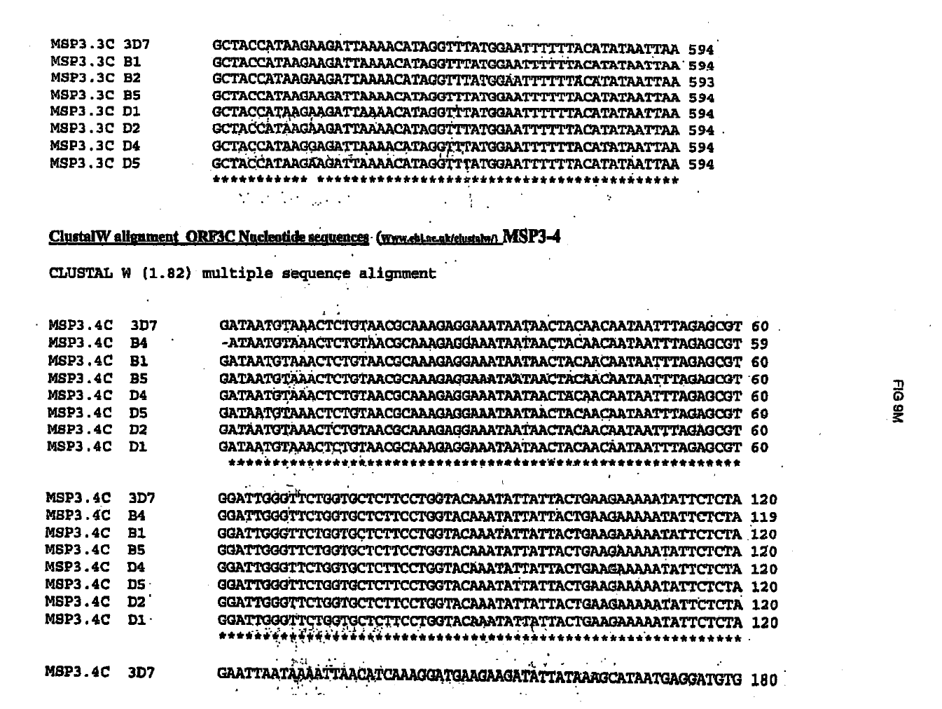

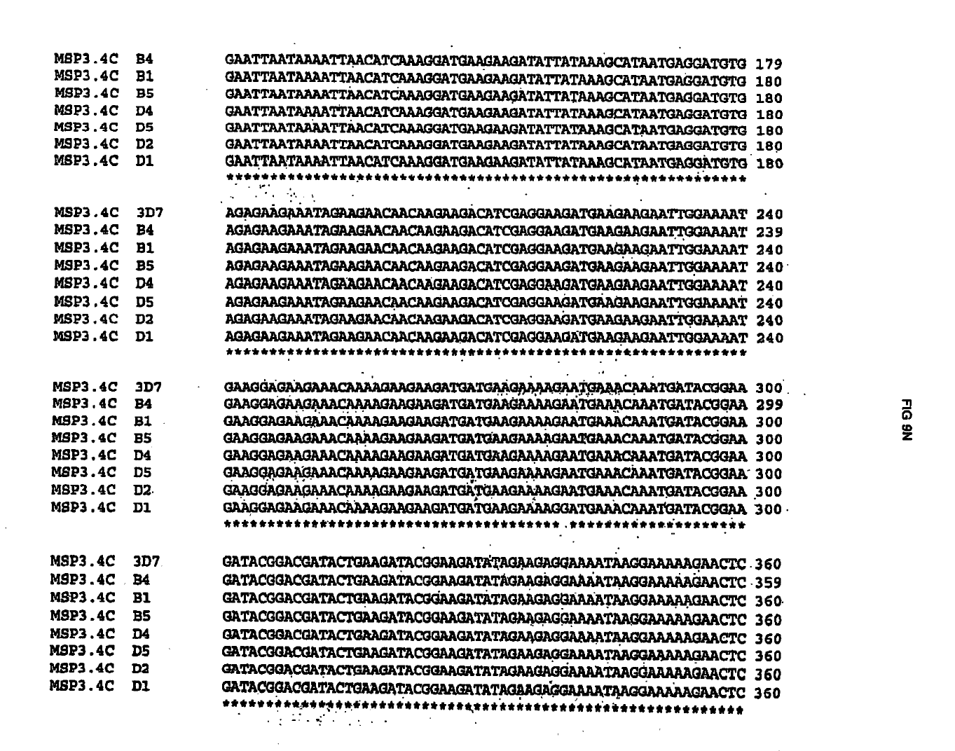

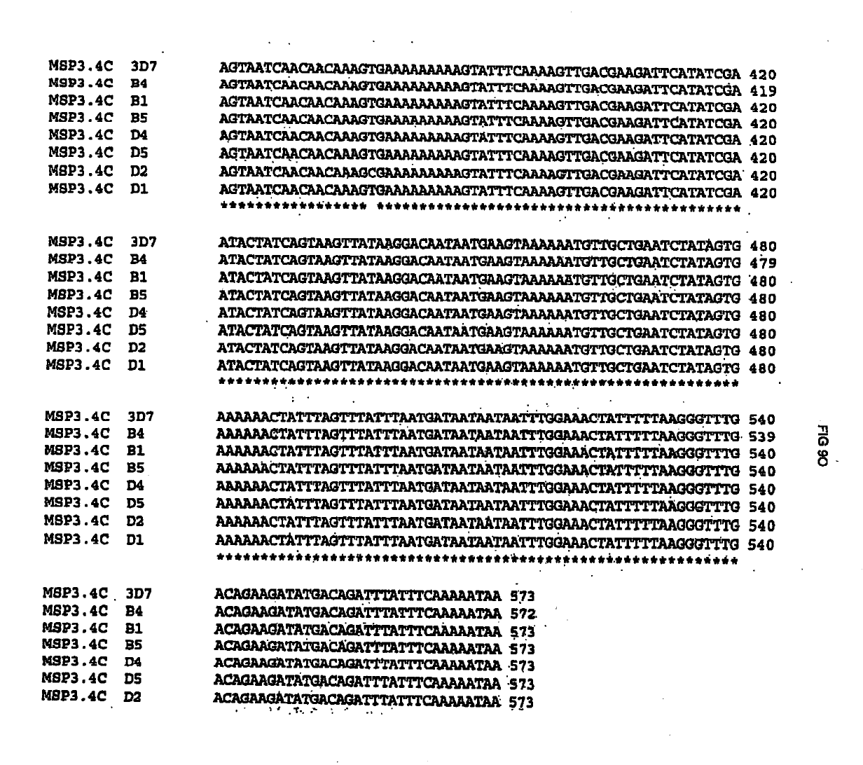

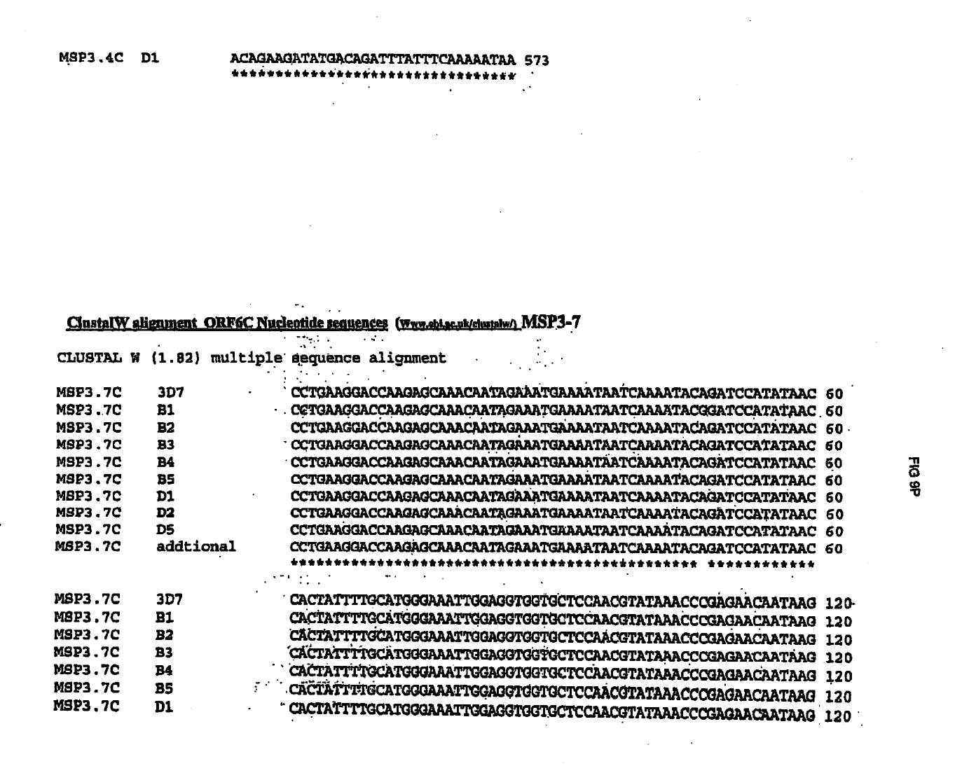

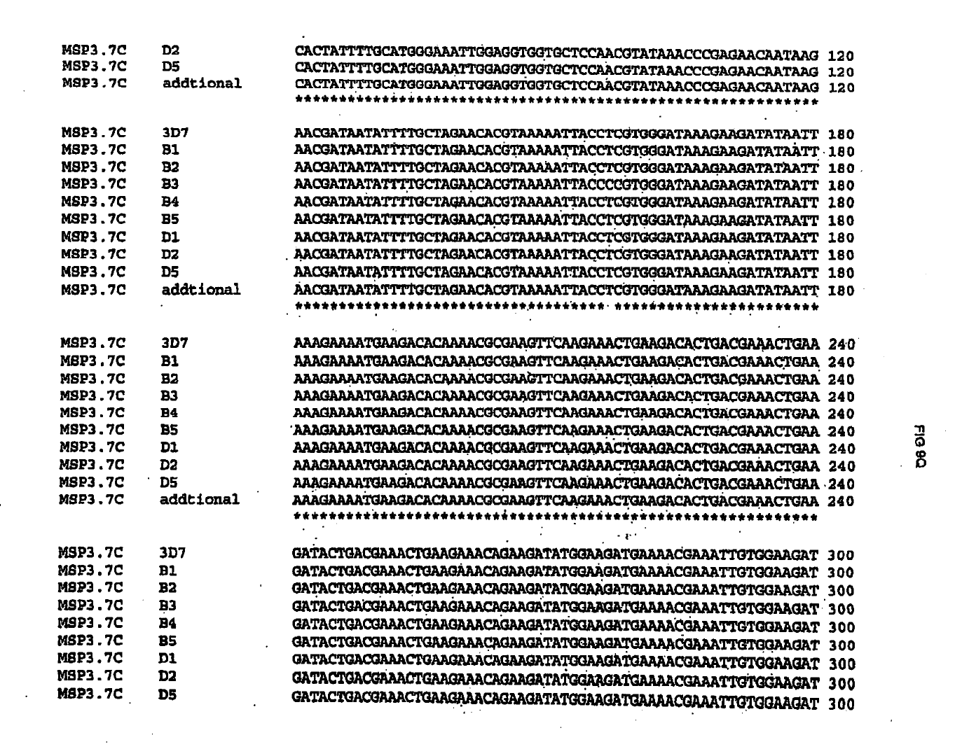

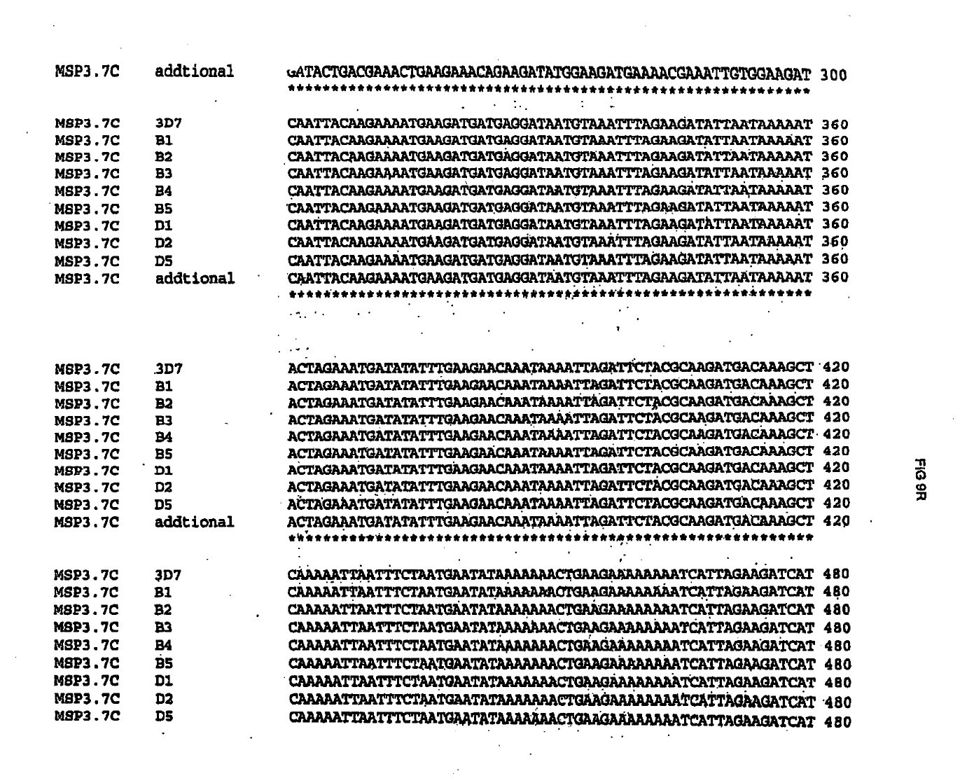

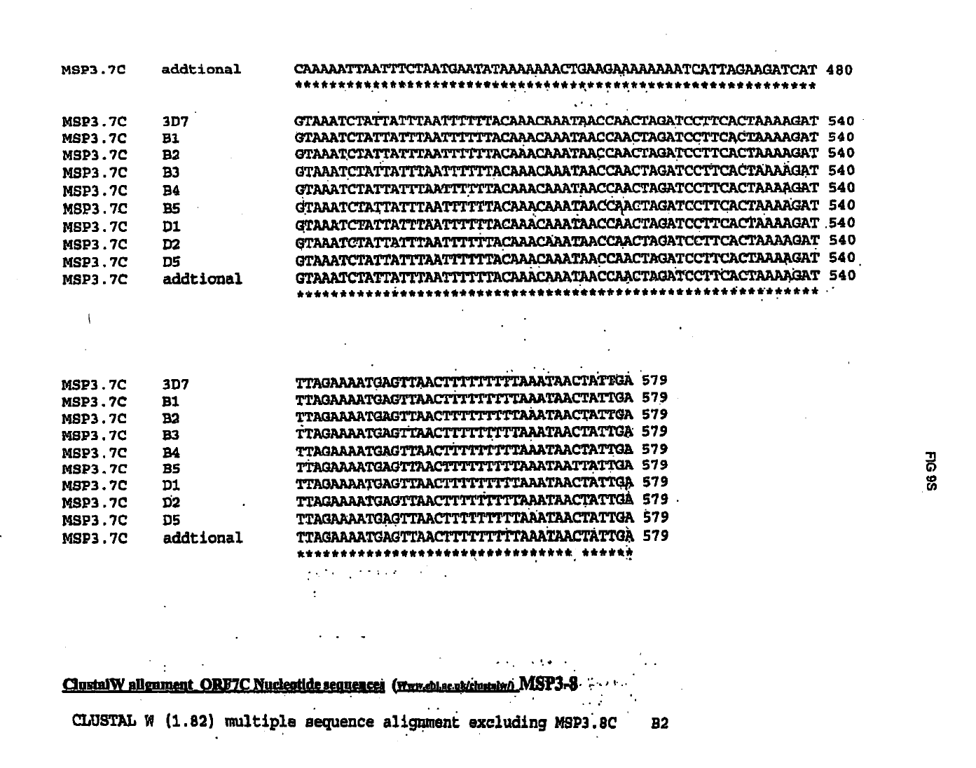

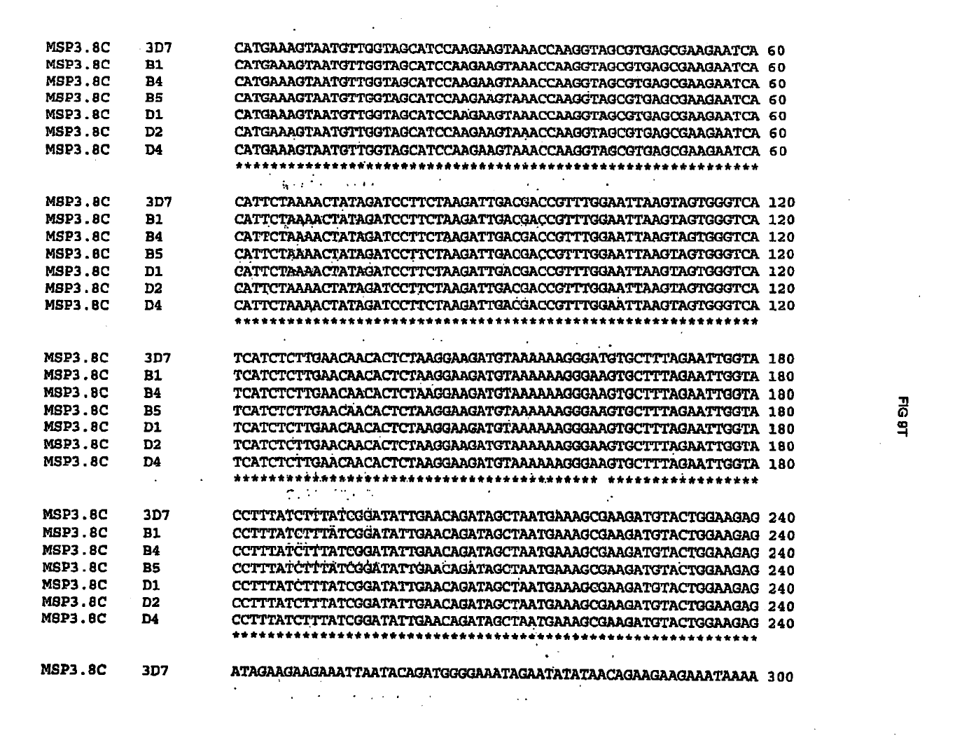

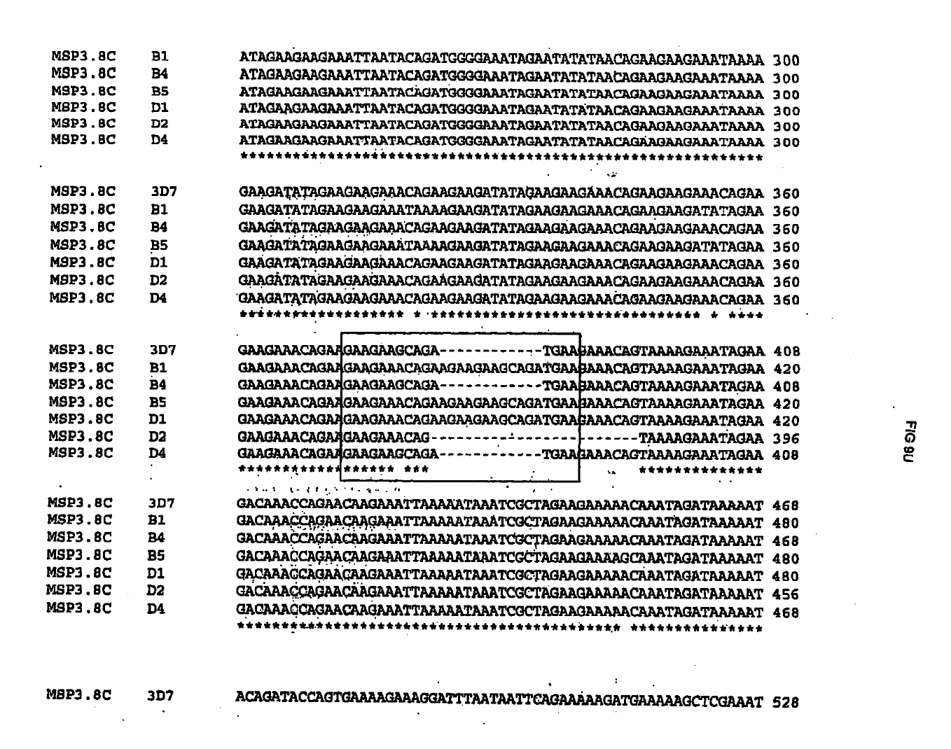

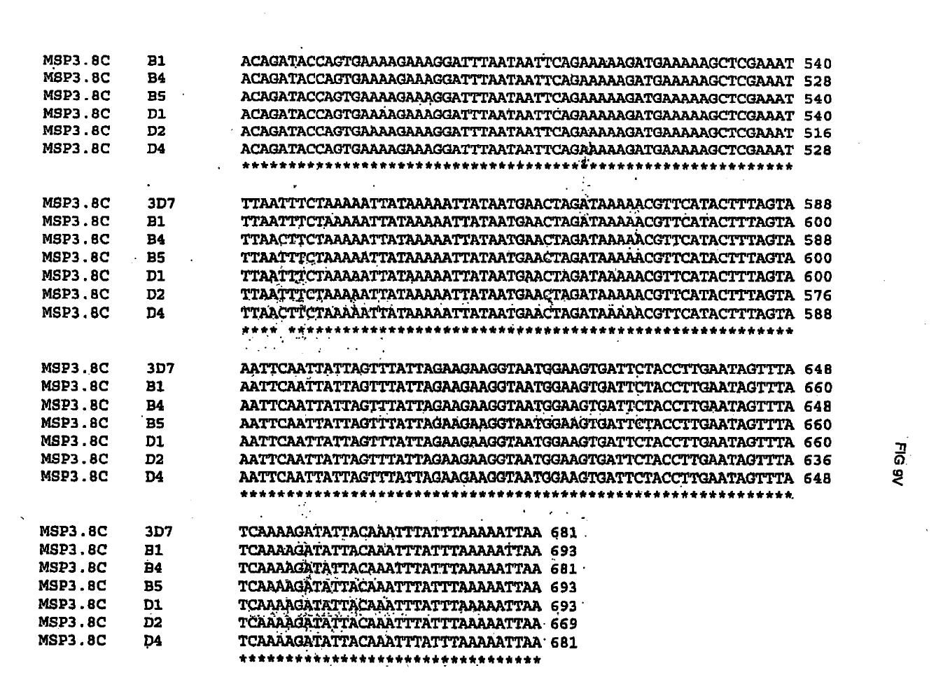

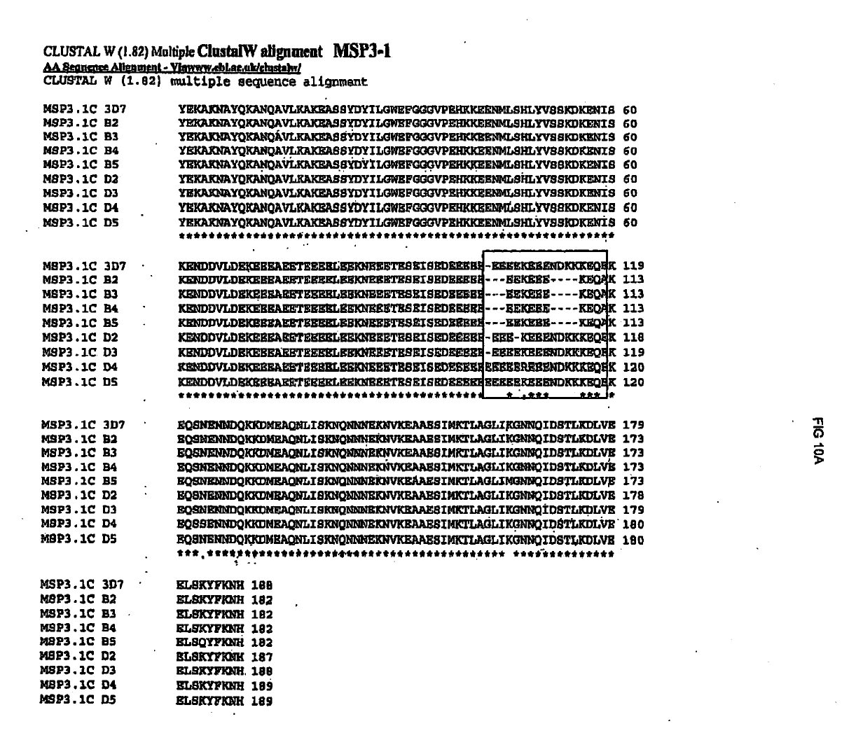

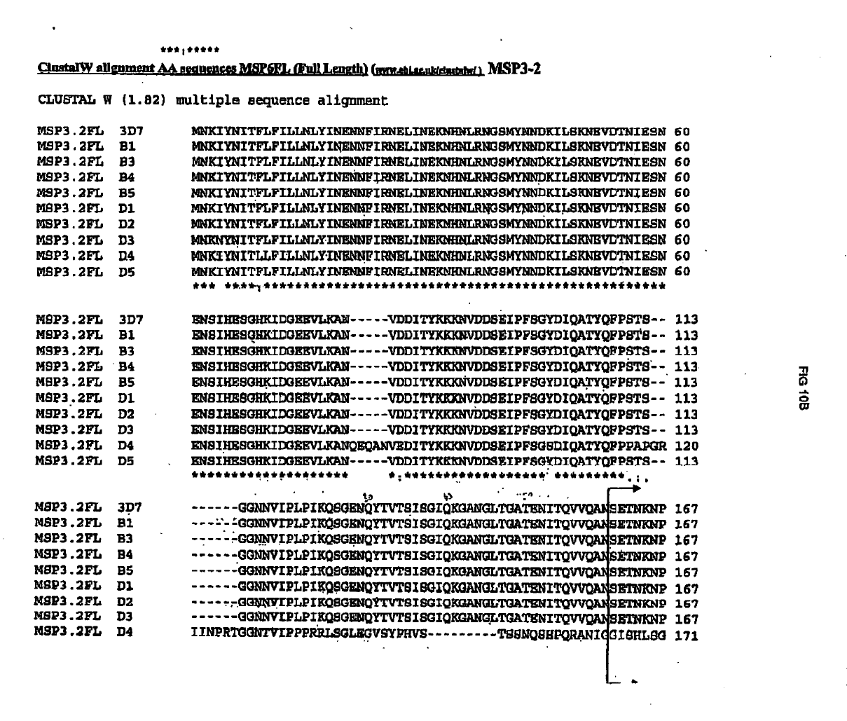

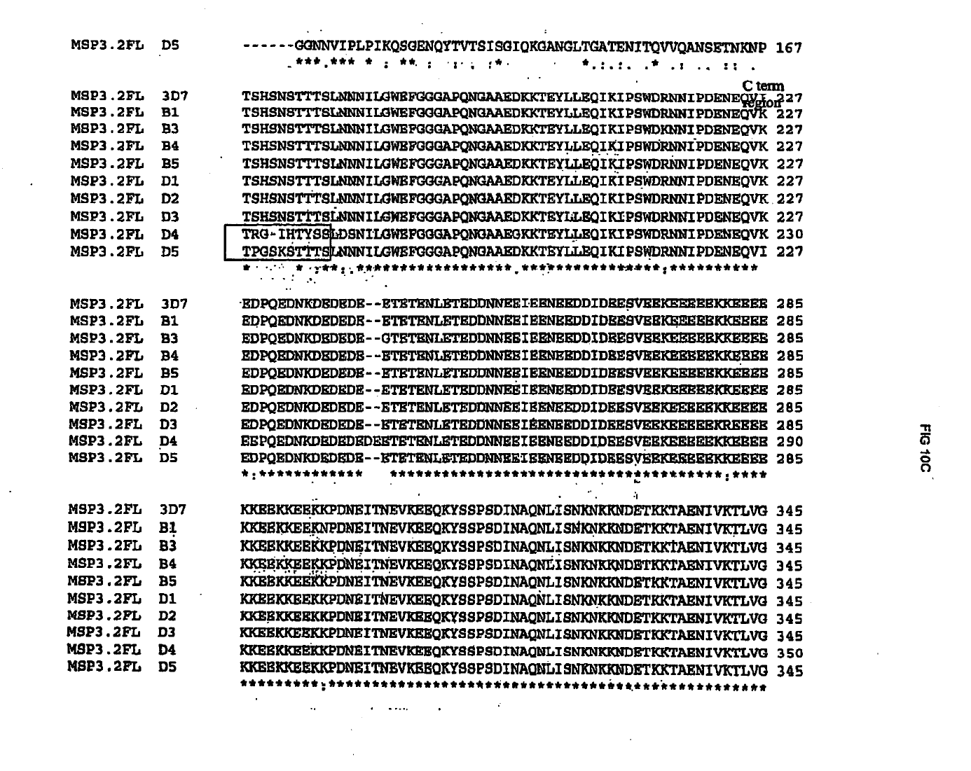

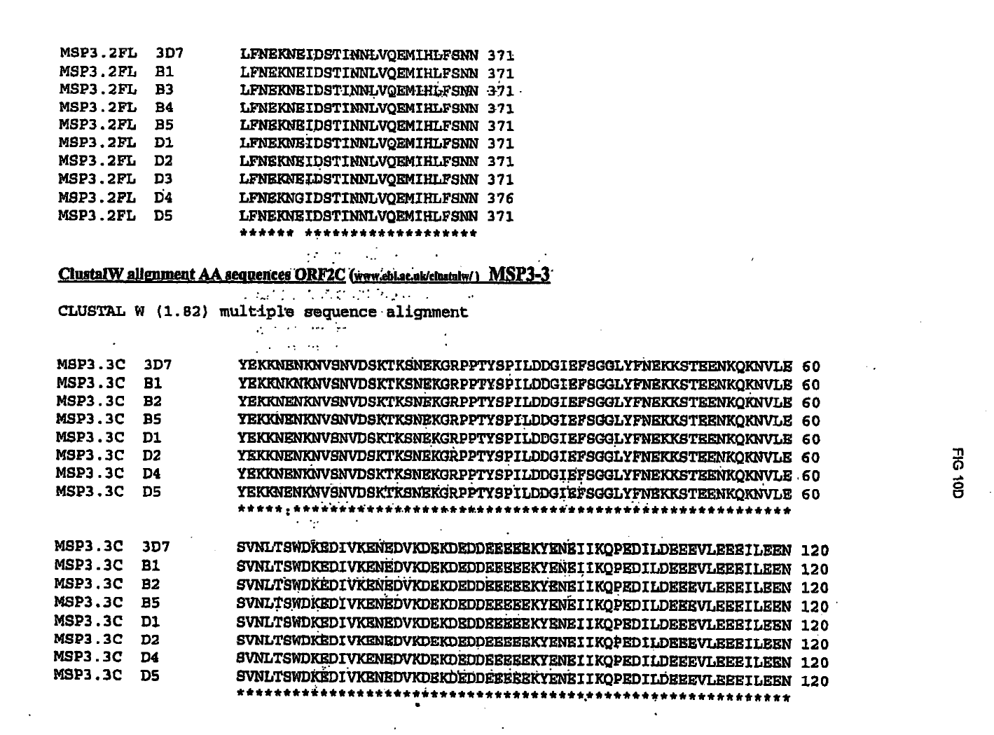

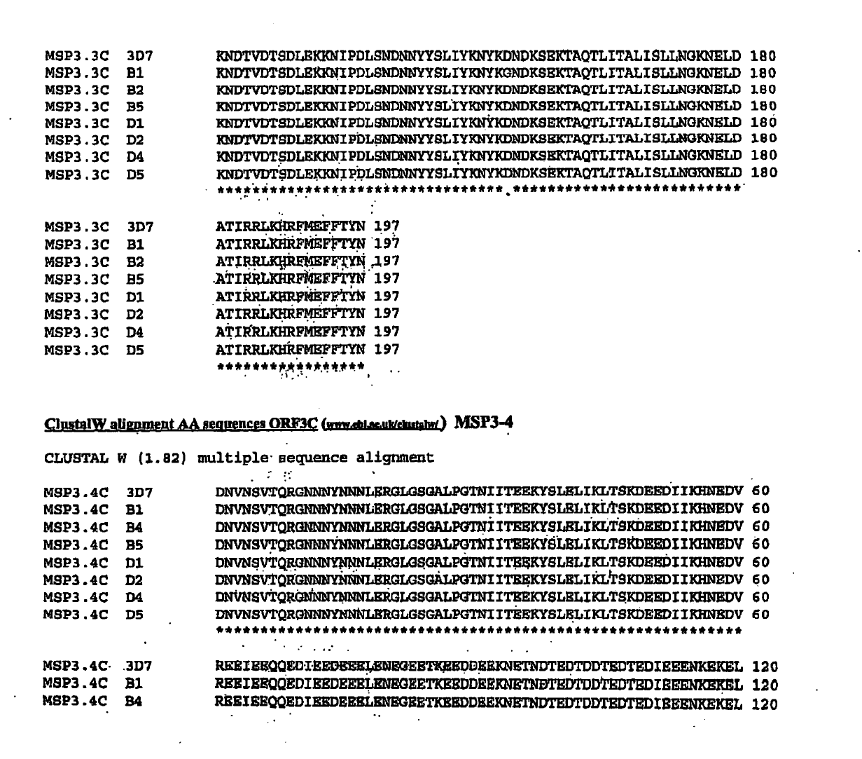

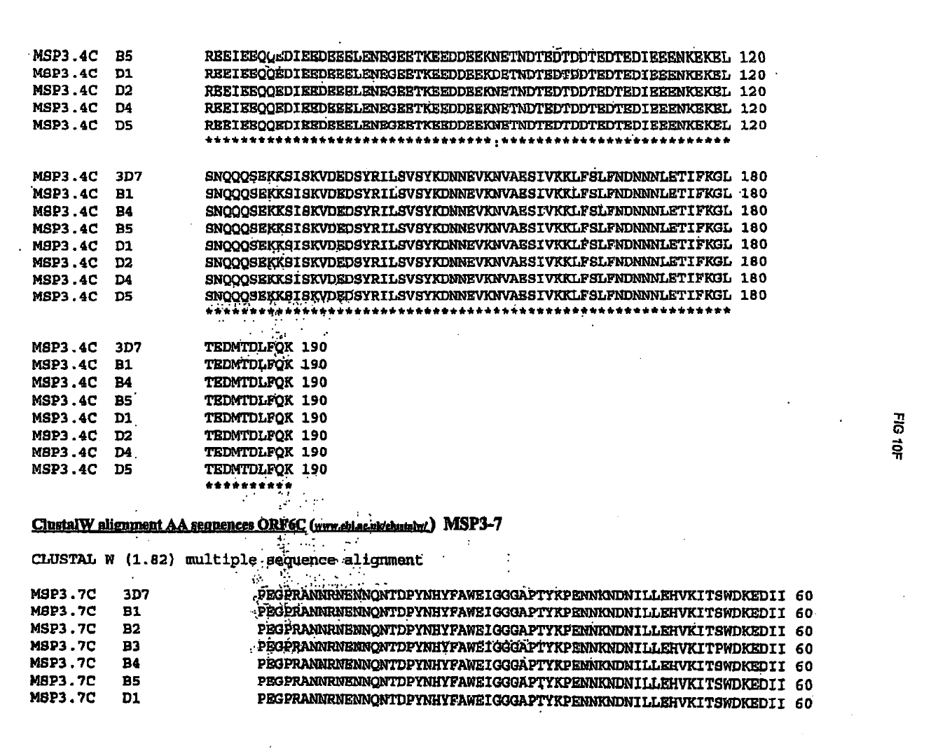

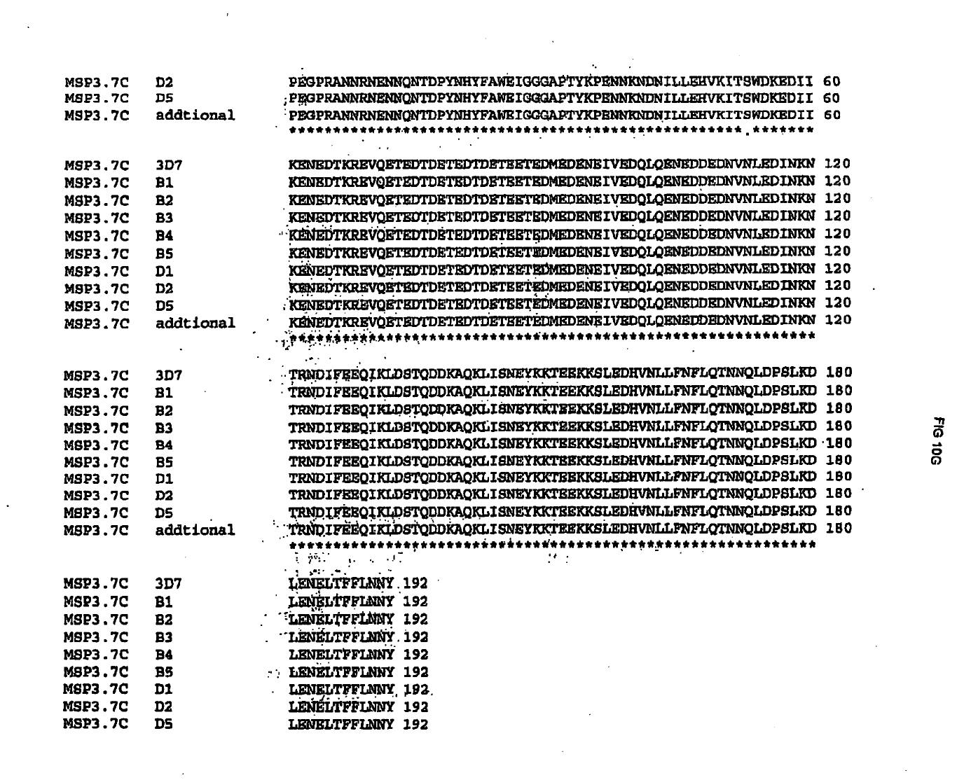

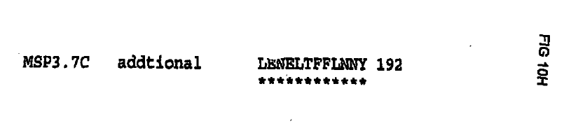

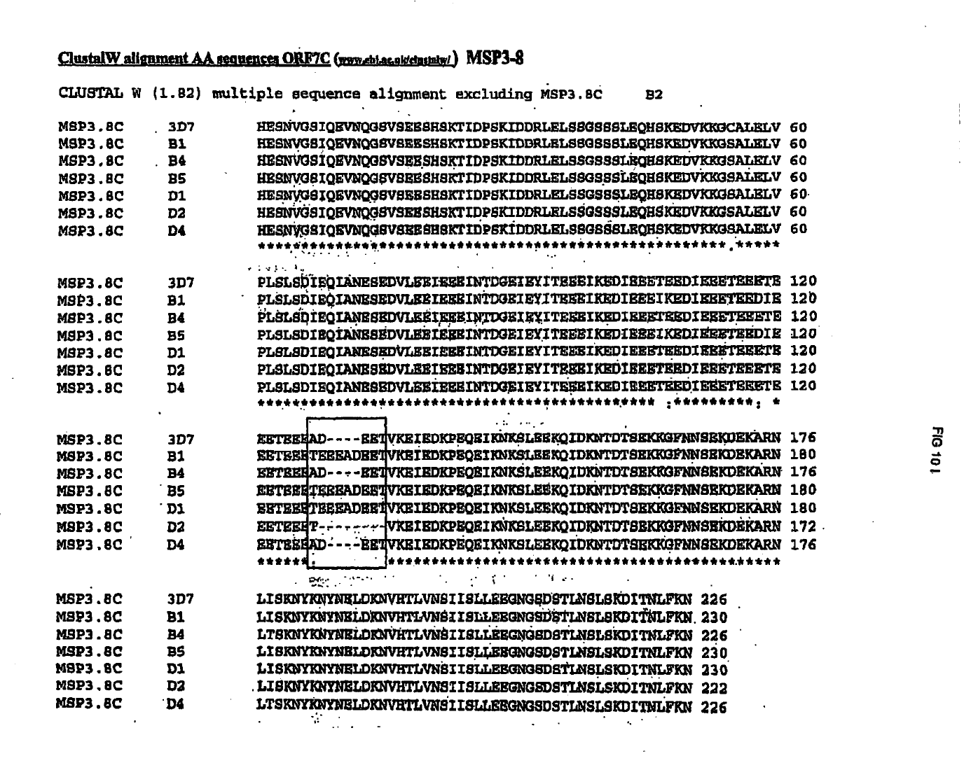

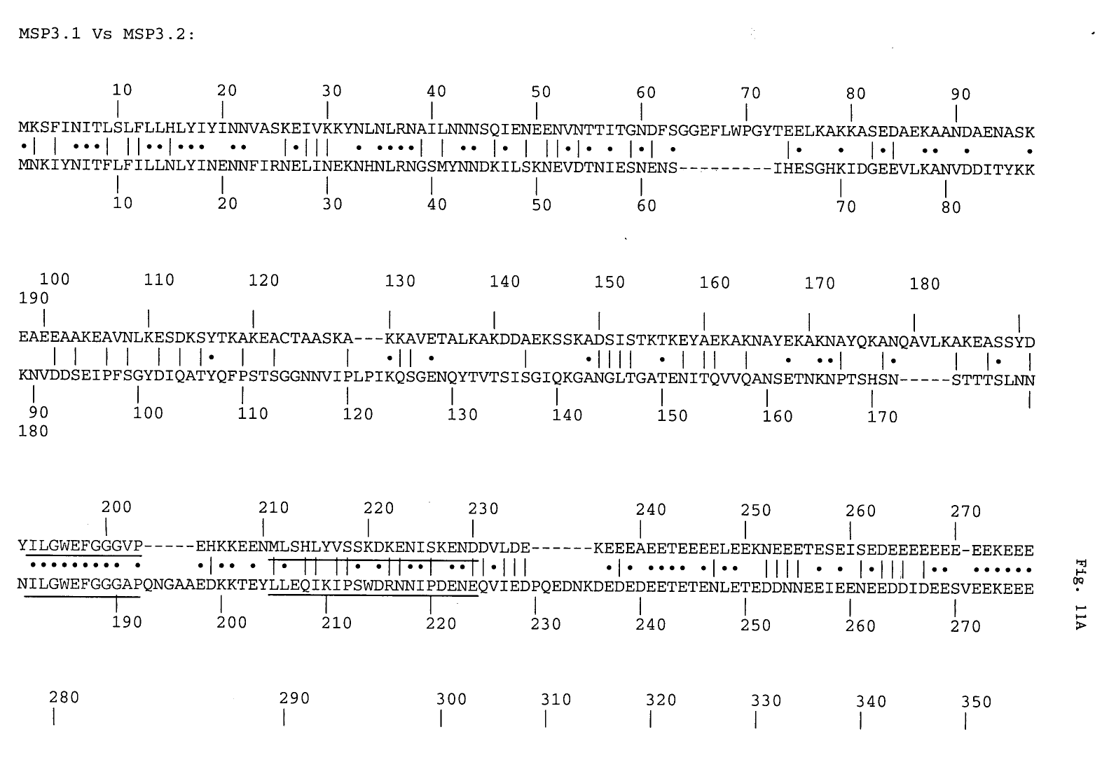

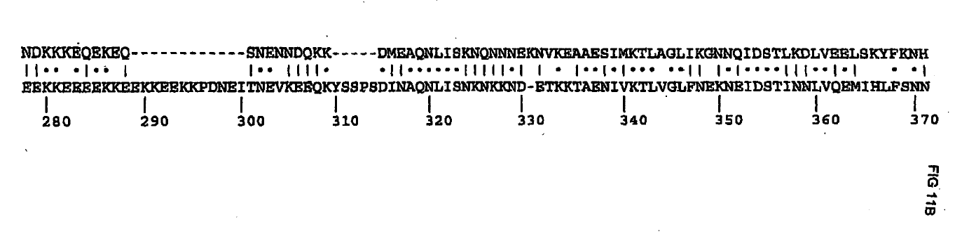

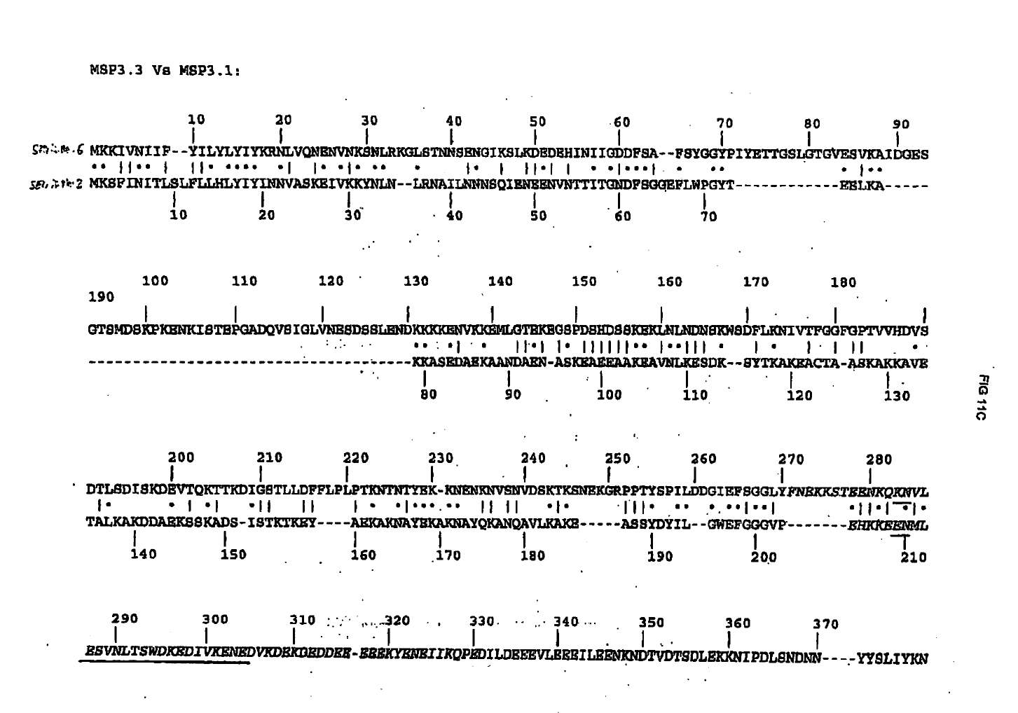

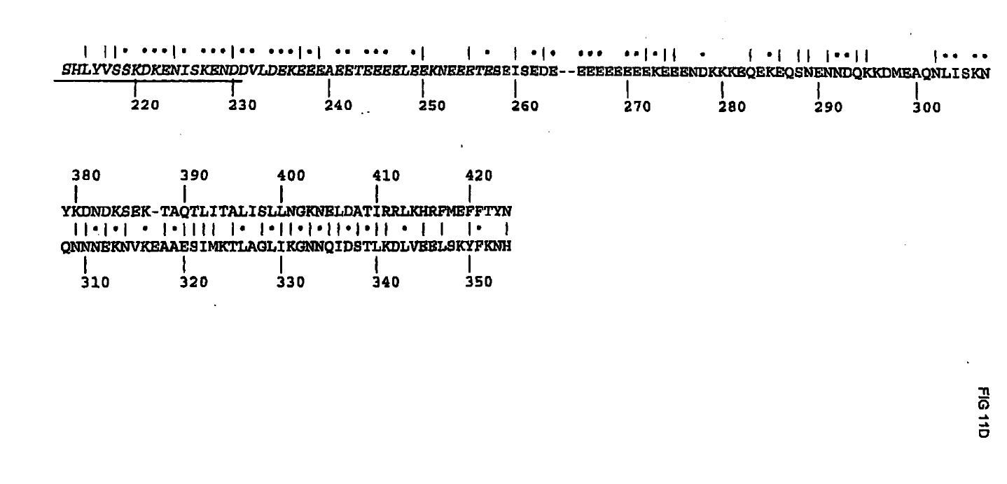

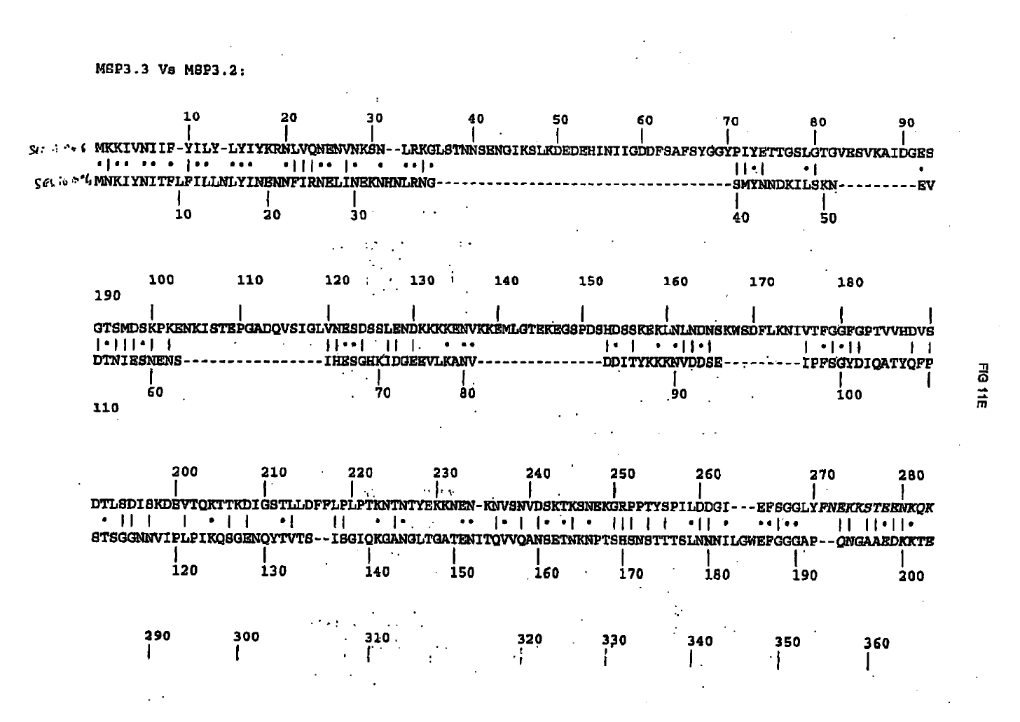

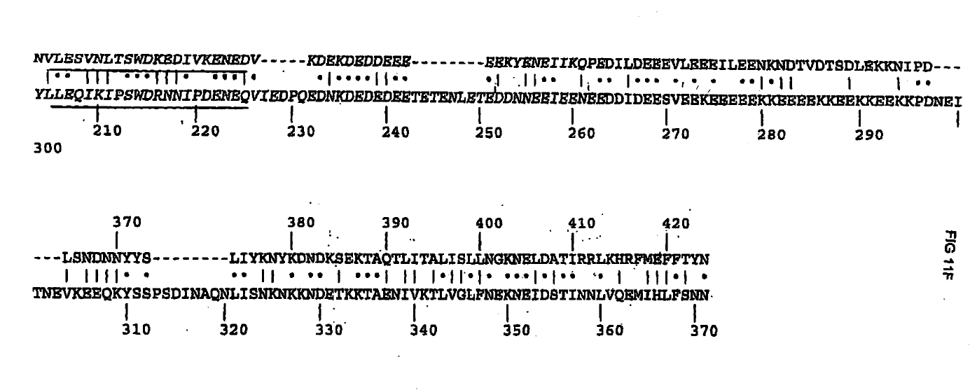

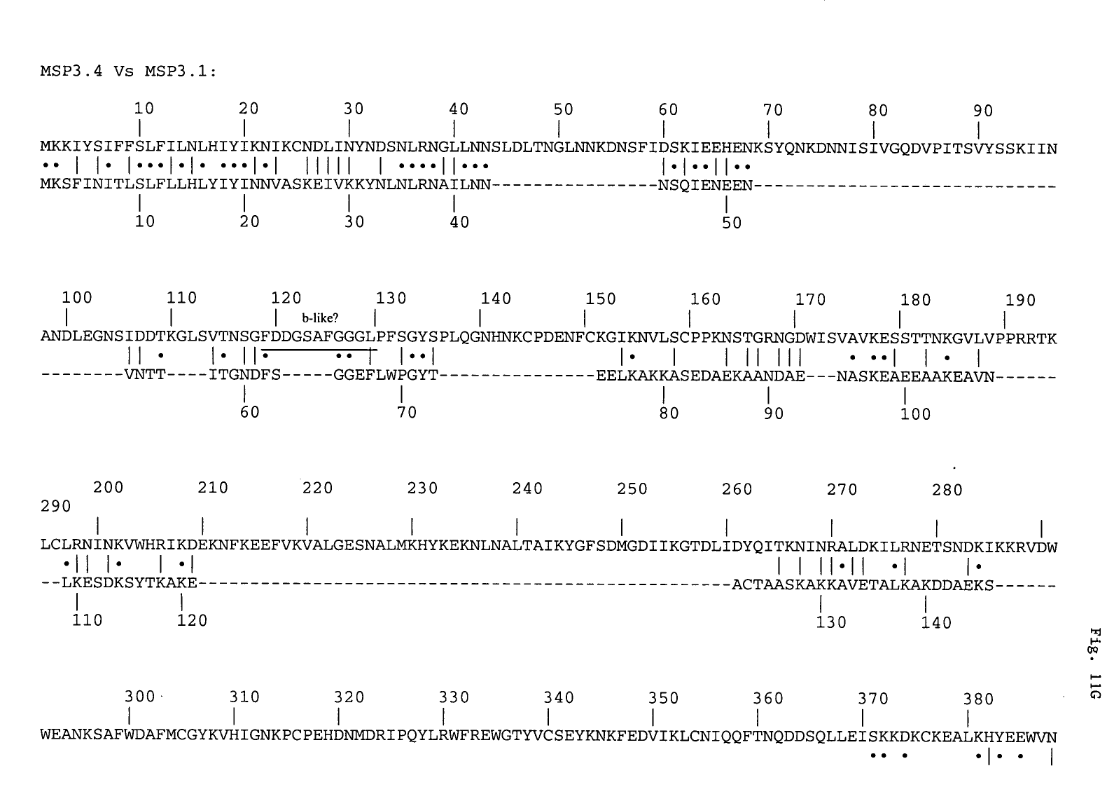

Further studies were conducted either with total IgG or with anti-MSP-3-b antibodies and are summarised in FIG. 8. It is noteworthy that in these experiments, the amount of anti-MSP-3-b antibodies in the total IgG or in the purified preparation was exactly the same. These experiments, which correspond to the mean ±SD of 6 mice treated by anti-MSP-3-b antibodies and 6 mice treated by total African purified IgG clearly confirmed that there was:
a much faster effect of anti-MSP-3-b antibodies,
a more complete effect of anti-MSP-3-b antibodies, since they led to a full clearance of the parasitemia in mice, whereas immune IgG led to a decrease but without sterilising effect, as was the case with the same preparation when injected into human volunteers (and, as is the case, in African adults donors who keep a chronic, low-grade parasitemia).

This observation implies that there are other antibodies present in the immune African IgG which compete or block the inhibitory effect of anti-MSP-3 antibodies. This negative interaction between different antibodies is reminiscent of that reported, for instance, by Blackman and collaborators for anti-MSP-1 antibodies. It can also be related to the interference of non-cytophylic antibodies directed to other merozoite surface antigens and which could act indirectly, for instance, by steric hindrance, reducing the access to MSP-3 antigens of anti-MSP-3 antibodies.

Anyhow, this observation has also very important implications for vaccine development: it can be taken as an indication that immunisation by selected malarial antigens may elicit stronger protective responses than those resulting from exposure to all malarial proteins, or at least to several of them.

In other words, the immunisation, by molecules which are identified as targets of protective mechanisms may lead to induce a stronger protection than that developed by natural exposure, which is already the strongest protection known against asexual blood stages in human beings. It is thus extremely promising for the development of a future, efficient, malarial vaccine.

### Example 5

### Epitope Conservation in the MSP-3 Family

The materials and methods used to perform the experiments described in the present example are described in (Brahimi, Perignon et al. 1993; Badell, Oeuvray et al. 2000). In particular, the methods to obtain the antibodies have been described by Brahimi et al.

As a consequence of the structural homologies between members of the gene family, the existence of immunological cross-reactivity between the corresponding proteins was confirmed: human antibodies were affinity purified on the product of each gene and reacted to all of the remaining.

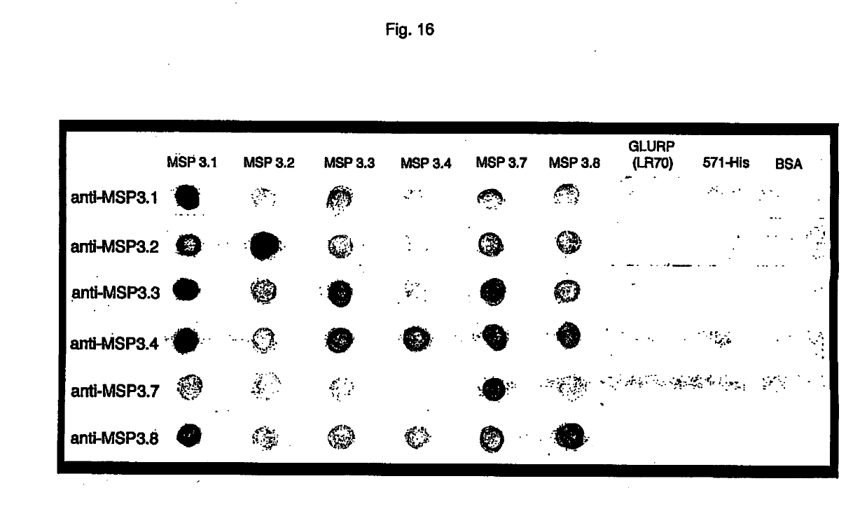

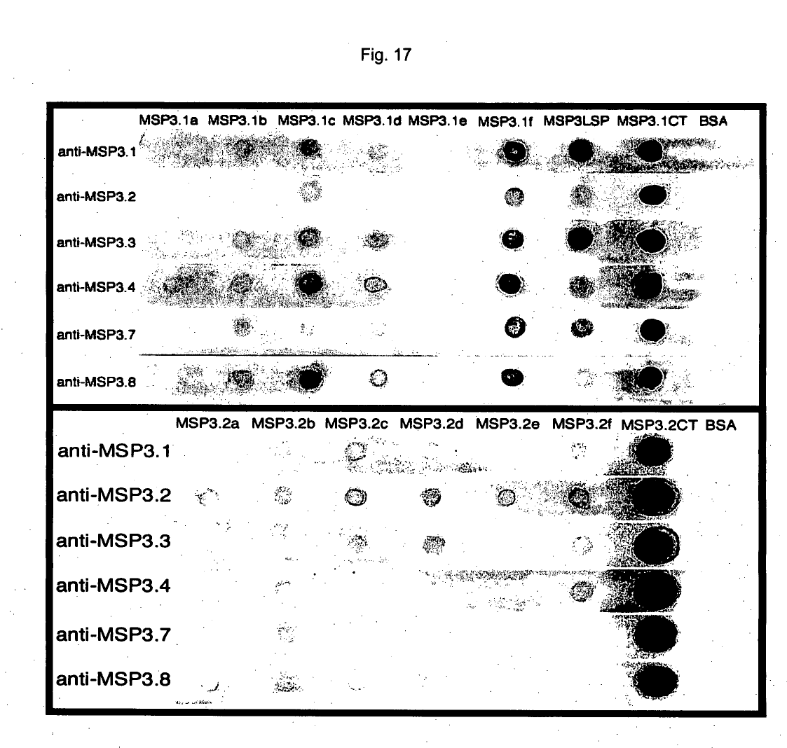

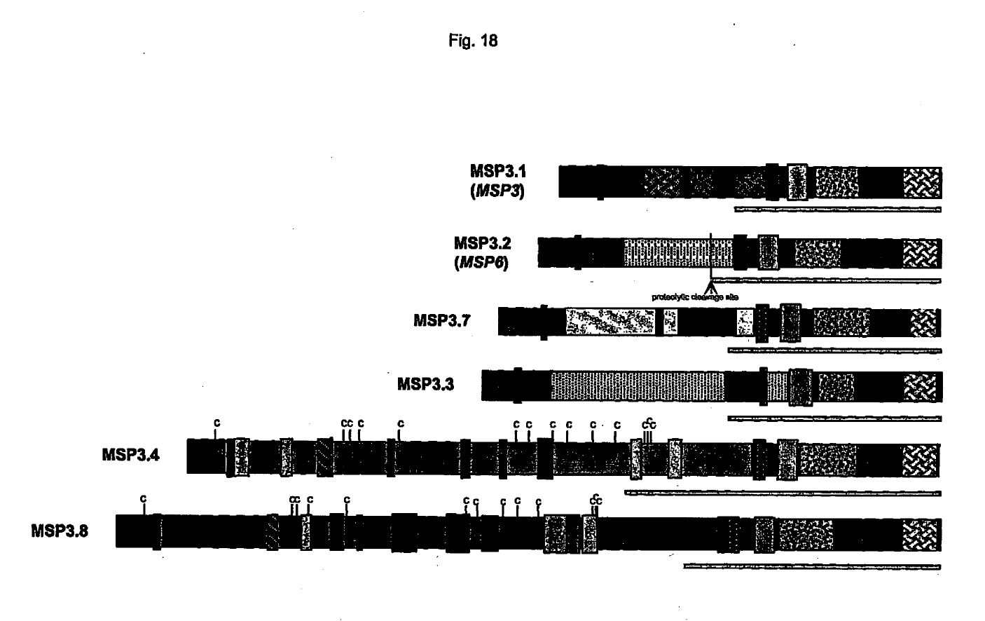

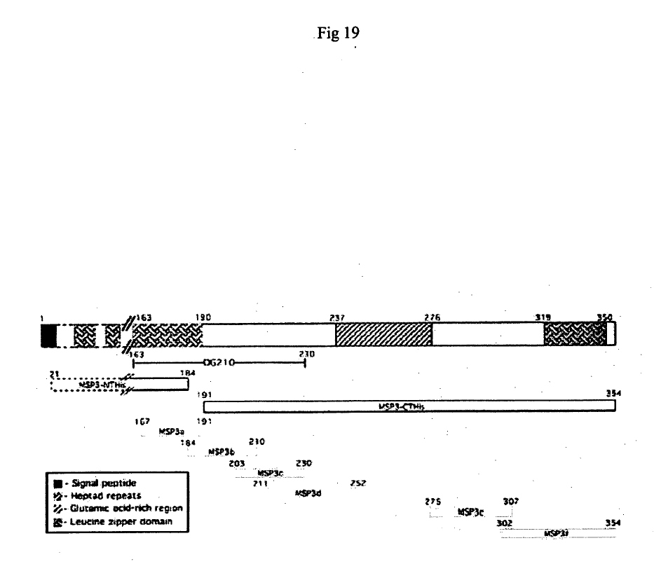

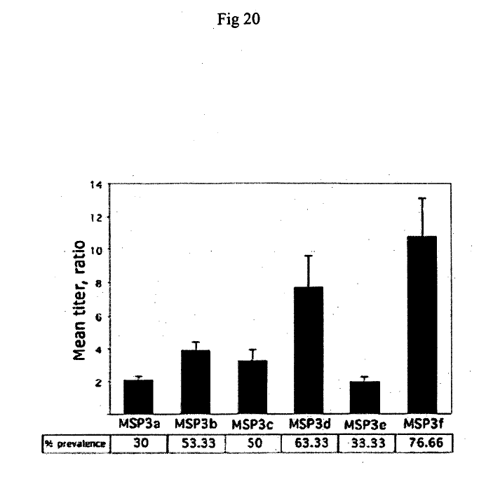

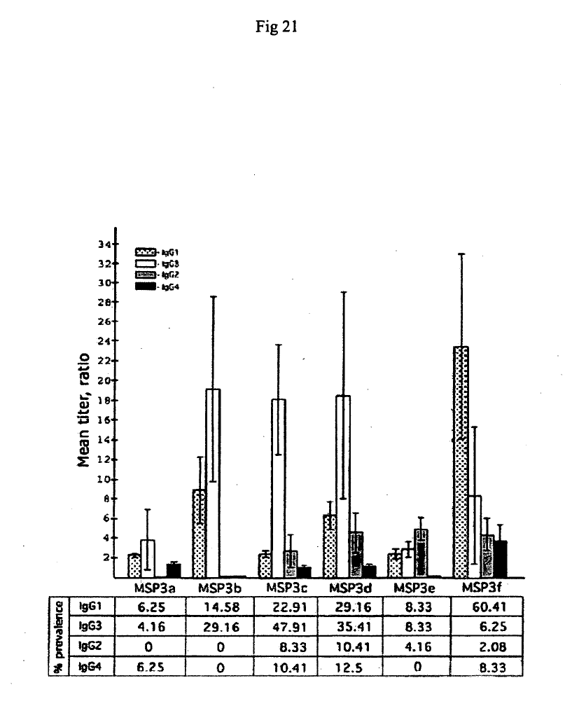

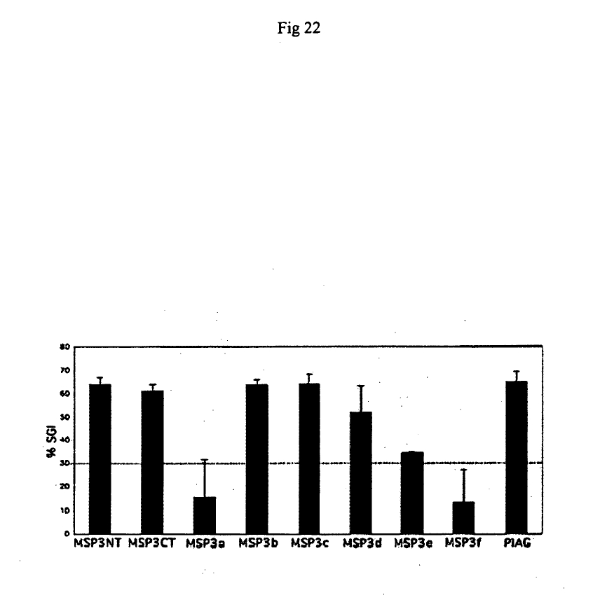

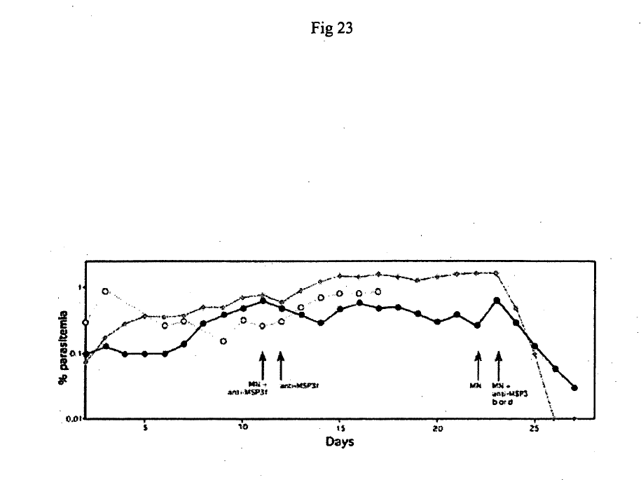

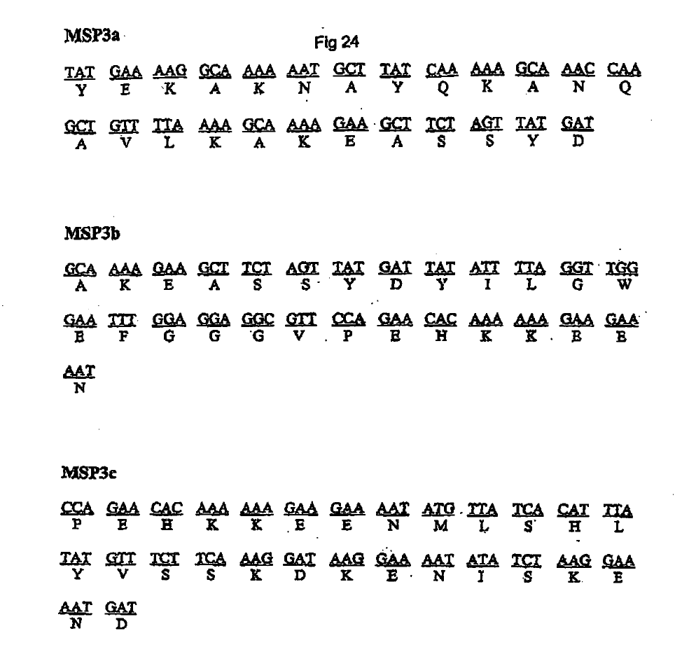

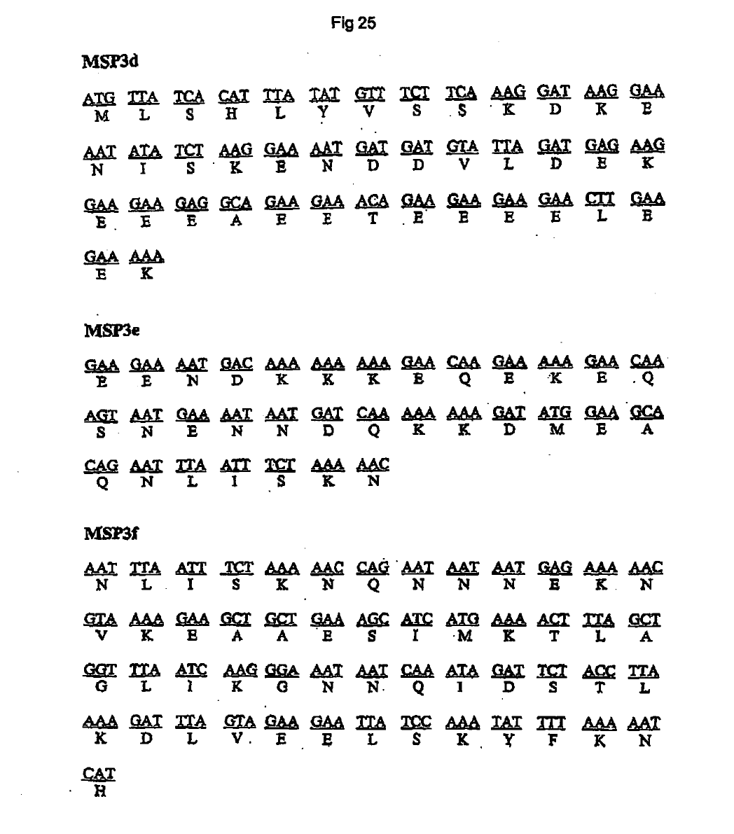

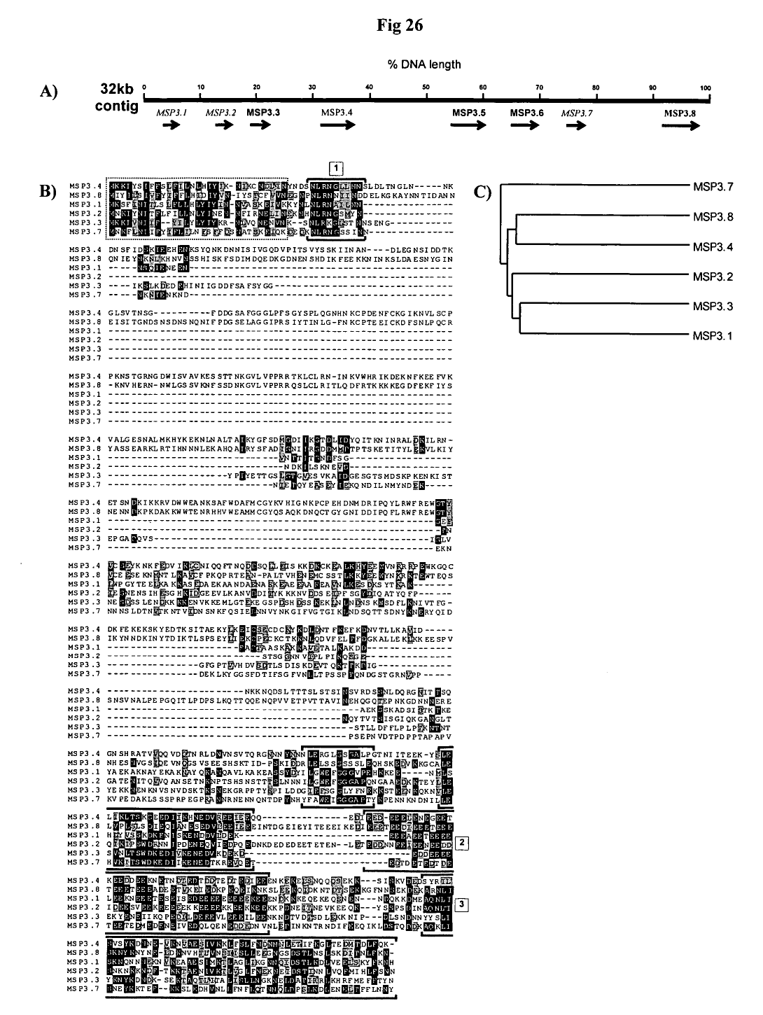

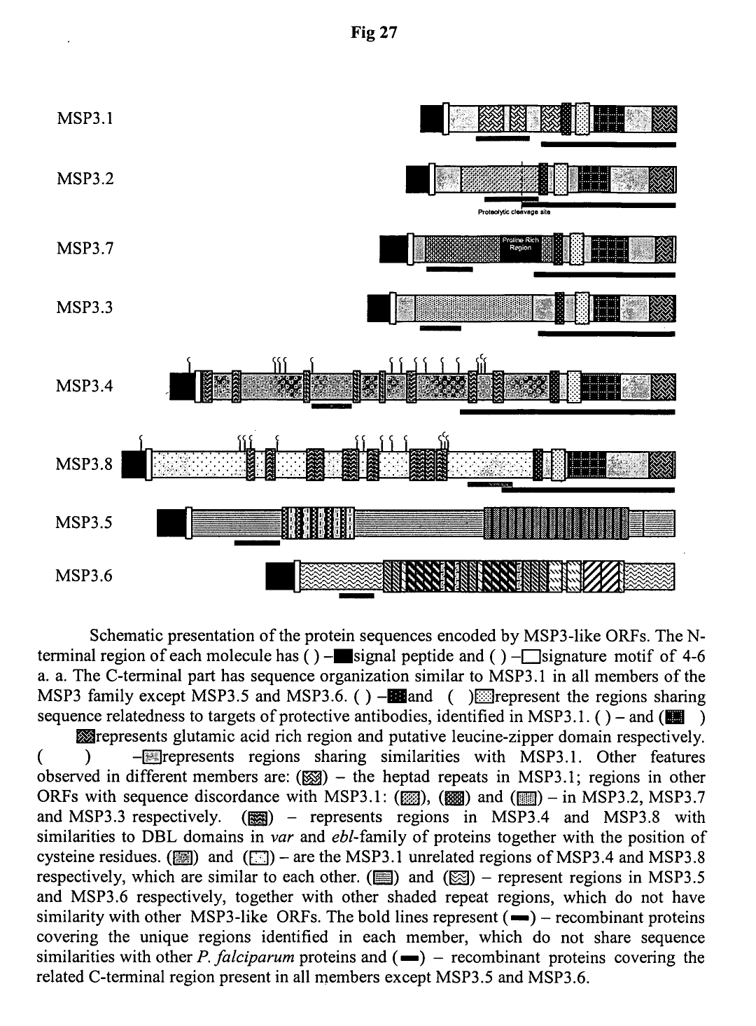

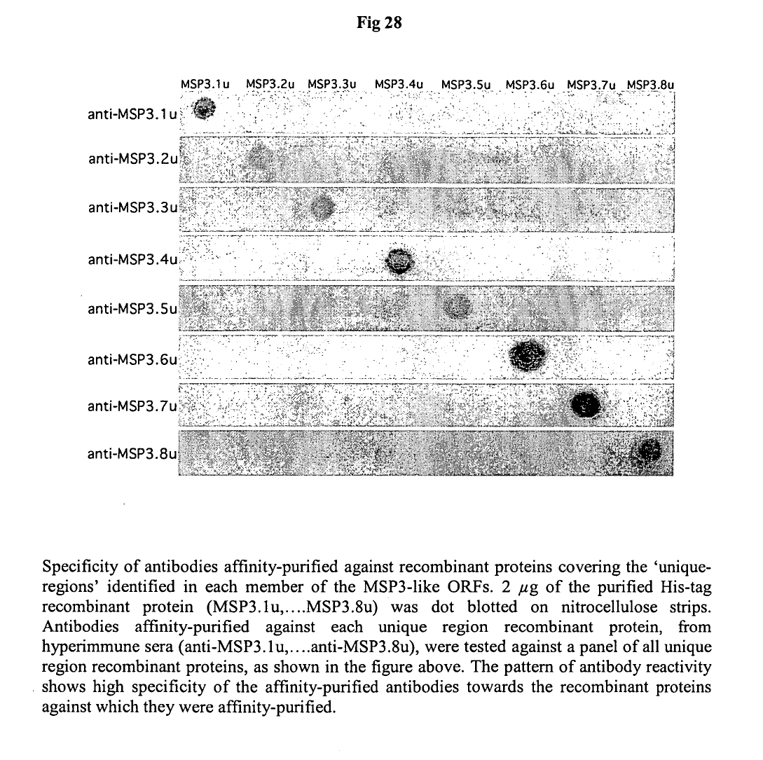

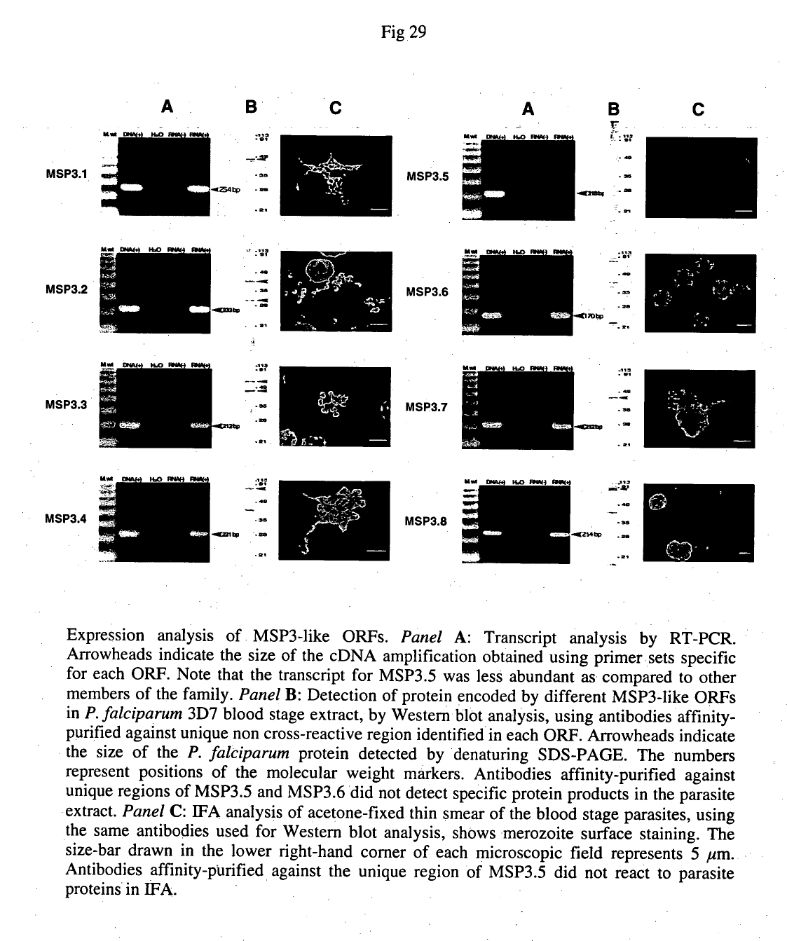

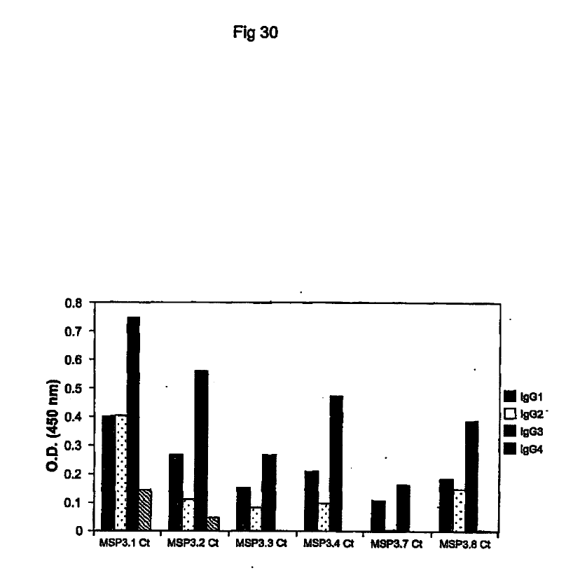

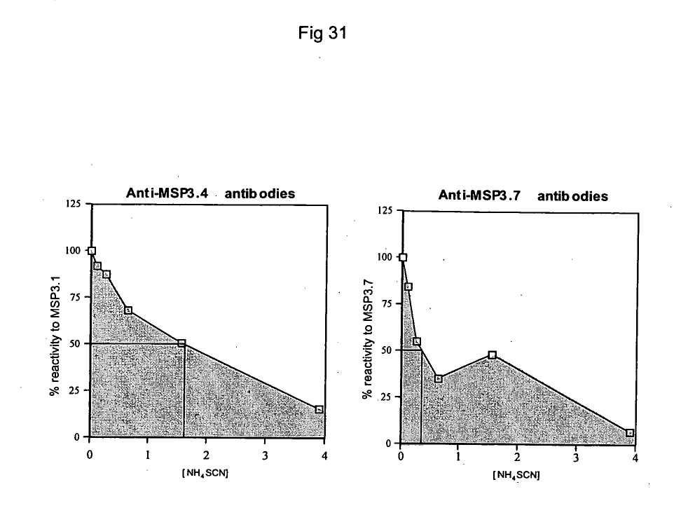

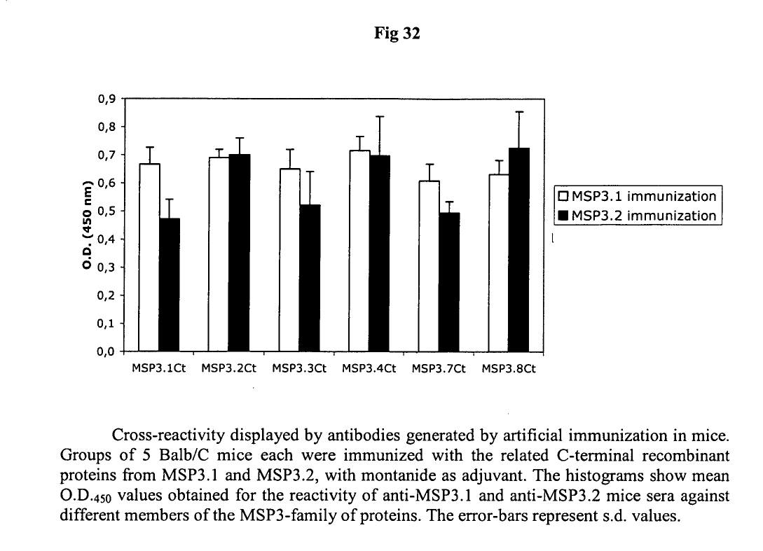

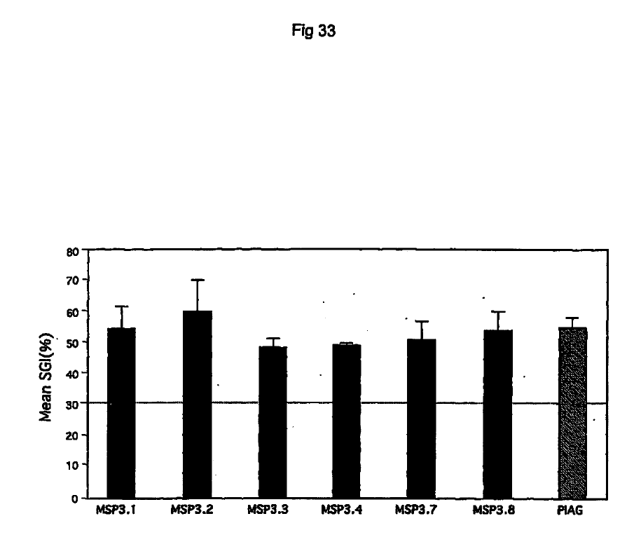

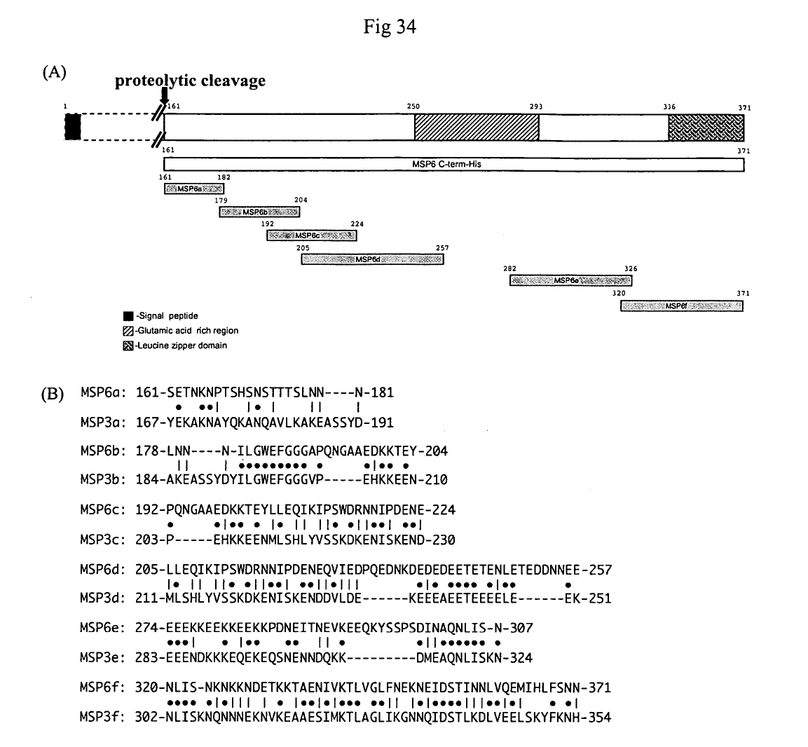

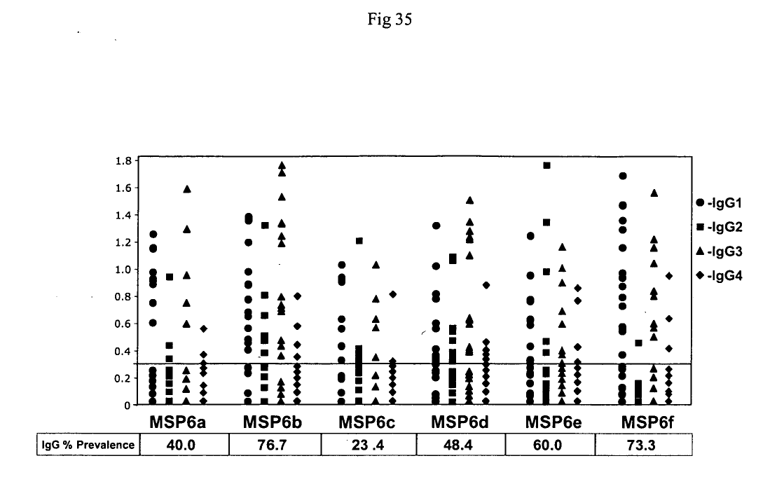

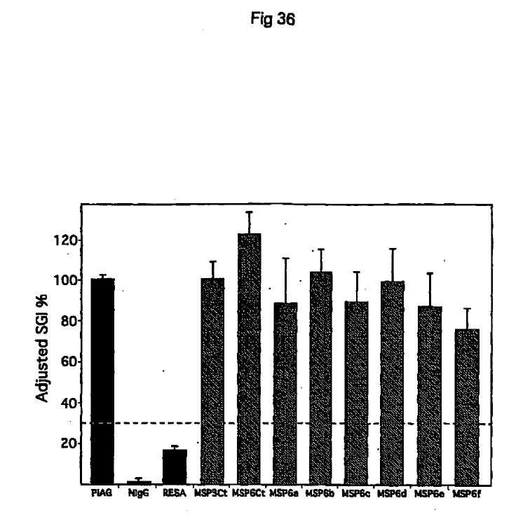

The results show that antibodies induced against one single protein of the MSP-3 family also react with other antigens, in immunoblot (FIGS. 16 and 17), and in ELISA (Table 2 below).

TABLE 2
Antibodies to the seven gene products are all effective at mediating
P. falciparum blood stage killing, in the monocyte-dependent, antibody-
mediated ADCI mechanism, under in vitro conditions.

| | MSP3.1 CT | MSP3.2 CT | MSP3.3 CT | MSP3.4 CT | MSP3.7 CT | MSP3.8 CT | 571-His | BSA |
|---|---|---|---|---|---|---|---|---|
| Anti- | 100 | 6 | 33 | 4 | 35 | 23 | 3 | 4 |
| MSP3.1 CT | | | | | | | | |
| Anti- | 54 | 100 | 22 | 4 | 39 | 37 | 4 | 4 |
| MSP3.2 CT | | | | | | | | |
| Anti- | 117 | 45 | 100 | 5 | 100 | 47 | 5 | 5 |
| MSP3.3 CT | | | | | | | | |
| Anti- | 216 | 44 | 130 | 100 | 147 | 103 | 10 | 10 |
| MSP3.4 CT | | | | | | | | |
| Anti- | 32 | 4 | 3 | 3 | 100 | 5 | 4 | 4 |
| MSP3.7 CT | | | | | | | | |
| Anti- | 73 | 23 | 26 | 8 | 53 | 100 | 6 | 5 |
| MSP3.8 CT | | | | | | | | |

Results show that antibodies to each of those regions are equally effective at achieving P. falciparum erythrocytic stage growth inhibition under in vitro conditions.

This study, which is still ongoing, showed that antibodies affinity purified on the product of one gene cross-reacted with the products of the other genes and vice-versa for each of them, which is also indirectly shown by the results obtained in ADCI (Example 1).

The practical consequence at immunological and vaccine development level is that immunisation by any of the members of the gene family will induce antibodies reactive to the same and to all of the remaining gene products.

Therefore, this constitutes a very particular type of multi-gene family where, instead of epitope polymorphism, which is usually the feature of multi-gene families described to-date, epitope conservation is here the main characteristic and where, in case of deletion, mutation in one given gene, another or all other members of the family can take over the antigenic function. In addition all genes are sumultaneously expressed by one given parasite.

It is herein proposed that this constitutes not only a preferential vaccine family but also a mechanism developed by the parasite to ensure its survival. The parasite can only survive provided it does not kill its host: by inducing antibodies that reduce parasitemia through the ADCI mechanism, the parasite ensures a sufficient degree of protection of the immune host and therefore ensures its own survival. The epitope duplication provided by the gene family ensures that more than one gene product can fulfil this essential task.

### Example 6

### Results Obtained in ADCI with MSP-3-2 Peptides

The results obtained in ADCI with MSP-3-2 peptides a, b, c, d, e and F are the same as those obtained with the same peptides from MSP-3-1, i.e., the antibodies directed against the peptides MSP-3-2 b, c, d and e have an ADCI activity, whereas those directed against the peptides MSP-3-2 a and f do not.

### Example 7

### Complementarity between Responses to MSP3 and GLURP Shown in a Longitudinal Clinical and Parasitological Follow-Up Study

7.A. Materials and Methods

7.A.1. Study Area, Population and Clinical Surveillance

OoDo village is a re-settled forested region of Myanmar with a tropical climate characterized by hot dry, monsoon and cool dry seasons. In this area, malaria was found to be stable and hyper-endemic with seasonal variation, the majority of infections were due to Plasmodium falciparum (98%) and Plasmodium vivax was responsible for the remaining 2%. A malaria attack was defined according to 4 concomitant criteria: i)—corrected axillary temperature≧38.0° C., ii)—absence of other clinical diseases, iii)—presence of asexual P. falciparum forms in thick-films, and iv)—clinical and parasitological improvement after chloroquine treatment. Two febrile attacks were regarded as two different malaria episodes if they were separated by ≧72 h. The results of the first 33 months of follow-up have recently been published (Soe, Khin Saw et al. 2001). The same study population was followed-up for one additional year, up to 31^{st} Dec. 1998, using the same protocol. Venous blood samples were drawn during September 1998, and malarial attack rates recorded from Jan. 1^{st} to Dec. 31^{st} 1998 were used to analyze the relationship with clinical protection.

7.A.2. Blood Sampling and Parasitological Study

Surveillance of malarial infection was carried out by systematic monthly examination of thick and thin blood films from finger-prick. A slide was regarded as negative if no parasite was visualized in 200 oil-fields in Giemsa stained thick film. For febrile cases two finger-prick films before and after chloroquine treatment were examined. Venous samples were collected in vacutainers, sera aliquoted aseptically, and stored at −20° C. until tested. Samples taken from a representative subgroup of 116 villagers from whom more than 60% of the monthly blood films were available for parasitological data were selected from the larger cohort of 292 residents.

7.A.3. Antigens

The three recombinant GLURP antigens were derived from the N-terminal non-repeat region R0 (GLURP₂₇₋₅₀₀), the central repeat region R1 (GLURP₄₈₉₋₇₀₅), and the C-terminal repeat region R2 (GLURP₇₀₅₋₁₁₇₈) of P. falciparum F32 (Oeuvray, Theisen et al. 2000). The C-terminal 19-kDa fragment of MSP1, MSP1₁₉, from the Wellcome strain (MSP1-W-19) was produced as a recombinant GST-fusion protein in Escherichia coli and was a kind gift from Dr. A. Holder, UK. The GST-tag was removed by enzymatic cleavage and subsequent affinity chromatography before use. The MSP3b synthetic peptide (184-AKEASSYDYILGWEFGGGVPEHKKEEN-210, SEQ ID No:5) contained the MSP3b B-cell epitope which reacts with ADCI-effective human antibodies (Oeuvray, Bouharoun-Tayoun et al. 1994).

7.A.4. Antibody Assays

The levels of antibodies to the three P. falciparum-derived antigens were measured by enzyme-linked immunosorbent assay (ELISA) as previously described (Oeuvray, Theisen et al. 2000). Briefly, microtiter plates (Maxisorb, Nunc, Denmark) were coated overnight at 4° C. with recombinant proteins or synthetic peptide at the following concentrations: 0.5 μg/ml (R0 and R2), 1 μg/ml (R1 and MSP1) and 5 μg/ml (MSP3b). For GLURP antigens 0.05 M Na₂CO₃, pH 9.6 and for MSP1 and MSP3 phosphate buffered saline (PBS) pH 7.4 were used as coating buffers. The next day the plates were washed with PBS plus 0.05% Tween 20 (PBST) and blocked with 2.5% non-fat milk in PBS for 2 h. Sera diluted in PBST containing 1.25% (w/v) non-fat milk, were added to duplicate wells and incubated for 1 h at room temperature. Various dilutions of sera were made for each antigen: 1:200 for GLURP, 1:100 for MSP1 and 1:20 for MSP3. These dilutions were selected after preliminary pilot studies, which revealed more than a 10-fold difference between control and test samples. Bound antibody was detected by peroxidase-conjugated goat anti-human immunoglobulin (Caltag Laboratories), diluted 1:3000. Color was revealed by O-phenylenediamine (Sigma, St. Louis, Mo.) and H₂O₂ in citrate buffer pH 5 for 30 min. The optical density (OD) at 492 nm was determined in a plate reader (Titertek Multiskan MCC 1340). The plates were washed extensively with PBST between each incubation step. All ELISA tests included 6 control sera, randomly selected among 100 French blood donors never exposed to malaria.

For subclasses determination of IgG1-4, monoclonal mouse anti-human subclasses (clones NL16=IgG1 (Boehringer®), HP6002=IgG2 (Sigma®), Zg4=IgG3, and RJ4=IgG4 (both from Immunotech®)) were used. They were diluted 1:2,000, 1:10,000, 1:10,000, and 1:1,000, respectively in 1.25% (w/v) non-fat milk in PBST and incubated for 1 h at room temperature. Goat anti-mouse IgG conjugated to peroxidase (Caltag Laboratories®), diluted 1:3000 in 1.25% (w/v) non-fat milk in PBST was added and incubated for 1 h. Bound labeled antibody was revealed as described above. The dilutions of each isotype-specific monoclonal antibody (MAb) had been determined previously as those discriminating between human Ig sub-classes, i.e. yielding no cross-reactions between subclasses (Oeuvray, Theisen et al. 2000). The results for total IgG as well as subclass antibody levels were expressed as ratios of antibody response which were calculated by dividing the mean OD of test with the mean plus 3 SD of the 6 normal controls run simultaneously. A sample with a ratio of ≧1 was considered positive.

7.A.5. Statistical Analysis

The Mann-Whitney U-test and Spearman's rank-order correlation coefficient were used for the calculations of P-values. The association between the risk of malaria attack during 1998 and the levels of antibodies (expressed in ratios) were tested with JMP® software, using either a Poisson regression model where the effect of confounding factors such as age, gender, time spent in the village and transmission were controlled or a logistic regression analysis (with or without occurrence of malaria attack).

7.B. Results

7.B.1. P. falciparum Infections in the Study Cohort

All 116 subjects in the study cohort were from OoDo village situated in Myanmar, South-East Asia, where malaria is hyper-endemic (Soe, Khin Saw et al. 2001). The prevalence of P. falciparum parasitemia fluctuated around 40% from January to July and dropped to around 20% from August to December in 1998. The incidence of clinical malaria, which was calculated as the average number of attacks per month in the study cohort and expressed in percentage varied considerably over the year, peaking in June. The infective inoculation rate has not been determined for OoDo, however, Tun-Lin, W et al (Tun-Lin, Thu et al. 1995) found 13.7 infective bites per person per year in a village, which is located 15 km East of OoDo village. The finding agrees well with an estimated number of 11 infective bites per person per year as calculated by the method of (Beier, Killeen et al. 1999). Most infections (98%) were due to P. falciparum (Soe, Khin Saw et al. 2001). During the 12-month period of continuous clinical surveillance, 86 (74%) of the 116 villagers had at least one malaria attack as defined in the Material and Method section and these individuals were considered to be susceptible to malaria. During the same 12-month period, 30 (26%) of the villagers had no episode of clinical malaria, and these individuals were regarded as clinically protected.

7.B.2. Antibody Recognition of P. falciparum-Derived MSP3, GLURP, and MSP1 Antigens

Levels of IgG, and IgG subclasses against the MSP3b₁₈₄₋₂₁₀ peptide (MSP3b) and the four recombinant proteins representing the GLURP₂₇₋₅₀₀ (R0), GLURP₄₈₉₋₇₀₅ (R1), GLURP₇₀₅₋₁₁₇₉ (R2), and MSP1 -19-kDa C-terminal regions were determined in the 116 sera collected during September 1998. R2 was the most frequently recognized antigen by IgG antibodies (67.2%) followed by R1, MSP3b and MSP1 (all at 62%), and R0 (58.6%). The highest OD values were obtained against R0 and R2, whereas MSP3b yielded lower OD values. Levels of IgG against all three GLURP regions and MSP1 were significantly associated with age (Spearman's rank-order correlation coefficient, R=0.51, 0.26, 0.41, and 0.43 for R0, R1, R2, and MSP1, respectively, P<0.05) while the IgG response against MSP3 was independent of age (R=0.16, P=0.17). As for the subclass responses, IgG1 and IgG4 against MSP1, IgG2 against MSP3, IgG1 and IgG3 against R0, IgG2 and IgG3 against R1 and IgG4 against R2, were found significantly associated with age (Table 3). Neither level nor prevalence of positive antibody response varied with gender for any of the antigens tested.

TABLE 3
Relationship between age and the level of subclass antibody
responses to each of the antigens studied. P and R-values were calculated
by the Sperman's Rank correlation Coefficient.

| Antigens | IgG Subclasses | P | R |
|---|---|---|---|
| MSP1 | IgG1 | 0.0002 | 0.344 |
| | IgG4 | 0.0009 | 0.309 |
| MSP3 | IgG2 | 0.0090 | 0.242 |

GLURP antigens

| | | | |
|---|---|---|---|
| R0 | IgG1 | 0.0220 | 0.213 |
| | IgG3 | 0.0040 | 0.268 |
| R1 | IgG2 | 0.0040 | 0.268 |
| | IgG3 | 0.0008 | 0.313 |
| R2 | IgG4 | 0.0140 | 0.231 |

7.B.3. Antibody Responses and Clinical Protection

A striking difference between IgG subclass responses and protection was observed for the three different antigens (Table 4). For example, the IgG response against the C-terminal 19-kDa fragment of MSP1 was almost exclusively of the IgG1 subclass with a median value 8.6 times higher in the protected than in the susceptible group whereas, IgG3 antibodies predominated against the MSP3b epitope in protected individuals with a median value 6.5 times higher than that found in susceptible individuals. Although less pronounced, a similar dissimilarity in the cytophilic IgG subclasses response was also observed for different regions of GLURP, IgG1 antibodies predominating against the non-repeat R0-region and IgG3 antibodies prevailing against the R2 repeat-region.

TABLE 4
Median levels (and interquartile range) of IgG subclass antibodies to
MSP1, MSP3 and GLURP antigens found in villagers from OoDo
considered as either protected from, or susceptible to, P. falciparum
malaria attacks over 1 year of active and continuous follow-up.

| | | | Susceptible | | Fold |
|---|---|---|---|---|---|
| | IgG | Protected group | group | P | dif- |
| Antigen | Subclass | (n = 30) | (n = 86) | values | ference |
| MSP1 | IgG1 | 9.5 (0.8-25.03) | 1.1 (0.5-8.22) | .003# | 8.6 |
| | IgG2 | 0.9 (0.8-1.26) | 0.9 (0.8-1.09) | >.05 | |
| | IgG3 | 0.9 (0.1-2.63) | 0.4 (0.0-0.81) | >.05 | |
| | IgG4 | 1.2 (0.8-3.01) | 1.0 (0.7-1.47) | .01 | |
| MSP3 | IgG1 | 1.4 (0.8-2.4) | 0.7 (0.4-7.0) | <.001* | 2.0 |
| | IgG2 | 1.1 (0.9-1.57) | 0.8 (0.7-1.0) | >.05 | |
| | IgG3 | 6.5 (2.5-14.03) | 1.0 (0.6-1.49) | <.001* | 6.5 |
| | IgG4 | 1.3 (1.0-1.82) | 1.0 (0.9-1.35) | <.001* | 1.3 |
| R0 | IgG1 | 3.9 (2.4-7.62) | 1.8 (1.0-3.15) | <.001* | 2.2 |
| | IgG2 | 0.9 (0.4-2.5) | 0.8 (0.4-1.33) | >.05 | |
| | IgG3 | 1.3 (0.6-3.76) | 0.9 (0.3-1.44) | .019 | |
| | IgG4 | 0.2 (0.2-2.05) | 0.6 (0.2-1.07) | >.05 | |
| R1 | IgG1 | 0.3 (0.1-0.9) | 0.2 (0.1-0.4) | >.05 | |
| | IgG2 | 0.9 (0.4-1.64) | 0.6 (0.4-0.91) | >.05 | |
| | IgG3 | 1.2 (0.4-2.64) | 0.5 (0.2-0.91) | .039 | |
| | IgG4 | 0.2 (0.2-0.52) | 0.7 (0.2-0.94) | .021 | |
| R2 | IgG1 | 2.0 (0.9-5.4) | 1.1 (0.3-3.11) | .01 | |
| | IgG2 | 2.0 (1.0-4.02) | 0.9 (0.2-1.73) | <.001* | 2.2 |
| | IgG3 | 6.4 (1.7-12.01) | 0.9 (0.3-2.83) | <.001* | 7.1 |
| | IgG4 | 1.0 (0.6-1.2) | 0.6 (0.4-0.85) | .003 | |

Given the number of statistical tests carried out, the Bonferroni's correction factor was applied to determine the level of significance and only P values < .0025 were considered significant (*).
P values were determined from the non-parametric Mann-Whitney U-test. Fold difference refers to the ratio of median values from the two groups.
#Values marginally different.

Since the antibody titers against GLURP and MSP1 increased as a function of age, the correlation of clinical status of the villagers with various antibodies was reexamined in a logistic-regression model considering age and all the antibody responses (log transformed) as explanatory variables. When testing these parameters in the model and in particular when age was controlled for, among all antibody responses, the strongest predictors of malaria protection identified were increased levels of IgG3 against MSP3b (F ratio=67.5; P<0.0001) and against GLURP-R0 (F ratio=23.1; P<0.0001). Other antibodies were not significantly associated with protection. In contrast, the analysis indicated that levels of IgG4 against R0 (F ratio=4.4; P=0.038) and R1 (F ratio=3.9; P=0.051) increased with the number of malaria attacks, i.e. they were to some extent predictive of susceptibility to malaria.

7.B.4. Antigen Specificity of IgG3 Responses in Protected Villagers

FIG. 14A shows the general pattern of IgG3 antibody responses found against the different blood stage antigens in OoDo. The range of values was large for most antigen-specific antibody responses and this suggested that different subgroups of “responders” might exist. Sera of villagers who were protected from clinical malaria did not all show high IgG3 values against both MSP3b and GLURP-R0. Some individuals displayed an unexpectedly low IgG3 reactivity against either one of these 2 antigens. In an attempt to understand how these villagers were protected against malaria attacks, two sub-groups were identified, characterized by : a)—low IgG3 responses against MSP3 (7 out of 30 cases) or b)—low IgG3 responses against R0 (15 out of 30 cases). The levels of IgG3 antibodies against the other three antigens were estimated (FIG. 14B). The 7 protected individuals (mean age±1std error=33.9±7.0 years) with a low IgG3 response to MSP3b (IgG3 ratio=1.26±22) were found to have a strong IgG3 response to R0 (IgG3 ratio=9.09±3.41) and to R2 (IgG3 ratio=8.36±5.86). In the 2^{nd} subgroup of 15 other individuals (24.7±4.3 years of age) also protected despite a low IgG3 response to GLURP-R0 (IgG3 ratio=0.55±0.08), the reverse situation was found (FIG. 14C ): a high IgG3 antibody response against MSP3b was observed (IgG3 ratio=10.45±2.07) and to a lesser extent against GLURP-R2 (IgG3 ratio=4.43±80). The titers for those responding to only one antigen tended to be higher than those responding to both antigens (Table 4). The number of years spent in OoDo village did not significantly differ between the groups of low-responders to MSP3 (20.43±4.70 years of residence) and R0 (18.7±10.6 years of residence).

Sera from 13 of the 30 individuals considered as protected in 1998 had also been sampled in 1993, and therefore were used to compare at 5 years interval the relative levels of anti-MSP3 and anti-R0 IgG1 and IgG3 antibodies. As shown in FIG. 14D and 14E, there was no major change detectable in the levels of specific IgG1 against the two antigens. In contrast, levels of IgG3 antibodies to both MSP3 and GLURP increased from 1993 to 1998. In the subgroup of 7 individuals with elevated IgG3 against MSP3 in 1998 (FIG. 14D), the difference corresponded to a 1.67 times increase (P=0.11). In the subgroup of 6 subjects with elevated IgG3 against R0 in 1998 (FIG. 14E), the difference corresponded to a more important, ie. 3.93 times increase (P=0.05). The 6 individuals with high IgG3 responses against MSP3 in 1998 also had high titers 5 years earlier, suggesting that they were already protected via a sustained anti-MSP3 IgG3 response in 1993, when they were 18.2±9.8 years of age. In contrast, for GLURP the 7 individuals with a strong anti-R0 IgG3 response detectable in 1998 had substantially lower anti-R0 IgG3 responses 5 years earlier (P=0.0157), when they were 23.7±6.9 years of age. Thus, there was a drastic change in those 7 individuals protected via IgG3 to R0 in 1998 and their protection 5 years earlier was possibly related to IgG3 against MSP3.

7.C. Discussion

The present study is the first one to show an association between antigen-specific antibody responses and protection from clinical malaria in S-E Asia. The prevalence of positive antibody responses against GLURP and MSP3 was high in OoDo, ranging from 58.6% (R0) to 67.2% (R2). This observation is in-keeping with the finding that B-cell epitopes within GLURP and MSP3 are highly conserved among P. falciparum laboratory lines and field isolates from Africa and Asia (Huber, Felger et al. 1997),(McColl and Anders 1997; de Stricker, Vuust et al. 2000). The prevalence of antibodies to MSP1-W-19 was also high, being almost twice the values found in the Gambia and Sierra Leone (Egan, Morris et al. 1996) and in Ghana (Dodoo, Theander et al. 1999) suggesting that strains related to the Wellcome strain might be prevalent in OoDo.

The highest ELISA-titers were found against the recombinant GLURP R0- and R2-regions and MSP1. The differences in GLURP-R0, -R1, -R2 and MSP1 ELISA-titers very likely reflected differences in serum antibody reactivity. In contrast, the MSP3-ELISA gave comparatively lower signals, however this discrepancy might be at least in part, related to the use of a short synthetic peptide defining a single or limited number of epitopes, as compared to recombinant proteins in the case of GLURP and MSP1 which are known to define several epitopes (Theisen, Soe et al. 2000).

Levels of IgG against all GLURP regions and MSP1 were significantly associated with age (P<0.05) while in contrast to this situation, the IgG response against MSP3 was found independent of age. Regarding IgG subclass responses, several of them were also found significantly associated with age and these variations could reflect the duration of exposure to the malaria parasites as well as the gradual maturation of the immune system over time.

There was a statistically significant increase in the levels of IgG3 against R0 and MSP3 among the protected individuals living in OoDo as compared to the non-protected ones. These results are in agreement with those of Dodoo et al. (Dodoo, Theisen et al. 2000), who found that cytophilic antibody responses against R0 and R2 were strong predictors of protection in Ghanaian children, and those of Oeuvray et al., who found a consistent correlation between protection and elevated IgG3 against both GLURP-R0 and R2 in Dielmo, West Africa (Oeuvray, Theisen et al. 2000). Similarly MSP3-specific IgG3 responses have previously been associated with protection against clinical malaria in Dielmo. Altogether, these results suggest that the same subclass of IgG response to the same critical epitopes are involved in the gradual development of protection against P. falciparum malaria in African as well as in Asian populations living in malaria endemic areas. In addition, the present study found a significant negative correlation between the levels of non-cytophilic antibodies against R0 and R1 and clinical protection. Therefore, on the one hand, there is a positive association between cytophilic IgG subclass responses and protection and on the other hand, a negative association between non-cytophilic subclass responses with the same epitope specificity and protection. This epidemiological result is in agreement with the in vitro observation that non-cytophilic antibodies can inhibit the bridging of merozoites and human monocytes by cytophilic antibodies against the same antigenic target and thereby reduce the ability of the latter to control parasite multiplication by the ADCI mechanism (Bouharoun-Tayoun and Druilhe 1992).

Whereas most of the protected residents of OoDo had high IgG3 responses against both MSP3 and GLURP, a number of individuals with low or almost no IgG3 responses against either one of these antigens also appeared to be protected. The inventors found that all the protected individuals with low GLURP-R0 specific IgG3 response had significantly elevated levels of specific anti-MSP3 IgG3 antibodies, and vice versa. This observation suggests that antibodies against GLURP and MSP3 may act in a complementary manner to control parasite multiplication in immune individuals. This is relevant in consideration of the role of these antibodies in ADCI mechanism. Indeed, only the simultaneous assessment of several antigens disclosed this complementary effect. This finding is in favor of testing simultaneously several antigens for complementary as for possible antagonistic effects that could have consequences on the design of combined vaccines.

In conclusion, the present study shows that (1)—the critical epitopes in the MSP3 and GLURP antigens which are most conserved, are targets of protective antibodies in geographically distant endemic areas of the world. (2)—IgG3 antibodies to MSP3 and GLURP-R0 are the strongest predictors of protection from clinical malaria in an African and also an Asian setting. (3)—To reach a state of premunition in Asia as well as in Africa, it is needed to produce a cytophilic subclass of antibody against critical antigens (namely, MSP3b and GLURP which both induce antibodies active in ADCI). (4)—There appears to be a complementation effect between these two antigens. IgG3 responses might have similar effects against the risk of malarial attacks, provided they are present against one antigen when responses to the other are low or almost absent. (5)—The responses to different B cell epitopes on a given antigen appear to evolve independently and the level of recognition can change over time.

The complementarity of responses observed to the two main targets of ADCI identified to date provide the first rational basis for combining these two antigens in a hybrid vaccine formulation. Moreover, immunogenicity studies performed in pre-clinical animal models with the hybrid vaccine lend further support to this antigen combination by showing improved immunogenicity with well-balanced, equilibrated responses to each molecule.

### Example 8

### Identification of a Conserved Region of Plasmodium falciparum MSP3 Targeted by Biologically Active Antibodies to Improve Vaccine Design

In this example, MSP3 designates MSP3-1.

The merozoite surface protein-3 (MSP3) is a target of antibody-dependent cellular inhibition (ADCI), a protective mechanism observed in humans immune to Plasmodium falciparum malaria. From the C-terminal half of the molecule which is highly conserved, six overlapping peptides were chosen to characterize immune responses. Each of the six peptides defined at least one non-cross reactive B-cell epitope. However, distinct patterns of antibody responses, both in terms of levels and IgG subclass distribution, were observed in inhabitants of endemic area. Antibodies affinity-purified towards each peptide differed in their functional capacity to mediate parasite killing in ADCI assays: 3 out of the 6 overlapping peptides had major parasite growth inhibitory effect. This result was further confirmed by passive transfer of anti-MSP3 antibodies in vivo in the P. falciparum-infected immunocompromised BXN mouse model. T-helper cell epitopes were identified in each of the three peptides investigated. Thus, antigenicity and functional assays converge to identify a 70 amino acid conserved domain of MSP3, as a target of biologically active antibodies to be included in future vaccine constructs based on MSP3.

The asexual blood stage multiplication of the malarial parasite is responsible for the acute symptoms of malaria in humans. Epidemiological observations have shown that adults residing in the endemic areas, though constantly infected and frequently carrying parasites control the level of their parasitemia and show substantial clinical resistance as compared to children (Baird J K, Jones T R, Danudirgo E W, et al, 1991). Repeated infections and continued exposure to the parasite are required to reach this level of immunity against the disease (McGregor I A, Wilson, R J M., 1989). This state of naturally acquired immunity against the disease, a phenomenon called premunition (Sergent E, Parrot L., 1935), is not a sterile immunity and is marked by chronic low-grade parasitemia without clinical symptoms.

Passive transfer of serum immunoglobulin (IgG) from clinically immune individuals has been shown to be able to control disease and the level of parasitemia in non-protected individuals exposed to geographically diverse parasite strains (Cohen S, McGregor I A, Carrington S., 1961 ; Edozien J C, Gilles H M, Udeozo 10., 1962 ; Sabchareon A, Burnouf T, Ouattara D, et al., 1991). We have earlier found that the protective IgG has no major direct effect on the parasite invasion and growth in the red blood cells, but acts in association with blood monocytes, through an antibody dependent cellular inhibition (ADCI) mechanism that inhibits parasite development (Bouharoun-Tayoun H, Attanath P, Chongsuphajaisiddhi T, Druilhe P., 1990). The cytophilic nature of the protective IgG has been established (Bouharoun-Tayoun H, Druilhe P., 1992; Bouharoun-Tayoun H, Druilhe P, 1992) and the importance of these antibodies in protection against malaria has also been demonstrated in other independent studies (Oeuvray C, Theisen M, Rogier C, Trape J F, Jepsen S, Druilhe P., 2000; Groux H, Gysin J., 1990).

Our search for the targets of the protective antibodies, using ADCI as a functional assay, led us to identify MSP3 as one such target (Oeuvray C, Bouharoun-Tayoun H, Gras-Masse H, et al, 1994). MSP3 is associated with merozoite surface molecules possibly through the coiled-coil structures predicted to be formed by the heptad repeats and the C-terminal leucine zipper domain, (Mills K E, Pearce J A, Crabb B S, Cowman A F., 2002). The N-terminal part of the molecule consists of regions, which are polymorphic between different strains. In contrast, the C-terminal part of the molecule is highly conserved between the various isolates of the parasite tested (McColl D J, Anders R F., 1997 ; Huber W, Felger I, Matile H, Lipps H J, Steiger S, Beck H., 1997) and it is this region that was earlier identified by screening of a P. falciparum expression library using functional ADCI assays (Oeuvray C, Bouharoun-Tayoun H, Gras-Masse H, et al, 1994). Previous studies on MSP3 have focused only on a 27 amino acid region (a.a. 184-210 corresponding to the 3D7 strain, MSP3b) of the C-terminal part, which has been earlier identified as a target of protective antibody response in hyperimmune sera (Oeuvray C, Bouharoun-Tayoun H, Gras-Masse H, et al, 1994).

We decided to further characterize the antigenicity of other regions in the C-terminal part of the molecule. Six overlapping peptides were designed (MSP3a, MSP3b, MSP3c, MSP3d, MSP3e and MSP3f) (FIG. 24 and 25) each representing different regions of the conserved C-terminal part of the molecule. They were used to analyze the naturally occurring immune responses in individuals from the endemic village of Dielmo, Senegal, and their potential relationship with protection from malaria attack. The functional role of human antibodies specific to each region, was assessed under in vitro conditions in the ADCI assay and further confirmed by passive transfer in vivo in an immuno-deficient mouse model grafted with P. falciparum infected human RBCs [Badell E, Oeuvray C, Moreno A, et al, Med 2000; and Moreno A, Badell E, van Rooijen N, Druilhe P. Chemother 2001].

This process led us to identify a 70 amino acid region of MSP3 as the target for naturally occurring protective antibody responses.

8A—Material and Methods

Antigens.

MSP3 recombinant protein constructs and peptides were designed based on the P. falciparum 3D7 strain sequence (NCBI protein_id=“NP_{—}700818.1”). Two recombinant hexa-histidine tagged proteins, MSP3-NTHis₂₁₋₁₈₄ and MSP3-CTHis₁₉₁₋₃₅₄ were purified as previously described [Theisen M, Vuust J, Gottschau A, Jespen S, Hogh B. 1995]. The six peptides MSP3a₁₆₇₋₁₉₁, MSP3b₁₈₄₋₂₁₀, MSP3c₂₀₃₋₂₃₀, MSP3d_{211-252,} MSP3e₂₇₅₋₃₀₇ and MSP3f₃₀₂₋₃₅₄ correspond to the conserved region of MSP3 C-terminal region. A small region (a. a. 253-274; 72% glutamic acid) was excluded from this analysis, as glutamate-rich antigenic determinants exhibit cross-reactivity among several different P. falciparum antigens [Mattei D, Berzins K, Wahlgren M, et al, 1989]. The peptides were synthesized according to standard peptide synthesis procedures [Roggero M A, Servis C, Corradin G. 1997].

Human Serum and Lymphocyte Samples.

For affinity purification of antibodies specific of each MSP3 region, we relied on sera from thirty hyperimmune individuals from Ivory Coast which had been previously used for passive transfer experiments in Thai malaria patients and found to be effective in controlling their disease and parasitemia [Sabchareon A, Burnouf T, Ouattara D, et al. 1991].

For immuno-epidemiological studies we relied on plasma samples from 48 permanent residents of the Dielmo village from Senegal, West Africa, with various degree of exposure to malaria (age between 3.5 and 53.4 years; mean age=13.1±1.8 years; mean stay in the village 707 out of 730 days of follow up). In this region malaria transmission is intense and perennial (≈200 infected mosquito bites/person/year), over the 2-year period the mean number of malaria attacks was 2.4±5.4 episodes per person. 19 individuals had no malaria attack (mean age 15.7±3.1 years) whereas, 29 individuals had at least one malaria attack (mean age 11.4±2.2 years) during the following 2 years. All Dielmo inhabitants were actively followed-up by medical doctors on a daily basis for febrile episodes and those due to malaria were accurately diagnosed as described [Trape J F, Rogier C, Konate L, et al, 1994]. This allowed us to examine the pattern of IgG isotype response towards different regions of MSP3 in individuals clearly distinguished as ‘protected’ (no malaria attack) or ‘non-protected’ (≧1 malaria attack) over 2 years follow-up period of the present study. This group was representative of the whole village in terms of age distribution with respect to occurrence or absence of malaria attack.

Mononuclear cells obtained from Dielmo inhabitants, were carried back within 4 hours to Dakar laboratories and used for T-cell proliferation and IFN-γ against MSP3a, MSP3b and MSP3c peptides according to previously described methods [Behr C, Sarthou J L, Rogier C, et al, 1992; and Bottius E, BenMohamed L, Brahimi K, et al, 1996]. Briefly, the proliferative responses of the cells were assessed in quadruplicates in 96-well round bottomed plates (Nunclon®) by incubating for 6 days at 37° C. in 5% CO₂ in presence of each peptide used at 10 μg/ml, followed by addition of 1 μ Ci of [³H] TdR overnight and counting of incorporated radioactivity in a liquid scintillation counter. Unstimulated cultures served as negative controls and PPD and PHA as positive controls. The IFN-γ concentration in pooled supernatants from quadruplicate wells was assessed by a capture ELISA assay performed in duplicates, using anti-human IFN-γ mAb 350B10G6 and biotin-labeled mAb 67F12A8 (Biosource) for coating and revealing respectively, according to the manufacturer's instructions. The reaction was revealed using streptavidin-HRP and TMB chromogen and optical density was measured at 450 nm. For practical reasons, mainly the number of cells available per donor, the other 3 peptides used for antibody assays could not be included in T-cell assays. Lympho-proliferation studies were performed with samples from 61 inhabitants (29 females and 32 males, mean age 27.31 yr), and IFN-γ secretion was studied in 31 of them (19 females and 12 males, mean age 33.94 yr). The three peptides proved to induce no significant response in PBMC of 16-control non-malaria exposed donors (data not shown), indicating that they have no mitogenic or superantigenic effect.

Enzyme-Linked Immunosorbent Say (ELISA).

The assay was performed for detecting total IgG and the subclasses as described earlier [Bouharoun-Tayoun H, Druilhe P. 1992; and Bouharoun-Tayoun H, Druilhe P. 1992]. Monoclonal mouse anti-human subclasses IgG1 to IgG4 (clones NL16 (Boehringer), HP6002 (Sigma), Zg4 (Immunotech), and RJ4 (Immunotech)) were selected for their affinity and reactivity for African allotypes and were used as secondary antibodies at 1/2000, 1/5000, 1/5000, and 1/1000 dilutions respectively.

The specific reactivity of each serum was obtained by subtracting the OD value to a control protein (BSA; 0.25 μg/well) from that to the test antigens. For calculating the threshold of significance of antibody responses, a set of eight randomly selected sera from individuals never exposed to malaria was tested against each antigen, as controls. Results were expressed as the ratio of the mean OD from test sera to the mean OD of controls+3× standard deviation of the control sera. Sera were considered to be positive for ratios >1.

Affinity Purification of Antibodies.

Since the ADCI assay requires cooperation of antibodies with Fc-γ RII receptor [Bouharoun-Tayoun H, Attanath P, Chongsuphajaisiddhi T, Druilhe P. 1990], a group of 30 hyperimmune sera from Ivory Coast were first screened for IgG subclass distribution against different MSP3 peptides and recombinants. Sera were selected for affinity purification of antibodies against any given MSP3 construct based on high reactivity against that region with minimal reactivity towards the adjacent peptides, and high content of cytophilic IgG antibodies (IgG1+IgG3). Independent serum pools (each made up of 5 to 7 individual serum samples) were used to affinity purify antibodies to different regions of MSP3. The ratio of cytophilic to non-cytophilic IgG subclasses (IgG1+IgG3/IgG2+IgG4) of the serum pools used were 9.56 for MSP3NT, 4.25 for MSP3CT, 1.29 for MSP3a, 3.86 for MSP3b, 1.29 for MSP3c, 4.58 for MSP3d, 1.59 for MSP3e and 3.68 for MSP3f. Previous studies have shown that the profile of cytophilic antibodies observed in affinity purified antibodies was similar to that of the sera pool used for affinity purification.

Affinity purification was done as described earlier [Brahimi K, Perignon J L, Bossus M, Gras H, Tartar A, Druilhe P. 1993] using a 2.5% aqueous suspension of polystyrene beads (mean diameter of 10 μm, Polysciences, Ltd.) to coat the peptides or recombinant proteins. Specific antibodies were eluted using 0.2 M glycine pH 2.5 and were immediately neutralized to pH 7.0 using 2M aqueous Tris solution. Affinity-purified antibodies were dialyzed extensively against PBS followed by RPMI and concentrated using Centricon concentrators (Millipore), filter sterilized and following addition of 1% albumax (Gibco, BRL) stored at 4° C.

Affinity-purified antibodies were used at a concentration of 10 μg/ml in ELISA to ascertain their specificity and isotype distribution.

Immunofluroscence Assay (IFA).

Since the ability of the antibodies to recognize the native parasite protein is the critical factor in biological assays, IFA was used to adjust the concentration of affinity-purified antibodies. IFA was performed on air-dried, acetone-fixed, thin smears of P. falciparum mature schizonts as described earlier [Druilhe P, Khusmith S. 1987], to assess binding activity of affinity-purified antibodies to the parasite protein. The effective concentration of each antibody was adjusted to 1/200 IFA end-point titer for use in functional assays.

Functional in vitro Antibody Assays.

The Antibody-dependant Monocyte-mediated ADCI assays were performed in duplicates using laboratory maintained strain 3D7 and UPA (Uganda Palo-Alto) as described previously [Bouharoun-Tayoun H, Attanath P, Chongsuphajaisiddhi T, Druilhe P. 1990]. Monocytes from healthy, non-malaria exposed donors were prepared as previously described [Bouharoun-Tayoun H, Attanath P, Chongsuphajaisiddhi T, Druilhe P. 1990]. The affinity-purified antibodies, adjusted to a concentration yielding a 1/200 IFA end-point titer, were added at a rate of 10 μl in 90 μl of complete culture medium, i.e., used at a final titer of 1/20 in the ADCI assay. Following cultivation for 96 h, parasitemia was determined on Giemsa-stained thin smears from each well by microscopic examination of ≧50,000 erythrocytes. Monocyte-dependent parasite inhibition is expressed as the specific growth inhibition index (SGI): SGI=1-{(percentage of parasitemia with monocytes and test IgG/percentage of parasitemia with test IgG)/(percentage of parasitemia with monocytes and normal IgG/percentage of parasitemia with normal IgG)}×100. Although the SGI calculation takes into account a possible direct anti-parasite effect of monocytes, since this is observed with only 10-15% of monocyte preparations, we excluded as an additional safety measure monocyte preparations that had a direct anti-parasitic effect.

Passive Immunisation of P.falciparum-Infected Immunocompromised Mice.

The use of the P.f.-HuRBC-BXN mouse model for assessing the effect of antibodies to different blood stage antigens of P. falciparum has been detailed earlier [Badell E, Oeuvray C, Moreno A, et al, 2000]. 6-8 week old male Beige-Xid-Nude (BXN) mice (Charles River Laboratories) manipulated under pathogen free conditions were treated with liposomes containing dichloromethylenediphosphonate (CI₂MDP) (Roche Diagnostics Mannheim, Germany) and anti-polymorphonuclear neutrophil (PMN) monoclonal antibody NIMP-R14 (NIMR, London, UK) to reduce their innate immune response. P. falciparum infected human red blood cells were injected IP on day 0 and uninfected red blood cells injected at 4-day intervals. Blood parasitaemia was followed-up microscopically. Mice with stable parasitemia (in the range of 0.1-1%) were grafted IP with 3×10⁶ human peripheral blood monocytes, actively selected by CD14⁺ magnetic beads (MACS, Miltenyi Biotech) followed 24 hours later by 3×10⁶ monocytes together with 200 μl of affinity-purified antibodies to MSP3 at 1/200 IFA end-point titer as described earlier. Non-specific esterase staining [Bouharoun-Tayoun H, Attanath P, Chongsuphajaisiddhi T, Druilhe P. 1990] showed that >98% of CD14⁺ cells were made of monocytes.

Statistical Analysis.

Univariate analysis was performed using Mann-Whitney U test. Fisher's exact test was used for contingency table analysis. The association between the risk of malaria attack and the levels of antibodies was tested with JMP® software, using a stepwise regression model where the confounding effect of age was controlled for. The analysis of variance was applied to the regression model. The test of the null hypothesis was based on the variance ratio denoted by F, and departures from the null hypothesis tended to give values of F greater than unity.

8B -Results

Non-Cross Reactive B-Cell Epitope Defined by each of the 6 MSP3 C-Terminal Peptides.

IgG responses were measured against different regions of MSP3 C-term in a group of 30 hyperimmune sera from Ivory Coast. As shown in FIG. 20, there were differences in the levels and prevalence of IgG towards each region, but antibody responses were detected against each of the C-term peptides.

Antibodies were then affinity-purified from selected hyperimmune sera specific to each peptide, and studied for their reactivity against the other peptides. In this way, it was possible to affinity purify antibodies specific of each peptide which did not show cross-reactivity with other regions (table 5). These observations indicate that each of the peptides covering MSP3 C-term defines at least one B-cell epitope that does not share antigenic determinants with other regions. Each of the affinity-purified antibodies was also found to be positive in immunofluroscence assays on P. falciparum asexual blood stages indicating that anti-peptide antibodies were reactive with the native parasite protein (data not shown).

Distinct Isotype Patterns of the IgG Response toward Different MSP3 Peptides.

We analyzed plasma from 48 individuals, 3 to 53 years old, from the endemic village of Dielmo, Senegal, to study the distribution and the pattern of IgG isotype response against the different regions of C-terminal part of MSP3 defined by the peptides.

As shown in FIG. 21, both the levels of antibody response and the pattern of IgG isotypes were distinct against each region. The prevalence of responders varied for each region of MSP3 (from 6.25% to 60.41% for IgG1, 4.16% to 47.91% for IgG3, 0% to 10.41% for IgG2 and 0% to 12.5% for IgG4). We found that antibodies to MSP3a and MSP3e were less prevalent and when present, were only detected at low levels. Antibodies to MSP3b, MSP3c, MSP3d and MSP3f were the most prevalent and were predominantly of cytophilic subclasses. Among the cytophilic isotypes, IgG3 reactivity was found to be predominant against MSP3b, MSP3c and MSP3d. On the contrary, IgG1 reactivity against MSP3f was stronger and more prevalent than IgG3. This suggests that antibody response elicited to any region of MSP3 was not dependent on response to other regions.

It has been earlier observed that the cytophilic IgG response plays an important role in protection from malaria [Bouharoun-Tayoun H, Druilhe P. 1992; Bouharoun-Tayoun H, Druilhe P. 1992; Oeuvray C, Theisen M, Rogier C, Trape J F, Jepsen S, Druilhe P. 2000; Groux H, Gysin J. 1990]. We further addressed the relationship between clinical protection that had been monitored on a daily basis, and the pattern of isotype responses observed against each peptide. In the present study, ‘protection’ was defined as the absence of any clinical malaria attack during the two years following the plasma sampling. Higher IgG3 titers against MSP3b, MSP3c and MSP3d were observed among protected, as compared to non-protected, subjects. An association between the levels of IgG3 antibodies directed to MSP3b, and MSP3d, and protection from occurrence of malaria attack (‘p’ values of 0.037 and 0.057 respectively) was observed. In the case of MSP3c, this association did not reach statistical significance, however anti-MSP3c IgG3 antibodies were twice higher in individuals without than with malaria attacks. Association between levels of IgG1 and protection against malaria attack was observed to be significant for MSP3d (p=0.025) and a similar trend was observed for MSP3b (p=0.328), but not for MSP3c. Both IgG1 and IgG3 responses to MSP3f were not found to be associated with protection. IgG2 and IgG4 antibody responses against different regions of MSP3 were detected only at low levels, and were not found to be associated with protection.

In a further step, a multivariate stepwise regression analysis was performed so as to control for age, using dichotomous variables of both antibody response (classified as ‘responders’ or ‘non-responders’) and occurrence of malaria attack (classified as ‘protected’ or ‘non-protected’). A significant association of protection with IgG3 anti-peptide responses was observed against 3 out of the 6 peptides: MSP3b (F ratio=4.98, p=0.025), MSP3c (F ratio=3.02, p=0.082) and MSP3d (F ratio=6.57, p=0.01), but not against the other three peptides.

### Inhibition of Parasite Growth by Naturally Occurring Antibodies Against MSP3b, MSP3c and MSP3d in Functional in vitro ADCI Assays

In order to assess the function of naturally occurring human antibodies to different regions of MSP3 in ADCI assays, each affinity-purified antibody was adjusted to a concentration yielding the same reactivity to the native parasite protein. Results (FIG. 22), show that the level of parasite inhibition elicited by antibodies against the recombinant proteins MSP3NT and MSP3CT were comparable to that observed for the pool of African IgG (PIAG) previously used for passive transfer experiment in humans [Sabchareon A, Burnouf T, Ouattara D, et al. 1991].

Anti-MSP3b, MSP3c and MSP3d affinity-purified antibodies were found to exert a strong monocyte-mediated, anti-parasitic activity in ADCI, comparable to antibodies against MSP3CT and PIAG, whereas, anti-MSP3a and anti-MSP3f antibodies were not found to have parasite inhibitory activity (FIG. 22). Anti-MSP3e antibodies showed only marginal anti-parasite activity i.e., slightly higher than the threshold level of significance. Results were reproducible among four independent ADCI assays. No merozoite invasion inhibitory effect was recorded at 24-96 hours with any of the above antibodies at the concentrations employed.

Strong Reduction of P. Falciparum Parasitemia by Anti-MSP3b and Anti-MSP3d Antibodies in a Humanized Mouse Model.

The observation from the in vitro ADCI assays, that anti-MSP3b, MSP3c and MSP3d antibodies were strongly effective at inhibiting parasite growth, was further assessed in vivo using the P.f.-HuRBC-BXN mouse model. The value of this new mouse model for studying the in vivo effect of human antibodies and anti-malarial drugs upon the blood stage growth of P. falciparum has been recently documented [Badell E, Oeuvray C, Moreno A, et al, 2000; and Moreno A, Badell E, van Rooijen N, Druilhe P. 2001]. However, given the difficulty of handling of this new model, only antibodies found to have a marked anti-parasitic effect under in vitro conditions were evaluated in vivo in passive transfer experiments. Antibodies to MSP3d were compared to anti-MSP3b antibodies, used here as positive controls which anti-parasitic effect has been earlier demonstrated [Badell E, Oeuvray C, Moreno A, et al, 2000].

As seen in FIG. 23, the parasitemia increased and reached a plateau over the next 3 weeks. Injection of peripheral blood monocytes alone on day 22 did not affect the parasite growth, in keeping with earlier observations [Badell E, Oeuvray C, Moreno A, et al, 2000]. The injection of the affinity-purified anti-MSP3 human antibodies, on day 23, resulted in a sharp decrease of the parasitemia. Passive transfer of anti-MSP3b antibodies resulted in clearance of the parasites. The passive transfer of anti-MSP3d resulted in a decrease of greater than 95% (FIG. 23). Thus, results from the in vivo passive transfer in this mouse model confirmed the in vitro results and further validated the functional anti-parasite activity of naturally occurring antibodies against the 70 amino acids region covered by peptides MSP3b and MSP3d.

T-Cell Responses Against MSP3 Peptides in Malaria-Exposed Individuals.

T-lymphocyte responses could be studied only against three (MSP3a, MSP3b and MSP3c) of the six C-terminal peptides in inhabitants from the village of Dielmo, Senegal, due to practical limitations in field. Proliferative response determined using peripheral blood lymphocytes from 61 individuals (aged 1 to 84 yr; mean age 27.34 yr) showed that prevalence of T helper-cell responders were 16.4% against MSP3a, 28% against MSP3b and 21.3% against MSP3c respectively. IFN-γ secretion monitored in 31 of these individuals showed that prevalence of IFN-γ responders was 42% against MSP3a, 55% against MSP3b and 61.3% against MSP3c. These results indicate that each of the three MSP3 peptides tested defines at least one T-cell epitope. In addition, IFN-γ secretion suggests that at least some of the responding cells belonged to the Th1-like type.

8C—Discussion

In the search for malaria vaccine candidates, we focused our studies on antigens targeted by the most potent immunity, i.e. immunity acquired over the years by individuals living in hyperendemic areas. We have described that this non-sterilizing immunity (“premunition”) is mediated by IgG that are active through an indirect mechanism, implicating monocytes (ADCI). In the second step, ADCI was used to identify MSP3 as a target of protective IgG [Oeuvray C, Bouharoun-Tayoun H, Gras-Masse H, et al, 1994]. The present study was aimed at characterizing antigens within the conserved C-terminus of MSP3 and evaluating the function and biological effects of the corresponding antibodies.

Indeed, the C-terminal half of the molecule, starting from the third heptad repeat, is highly conserved in the different isolates tested, so far [McColl D J, Anders R F. 1997; and Huber W, Felger I, Matile H, Lipps H J, Steiger S, Beck H. 1997], whereas the N-terminal half of MSP3 shows an overall dimorphism (3D7-like and K1-like) [McColl D J, Anders R F. 1997; and Huber W, Felger I, Matile H, Lipps H J, Steiger S, Beck H. 1997]. Therefore, we decided to focus on the C-term region, because a part of it (DG210, FIG. 19) was identified to be a target of protective human antibodies in our initial screen [Oeuvray C, Bouharoun-Tayoun H, Gras-Masse H, et al, 1994], and second, because antigen conservation is a critical criterion for successful malaria vaccine development.

Using six overlapping synthetic peptides covering the conserved C-terminal half of MSP3 we show that antibody patterns to each region differ markedly in terms of prevalence, titer, isotype distribution, association with clinical protection, and anti-parasitic activity in vitro and in vivo. Antibody titers against MSP3a and MSP3e were low as compared to the remaining four peptides. Responses to MSP3b, MSP3c, MSP3d and MSP3f were made mostly of cytophilic IgG subclasses, however being predominantly of IgG1 isotype against MSP3f, and predominantly of IgG3 to the others. A similar difference of subclass response to distinct regions of a single protein has been reported for another merozoite surface protein of P. falciparum, MSP-1 [Cavanagh D R, Dobano C, Elhassan I M, et al, 2001]. These observations suggest that IgG class switching involved during the maturation of antibody response towards different regions of MSP3 C-term is regulated independently. The factors regulating the maturation of antibodies are not well understood but would be influenced by the nature of the antigen in conjunction with contact-dependent signals from T-cells particularly the cytokines they secrete [Stavnezer J. 1996]. Recent observations suggest however that the nature of the malaria antigen might be the major factor determining the antibody subclass [Garraud O, Perraut R, Diouf A, et al, 2002], which seems to be the case in our study.

Availability of very detailed clinical information, which is a major characteristic of the set-up in the village of Dielmo, Senegal, led us to address subclass patterns in relation to protection from the occurrence of malaria attacks. Taking in to account the confounding effect of age, we observed that IgG3 response to MSP3b, MSP3c and MSP3d were significantly associated with protection from the occurrence of malaria attacks. These results are in agreement with independent studies involving larger sample sizes [Soe S, Theisen M, Roussilhon C, Aye K S, Druilhe P. 2003; and Oeuray, C., et al, in preparation], which have shown association between IgG3 response against MSP3b and protection to malaria. For other merozoite surface vaccine candidates, a skewing towards IgG3 antibody response has been reported for MSP2 in various ethnic groups and different conditions of malaria transmission [Taylor R R, Smith D B, Robinson V J, McBride J S, Riley E M. 1995; and Rzepczyk C M, Hale K, Woodroffe N, et al, 1997], and could be correlated with clinical immunity to malaria [Taylor R R, Allen S J, Greenwood B M, Riley E M. 1998]. Similarly, the antibody response to the polymorphic ‘block 2’ region of MSP1, which has been identified as a target of immunity to clinical malaria, is also skewed towards IgG3 subclass [Polley S D, Tetteh K K, Cavanagh D R, et al, 2003]. However, at least in the latter case, the mechanism of action of these antibodies remains elusive, since it is generally assumed that biologically active anti-MSP1 antibodies are directed to the C-terminal part of the antigen [Egan A F, Burghaus P, Druilhe P, Holder M, Riley E M. 1999].

In contrast, in our study the use of functional in vitro ADCI assays provided information about the anti-parasitic, biological activity of antibodies towards various regions. Performed under conditions allowing for comparisons, they demonstrated critical differences in antibodies targeting different regions of MSP3. It is of interest that very different approaches led to similar conclusions, i.e., the in vitro ADCI assays pointed to the importance of exactly the same peptides (MSP3b, MSP3c and MSP3d), as those indicated by the immuno-epidemiological studies. The reasons for the lack of effect of antibodies to MSP3a and MSP3f remain to be investigated. In the case of MSP3f, it is possible that antibodies might not access this epitope on the merozoite surface, as this leucine-zipper domain forms coiled-coil interactions with other molecules [Mills K E, Pearce J A, Crabb B S, Cowman A F. 2002; and McColl D J, Anders R F. 1997].

The reliability of in vitro findings could also be confirmed under in vivo conditions [Badell E, Oeuvray C, Moreno A, et al, 2000]. Upon passive transfer in P. falciparum-infected mice grafted with human monocytes and with long-lasting stable parasitemia, anti-MSP3b and anti-MSP3d antibodies were found to be effective in reducing P. falciparum parasite load.

The vaccine potential of MSP3 was recently confirmed by the protection elicited against P. falciparum challenge in Aotus nancymai monkeys immunized with full-length MSP3 in Freund's adjuvant [Hisaeda H, Saul A, Reece J J, et al, Merozoite 2002]. This observation is in agreement with our epidemiological and biological findings. However, the present study provides additional information derived from the analysis of human immune responses for the design of future vaccine constructs. Indeed, the N-terminal of MSP3, though able to induce antibody with functional activity in ADCI, is of debatable value due to its polymorphism. Furthermore, its inclusion could divert the immune response away from the important conserved region. Within the C-terminal part, the region MSP3e-f was also found less valuable due to low prevalence and low levels of antibody responses to MSP3e and anti-MSP3f antibodies devoid of biological effect. Each of the 3 peptides, a, b, c, investigated proved to define a non-cross-reactive T-cell epitope for endemic area populations. Recent vaccine trials performed using the construct defined in the present study confirmed this finding and designated peptide “d” as an additional T-cell epitopic region (Audran et al, submitted).

In summary, immuno-epidemiological studies together with functional assays, led us to define a 70 amino acid region of the molecule. We found that antibodies with anti-parasitic effect develop against this region covering MSP3b to MSP3d in human beings naturally exposed to malaria. This information is of practical value for the rational design of sub-unit vaccine constructs derived from MSP3 for future clinical trials.

Table 5. Specificity of Affinity-Purified Human Anti-MSP3 Antibodies determined by ELISA

TABLE 5
Specificity of affinity-purified human anti-MSP3
antibodies determined by ELISA

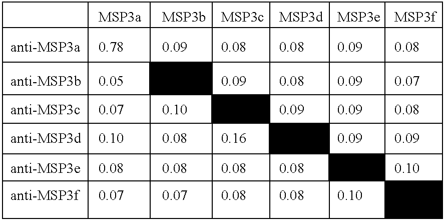

Mean O.D.₄₅₀ values from duplicate wells are shown. All the peptides were used under identical coating conditions. Shading represents positive reactivity.

### Example 9

### A Merozoite Surface Antigen Family of P. falciparum Ensures Parasite Survival

Among the molecules expressed at the surface of P. falciparum merozoite, Merozoite Surface Protein 3 (MSP3) is a novel vaccine candidate identified by screening whole genome expression products using an in vitro ADCI assay based on defense mechanism identified as essential for protection against malaria in humans (Oeuvray, et al., 1994). Anti-MSP3 antibodies inhibit the parasite growth by triggering the release of parasitostatic monokines (Bouharoun-Tayoun, et al., 1995). In several field settings, the IgG3 anti-MSP3 antibodies are strongly associated with the state of acquired immunity to malaria (Soe, et al., 2004; Singh, et al., 2004). A vaccine trial in 36 volunteers led to the induction of antibodies in humans that could trigger killing of P. falciparum both under in vitro and in vivo conditions (Druilhe, et al., manuscript under preparation).

Analysis of the P. falciparum genome data recently identified a novel MSP3 multi-gene family, members of which share structural homologies with MSP3 (Cowman & Crabb, 2002). Homologues of MSP3 identified in P. vivax and P. knowlesi have been reported to consist of several related molecules (Galinski, et al., 2001; David, et al., 1985). In P. falciparum, homologies among MSP3-like molecules concern a signature peptide in the N-term present in all related molecules, the overall organization of the C-terminal region of the molecules together with sub-domains of higher homology, which have been found in MSP3 to constitute the target of cytophilic antibodies associated with protection in the field and mediating parasite killing both in vitro and in vivo (Singh, et al., 2004).

In view of the vaccine potential of MSP3, particularly the encouraging results obtained in the clinical vaccine trial, we decided to investigate in detail the other members of the family with respect to gene expression, localization of the proteins encoded by them, the extent of antigenic relatedness, the conservation of their sequences and the functional role of antibodies against parasite growth.

Results show that this multi-gene family differs in many aspects from other P. falciparum multi-gene families described so far and suggests that they play an important role in eliciting immune responses involved in parasite density control and, in general, in defense mechanisms in the human host.

9A—Materials and Methods

Sequence Analysis

Searches of the P. falciparum 3D7 database were done using GenBank blasts at NCBI (http://www.ncbi.nlm.nih.gov/Malaria/plasmodiumbl.html). All BLAST searches were done without the low-complexity filter and with all other settings kept at default. Pairwise homology was performed between different protein sequences using Wilbur-Lipman algorithm, PAM 250 using the Gene Jockey II sequence analysis software. ClustalW was used to produce the multiple alignments (http://www.ebi.ac.uk/cgi-bin/newclustalwpl), which were copied into Boxshade Hofmann, Barron (at http://bioweb.pasteur.fr/seqanal.interfaces/boxshade.html#letters) to produce the alignments. Prediction of the signal peptides was done using iPsort and Signal P (at http://hypothesiscreator.net/iPSORT/predict.cgi and http://www.cbs.dtu.dk/services/signalp/#submission, respectively). Prediction and analysis of coiled-coil regions from amino acid sequences was performed with the COILS2.1 program (Lupas, et al., 1991).

Prediction of two and three-stranded coiled-coil regions was performed with the PAIRCOIL based MULTICOIL program (Wolf, et al., 1997). Leucine zipper predictions were based on the LZpred program (Bornberg-Bauer, et al., 1998) that combines a coiled-coil prediction algorithm with an approximate search for the characteristic leucine repeat. “Unique regions” represent regions of least relatedness between different members of the MSP3-family of proteins. They consist of around 50-80 amino acid residues (see table 6 A), which were identified by analyzing homology alignment between the amino-acid sequences of different members. The sequences of the “unique regions” used as queries in a BLASTP search, ascertained that they did not show any significant relatedness (‘score bit’ value to themselves in the BLAST was always greater than 100 with ‘E values’ in the range of 9e-23 to 1e-40; a few other hits obtained only against MSP3.3, MSP3.4 and MSP3.5 were with a low ‘score bit’ value of less than 40 with ‘E values’ not less than 1e-04) to the primary amino-acid sequence of any other P. falciparum protein in the database. Another set of recombinant proteins were designed to cover the related C-terminal regions of MSP3.1, MSP3.2, MSP3.3, MSP3.4, MSP3.7 and MSP3.8 as shown in FIG. 27 and table 6B).

Cloning and Expression of Recombinant Proteins

“Unique region” and “related carboxy-terminal” recombinant proteins were cloned from 3D7 strain genomic DNA and expressed and purified as N-terminal his-tagged recombinant proteins as described elsewhere (Theisen, etal., 1995).

RNA Analysis

RNA was extracted from asynchronous blood stage parasite culture (3D7, harvested at 10-15% parasitaemia) using TRIZOL (Life Technologies), according to the manufacturer's instructions. RNA pellets were stored at −20° C. To ensure that RNA was completely free of contaminating DNA, it was treated with DNasel using DNA-free kit (Ambion). First-strand of cDNA was synthesized from around 1 μg of DNA-free RNA using a set of random primers and M-MLV Reverse tanscriptase (Invitrogen) following the supplier's instructions. Amplification of the unique regions of MSP3-family of genes was done using the set of primers listed in table 6A. Controls consisting of genomic DNA as template and no nucleic acid (water as template) were included for each primer pair.

Western Blot and Dot-Blot Assays

Western blot analysis was performed against parasite proteins resolved on a 12% SDS-PAGE under denaturing conditions using standard protocols as described elsewhere (Bouharoun-Tayoun & Druilhe, et al., 1992). Dot-blot assay was performed using purified recombinant proteins on strips of nitrocellulose paper (Amersham). In order to obtain comparable protein distribution of proteins in each dot sample, both the concentration and volume was adjusted. Typically 2 μg/10 μl of purified recombinant protein was applied to the nitrocellulose membrane using a vacuum manifold (BioRad). Dot-blots were subsequently processed for antibody signal detection similar to the Western blot strips.

Indirect Immunofluorescence Assay (IFA)

IFA was performed on air-dried, acetone-fixed, thin smears of P. falciparum mature schizonts, as described elsewhere (Druilhe & Khusmith, 1987). IFA was used to detect subcellular localization of proteins and to adjust the functional concentration of the affinity-purified antibodies for use in ADCI assays, as described elsewhere (Singh, et al., 2004).

ELISA

ELISA was performed for the detection of total IgG and subclasses, as described elsewhere (Druilhe & Bouharoun-Tayoun, 2002) in a pool of hyperimmune were against different members of the MSP3-family of proteins. Sera from mice immunized with MSP3.1 and MSP3.2 recombinant proteins were also tested for their ability to cross-react with other members of the MSP3-family. The specific reactivity of each serum sample (human/mouse) was obtained by subtracting the optical density value of a control protein (0.25 μg of bovine serum albumin/well) from that of the test antigens.

Affinity Purification of Antibodies

Antibodies were affinity-purified against different members of the MSP3-family of proteins from a pool of hyperimmune sera obtained from the inhabitants of the village of Dielmo, Senegal, West Africa. Affinity purification was done as described elsewhere (Singh, et al., 2004) using purified recombinant protein adsorbed on the surface of polystyrene beads (mean diameter, 10 μm; Polysciences). Specific antibodies were eluted by use of 0.2 M glycine (pH 2.5) and were immediately neutralized to pH 7.0 using 2M aqueous Tris solution. Affinity-purified antibodies were dialyzed extensively against PBS followed by RPMI and were concentrated using Centricon concentrators (Millipore), filter sterilized, and, after addition of 1% albumax (Gibco BRL), stored at 4° C.

Cross-Reactivity Studies

The degree of antigenic relatedness between different carboxy-terminal recombinant proteins from members of the MSP-family of proteins was assessed by testing the cross-reactivity of antibodies generated against them. To test the existence of antigenic relatedness among different members of the MSP3 family of proteins, ELISA assays were performed using antibodies affinity-purified from hyperimmune sera at a concentration of 10 μg/ml in ELISA.

Cross-reactivity of mice sera generated against MSP3.1 and MSP3.2 C-terminal recombinant proteins, sera tested against other members of the MSP3-family of proteins by performing ELISA, using 1:50 dilution of the mice sera.

Avidity Studies

Antibody binding avidity was determined for naturally occurring human antibodies against different members of the MSP3-family of proteins using dot-blot assay. Identical strips of nitrocellulose membranes arrayed with equal amount of recombinant proteins were tested for residual antibody binding after treatment with increasing concentrations of chaotropic salt (NH4SCN: 0M, 0.1 M, 0.25 M, 0.62 M, 1.56 M and 3.9 M) for 20 min at room temperature. Values obtained for the antibody reactivity against any antigen in presence of 0M NH4SCN solution was considered to be 100%, and the residual antibody reactivity after treatment with higher concentrations of NH4SCN was expressed as fractions of this 100%. Quantitative assessment of antibody reactivity was done by Adobe Photoshop based image analysis after scanning the dot-blots using EPSON scanner (model: EU34; EPSON TWAIN software). The image was analysed with Adobe Photoshop software (version 6, Adobe Systems) using a Macintosh PowerPC G4 system. Briefly, a fixed pixel area was selected from the nitrocellulose membrane containing both the “dot-staining” due to the antibody reactivity together with a portion of “background” (surrounding unstained nitrocellulose membrane). This pixel-area was saved using the “save selection” option (Select-menu) and was used to generate histograms (Image-menu). The histograms were set to display statistical details of the selected pixel area in the “luminosity” channel. The “Std Dev” value in the histogram represented the level of contrast between the bright areas (nitrocellulose background) and the dark area (staining due to antibody reaction), and was used for comparing the levels of residual antibody reactivities.

Functional in vitro Antibody Assays

The antibody-dependent, monocyte-mediated ADCI assays were performed in duplicate by use of laboratory-maintained strains 3D7 and Uganda Palo-Alto, as described elsewhere (Bouharoun, et al., 1990). Monocytes from healthy, non-malaria-exposed donors were prepared as described elsewhere (Bouharoun, et al., 1990). The affinity-purified antibodies, adjusted to a concentration yielding a 1/200 IFA end-point titer, were added at a rate of 10 μL in 90 μL of complete culture medium, which yielded a final titer of 1/20 in the ADCI assay. After cultivation for 96 h, the level of parasitemia was determined on Giemsa-stained thin smears from each well by the microscopic examination of 50,000 erythrocytes. Monocyte-dependent parasite inhibition is expressed as the specific growth inhibition index (SGI): of parasitemia SGI=1−([percentage of parasitemia with monocytes and test IgG/percentage of parasitemia with test IgG)/(percentage of parasitemia with monocytes and normal IgG/percentage of parasitemia with normal IgG]). A positive control IgG, from the pool of serum samples from Ivory Coast used for passive-transfer experiments in humans (Sabchareon, et al., 1991) and a negative control IgG, from French donors who were never exposed to malaria infection, were included in the assay.

9B—Results

Six of the Eight ORFs Located in Tandem with MSP3 on Chr.10 Share Similar Sequence Organization.

Homologues of P. falciparum MSP3 have been identified in different species of malaria (Galinski, et al., 2001; David, et al., 1985), and in some species these homologues exist as multi-allelic gene family. Recently, another P. falciparum merozoite surface protein MSP6, related to MSP3 has been decribed. MSP3 and MSP6 share an ordered sequence organization in their C-terminal regions, consisting of antigenic domains targeted by protective antibodies followed by a glutamic-acid rich region and a coiled-coil region (Trucco, et al., 2001). All known MSP3-like genes in different parasite species share a 4-6 amino acid signature motif (NLRNA/NLRNG) in the N-terminal region, shortly after the predicted 22-24 amino acid signal peptide sequences.

Analysis of the P. falciparum genome (http://www.ncbi.nim.nih.gov/Malaria/plasmodiumbl.html) for genes with this signature-motif identified a contig of 32 kb on chromosome 10, containing 8 ORFs located in tandem on the same coding strand, but different reading frames. We propose to rename these genes, based on their sequence relatedness to MSP3, in accordance with their location on the coding strand (from 5′ to 3′ end), as shown in FIG. 26 A. Two of them are known to code for known merozoite surface proteins MSP3 and MSP6 (now renamed as MSP3.1 and MSP3.2 respectively), while others are ascribed to encode, yet uncharacterized hypothetical proteins in the database.

Their protein ids in the database are:

MSP3.1—MN35542.1; MSP3.2—MN35543.1; MSP3.3—MN35544.1; MSP3.4—MN35545.1; MSP3.5—MN35547.1; MSP3.6—AAN35548.1; MSP3.7—AAN35549.1 and MSP3.8—MN35552.1.

All these ORFs contain a related amino-terminal signal peptide region predicted by ip-SORT and Signal P programs. The most-likely cleavage site prediction was between amino acid positions for MSP3.1: 25-26; MSP3.5: 21-22; MSP3.6: 21-22; and for MSP3.7: 24-25. However, the prediction for cleavage was not optimal for MSP3.2, MSP3.3, MSP3.4 and MSP3.8.

Six out of these eight ORFs (MSP3.1, MSP3.2, MSP3.3, MSP3.4, MSP3.7 and MSP3.8) share the same ordered sequence organization of their C-terminal regions, whereas, the two others (MSP3.5 and MSP3.6) have unrelated sequences (FIG. 27). Each of these MSP3-like ORF have highly conserved C-terminal region in different P. falciparum isolates tested (see supplementary data, appendix-5). The Clustal-W alignement of the related sequences I shown in FIG. 26 B. BLASTP analysis (shown in table 6) revealed an overall about 28% identity and about 45% similarity of amino acid residues between these related ORFs whereas, within the related C-terminal regions these values sera about 32% and about 54% respectively. A cladogram (FIG. 26 C) shows the extent of sequence relatedness among these ORFs. Two of these ORFs (MSP3.4 and MSP3.8) consist of DBL-like domains along with 13 to 14 cysteine residues, an arrangement similar to those observed in members of the var or ebl gene families (FIG. 27).

The extreme C-terminal region of MSP3.1, which was reported to be leucine-zipper domain (McColl & Anders, 1997), was not confirmed using different algorithms such as Lzpred, in agreement with the observation in MSP3.2 (Trucco, et al., 2001). However, this extreme C-terminal region, both in MSP3.1 and MSP3.2, were predicted to form coiled-coil domain using COILS2.1 and PAIRCOILS. Existence of similar coiled-coil extreme C-terminal regions was also predicted for MSP3.3, MSP3.4, MSP3.7 and MSP3.8.

All Six MSP3-Like ORFs are Expressed as Merozoite Surface Proteins

We analyzed the RNA and protein expression for all these ORFs in the blood stage culture of P. falciparum. Unique regions (regions with least related amino acid stretch) were identified (ca 70-80 a.a.) within each ORFs, as shown in FIG. 27. BLASTP-analysis of P. falciparum genome using these unique regions as queries specifically matched with their respective ORFs with high alignment scores (100-200) and low ‘E’ values <e⁻³². Low alignment scores (<40) were observed with few other proteins using MSP3.3 and MSP3.4 unique regions.

Using specific primers, unique region from each ORF was amplified from genomic DNA and cDNA preparations from asynchronous asexual blood stages of P. falciparum. Results from the cDNA analysis, show that all the ORFs with related C-terminal regions are transcribed in the blood-stage of the parasite development (FIG. 29 panel A). Among the ORFs, which did not share the C-terminal relatedness, RNA expression was detected for MSP3.5 and MSP3.6. However, the low level of cDNA amplification observed for the unique region of MSP3.5 (FIG. 29, panel A, top right), suggests that the transcript for this ORF is less stable.

Antibodies were affinity-purified against the recombinant proteins designed for unique regions in each ORF, using a pool of African hyperimmune sera, as described elsewhere (Singh, et al., 2004). FIG. 28 shows the specificity of antibodies affinity-purified against each unique region determined by dot-blot analysis. The observed pattern of diagonal reactivity confirms the specificity of affinity-purified antibodies against their respective proteins.

Western blot analysis was performed, using these specific affinity-purified antibodies, to detect the expression of the respective ORF in the asexual blood-stage of the parasite (FIG. 29B). The expression of the native parasite protein was confirmed for the ORFs with related C-terminal region. Though the observed molecular weights of the parasite proteins were largely in agreement with their calculated molecular weights, in few cases like in MSP3.1 the observed molecular weight (about 48 kDa) was much higher than the calculated molecular weight (about 40 kDa), which is in accordance with previous studies (McColl, et al., 1994). Lower molecular weight proteins, recognized by some affinity-purified antibodies, could be due to the proteolytic processing of the nascent protein as known in case of MSP3.2 (Trucco, et al., 2001). However, instability of the protein preparation leading to degradation products cannot be ruled out. Antibodies affinity-purified against MSP3.5 and MSP3.6 antibodies did not recognize specific parasite proteins. Whereas, anti-MSP3.5 antibodies did not react to any parasite protein, anti-MSP3.6 antibodies reacted to several polypeptides, which did not match its calculated molecular weight of 65 k Da (FIG. 29, panel B).

Localization of these proteins in the blood stage of the 3D7 strain of parasite was assessed using indirect IFA, with affinity-purified antibodies against respective proteins (FIG. 29, panel C). Antibodies against the ORFs with related C-terminal region stained the surface of free merozoites, showing a pattern indistinguishable from that observed for MSP3 or MSP6. This pattern of IFA reactivity was also observed in two other laboratory strains of the parasite culture Palo-Alto (Uganda) and T23 (data not shown). Antibodies against MSP3.5 failed to react to the parasite protein (FIG. 29, panel C, top right). It is likely that owing to the less stable transcript, as observed for MSP3.5, it is not expressed in the erythrocytic stage. Antibodies against MSP3.6 reacted to mature schizonts and free merozoites (FIG. 29, panel C). However, since anti-MSP3.4 antibodies displayed obvious cross-reactivities to several parasite proteins in Western blot analysis, its expression remains to be confirmed using more precise antibodies.

Thus, the expression analysis shows that the ORFs, which share C-terminal regions related to MSP3, are expressed in the erythrocytic stage of parasite development as merozoite surface proteins and these constitute the MSP3-family of proteins in P. falciparum.

Antigenic Cross-Reactivity is Observed Against the Related C-Terminal Regions in MSP3-Family Members

In order to determine the extent of antigenic relatedness between different members of the MSP3-family, the related C-terminal regions were expressed as recombinant His-tag proteins, as indicated in FIG. 27. Antibodies were affinity-purified against these recombinant proteins from a pool of African hyperimmune sera. The reactivity of this pool against the different recombinant proteins is shown in FIG. 30. The varying levels and the patterns of antibody subclass reactivity observed against each recombinant protein indicate differences in their antigenic characteristics, as a result of the differences between their sequences.

ELISA determined the reactivity of antibodies affinity-purified against each recombinant protein towards other members of the family. Varying degree of cross-reactivity was displayed by antibodies affinity-purified against different members of the family, as seen in Table 7. While, anti-MSP3.1 antibodies exhibited least cross-reactivity to other members of the family, MSP3.1 was most widely recognized by antibodies affinity-purified against other members of the family. In contrast, though anti-MSP3.4 antibodies displayed highest level of cross-reactivity to other members of the family, MSP3.4 itself was less well recognized by antibodies affinity-purified against other family members. Antibodies against other members of the family displayed intermediate levels of cross-reactivity.

To determine binding avidity of the cross-reacting antibodies we performed dot-ELISA assay using antibodies affinity-purified against different recombinant proteins towards all members of the family under increasing concentrations of ammonium thiocyanate (NH4SCN) solutions. The reactivity observed for any given antigen-antibody combination in absence of NH4SCN was considered as 100%, and the reactivities observed under increasing concentrations of NH4SCN, were considered as fractions of that 100%. Heterogeneous patterns of binding avidity were observed for each affinity-purified antibody preparation against different members of the family. Moreover, for several antigen-antibody combinations, the presence of more than one slope indicated mixture of antibodies with varying binding avidities, as shown in FIG. 31. We, therefore, evaluated the ‘% area covered by the curve’ as an estimate for the avidity of antibody binding. As shown in Table 8, an estimation of ‘antigenicity’ of the different antigens and the ‘degree of cross-reactivity’ of the antibodies affinity-purified against them was obtained by summing the ‘% areas covered by the curves’ for the ability of each antigen to be recognized by different antibody preparations (along the columns of Table 8) and for reactivity of each antibody towards all members of the family (along the rows of Table 8) respectively. As observed MSP3.1C-term was found to be most antigenic displaying high binding strength with antibodies affinity-purified against different members of the family (table 9). In contrast, MSP3.4 C-term was found to be least antigenic and displayed relatively weaker binding with different antibodies. The degree of cross-reactivity displayed by each affinity-purified antibodies was also varying. Anti-MSP3.1 antibodies were found to be least cross-reactive, in contrast to the highest degree of cross-reactivity displayed by anti-MSP3.2 and anti-MSP3.4 antibodies.

These results strongly suggest a network of antigenic cross-reactivity exhibited by naturally occurring antibodies against different members of the MSP3 family of proteins.

We immunized mice with the C-terminal recombinant proteins from two of these members, MSP3.1 and MSP3.2, in order to determine the cross-reactivity displayed by antibodies induced through artificial immunizations. Antibodies generated against both MSP3.1 and MSP3.2 were cross-reactive to all members of the family, FIG. 32, further demonstrating the antigenic properties shared between different members of the MSP3 family of proteins.

All Members of the MSP3 Family of Proteins Elicit Naturally Occurring Antibodies Effective in Parasite Killing through ADCI

We have earlier found that anti-MSP3.1 and anti-MSP3.2 antibodies mediated monocyte dependent inhibition of parasite growth (Singh, et al., 2004; Singh, et al., manuscript communicated). In order to evaluate anti-parasite effect of naturally occurring antibodies against other members of the family, affinity-purified antibodies from hyperimmune sera, against the related C-terminal part of the molecules, were tested in ADCI assay in vitro. Each affinity-purified antibody was adjusted to an equal effective concentration yielding the same reactivity to the native parasite protein, as determined by the end-point titer of each antibody preparation in IFA (data not shown). Results (FIG. 33) show that antibodies against each member of the MSP3 family of proteins elicited strong parasite inhibition. The level of inhibition was comparable to that observed for the pool of African IgG (PIAG) previously used for passive transfer experiment in humans (Sabchareon, et al., 1991). The results demonstrate that each member of the MSP3-family of proteins serves as a target of naturally occurring antibodies with anti-parasite effect, which is in accordance with the antigenic similarities and the network of cross-reactivity displayed by antibodies against them.

9C—Discussion

Based on their sequence relatedness P. falciparum proteins could be grouped into different families. Several gene-families are expressed in the asexual blood stage of the parasite. The members of the highly variable gene families such as PfEMP1 (var), rifin and stevor are dispersed in the recombinogenic subtelomeric regions of different chromosomes. They are expressed on the surface of the infected RBC and are involved in antigenic variation and cytoadherance (Su, et al., 1995; Fernandez, et al., 1999). Members of the RBL, EBL and RAP are expressed in the merozoite secretory organelles and exhibit considerable gene redundancy (Reed, et al., 2000; Kaneko, et al., 2000; Duraisingh, et al., 2003). They are secreted during the merozoite invasion of the RBC and are known to have mechanistic roles in alternate invasion pathways of P. falciparum (Barnwell, 1999).

Merozoite surface proteins could be classified into membrane-anchored proteins and membrane-associated proteins. Whereas, the GPI-anchored proteins with single EGF-like domains: MSP2, MSP5, and MSP4 are located on the same chromosomal locus, those with double EGF-like domains such as MSP1, MSP8, MSP8-like and MSP10 are located on different chromosomes (Burns, et al., 2000; Black, et al., 2001; Black, et al., 2003). On the contrary, most of the members belonging to the membrane-associated proteins families such as MSP3, MSP7 and SERA (except SERA9) are clustered in tandem on the same chromosome (Mello, et al., 2002; Aoki, et al., 2002; Miller, et al., 2002).

MSP3 family of proteins differs in several characteristics as compared to other multi-gene families. All MSP3 genes have single exon structure unlike two or more exon structures observed for members of other gene families such as PfEMP1, rifin, stevor, PfRBL, EBL, and SERA gene families. All members of the MSP3 family of proteins are simultaneously expressed on the merozoite surface. This is not common for other gene families like var genes, where only one is expressed at any one time (Scherf, et al., 1998) or SERA where peripheral genes in the cluster are not expressed (Aoki, et al., 2002; Miller, et al., 2002).

Though members of other gene families share the general sequence organization, they are quite diverse and do not share cross-reactive epitopes. For e.g., naturally occurring antibodies against the EGF-like domains of different MSPs are not cross-reactive (Black, et al., 1999; Black, et al., 2003). However, some degree of cross-reactivity has recently been reported between different members of the var gene family (Chattopadhyay, et al., 2003). The C-terminal regions of the MSP3 family of proteins are quite related and share cross-reactive epitopes, which are highly conserved in different parasite, isolate and are target of naturally occurring antibodies, which mediate ADCI.

All genes of the MSP3 family are transcribed and the corresponding proteins are simultaneously expressed on the surface of P. falciparum merozoites. This broadens very much the number of MSP3-like epitopes expressed simultaneously on the merozoite surface and thereby the number of epitopes that can be targeted by MSP3 induced antibodies i.e., immunization with the MSP3-LSP vaccine construct in volunteers induced antibodies that were able to react with all members of the family and therefore played a role in bio-assays reflecting protection by interacting not only with MSP3.1, but also with other members of the family.

The gene duplication and the expression of relevant homologous epitopes may also explain why the knockout experiments performed with MSP3.1 (Mills, et al., 2002) and MSP3.2 (Mills & Cowman, personal communication) had little consequences on parasite survival. Homologous structures expressed by the remaining members of the MSP3 family could compensate for the loss of either MSP3.1 or MSP3.2. In other words, simultaneous expression of all members provides the parasite, with available functional spare-wheels.

The related C-terminal region from each member of the family was found to inhibit parasite growth in cooperation with monocytes and of similar magnitude as that mediated by antibodies to the original MSP3.1 protein. This broadens the scope of antigens involved in naturally acquired protection and vaccine constructs based on them could be formulated. Despite the shared sequence organization in the related C-terminal regions with higher degree of homology in critical regions identified as for protection, the various MSP3 genes also show substantial diversity in sequence. A detailed analysis of MSP3.1 had led to identify 3 epitopic regions as targets of protective antibodies, both on epidemiological and clinical grounds as well as in assays reflecting antibody mediated protection (Singh, et al., 2004).

Detailed antigenic analysis of the MSP3 family members shows that, differences among their primary amino-acid sequences, the antigenic properties are sufficiently conserved to generate cross-reactive antibodies. The extent of cross-reactivity is such that when detecting an antibody response in humans to one member of the family, any other member of the multi-gene family could have elicited these antibodies. However, antibodies affinity-purified on one given gene product differed in its binding avidity to other gene products. Results indicate existence of a complex pattern of molecular interactions between antibodies generated against one gene product with the remaining members of the family.

Polymorphism in malaria genes can be generated by random mutations and is usually considered as a major bottleneck for vaccine development as it frequently concerns epitopic regions involved in protection. However, in the case of the MSP3 multi-gene family, the differences between different members do not seem to be related to random mutations. Indeed, the full sequence conservation of each MSP3 gene among several distinct isolates is extremely striking and most unusual. It indicates that the existing differences between different members are not randomly generated, and conversely suggest that these differences might be conserved for important functions. To summarize, the members of the MSP3-family of proteins in P. falciparum show highly conserved divergences among themselves while still retaining antigenic relatedness. The main question that arises from these findings is what are the reasons for keeping a strong conservation of this diversity? Two main hypotheses can be formulated.

1. This diversity generates a wider range of antibodies species reactive to the related antigenic network than would a single antigen, i.e. with a wider range of diversity in the affinity, avidity and fine-specificity, of the antibody repertoire essential to ensure reactivity to the original and related epitopes and mediate monocyte-dependent parasite killing.

2. The differences in the sequence also provide epitope diversity to ensure that essential antibody responses are induced in a wider range of human genetic backgrounds, i.e. each gene sequence would be better fitted to a given MHC class-II subset. Hence, in this case, the conservation of the diversity serves the purpose of generating in every single individual the same type of essential antibodies. The results obtained in the MSP3.1 vaccine trial are in support of this hypothesis (as all volunteers did not developed Abs reactive with native parasite proteins)

In the latter case, the driving pressure on sequence conservation of a gene is that if a parasite mutates, it fails to induce this type of antibodies in some hosts, therefore leading to a rising high parasitemia in this particular host, his potential death and therefore of this particular mutated parasite, i.e. the frequency of such mutants would spontaneously decrease in the human population.

In both cases, the results point to the importance of anti-MSP3 cross-reactive antibodies that have the ability to control parasite multiplication, i.e. to ensure low to moderate parasite densities in every given human host, ensuring in this manner the survival of both the host and the parasite.

This finding brings new perspectives on the function of merozoite surface antigens. They have fundamental implications in the natural host-parasite interaction to maintain the homeostasis between P. falciparum and human beings. They have also practical consequences for vaccine development, and strongly suggest that an improved MSP3 vaccine should combine the various C-terminus regions that generate a wider range of antibodies acting on each of the various multi-gene family protein products and also improve the immunogenicity in various human genetic backgrounds.

TABLE 6

(A) Pairs of primer pairs used for cloning the unique region sequences,
and (B) the related carboxy-terminal regions, from each member of the MSP3-
family of proteins. The column on the right shows amino acid sequences of the
unique regions. The amino-acids have been numbered with respect to the 3D7
sequence. The related carboxy-terminal recombinants were not designed from
MSP3.4 and MSP3.5 as these sequences do not share sequence relatedness
with other members of the family.

| | | Amino acid sequences of the unique |
|---|---|---|
| | Oligonucleotide primer pairs used for PCR | regions (numbers show a.a. positions |
| Sequence | amplifications | of the 3D7 sequences) |
| (A) | | |
| MSP3.1 unique | F: 5′-CGCAAGATCTGGTTATACGGAAGAATTAAAAGC-3′ | 71-GYTEELKAKKASEDAEKAANDAENASKEAEEAAKEAVN |
| | R: 5-CGCACCCATGGTATGAAGATTTTTCAGCATCATC-3′ | LKESDKSYTKAKEACTAASKAKKAVETALKAKDDAEKSS- |
| | | 147 |
| MSP3.2 unique | F: 5′-CGCAAGATCTACATCAAGGAGGAAATAATGTAATTCC-3′ | 112-TSGGNNVIPLPIKQSGENQYTVTSISGIQKGANGLTG |
| | R: 5′-CGCACCATGGCTAATTATTATTCAGAGAAGTTGTAG-3′ | ATENITQVVQANSETNKNPTSHSNSTTTSLNNN-181 |
| MSP3.3 unique | F: 5′-CGCAAGATCTATTTATGAAACTACAGGAAGTCTAAGG-3′ | 72-IYETTGSLGTGVESVKAIDGESGTSMDSKPKENKISTE |
| | R: 5′-CGCACCATGGCTAATCATTTTCTAAACTACTATCAG-3′ | PGADQVSIGLVNESDSSLEND-130 |
| SP3.4 unique | F: 5′-CGCAAGATCTGATTCTCTAACAACCACTTCTTTATCAACG-3′ | 459-DSLTTTSLSTSINSVRDSSNLDQRGNITTSQGNSHRA |
| | R: 5′-CGCACCATGGCTAATTATTGTTGTAGTTATTATTTCC-3′ | TVVQQVDQTNRLDNVNSVTQRGNNNYNNN-524 |
| MSP3.5 unique | F: 5′-CGCAAGATCTCAATCCAAAGGAAATAGTGGTACTAAGG-3′ | 210-QSKGNSGTEGDGSSVFGSIFGSLLTPIDSLLEKFIGS |
| (odd member) | R: 5′-CGCACCATGGCTAATCTAAGTATATATTATTGTCG-3′ | NNTNSDSNVKNTSMGNGQNKYDNN IYLD-274 |
| MSP3.6 unique | F: 5′-CGCAAGATCTCTTGATATCTTTTACT-3′ | 95-LDIFTENKEQKNEEVPMKIEVVNDGEEVKTEYVSEKNE |
| (odd member) | R: 5′-CGCACCATGGCTAACCTATTTCAGTTTCCG-3′ | EVENKSETEIG-143 |
| MSP3.7 unique | F: 5′-CGCAAGATCTTATGAAGCTTCAGAATATATAGA-3′ | 60-YEASEYIEKQNDILNMYNDEKEKNNNNSLDTNVTKNTV |
| | R: 5′-CGCACCATGGCTACCCAGTACCTACAAATATACC-3′ | IDNSNKFQSIEDNNVYNKGIFVGTG-122 |
| MSP3.8 unique | F: 5′-CGCAAGATCTGTGAGTAATAGTGTGAATGCCTTACC-3′ | 475-VSNSVNALPEPGQITLPDPSLKQTTQQENQPVVETPV |
| | R: 5′-CGCACCATGGCTAGCTACCTTGGTTTACTTCTTGG-3′ | TTAVINEHQGQTEPNKGDNNNERENHESNVGSIQEVNQGS- |
| | | 551 |

| | Oligonucleotide primer pairs used for PCR | Amino acid sequences for C-term |
|---|---|---|
| Sequence | amplifications | (numbers show a.a. positions in 3D7) |
| (B) | | |
| MSP3.1CT | F: 5′-CGCAAGATCTTATGAAAAGGCAAAAAATGCT-3′ | 167-YEKAKNAYQKANQAVLKAKEASSY . . . |
| | R: 5′-CGCACCATGGTTAATGATTTTTAAAATATTTGGA-3′ | . . . GNNQIDSTLKDLVEELSKYFKNH-371 |
| MSP3.2CT | F: 5′-CGCAAGATCTTCTGAAACAAATAAAAATCCTACTTCTCAT-3′ | 161-SETNKNPTSHSNSTTTSLNNNILGWE . . . |
| | R: 5′-CGCACCATGGTTAATTATTACTAAATAGATGGATCATTTCTTG-3′ | . . . NEKNEIDSTINNLVQEMIHLFSNN-371 |
| MSP3.3CT | F: 5′-CGCAAGATCTTATGAGAAGAAAAATGAAAATA-3′ | 228-YEKKNENKNVSNVDSKTKSNEKGR . . . |
| | R: 5′-CGCACCATGGTTAATTATATGTAAAAAATTCCAT-3′ | . . . LNGKNELDATIRRLKHRFMEFFT YN-424 |
| MSP3.4CT | F: 5′-CGCAAGATCTGATAATGTAAACTCTGTAACG-3′ | 508-DNVNSVTQRGNNNYNNNLERGLGS . . . |
| | R: 5′-CGCACCATGGTTATTTTTGAAATAAATCTGTCAT-3′ | . . . FNDNNNLETIFKGLTEDMTDLFQK-097 |
| MSP3.7CT | F: 5′-CGCAAGATCTCCTGAAGGACCAAGAGCAAA-3′ | 214-PEGPRANNRNENNQNTDPYNHYFA . . . |
| | R: 5′-CGCACCATGGTCAATAGTTATTTAAAAAAAAAGT-3′ | . . . QTNNQLDPSLKDLENELTFFLNNY-405 |
| MSP3.8CT | F: 5′-CGCAAGATCTCATGAAAGTAATGTTGGTAG-3′ | 537-HESNVGSIQE VNQGS VSEESHSKTI . . . |
| | R: 5′-CGCACCATGGTTAATTTTTAAATAAATTTGTAAT-3′ | . . . LEEGNGSDSTLNSLSKDITNLFKN-762 |

TABLE 7
BLASTP comparison of the P. falciparum MSP3 family of proteins

| | E value (% identity, % similarity) | | | | | |
|---|---|---|---|---|---|---|
| Gene product | MSP3.1 | MSP3.2 | MSP3.3 | MSP3.4 | MSP3.7 | MSP3.8 |
| MSP3.1 | 0.0 | 9 × 10⁻⁴¹ | 3 × 10⁻²¹ | 3 × 10⁻²¹ | 8 × 10⁻²⁴ | 2 × 10⁻¹⁷ |
| | | (31, 48) | (25, 39) | (26, 48) | (25, 40) | (26, 41) |
| MSP3.2 | 8 × 10⁻⁴¹ | 0.0 | 1 × 10⁻²¹ | 2 × 10⁻²³ | 7 × 10⁻²⁹ | 9 × 10⁻²³ |
| | (31, 48) | | (30, 47) | (27, 45) | (25, 43) | (31, 51) |
| MSP3.3 | 9 × 10⁻²⁷ | 9 × 10⁻²² | 0.0 | 1 × 10⁻¹⁷ | 2 × 10⁻²⁰ | 1 × 10⁻¹⁷ |
| | (27, 43) | (30, 47) | | (32, 51) | (25, 41) | (27, 45) |
| MSP3.4 | 1 × 10⁻²¹ | 1 × 10⁻²⁴ | 5 × 10⁻¹⁸ | 0.0 | 2 × 10⁻¹⁷ | 1 × 10⁻⁹² |
| | (26, 48) | (27, 45) | (32, 51) | | (31, 52) | (31, 47) |
| MSP3.7 | 7 × 10⁻²⁴ | 3 × 10⁻²⁹ | — | 3 × 10⁻¹⁷ | 0.0 | 9 × 10⁻¹² |
| | (25, 40) | (27, 44) | | (31, 52) | | (25, 40) |
| MSP3.8 | 7 × 10⁻¹⁹ | 2 × 10⁻²³ | 1 × 10⁻¹⁸ | 6 × 10⁻⁹³ | 2 × 10⁻¹² | 0.0 |
| | (25, 42) | (31, 51) | (27, 45) | (31, 47) | (25, 40) | |

The sequences indicated in bold were used as queries in a custom blast at the Malaria Genetics/Genomic database at NCBI.

TABLE 8
Naturally occurring antibodies against related C-terminal regions
of the MSP3 family of proteins exhibit cross-reactivity.

| | MSP3.1 Ct | MSP3.2 Ct | MSP3.3 Ct | MSP3.4 Ct | MSP3.7 Ct | MSP3.8 Ct | 571-His | BSA |
|---|---|---|---|---|---|---|---|---|
| anti-MSP3.1Ct | 0.156 | 0.009 | 0.052 | 0.007 | 0.054 | 0.036 | 0.005 | 0.006 |
| | (100%) | (5.8%) | (33.3%) | (4.5%) | (34.6%) | (23.1%) | (3.2%) | (3.8%) |
| anti-MSP3.2 Ct | 0.073 | 0.134 | 0.029 | 0.005 | 0.052 | 0.05 | 0.005 | 0.006 |
| | (54.5%) | (100%) | (21.6%) | (3.7%) | (38.8%) | (37.3%) | (3.7%) | (4.5%) |
| anti-MSP3.3 Ct | 0.135 | 0.052 | 0.115 | 0.006 | 0.115 | 0.054 | 0.006 | 0.006 |
| | (117.4%) | (45.2%) | (100%) | (5.2%) | (100%) | (47.0%) | (5.2%) | (5.2%) |
| anti-MSP3.4 Ct | 0.158 | 0.032 | 0.095 | 0.073 | 0.107 | 0.075 | 0.007 | 0.007 |
| | (216.4%) | (43.8%) | (130.1%) | (100%) | (146.6%) | (102.7%) | (9.6%) | (9.6%) |
| anti-MSP3.7 Ct | 0.047 | 0.006 | 0.005 | 0.005 | 0.147 | 0.007 | 0.006 | 0.006 |
| | (32.0%) | (4.1%) | (3.4%) | (3.4%) | (100%) | (4.8%) | (4.1%) | (4.1%) |
| anti-MSP3.8 Ct | 0.085 | 0.027 | 0.031 | 0.009 | 0.062 | 0.117 | 0.007 | 0.006 |
| | (72.6%) | (23.1%) | (26.5%) | (7.7%) | (53.0%) | (100%) | (6.0%) | (5.1%) |

Antibodies affinity-purified against C-terminal recombinant protein from each member of MSP3-family of proteins were assessed for their cross-reactivity towards other members by ELISA. O.D.450 values obtained for the reactivity of affinity-purified antibodies towards each recombinant protein are shown. The shaded boxes represent reactivity of the antibodies affinity-purified against their respective recombinant proteins, which was considered to be 100%. The degree of cross-reactivity towards other members of the family is expressed as fractions of 100%, shown in bold.

TABLE 9
Relationship between antigenicity and cross-reactivity deduced from the antibody binding avidity.

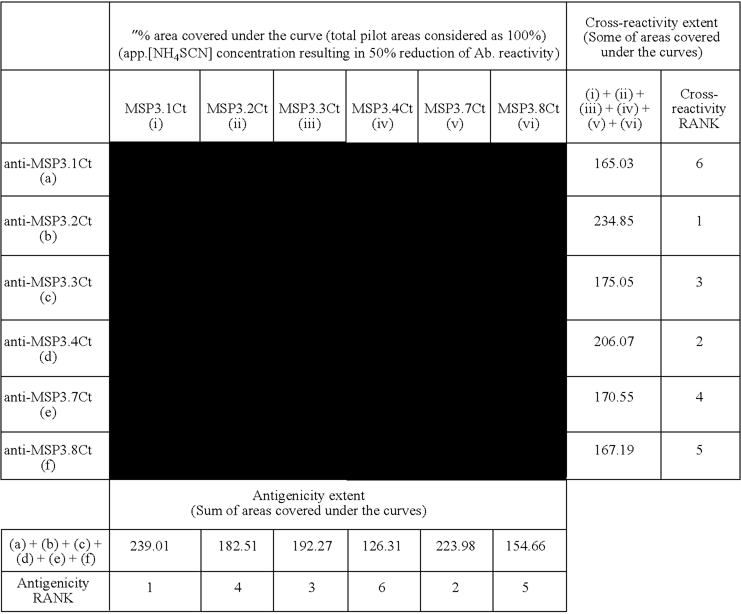

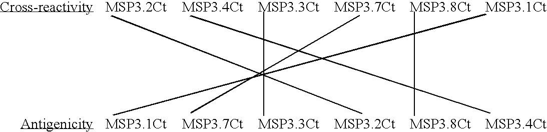

Antibody binding avidity for each antigen-antibody reaction is expressed in terms of “% area covered by the curve’ (as explained in the text and FIG. 6). Summing the binding avidity displayed by different antibody-preparations towards any given antigen provides an estimate of its ‘antigenicity’, obtained here along the columns. Similarly, summing the binding avidity displayed by any antibody-preparation towards different antigens provides an estimate about the degree of ‘cross-reactivity’ for that antibody preparation, obtained here across the rows. Arranging the molecules in the order of their increasing ‘antigenicity’ and the degree of ‘cross-reactivity’ displayed by affinity-purified antibodies shows MSP3.1 to be the most while MSP3.4 being the least antigenic molecules in the family. Conversely, anti-MSP3.1 antibodies displayed least cross-reactive, in contrast to higher degree of cross-reactivity displayed by anti-MSP3.2 and anti-3.4 antibodies.

### Example 10

### Plasmodium falciparum Merozoite Surface Protein 6 Displays Multiple Targets for Naturally Occurring Antibodies Mediating Monocyte-Dependent Parasite Killing

In this example, MSP6 designates MSP3-2, MSP3 designates MSP3-1. Plasmodium falciparum MSP6 is a merozoite surface antigen that shows organization and sequence homologies similar to MSP3. It presents, within its C-terminus conserved region, epitopes that are cross-reactive with MSP3 and others that are not, both being targets of naturally occurring antibodies that block P. falciparum erythrocytic cycle in co-operation with monocytes.

P. falciparum MSP6 is a recently described merozoite surface molecule, structurally related in its overall sequence organization to previously described MSP3 (Pearce, J. A., T. Triglia, A. N. Hodder, D. C. Jackson, A. F. Cowman, and R. F. Anders, 2004.; Trucco, C., D. Fernandez-Reyes, S. Howell, W. H. Stafford, T. J. Scott-Finnigan, M. Grainger, S. A. Ogun, W. R. Taylor, and A. A. Holder, 2001). The C-terminal part of the protein shows homology with MSP3 (ca. 50% identity and 85% similarity of amino acid residues) and an identity for a 11 amino acid stretch (ILGWEFGGG[A/V]P) previously identified as a target of antibodies with strong anti-parasite activity (Oeuvray, C., H. Bouharoun-Tayoun, H. Gras-Masse, E. Bottius, T. Kaidoh, M. Aikawa, M. C. Filgueira, A. Tartar and P. Druilhe., 1994 ; Singh, S., S. Soe, J. P. Mejia, C. Roussilhon, M. Theisen, G. Corradin and P. Druilhe, 2004). Moreover, the C-terminal part of the molecule is highly conserved in MSP6, as in case of MSP3, whereas, the N-terminal part is proteolytically cleaved, more polymorphic and less antigenic than the C-terminal part (Pearce, J. A., T. Triglia, A. N. Hodder, D. C. Jackson, A. F. Cowman, and R. F. Anders, 2004 ; Wang, L., L. Crouch, T. L. Richie, D. H. Nhan, and R. L. Coppel, 2003).

MSP3 has been identified as a target of protective antibodies using ADCI (antibody dependent cellular inhibition) assays, a mechanism found to reflect best the protection that can be passively transferred by antibodies in P. falciparum infected patients (Oeuvray, C., H. Bouharoun-Tayoun, H. Gras-Masse, E. Bottius, T. Kaidoh, M. Aikawa, M. C. Filgueira, A. Tartar and P. Druilhe, 1994). It has been pursued for human vaccine trials based on a series of findings suggesting that anti-MSP3 antibodies contribute to protection against malaria: i) immuno-epidemiological studies showed a significant correlation of IgG3 antibodies with protection acquired by natural exposure to the parasite (Singh, S., S. Soe, J. P. Mejia, C. Roussilhon, M. Theisen, G. Corradin and P. Druilhe, 2004, Soe, S., M. Theisen, C. Roussilhon, K. S. Aye, and P. Druilhe, 2004); ii) either naturally occurring or artificially raised antibodies have a strong monocytedependent antibody ADCI effect (Oeuvray, C., H. Bouharoun-Tayoun, H. Gras-Masse, E. Bottius, T. Kaidoh, M. Aikawa, M. C. Filgueira, A. Tartar and P. Druilhe, 1994; Singh, S., S. Soe, J. P. Mejia, C. Roussilhon, M. Theisen, G. Corradin and P. Druilhe, 2004); iii) immunity can be actively elicited in primates against a P. falciparum challenge and correlates with pre-challenge antibody titers (Hisaeda, H., A. Saul, J. J. Reece, M. C. Kennedy, C. A. Long, L. H. Miller, and A. W. Stowers, 2002); iv) immunity can be passively transferred by antibodies in P. falciparum infected SCID mice (Badell, E., C. Oeuvray, A. Moreno, S. Soe, N. van Rooijen, A. Bouzidi, and P. Druilhe, 2000 ; Singh, S., S. Soe, J. P. Mejia, C. Roussilhon, M. Theisen, G. Corradin and P. Druilhe, 2004) and P. reichnowi-infected chimpanzees (Druilhe P., et al, submitted for publication).

Given the homologies of MSP6 with MSP3, we therefore performed a detailed study of the antigenicity of MSP6 and assessed the anti-parasite role of the naturally occurring anti-MSP6 antibodies.

10A—Antigenicity in Endemic Area Populations

The C-terminal part of MSP6 (amino acids 161-371 in 3D7 clone) was cloned and expressed as a recombinant histidine-tagged protein (MSP6-CT), as described earlier (Theisen, M., J. Vuust, A. Gottschau, S. Jespen, and B. Hogh, 1995). Six overlapping peptides (MSP6a161-182, MSP6b179-204, MSP6c192-224, MSP6d205-257, MSP6e282-326 and MSP6f320-371) were designed in similar manner as those from MSP3 (Singh, S., S. Soe, J. P. Mejia, C. Roussilhon, M. Theisen, G. Corradin and P. Druilhe, 2004), each representing different regions of the C-terminal part, as shown in FIG. 34. A small glutamic acid rich region (a.a. 258 to a.a. 281; 54% glutamic acid rich) was excluded to avoid cross-reactivity exhibited by glutamate rich epitopes present in several P. falciparum antigens (Mattei, D., K. Berzins, M. Wahlgren, R. Udomsangpetch, P. Perlmann, H. W. Griesser, A. Scherf, B. Muller-Hill, S. Bonnefoy, M. Guillotte, G. Langsley, L. H. Pereira da Silva and O. Mercereau-Puijalon, 1989). ELISA assays were performed, as described earlier (Singh, S., S. Soe, J. P. Mejia, C. Roussilhon, M. Theisen, G. Corradin and P. Druilhe, 2004), to determine the level of total IgG and the subclass distribution against each peptide in sera from 30 malaria-protected African Adults from Ivory Coast. Passive transfer of IgG purified from these sera was earlier found to markedly reduce the level of parasitemia in malaria patients (Sabchareon, A., T. Burnouf, D. Ouattara, P. Attanath, H. Bouharoun-Tayoun, P. Chantavanich, C. Foucault, T. Chongsuphajaisiddhi, and P. Druilhe, 1991).

FIG. 35 summarizes antibody subclass reactivity recorded against the 6 peptides covering the various regions of MSP6-Cterm. Though the prevalence of IgG against different regions varied, the pattern of antibody subclass reactivity to each peptide was rather homogeneous with an overall dominance of cytophilic antibodies IgG1 and IgG3. Substantial levels of IgG2 antibodies were also detected against some of the peptides, eg. MSP6e. This antibody subclass pattern differs from that observed against several blood stage antigens. For instance, the corresponding regions of MSP3 showed a predominance of IgG3 against MSP3b, MSP3c and MSP3d peptides whereas IgG1 predominated against MSP3f (Singh, S., S. Soe, J. P. Mejia, C. Roussilhon, M. Theisen, G. Corradin and P. Druilhe, 2004). Differential patterns of antibody subclass have also been found against distinct regions within a single protein, e.g., a predominance of IgG3 against MSP-1 ‘block2’ in contrast to IgG1 against MSP-119 (Cavanagh, D. R., C. Dobano, I. M. Elhassan, K. Marsh, A. Elhassan, L. Hviid, E. A. Khalil, T. G. Theander, D. E. Arnot, and J. S. McBride, 2001), and between different proteins, e.g., predominance of IgG3 against MSP-2 (Rzepczyk, C. M., K. Hale, N. Woodroffe, A. Bobogare, P. Csurhes, A. Ishii, and A. Ferrante, 1997; Taylor, R. R., S. J. Allen, B. M. Greenwood, and E. M. Riley, 1995) as compared to IgG1 against RAP-1 (Fonjungo, P. N., I. M. Elhassan, D. R. Cavanagh, T. G. Theander, L. Hviid, C. Roper, D. E. Arnot, and J. S. McBride, 1999) and AMA-1 (Singh S., et al, unpublished results). Within, the family of another merozoite surface antigens, IgG3 is predominant against MSP-4 as compared to IgG1 against MSP-5 (Wang, L., L. Crouch, T. L. Richie, D. H. Nhan, and R. L. Coppel, 2003; Weisman, S., L. Wang, H. Billman-Jacobe, D. H. Nhan, T. L. Richie and R. L. Coppel. 2001). The factors responsible for distinct human subclass response to different antigens are not fully understood, however the nature of the antigen itself (Gerraud, O., R. Perraut, A. Diouf, W. S. Nambei, A. Tall, A. Spiegel, S. Longacre, D. C. Kaslow, H. Jouin, D. Mattei, G. M. Engler, T. B. Nutman, E. M. Riley, and O. Mercereau-Puijalon, 2002) and the cytokine milieu experienced by the responding B-cells (Gerraud, O., and T. B. Nutman, 1996) may both influence the outcome.

10B—Antimalarial Activity of Anti-MSP6 Antibodies

To assess the functional activity of human antibodies towards different regions of MSP6, we affinity-purified antibodies against each of the 6 peptides, using independent serum pools (each made up of 5 to 7 individual serum samples), selected as described earlier (Singh, S., S. Soe, J. P. Mejia, C. Roussilhon, M. Theisen, G. Corradin and P. Druilhe, 2004) on the basis of high content of cytophilic antibodies (IgG1+IgG3) and minimal reactivity towards the adjacent regions. The affinity-purified antibodies proved to be specific against the respective peptides, as no cross-reactivity was observed to other regions of the molecule (Table 10 A). Thus, each of the MSP6 peptides was found to define at least one B-cell epitope that does not share antigenic determinants with other regions of the molecule. Indirect immunofluorescence assays (IFA) on acetone-fixed thin smears of P. falciparum asexual blood stage parasites, indicated that each anti-peptide antibody was reactive with the native parasite protein (data not shown). The anti-parasite activity was thereafter assessed in vitro using monocyte-dependent ADCI assays. To this end, each peptide-specific antibody was adjusted to an equal effective concentration by testing reactivity to the parasite protein (1/200 IFA end-point titer), as previously described (Singh, S., S. Soe, J. P. Mejia, C. Roussilhon, M. Theisen, G. Corradin and P. Druilhe, 2004) for testing in ADCI assays. The assays were performed in duplicates over 96-hour period, using the 3D7 strain of parasites and monocytes from healthy donors, as described earlier (Bouharoun-Tayoun, H., P. Attanath, A. Sabchareon, T. Chongsuphajaisiddhi, and P. Druilhe, 1990). The affinity-purified antibodies, dialyzed against RPMI medium, were added at a ratio of 10% (v/v) of the complete culture medium, thus each of them was used at a final IFA titer of 1/20 in the ADCI assay. Parasitemia, determined at the end of 96 h by microscopic examination of jÝ10,000 erythrocytes on Giemsa-stained thin smears, was used to calculate the specific growth inhibitory index as follows: % SGI=1−{(percentage of parasitemia with monocytes and test IgG/percentage of parasitemia with test IgG)/(percentage of parasitemia with monocytes and normal IgG/percentage of parasitemia with normal IgG)}×100. Results from the ADCI assays (FIG. 36) show that antibodies affinity-purified against each of the 6 peptides were able to exert a strong monocyte-dependent inhibition of the parasite growth. This result differs markedly from those obtained with MSP3, where only three of the six peptide-specific antibodies were effective (Singh, S., S. Soe, J. P. Mejia, C. Roussilhon, M. Theisen, G. Corradin and P. Druilhe, 2004). No significant direct effect upon parasite growth (in the absence of monocytes) was observed at the antibody concentrations employed (data not shown).

10C—MSP6 and MSP3 Share Cross-Reactive Epitopes

Cross-reactivity was examined using anti-MSP6 affinity-purified antibodies against homologous peptides from MSP3. As shown in Table 10 B, four regions were found cross-reactive. Anti-MSP6 “b” and “f” antibodies were fully cross-reactive, whereas anti-MSP6 “d” and anti-MSP6 “e” antibodies displayed partial cross-reactivity. In contrast, anti-MSP6a and anti-MSP6c did not show cross-reactivity to the corresponding MSP3 regions. These results are in overall agreement with the sequence homologies (FIG. 34B) and suggest that the anti-parasite effect mediated by some of the anti-MSP6 antibodies, could also be due to the binding to cross reactive regions in MSP3. However, parasite inhibition mediated by non cross-reactive MSP6 antibodies, such as anti-MSP6a and anti-MSP6c, demonstrate that MSP6 is also a target of ADCI on its own.

TABLE 10
Specificity of affinity-purified human anti-MSP6 antibodies
determined by ELISA

| | | | | | |
|---|---|---|---|---|---|
| anti-MSP6a | anti-MSP6b | anti-MSP6c | anti-MSP6d | anti-MSP6e | anti-MSP6f |

(A)

| | | | | | | |
|---|---|---|---|---|---|---|
| MSP6a | 0.118 | 0.009 | 0.008 | 0.008 | 0.009 | 0.008 |
| MSP6b | 0.007 | 0.111 | 0.007 | 0.008 | 0.007 | 0.009 |
| MSP6c | 0.009 | 0.008 | 0.084 | 0.018 | 0.006 | 0.007 |
| MSP6d | 0.010 | 0.007 | 0.008 | 0.116 | 0.007 | 0.009 |
| MSP6e | 0.009 | 0.009 | 0.009 | 0.008 | 0.085 | 0.008 |
| MSP6f | 0.007 | 0.008 | 0.007 | 0.008 | 0.009 | 0.092 |

(B)

| | | | | | | |
|---|---|---|---|---|---|---|
| MSP3a | 0.007 | 0.007 | 0.008 | 0.010 | 0.007 | 0.012 |
| MSP3b | 0.010 | 0.105 | 0.008 | 0.008 | 0.009 | 0.007 |
| MSP3c | 0.008 | 0.009 | 0.010 | 0.008 | 0.008 | 0.009 |
| MSP3d | 0.010 | 0.009 | 0.007 | 0.049 | 0.008 | 0.008 |
| MSP3e | 0.008 | 0.008 | 0.009 | 0.009 | 0.045 | 0.010 |
| MSP3f | 0.009 | 0.009 | 0.011 | 0.009 | 0.008 | 0.085 |

Antibodies affinity-purified against different regions of MSP6 were tested (A) for specificity, and (B) for cross-reactivity to related regions in MSP3. Mean O.D.₄₅₀ values from duplicate wells are shown. All the peptides were used under identical coating conditions. Shading represents positive reactivity.

### REFERENCES

Aoki S, Li J, et al (2002) “Serine repeat antigen (SERA5) is predominantly expressed among the SERA multigene family of Plasmodium falciparum, and the acquired antibody titers correlate with serum inhibition of the parasite growth.” J Biol Chem. 277(49): 47533-40.
Badell, E., C. Oeuvray, et al. (2000). “Human malaria in immunocompromised mice: an in vivo model to study defense mechanisms against Plasmodium falciparum.” J Exp Med 192(11): 1653-60.
Baird J K, Jones T R, et al, (1991). “Age-dependant acquired protection against Plasmodium Falciparum in people having two years exposure to hyperendemic malaria.” Am J Top Med Hyg 45: 65-76.
Barnwell J W, (1999). “A new escape and evasion tactic” Nature 398(6728): 562-3.
Beier, J. C., G. F. Killeen, et al. (1999). “Short report: entomologic inoculation rates and Plasmodium falciparum malaria prevalence in Africa.” Am J Trop Med Hyg 61(1): 109-13.
BenMohamed, L., Y. Belkaid, et al. (2002). “Systemic immune responses induced by mucosal administration of lipopeptides without adjuvant.” Eur J Immunol 32(8): 2274-81.
Behr C, Sartou J L, et al, (1992). “Antibodies and reactive T cells against the malaria heat-shok protein Pf72/Hsp70-1 and derived peptides in individuals continuously exposed to Plasmodium Falciparum.” J Immunol 149: 3321-30.
Black C G, Wang L, Hibbs A R, Werner E, Coppel R L. Identification of the Plasmodium chabaudi homologue of merozoite surface proteins 4 and 5 of Plasmodium falciparum. Infect Immun. 1999;67(5):2075-81.
Black C G, Wu T, Wang L, Hibbs A R, Coppel R L. Merozoite surface protein 8 of Plasmodium falciparum contains two epidermal growth factor-like domains. Mol Biochem Parasitol. 2001;114(2):217-26.
Black C G, Wang L, Wu T, Coppel R L. Apical location of a novel EGF-like domain containing protein of Plasmodium falciparum. Mol Biochem Parasitol. 2003; 127(1): 59-68.
Bornberg-Bauer E, Rivals E, Vingron M. Computational approaches to identify leucine zippers. Nucleic Acids Res. 1998;26(11):2740-6.
Bottius, E., L. BenMohamed, et al. (1996). “A novel Plasmodium falciparum sporozoite and liver stage antigen (SALSA) defines major B, T helper, and CTL epitopes.” J Immunol 156(8): 2874-84.
Bouharoun-Tayoun, H., P. Attanath, A. Sabchareon, T. Chongsuphajaisiddhi, and P. Druilhe. 1990. Antibodies which protect man against P. falciparum blood stages do not on their own inhibit parasite growth and invasion in vitro but act in co-operation with monocytes. J. Exp. Med. 172: 1633-1641.
Bouharoun-Tayoun, H. and P. Druilhe (1992). “Antibodies in falciparum malaria: what matters most, quantity or quality?” Mem Inst Oswaldo Cruz 87 Suppl 3: 229-34.
Bouharoun-Tayoun, H. and P. Druilhe (1992). “Evidence for an isotype imbalance, which may be responsible for the delayed acquisition of protective immunity.” Infect immun 60: 1473-81.
Bouharoun-Tayoun, H., C. Oeuvray, et al. (1995). “Mechanisms underlying the monocyte-mediated antibody-dependent killing of Plasmodium falciparum asexual blood stages.” J Exp Med 182(2): 409-18.
Brahimi, K., J. L. Perignon, et al. (1993). “Fast immunopurification of small amounts of specific antibodies on peptides bound to ELISA plates.” J Immunol Methods 162(1): 69-75.
Burns J M Jr, Belk C C, Dunn P D. A protective glycosylphosphatidylinositol-anchored membrane protein of Plasmodium yoelii trophozoites and merozoites contains two epidermalgrowth factor-like domains. Infect Immun. 2000;68(11):6189-95.
Cavanagh, D. R., C. Dobano, I. M. Elhassan, K. Marsh, A. Elhassan, L. Hviid, E. A. Khalil, T. G. Theander, D. E. Arnot, and J. S. McBride. 2001. Differential patterns of human immunoglobulin G subclass responses to distinct regions of a single protein, the merozoite surface protein 1 of Plasmodium falciparum. Infect. Immun. 69:1207-1211.
Chattopadhyay R, Sharma A, Srivastava V K, Pati S S, Sharma S K, Das B S, Chitnis C E. Plasmodium falciparum infection elicits both variant-specific and cross-reactive antibodies against variant surface antigens. Infect Immun. 2003;71 (2):597-604.
Cohen S, McGregor I A, Carrington S. Gamma globulin and acquired immunity to human malaria. Nature 1961; 192: 733-7.
Cowman A F, Crabb B S. The Plasmodium falciparum genome—a blueprint for erythrocyte invasion. Science. 2002;298(5591):126-8.
Dame, J. B., J. L. Williams, et al. (1984). “Structure of the gene encoding the immunodominant surface antigen on the sporozoite of the human malaria parasite Plasmodium falciparum.” Science 225(4662): 593-9.
Daubersies, P., A. W. Thomas, et al. (2000). “Protection against Plasmodium falciparum malaria in chimpanzees by immunization with the conserved pre-erythrocytic liver-stage antigen 3.” Nat Med 6(11): 1258-63.
David P H, Hudson D E, Hadley T J, Klotz F W, Miller L H. Immunization of monkeys with a 140 kilodalton merozoite surface protein of Plasmodium knowlesi malaria: appearance of alternate forms of this protein. J Immunol. 1985;134(6):4146-52.
De Stricker, K., J. Vuust, et al. (2000). “Conservation and heterogeneity of the glutamate-rich protein (GLURP) among field isolates and laboratory lines of Plasmodium falciparum.” Mol Biochem Parasitol 111(1): 123-30.
Dodoo, D., T. G. Theander, et al. (1999). “Levels of antibody to conserved parts of Plasmodium falciparum merozoite surface protein 1 in Ghanaian children are not associated with protection from clinical malaria.” Infect Immun 67(5): 2131-7.
Dodoo, D., M. Theisen, et al. (2000). “Naturally acquired antibodies to the glutamate-rich protein are associated with protection against Plasmodium falciparum malaria.” J Infect Dis 181(3): 1202-5.
Druilhe P, Bouharoun-Tayoun H. Human antibody subclass ELISA. Methods Mol Med. 2002;72:457-9.
Druilhe P, Khusmith S. Epidemiological correlation between levels of antibodies promoting merozoite phagocytosis of Plasmodium falciparum and malaria-immune status. Infect Immun. 1987;55(4):888-91.
Druilhe P, Spertini F, Soe Soe D, Corradin G P, Mejia P, Singh S, Audran R, Cachat M, Leroy 0, Oeuvray C. A malaria vaccine that elicits antibodies able to kill P. falciparum (manuscript under preparation).
Duraisingh M T, Triglia T, Ralph S A, Rayner J C, Barnwell J W, McFadden G I, Cowman A F. Phenotypic variation of Plasmodium falciparum merozoite proteins directs receptor targeting for invasion of human erythrocytes. EMBO J. 2003;22(5):1047-57.
Edozien J C, Gilles H M, Udeozo 10. Adult and cord-blood gamma globulin and immunity to malaria in Nigerians. Lancet 1962; ii: 951-5.
Egan, A. F., J. Morris, et al. (1996). “Clinical immunity to Plasmodium falciparum malaria is associated with serum antibodies to the 19-kDa C-terminal fragment of the merozoite surface antigen, PfMSP-1.” J Infect Dis 173(3): 765-9.
Egan A F, Burghaus P, Druilhe P, Holder A A, Riley E M. Human antibodies to the 19 kDa C-terminal fragment of Plasmodium falciparum merozoite surface protein 1 inhibit parasite growth in vitro. Parasite Immunol. 1999; 21: 133-9.
Escalante, A. A., H. M. Grebert, et al. (2001). “Polymorphism in the gene encoding the apical membrane antigen-1 (AMA-1) of Plasmodium falciparum. X. Asembo Bay Cohort Project.” Mol Biochem Parasitol 113(2): 279-87.
Fernandez V, Hommel M, Chen Q, Hagblom P, Wahlgren M. Small, clonally variant antigens expressed on the surface of the Plasmodium falciparum-infected erythrocyte are encoded by the rif gene family and are the target of human immune responses. J Exp Med. 1999;190(10):1393-404.
Fidock, D. A., E. Bottius, et al. (1994). “Cloning and characterization of a novel Plasmodium falciparum sporozoite surface antigen, STARP.” Mol Biochem Parasitol 64(2): 219-32.
Fonjungo, P. N., I. M. Elhassan, D. R. Cavanagh, T. G. Theander, L. Hviid, C. Roper, D. E. Arnot, and J. S. McBride. 1999. A longitudinal study of human antibody responses to Plasmodium falciparum rhoptry-associated protein 1 in a region of seasonal and unstable malaria transmission. Infect. Immun. 67: 2975-2985.
Galinski M R, Ingravallo P, Corredor-Medina C, Al-Khedery B, Povoa M, Barnwell J W. Plasmodium vivax merozoite surface proteins-3beta and-3gamma share structural similarities with P. vivax merozoite surface protein-3alpha and define a new gene family. Mol Biochem Parasitol. 2001;115(1):41-53.
Gerraud, O., and T. B. Nutman. 1996. The roles of cytokines on human B-cell differentiation into immunoglobulin secreting cells. Bull. Inst. Pasteur 94: 285-309.
Gerraud, O., R. Perraut, A. Diouf, W. S. Nambei, A. Tall, A. Spiegel, S. Longacre, D. C. Kaslow, H. Jouin, D. Mattei, G. M. Engler, T. B. Nutman, E. M. Riley, and O. Mercereau-Puijalon. 2002. Regulation of antigen-specific immunoglobulin G subclasses in response to conserved and polymorphic Plasmodium falciparum antigens in an in vitro model. Infect. Immun. 70: 2820-2827.
Gras-Masse, H., B. Georges, et al. (1999). “Convergent peptide libraries, or mixotopes, to elicit or to identify specific immune responses.” Curr Opin Immunol 11(2): 223-8.
Groux H, Gysin J. Opsonisation as an effector mechanism in human protection against asexual blood stages of Plasmodium falciparum: functional role of IgG subclasses. Res Immunol 1990; 141: 532-42.
Guerin-Marchand, C., P. Druilhe, et al. (1987). “A liver-stage-specific antigen of Plasmodium falciparum characterized by gene cloning.” Nature 329(6135): 164-7.
Huber, W., I. Felger, et al. (1997). “Limited sequence polymorphism in the Plasmodium falciparum merozoite surface protein 3.” Mol Biochem Parasitol 87(2): 231-4.
Hisaeda, H., A. Saul, J. J. Reece, M. C. Kennedy, C. A. Long, L. H. Miller, and A. W. Stowers. 2002. Merozoite surface protein-3 and protection against malaria in Aotus nancymai monkeys. J. Infect. Dis. 185: 657-64.
Kaneko O, Fidock D A, Schwartz O M, Miller L H. Disruption of the C-terminal region of EBA-175 in the Dd2/Nm clone of Plasmodium falciparum does not affect erythrocyte invasion. Mol Biochem Parasitol. 2000; 110(1): 135-46.
Khusmith, S. and P. Druilhe (1983). “Cooperation between antibodies and monocytes that inhibit in vitro proliferation of Plasmodium falciparum.” Infect Immun 41(1): 219-23.
Knapp, B., E. Hundt, et al. (1989). “Molecular cloning, genomic structure and localization in a blood stage antigen of Plasmodium falciparum characterized by a serine stretch.” Mol Biochem Parasitol 32(1): 73-83.
Lunel, F. and P. Druilhe (1989). “Effector cells involved in nonspecific and antibody-dependent mechanisms directed against Plasmodium falciparum blood stages in vitro.” Infect Immun 57(7): 2043-9.
Lupas A, Van Dyke M, Stock J. Predicting coiled coils from protein sequences. Science. 1991;252(5010):1162-4.
Marshall, V. M., W. Tieqiao, et al. (1998). “Close linkage of three merozoite surface protein genes on chromosome 2 of Plasmodium falciparum.” Mol Biochem Parasitol 94(1): 13-25.
Mattei, D., K. Berzins, M. Wahigren, R. Udomsangpetch, P. Perlmann, H. W. Griesser, A. Scherf, B. Muller-Hill, S. Bonnefoy, M. Guillotte, G. Langsley, L. H. Pereira da Silva and O. Mercereau-Puijalon. 1989. Cross-reactive antigenic determinants present on different Plasmodium falciparum blood-stage antigens. Parasite Immunol. 11:15-29.
McColl, D. J. and R. F. Anders (1997). “Conservation of structural motifs and antigenic diversity in the Plasmodium falciparum merozoite surface protein-3 (MSP-3).” Mol Biochem Parasitol 90(1): 21-31.
McColl D J, Silva A, Foley M, Kun J F, Favaloro J M, Thompson J K, Marshall V M, Coppel R L, Kemp D J, Anders R F. Molecular variation in a novel polymorphic antigen associated with Plasmodium falciparum merozoites. Mol Biochem Parasitol. 1994;68(1):53- 67.
McGregor I A, Wilson, R J M. Specific immunity: acquired in man. In: Wernsdorfer W H, McGregor I A ed. Malaria: Principles and Practice of Malariology. Churchill Livingstone 1989: 559-619.
Mello K, Daly T M, Morrisey J, Vaidya A B, Long C A, Bergman L W. A multigene family that interacts with the amino terminus of plasmodium MSP-1 identified using the yeast two-hybrid system. Eukaryot Cell. 2002;1(6):915-25.
Miller S K, Good R T, Drew D R, Delorenzi M, Sanders P R, Hodder A N, Speed T P, Cowman A F, de Koning-Ward T F, Crabb B S. A subset of Plasmodium falciparum SERA genes are expressed and appear to play an important role in the erythrocytic cycle. J Biol Chem. 2002;277(49):47524-32.
Miller, L. H., T. Roberts, et al. (1993). “Analysis of sequence diversity in the Plasmodium falciparum merozoite surface protein-1 (MSP-1).” Mol Biochem Parasitol 59(1): 1-14.
Mills K E, Pearce J A, Crabb B S, Cowman A F. Truncation of merozoite surface protein 3 disrupts its trafficking and that of acidic-basic repeat protein to the surface of Plasmodium falciparum merozoites. Mol Microbiol. 2002;43(6):1401-11.
Moreno A, Badell E, van Rooijen N, Druilhe P. Human malaria in immunocompromised mice: new in vivo model for chemotherapy studies. Antimicrob Agents Chemother 2001; 45: 1847-53.
Oeuvray, C., H. Bouharoun-Tayoun, et al. (1994). “Merozoite surface protein-3: a malaria protein inducing antibodies that promote Plasmodium falciparum killing by cooperation with blood monocytes.” Blood 84(5): 1594-602.
Oeuvray, C., M. Theisen, et al. (2000). “Cytophilic immunoglobulin responses to Plasmodium falciparum glutamate-rich protein are correlated with protection against clinical malaria in Dielmo, Senegal.” Infect Immun 68(5): 2617-20.
Pearce, J. A., T. Triglia, A. N. Hodder, D. C. Jackson, A. F. Cowman, and R. F. Anders. 2004. Plasmodium falciparum merozoite surface protein 6 is a dimorphic antigen. Infect. Immun. 72: 2321-2328.
Peterson, M. G., V. M. Marshall, et al. (1989). “Integral membrane protein located in the apical complex of Plasmodium falciparum.” Mol Cell Biol 9(7): 3151-4.
Pleass, R. J. and J. M. Woof (2001). “Fc receptors and immunity to parasites.” Trends Parasitol 17(11): 545-51.
Polley S D, Tetteh K K, Cavanagh D R, et al, Repeat sequences in block 2 of Plasmodium falciparum merozoite surface protein 1 are targets of antibodies associated with protection from malaria. Infect Immun 2003; 71: 1833-42.
Reed M B, Caruana S R, Batchelor A H, Thompson J K, Crabb B S, Cowman A F. Targeted disruption of an erythrocyte binding antigen in Plasmodium falciparum is associated with a switch toward a sialic acid-independent pathway of invasion. Proc Natl Acad Sci U S A. 2000;97(13):7509-14.
Robson, K. J., J. R. Hall, et al. (1988). “A highly conserved amino-acid sequence in thrombospondin, properdin and in proteins from sporozoites and blood stages of a human malaria parasite.” Nature 335(6185): 79-82.
Roggero M A, Servis C, Corradin G. A simple and rapid procedure for the purification of synthetic polypeptides by a combination of affinity chromatography and methionine chemistry. FEBS Lett 1997; 408: 285-8.
Roussillon, C. (1999). “Correlates of Immune protection in malaria.” MIM African Malaria Conference, 14-19 March, Durban South Africa.
Rzepczyk, C. M., K. Hale, N. Woodroffe, A. Bobogare, P. Csurhes, A. Ishii, and A. Ferrante. 1997. Humoral immune responses of Solomon Islanders to the merozoite surface antigen 2 of Plasmodium falciparum show pronounced skewing towards antibodies of the immunoglobulin G3 subclass. Infect. Immun. 65:1098-1100.
Sabchareon A, Burnouf T. Ouattara D, Attanath P, Bouharoun-Tayoun H, Chantavanich P, Foucault C, Chongsuphajaisiddhi T, Druilhe P. Parasitologic and clinical human response to immunoglobulin administration in falciparum malaria. Am J Trop Med Hyg. 1991;45(3):297-308.
Scherf A, Hernandez-Rivas R, Buffet P, Bottius E, Benatar C, Pouvelle B, Gysin J, Lanzer M. Antigenic variation in malaria: in situ switching, relaxed and mutually exclusive transcription of var genes during intra-erythrocytic development in Plasmodium falciparum. EMBO J. 1998;17(18):5418-26.
Sergent E, Parrot L. L'immunité, la prémunition et la résistance innée. Arch Inst Pasteur Alger 1935; 23: 279-319.
Simmons, D., G. Woollett, et al. (1987). “A malaria protein exported into a new compartment within the host erythrocyte.” Embo J 6(2): 485-91.
Singh S, Soe S, Mejia J P, Roussilhon C, Theisen M, Corradin G, Druilhe P. Identification of a Conserved Region of Plasmodium falciparum MSP3 Targeted by Biologically Active Antibodies to Improve Vaccine Design. J Infect Dis. 2004;190(5):1010-8.
Singh S, Soe S, Mejia J P, Roussilhon C, Theisen M, Corradin G, Druilhe P. Plasmodium falciparum Merozoite Surface Protein-6 displays multiple targets for naturally occurring antibodies mediating monocyte-dependent parasite killing. (Manuscript communicated, article-II, this report).
Soe, S., A. Khin Saw, et al. (2001). “Premunition against Plasmodium falciparum in a malaria hyperendemic village in Myanmar.” Trans R Soc Trop Med Hya 95(1): 81-4.
Soe S, Theisen M, Roussilhon C, Aye K S, Druilhe P. Association between protection against clinical malaria and antibodies to merozoite surface antigens in an area of hyperendemicity in Myanmar: complementarity between responses to merozoite surface protein 3 and the 220-kilodalton glutamate-rich protein. Infect Immun. 2004;72(1):247-52.
Stavnezer J. Antibody class switching. Adv Immunol 1996; 61: 79-146.
Su X Z, Heatwole V M, Wertheimer S P, Guinet F, Herrfeldt J A, Peterson D S, Ravetch J A, Wellems T E. The large diverse gene family var encodes proteins involved in cytoadherence and antigenic variation of Plasmodium falciparum-infected erythrocytes. Cell. 1995;82(1):89-100.
Taylor, R. R., S. J. Allen, B. M. Greenwood, and E. M. Riley. 1995. IgG3 antibodies to Plasmodium falciparum merozoite surface protein 2 (MSP2): increasing prevalence with age and association with clinical immunity to malaria. Infect. Immun. 63: 4382-4388.
Taylor R R, Smith D B, Robinson V J, McBride J S, Riley E M. Human antibody response to Plasmodium falciparum merozoite surface protein 2 is serogroup specific and predominantly of the immunoglobulin G3 subclass. Infect Immun 1995; 63: 4382-8.
Theisen, M., D. Dodoo, et al. (2001). “Selection of glutamate-rich protein long synthetic peptides for vaccine development: antigenicity and relationship with clinical protection and immunogenicity.” Infect Immun 69(9): 5223-9.
Theisen, M., S. Soe, et al. (2000). “Identification of a major B-cell epitope of the Plasmodium falciparum glutamate-rich protein (GLURP), targeted by human antibodies mediating parasite killing.” Vaccine 19(2-3): 204-12.
Theisen, M., S. Soe, et al. (1998). “The glutamate-rich protein (GLURP) of Plasmodium falciparum is a target for antibody-dependent monocyte-mediated inhibition of parasite growth in vitro.” Infect Immun 66(1): 11-7.
Theisen M, Vuust J, Gottschau A, Jepsen S, Hogh B. Antigenicity and immunogenicity of recombinant glutamate-rich protein of Plasmodium falciparum expressed in Escherichia coli. Clin Diagn Lab Immunol. 1995;2(1):30-4.
Thomas, A. W., D. A. Carr, et al. (1990). “Sequence comparison of allelic forms of the Plasmodium falciparum merozoite surface antigen MSA2.” Mol Biochem Parasitol 43(2): 211-20.
Trape J F, Rogier C, Konate L, et al, The Dielmo project: a longitudinal study of natural malaria infection and the mechanisms of protective immunity in a community living in a holoendemic area of Senegal. Am J Trop Med Hyg 1994; 51:123-37.
Trucco, C., D. Fernandez-Reyes, et al. (2001). “The merozoite surface protein 6 gene codes for a 36 kDa protein associated with the Plasmodium falciparum merozoite surface protein-1 complex.” Mol Biochem Parasitol 112(1): 91-101.
Tun-Lin, W., M. M. Thu, et al. (1995). “Hyperendemic malaria in a forested, hilly Myanmar village.” J Am Mosci Control Assoc 11(4): 401-7.
Wang, L., L. Crouch, T. L. Richie, D. H. Nhan, and R. L. Coppel. 2003. Naturally acquired antibody responses to the components of the Plasmodium falciparum merozoite surface protein I complex. Parasite Immunol. 25: 403-412.
Weisman, S., L. Wang, H. Billman-Jacobe, D. H. Nhan, T. L. Richie and R. L. Coppel. 2001. Antibody responses in patients infected with strains of Plasmodiumfalciparum expressing diverse forms of merozoite surface protein 2. Infect. Immun. 69: 959-967.
Wolf E, Kim P S, Berger B. MultiCoil: a program for predicting two- and three-stranded coiled coils. Protein Sci. 1997;6(6):1179-89.

1. An antigenic polypeptidic composition, comprising at least one MSP-3-b-like motif and at least one MSP-3-c/d-like motif.

2. The antigenic polypeptidic composition according to claim 1, comprising at least two different MSP-3-b-like motifs and/or at least two different MSP-3-c/d-like motifs.

3. An antigenic composition according to claim 1 comprising at least two different MSP3-b-like motifs and at least one MSP3-c/d like motif.

4. An antigenic polypeptidic composition according to anyone of claims 1 to 3, wherein the MSP3-b-like motifs are comprised in polypeptidic components having from 10 to 80 amino acid residues.

5. An antigenic polypeptidic composition according to anyone of claim 1 to 3, wherein the MSP3-c/d-like motifs are comprised in polypeptididic components having from 20 to 80 amino acid residues.

6. An antigenic polypeptidic composition according to anyone of claims 1 to 5, wherein MSP3-b-like motif(s) and the MSP3-c/d-like motif(s) are comprised in a unique polypeptidic component.

7. An antigenic polypeptidic composition according to anyone of claims 1 to 6, wherein the MSP3-b-like motifs and/or the MSP3-c/d-like motifs are separated in the polypeptidic component, by the aminoacid sequence naturally contained between them in the MSP3-like protein from which they derive.

8. An antigenic polypeptidic composition according to claims 4 to 7, wherein the polypeptidic components, or wherein each of the polypeptidic component comprise or consist of an amino-acid sequence derived from one or several MSP3-like proteins, said amino-acid sequence consisting of all or part of the C-terminal sequence of one or several MSP3-like proteins selected among MSP3-1, MSP3-2, MSP3-3, MSP3-4, MSP3-7 and MSP3-8 proteins of Plasmodium, especially of Plasmodium falciparum.

9. An antigenic polypeptidic composition according to anyone of claims 1 to 8, wherein the polypeptidic component(s) consists of the C-terminal sequences of MSP3-like proteins including at least MSP3-1 and MSP3-2 or fragments of said C-terminal sequences comprising or consisting of the MSP3-1-b, MSP3-1 c/d, MSP3-2-b and MSP3-2 c/d motifs.

10. An antigenic polypeptidic composition according to claim 9, wherein the polypeptidic component(s) further comprise amino-acid sequences consisting of the C-terminal sequences of MSP3-like proteins selected among MSP3-3, MSP3-4, MSP3-7 and MSP3-8 or fragments of said C-terminal sequences comprising or consisting of the MSP3-b-like and the MSP3-c/d-like motifs.

11. An antigenic polypeptidic composition according to anyone of claims 1 to 10, wherein the polypeptidic components are several fusion polypeptides wherein each fusion polypeptide comprises or consists of a polypeptide having the sequence consisting of:
(i) the C-terminal sequence of at least two MSP3-like proteins selected among MSP3-1, MSP3-2, MSP3-3, MSP3-4, MSP3-7 and MSP3-8 or;
(ii) several peptide fragments of the C-terminal sequence of at least two MSP3-like proteins selected among MSP3-1, MSP3-2, MSP3-3, MSP3-4, MSP3-7 and MSP3-8, wherein each peptide fragment comprises or consists of a least one MSP3-b-like motif or at least one MSP3-c/d-like motif.

12. An antigenic polypeptidic composition according to anyone of claims 1 to 12, which comprises or consists of a fusion polypeptide comprising or consisting of:
(i) the C-terminal sequence of each of the MSP3-like proteins selected among MSP3-1, MSP3-2, MSP3-3, MSP3-4, MSP3-7 and MSP3-8 or;
(ii) one or several peptide fragments of the C-terminal sequence of each of the MSP3-like proteins selected among MSP3-1, MSP3-2, MSP3-3, MSP3-4, MSP3-7 and MSP3-8, wherein each peptide fragment comprises or consists of a least one MSP3-b-like motif or at least one MSP3-c/d-like motif, wherein the fragments of the C-terminal sequences of the various MSP3-like proteins form a unique amino-acid sequence.

13. An antigenic polypeptidic composition according to claim 11 or 12, wherein, the peptide fragments of the C-terminal sequence of at least two MSP3-like proteins selected among MSP3-1, MSP3-2, MSP3-3, MSP3-4, MSP3-7 and MSP3-8 contain at least one MSP3-b-like motif and one or several further motif selected among the MSP3-a, -c/d, -e and -f-like motifs and said motifs are contiguous or not in said peptide fragments.

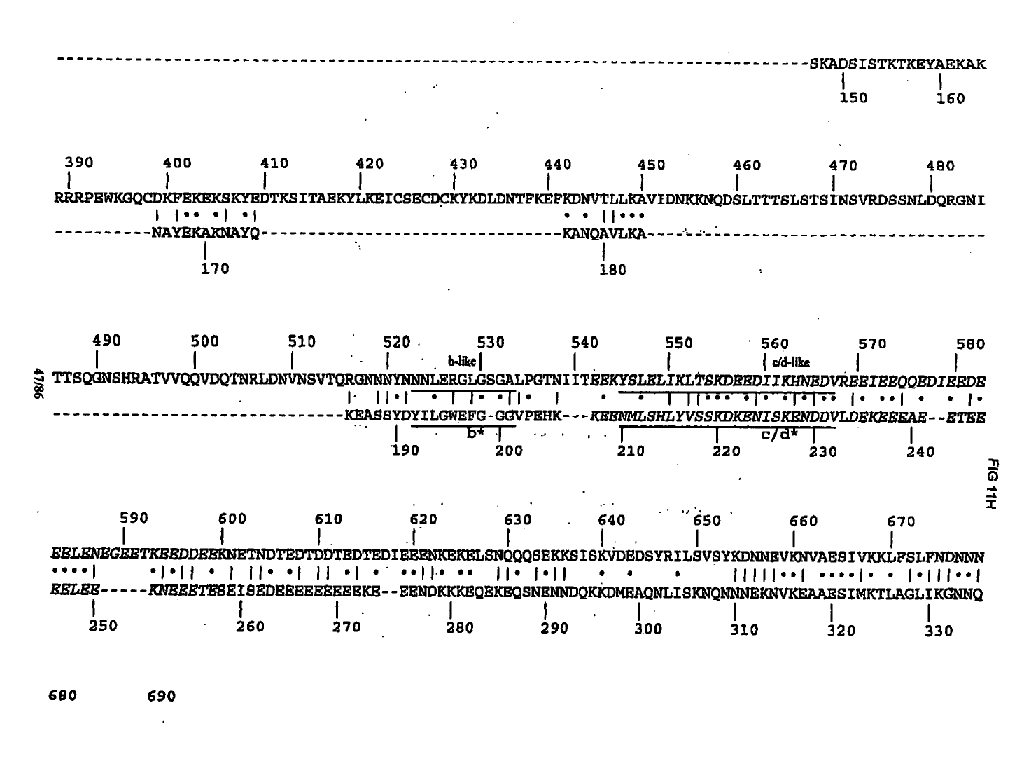

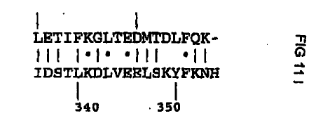

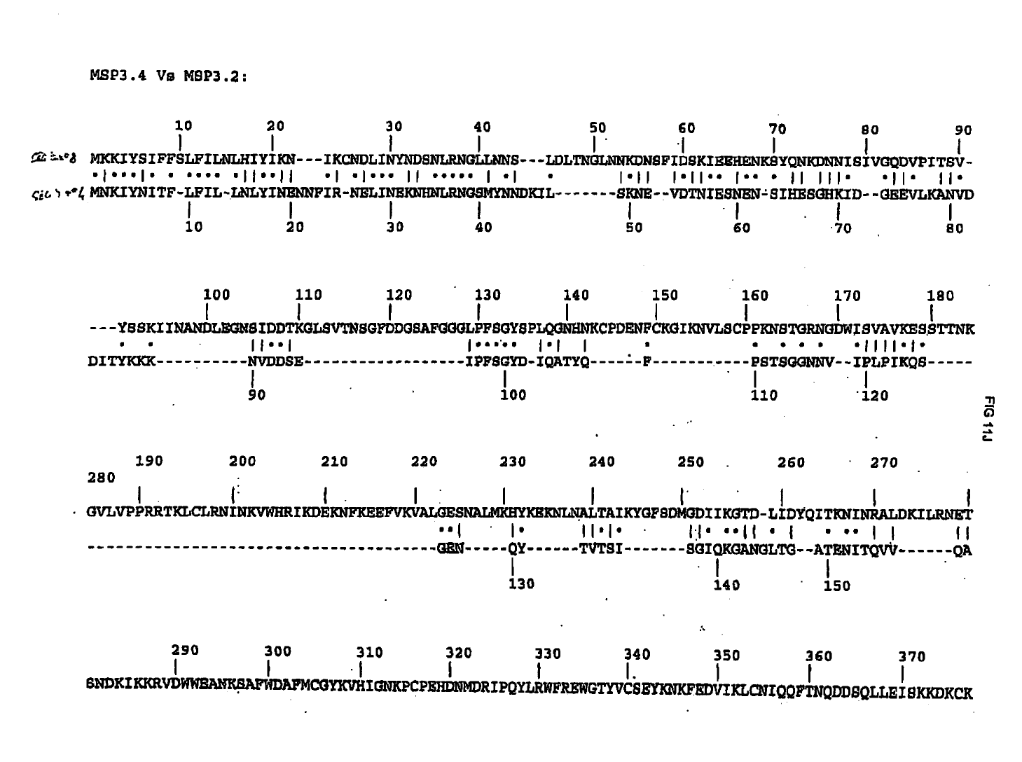

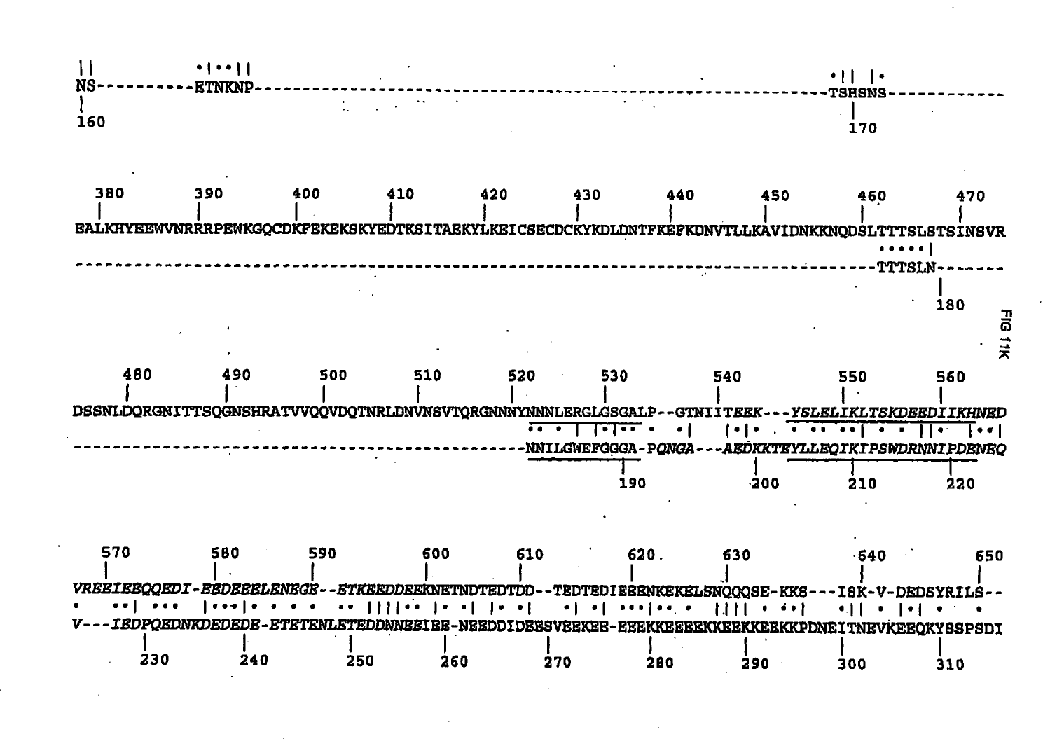

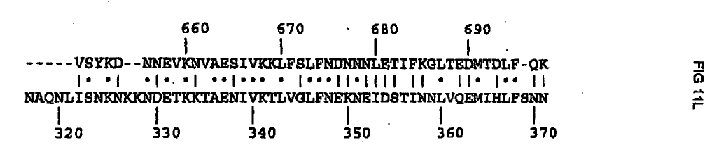

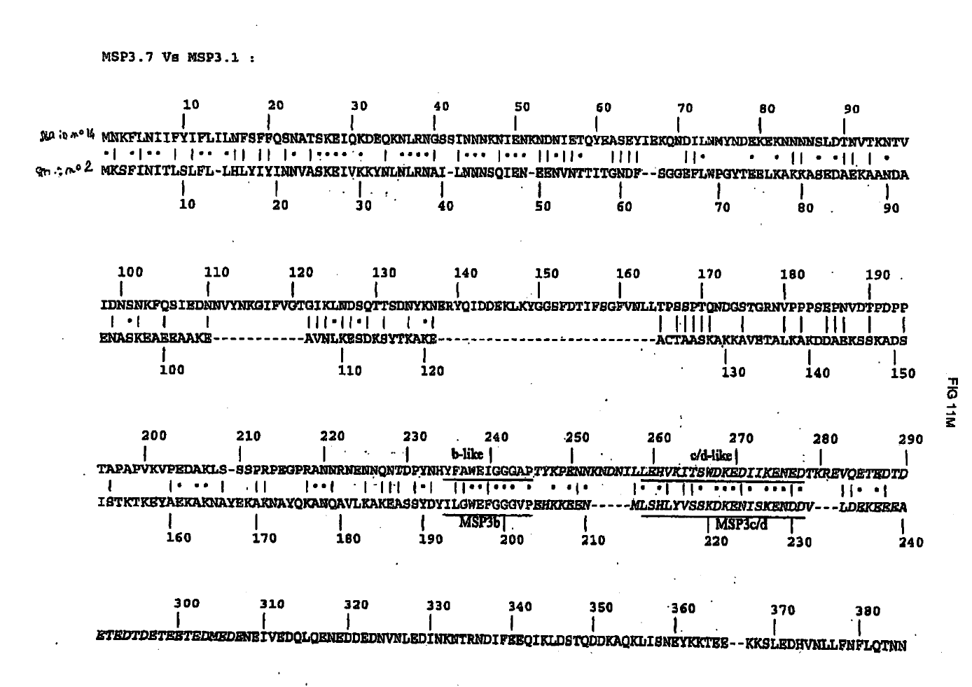

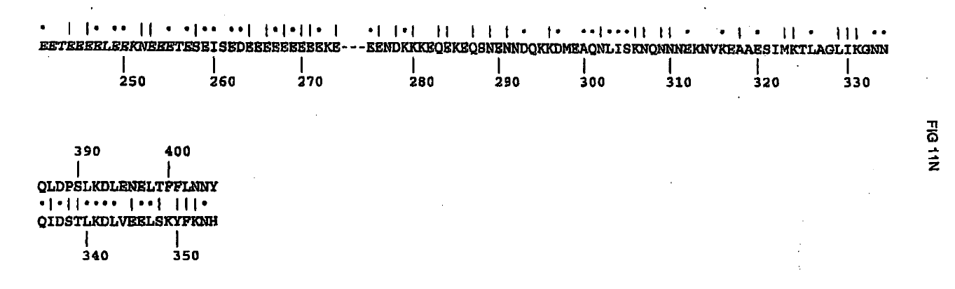

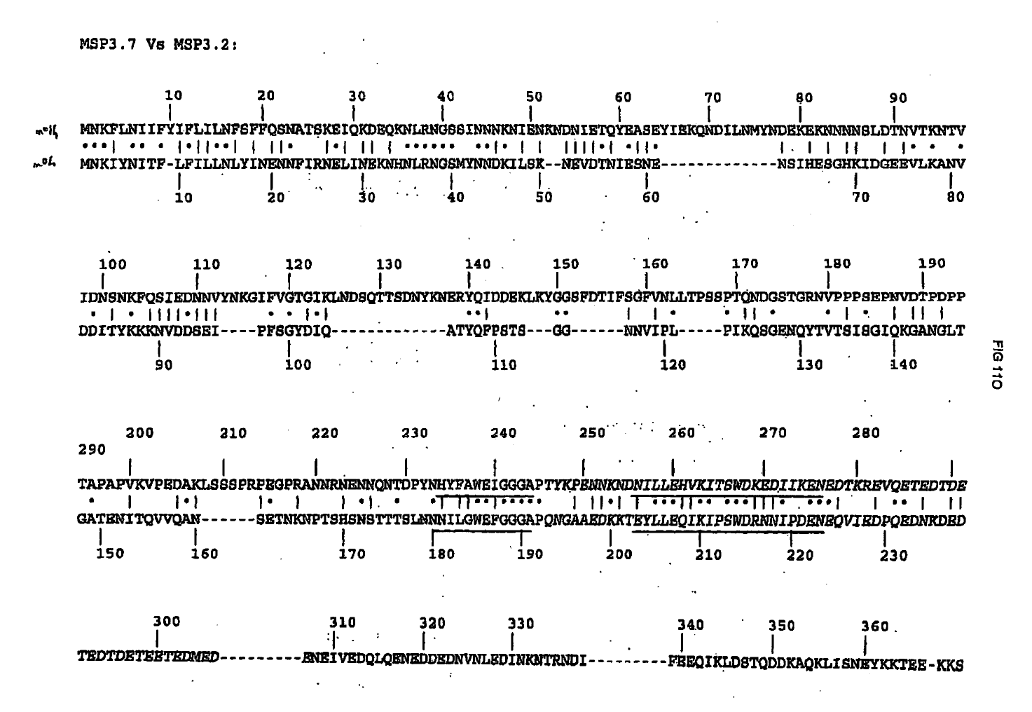

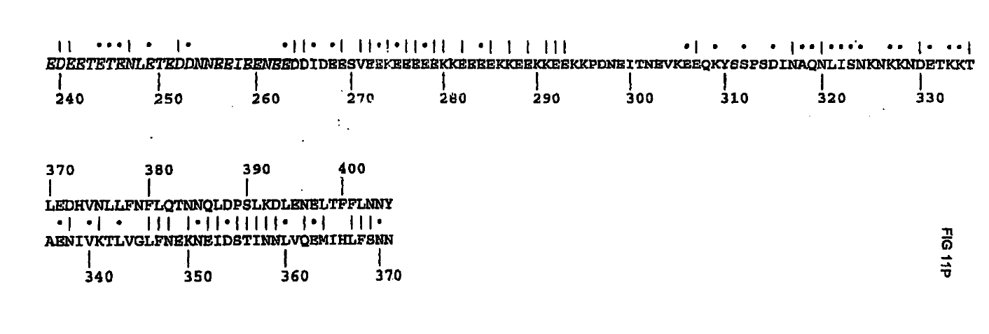

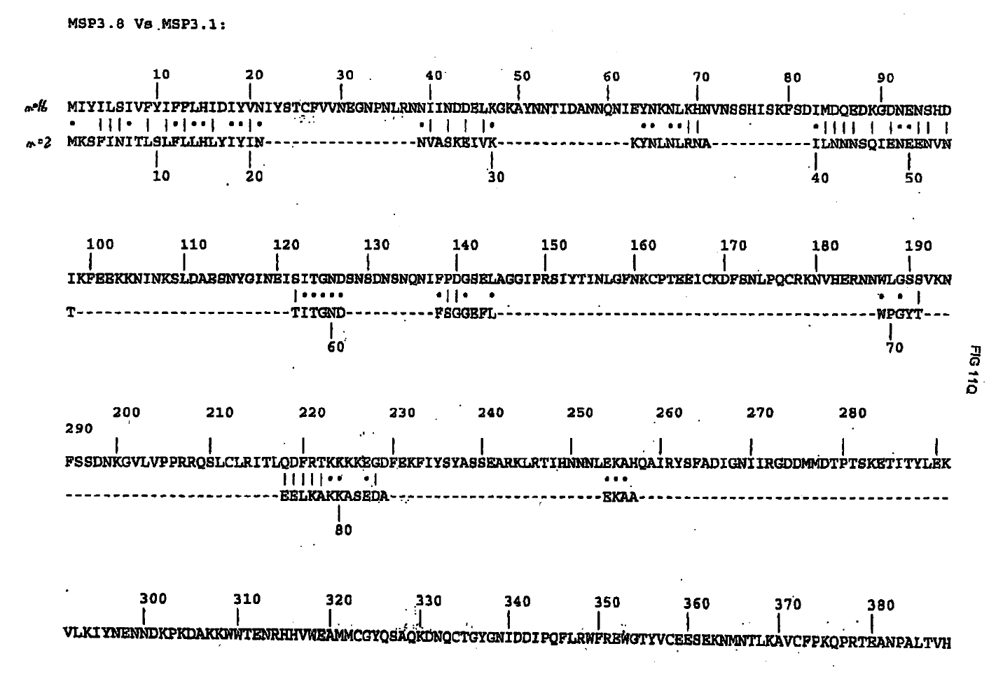

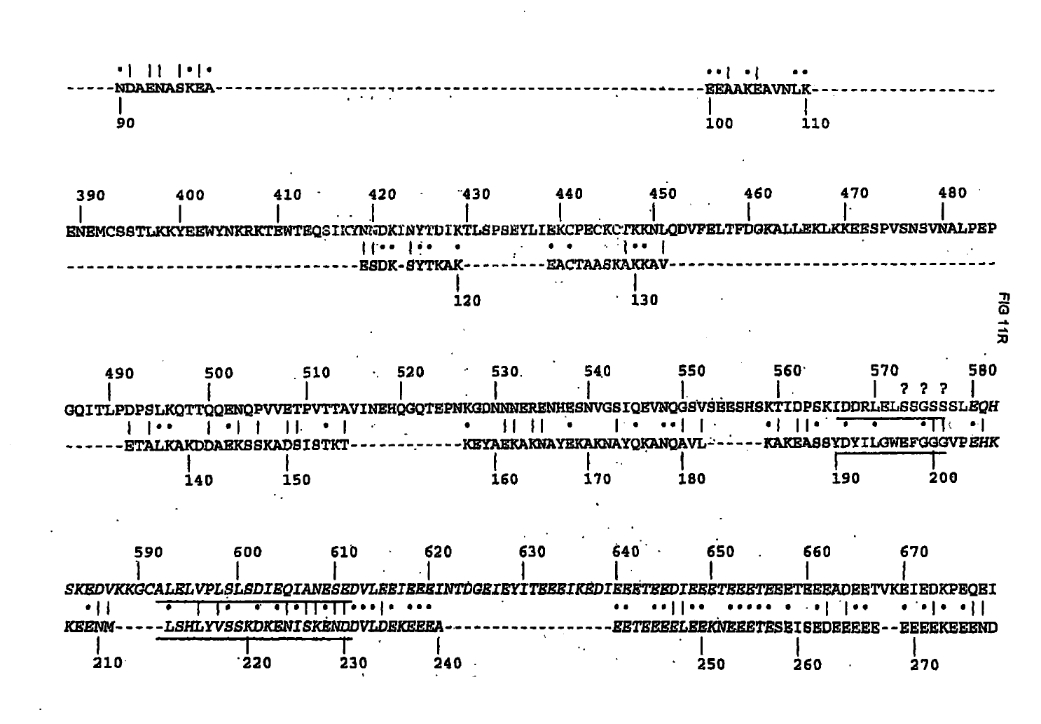

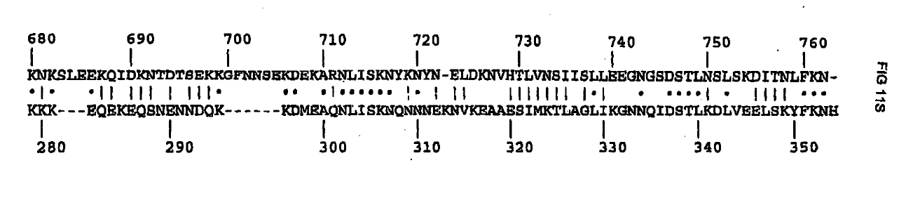

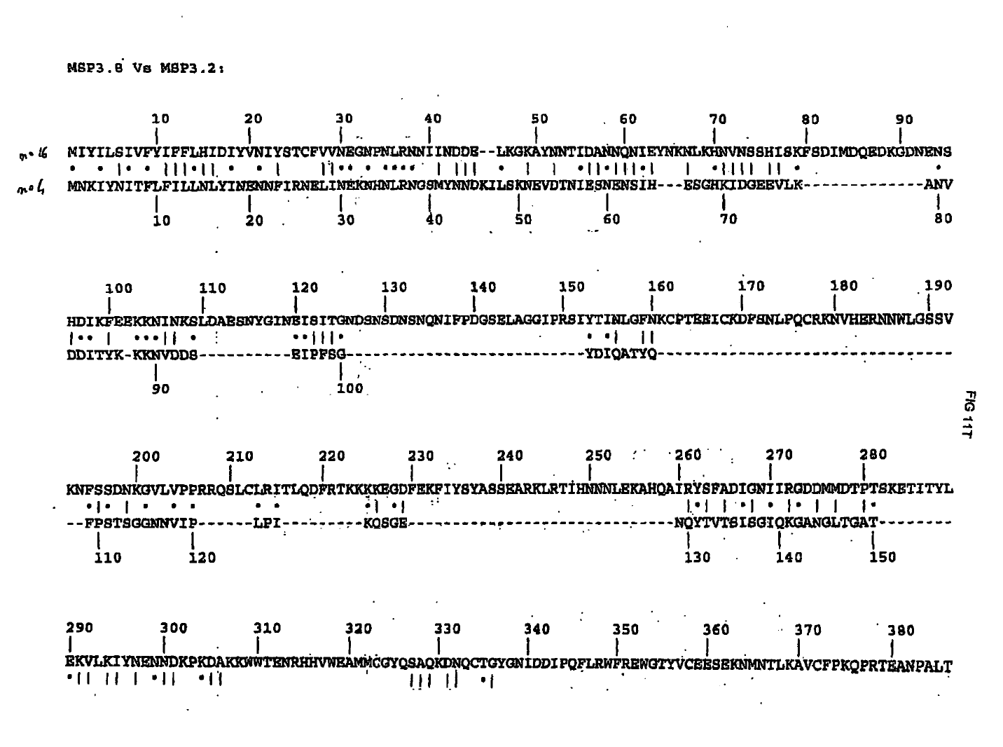

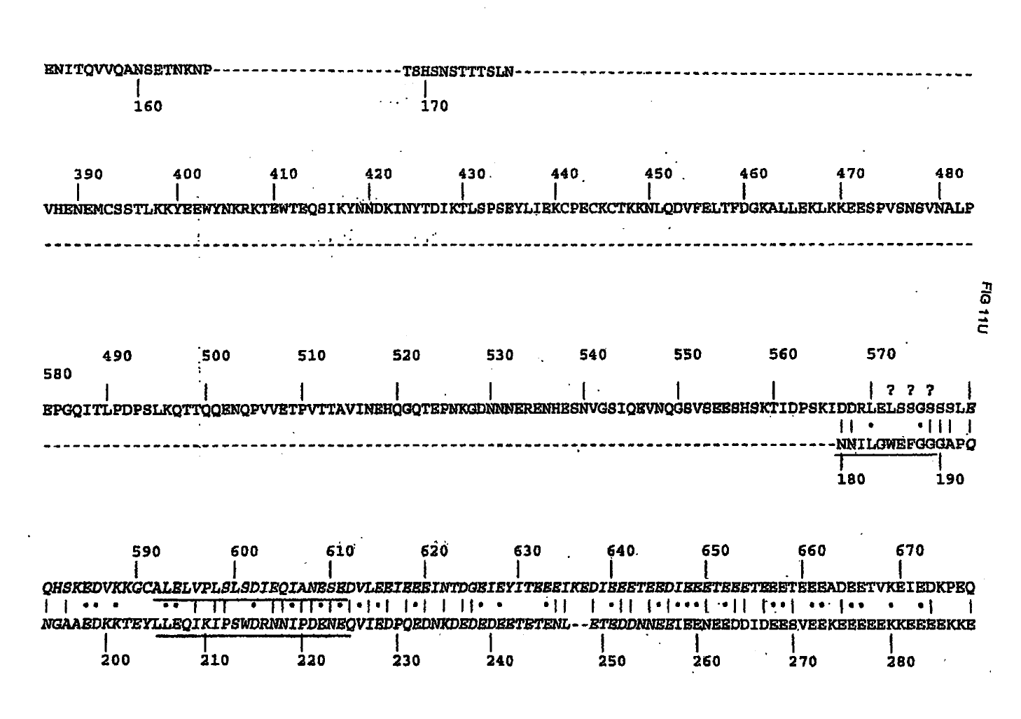

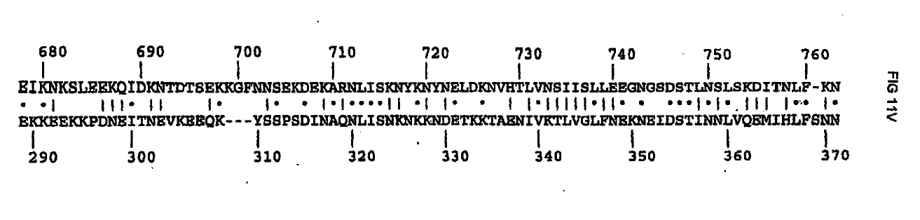

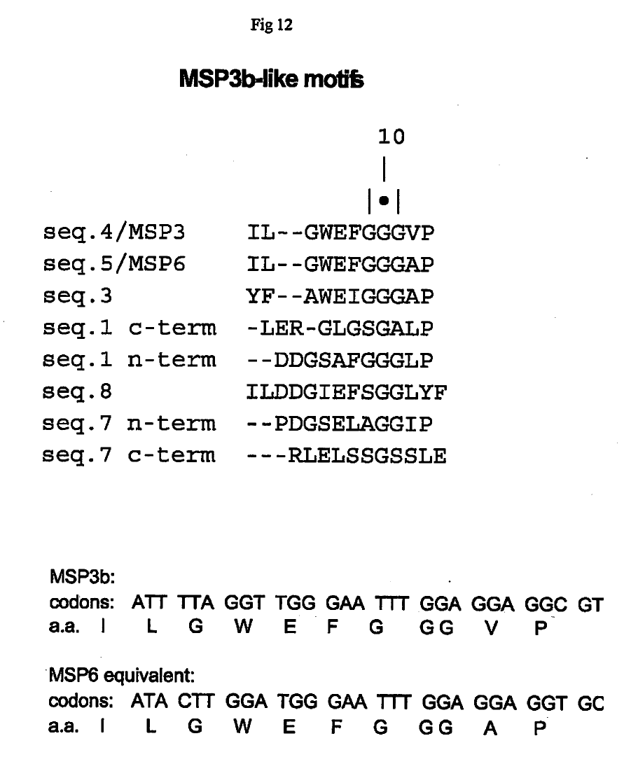

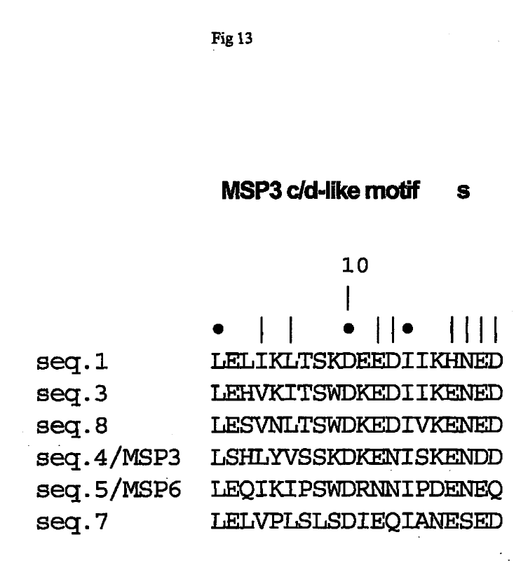

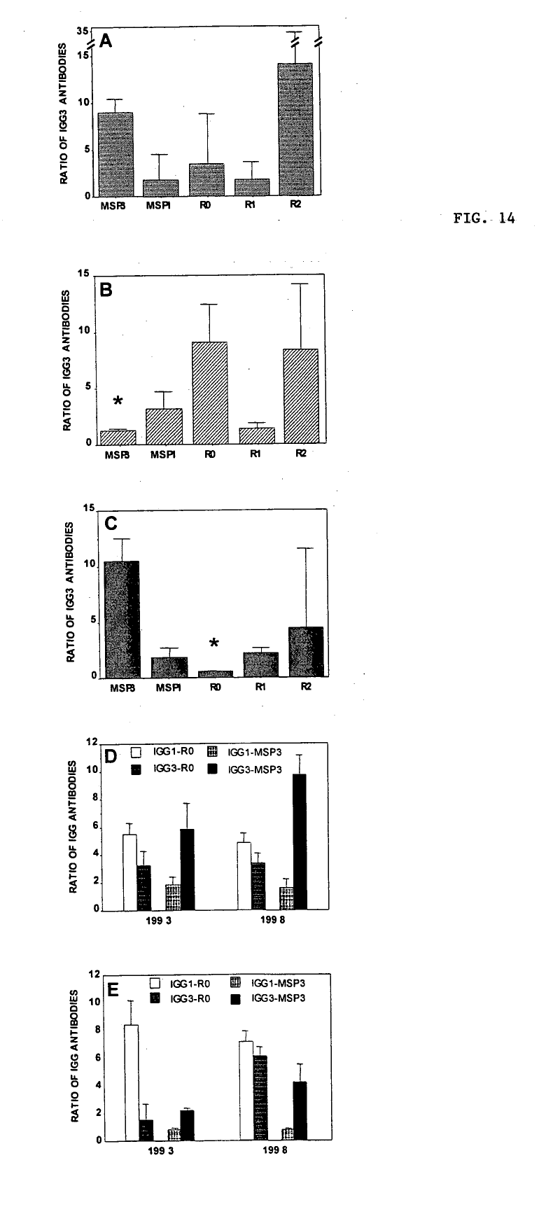

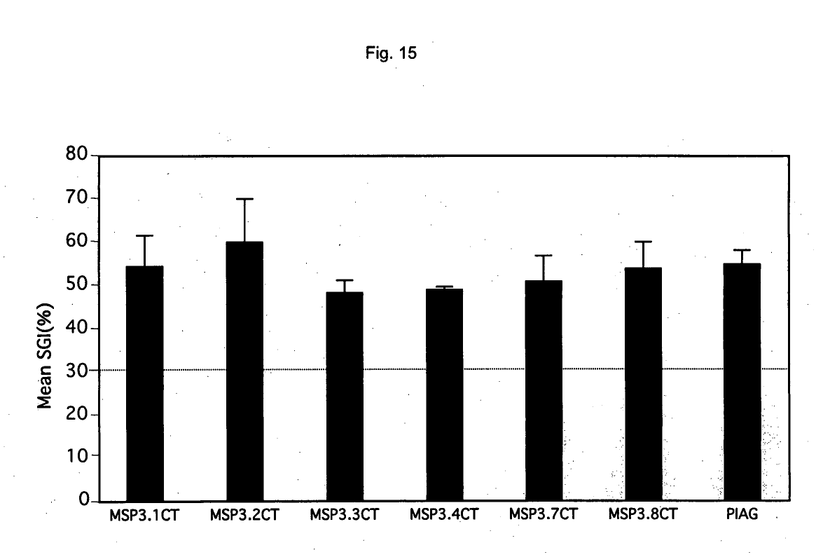

14. An antigenic polypeptidic composition according to anyone of claims 1 to 13, wherein the C-terminal sequence of the MSP3-like proteins are the following sequences:
(i) for MSP3-1, any sequence of FIG. 10A, and especially the sequence of strain 3D7;
(ii) for MSP3-2, any sequence of FIGS. 10-B-D and especially the sequence of strain 3D7; or a fragment of any of said sequences starting at amino-acid residue 161 (or 165 for sequences MSP3.2FL D4) and ending at amino acid residue 371 (or 376 for sequence MSP3.2FL D4),
(iii) for MSP3-3, any sequence of FIGS. 10D-E, and especially the sequence of strain 3D7;
(iv) for MSP3-4, any sequence of FIGS. 10E-F, and especially the sequence of strain 3D7;
(v) for MSP3-7, any sequence of FIGS. 10F-H, and especially the sequence of strain 3D7;
(vi) for MSP3-8, any sequence of FIG. 10I, and especially the sequence of strain 3D7.

15. The antigenic polypeptidic composition according to anyone of claims 1 to 14, wherein the at least two different MSP-3-b-like motifs are selected amongst the sequences of SEQ ID Nos: 17 to 24, and/or the at least two different MSP-3-c/d-like motifs are selected amongst the sequences of SEQ ID Nos: 25 to 30.

16. The antigenic polypeptidic composition according to any of claims 1 to 15, further comprising an antigenic polypeptide comprising at least 10 consecutive amino acid residues from the R0 region of GLURP.

17. The antigenic polypeptidic composition according to any of claims 1 to 15, which comprises at least two synthetic peptides comprising or corresponding to the sequence
(SEQ ID No:31)
X₁-X₂-X₃-X₄-X₅-X₆-X₇-X₈-X₉-G-X₉-X₁₀-X₁₁-X₁₂,
wherein:
X₁=I, Y or none;
X₂=L, F or none;
X₃=E, D, P or none;
X₄=R, D or none;
X₅=G, A, L or none;
X₆=W, G, S, I or E;
X₇=E, L or A;
X₈=F, I, G, L or S;
X₉=G, S or A;
X₁₀=V, A, L, I or S;
X₁₁=P, Y or L;
X₁₂=E, F or none.

18. The antigenic polypeptidic composition according to any of claims 1 to 15, which comprises at least two synthetic peptides comprising or corresponding to the sequence
(SEQ ID No:32)
X₁-X₂-X₃-W-E-X₄-G-G-G-X₅-P,
wherein:
X₁=I or Y:
X₂=L or F;
X₃=G or A;
X₄=F or I; and
X₅=V or A.

19. The antigenic polypeptidic composition according to any of claims 1 to 15, which comprises at least two synthetic peptides comprising or corresponding to the sequence
(SEQ ID No:33)
L-X₁-X₂-X₃-X₄-X₃-X₅-X₆-X₇-D-X₈-X₉-X₁₀-I-X₁₁-X₁₂-
X₁₃-X₁₄-X₁₅-X₁₆,
(SEQ ID No:33),
wherein:
X₁=E, or S;
X₂=L, H, S or Q;
X₃=I, V or L;
X₄=K, N, Y or P;
X₅=T, S or P;
X₆=S or L;
X₇=K, W or S;
X₈=E, K, R or I;
X₉=E or N;
X₁₀=D, N or Q;
X₁₁=I, V, S, P or A;
X₁₂=K, D or N;
X₁₃=H or E;
X₁₄=N or S;
X₁₅=E or D;
X₁₆=D or Q.

20. The antigenic polypeptidic composition according to claim 17 to 19, which is a mixotope, especially a mix of at least 50, at least 100, or at least 500 peptides of different sequences.

21. The antigenic polypeptidic composition according to any of claims 1 to19, wherein the at least two different MSP-3-b-like motifs are SEQ ID Nos: 17 and 19, or SEQ ID Nos: 17 and 20, or SEQ ID Nos: 17 and 22, or SEQ ID Nos: 20 and 23, or SEQ ID Nos: 20 and 24, or a combination thereof.

22. The antigenic polypeptidic composition according to any of claims 1 to 19, wherein the at least two different MSP-3-c/d-like motifs are SEQ ID Nos: 25 and 27, or SEQ ID Nos: 25 and 28, or SEQ ID Nos: 28 and 30, or SEQ ID Nos: 25 and 29, or a combination thereof.

23. The antigenic polypeptidic composition according to claim 1 to 19, wherein the polypeptidic components comprise sequences of the MSP3-like proteins which consist of the C-terminal sequence of MSP3-1 and of MSP3-7.

24. The antigenic polypeptidic composition according to claim 23 which further comprises the polypeptidic component which consists of the C-terminal sequence of MSP3-3.

25. The antigenic polypeptidic composition according to claim 23 or 24, which further comprises the polypeptidic component which consists of the C-terminal sequence of MSP3-2.

26. The antigenic polypeptidic composition according to any of claims 23 to 25, which further comprises the polypeptidic component which consists of the C-terminal sequence of MSP3-8 or/and MSP3-4.

27. A family of purified genes which have the following properties:
they are located on chromosome 10 of Plasmodium falciparum;
they are highly conserved in Plasmodium falciparum strains;
they are simultaneously expressed in Plasmodium falciparum at the erythrocytic stages;
they encode proteins which have a NLRN or NLRK signature at their N-terminal extremity and which are located at the merozoite surface, wherein said family comprises at least 3 genes.

28. A family of polynucleotides of the family of genes according to claim 27, wherein the polynucleotides are derived from said genes, and in particular a family of polynucleotides encoding the C-terminal part of said genes, or polynucleotide fragments of said C-terminal part, in particular polynucleotides having 30 to 500 nucleotides, especially 30 up to 250, or to 240, or to 210, or to 180, or to 150, or to 120 or to 90 nucleotides.

29. The family of genes according to claim 27 or 28, wherein said genes further have a conserved C-terminal sequence which encodes T-epitopes which are conserve among the genes of the family and wherein said terminal sequence further comprises conserved divergences among the genes of the family.

30. The family of genes according to any of claims 27 to 29, wherein said genes further have the following property:
antibodies to the products of said genes mediate Plasmodium falciparum blood stage killing, in the monocyte-dependent, antibody-mediated ADCI mechanism, under in vitro conditions.

31. The family of genes according to any of claims 27 to 29, wherein said genes further have the following property: antibodies to the products of said genes mediate Plasmodium falciparum growth inhibition in mice infected by P. falciparum.

32. The family of genes according to claim 27 to 30, which comprises the sequences encoding the C-terminal sequence of MSP3-like proteins of Plasmodium falciparum strain 3D7 represented on FIG. 10, or their homologues in other Plasmodium strains represented on FIG. 10.

33. The family of genes according to any of claims 27 to 31, which comprises the genes of SEQ ID Nos: 1, 3, 5, 7, 13 and 15, or their homologues in Plasmodium strains.

34. A Plasmodium falciparum gene isolated from a family according to any of claims 27 to 32, which has the sequence of SEQ ID No:5, or its homologue in a Plasmodium strain.

35. A Plasmodium falciparum gene isolated from a family according to any of claims 27 to 32, which has the sequence of SEQ ID No:7, or its homologue in a Plasmodium strain.

36. A Plasmodium falciparum gene isolated from a family according to any of claims 27 to 32 which has the sequence of SEQ ID No:13, or its homologue in a Plasmodium strain.

37. A Plasmodium falciparum gene isolated from a family according to any of claims 27 to 32, which has the sequence of SEQ ID No:15, or its homologue in a Plasmodium strain.

38. A polynucleotide sequence which hybridizes in stringent conditions with a Plasmodium falciparum gene according to anyone of claims 34 to 37 or with a family of genes according anyone of claims 27 to 33.

39. A protein which is encoded by a gene according to any of claims 34 to 38.

40. An antigenic polypeptide comprising or consisting of a fragment of at least 10, preferably at least 15, consecutive amino acids from a protein according to claim 39.

41. The antigenic polypeptide according to claim 40, which comprises at least one MSP-3-b-like motif.

42. The antigenic polypeptide according to claim 39 or 40, which comprises at least one MSP-3-c/d-like motif.

43. The antigenic polypeptide according to any of claims 39 to 42, or the antigenic polypeptidic composition according to any of claims 1 to 26, wherein a lipidic molecule is linked to at least part of the polypeptidic molecules.

44. The antigenic polypeptide or polypeptidic composition according to claim 43, wherein the lipidic molecule is a, C-terminal palmitoylysylamide residue.

45. The antigenic polypeptide according to any of claims 39 to 44, or the antigenic polypeptidic composition according to any of claims 16 to 25, wherein at least part of the polypeptidic molecules are bound to a support.

46. The antigenic polypeptide or polypeptidic composition according to claim 45, wherein the support is viral particles, or nitrocellulose or polystyrene beads, or a biodegradable polymer such as lipophosphoglycanes or poly-L lactic acid.

47. An immunogenic composition comprising as an immunogen a recombinant protein according to claim 37, or a polypeptide according to any of claims 39 to 46, or a polypeptidic composition according to any of claims 1 to 26.

48. A vaccine against malaria comprising as an immunogen a recombinant protein according to claim 39, or a polypeptide according to any of claims 40 to 46, or a polypeptidic composition according to any of claims 1 to 26, in association with a suitable pharmaceutical vehicle.

49. The immunogenic composition of claim 47 or the vaccine of claim 48, further comprising at least one antigen selected amongst LSA-1, LSA-3, LSA-5, SALSA, STARP, TRAP, PfEXP1, CS, MSP1, MSP2, MSP4, MSP5, AMA-1, SERP and GLURP.

50. The immunogenic composition or the vaccine according to any of claims 47 to 49, which is formulated for intradermal or intramuscular injection.

51. The immunogenic composition or vaccine of claim 50, comprising between 1 and 100 μg of immunogen per injection dose, preferably between 2 and 50 μg.

52. The immunogenic composition or vaccine of any of claims 47 to 50, further comprising SBAS2 and/or Alum and/or Montanide as an adjuvant.

53. Use of a recombinant protein according to claim 39, or a polypeptide according to any of claims 40 to 46, or a polypeptidic composition according to any of claims 1 to 26, for the preparation of a vaccine composition against malaria.

54. A synthetic or recombinant purified antibody or fragment of antibody which cross-reacts with several proteins according to claim 39, and which mediates Plasmodium falciparum blood stage killing, in the monocyte-dependent, antibody-mediated ADCI mechanism, under in vitro conditions.

55. A pool of antibodies or fragments of antibodies directed against several proteins according to claim 39 and/or polypeptides according to any of claims 40 to 46.

56. A pool of antibodies or fragments of antibodies directed against a polypeptidic composition according to any of claims 1 to 26.

57. An antibody according to claim 54, or a pool of antibodies according to claims 55 or 56, wherein said antibodies are human or humanized antibodies.

58. Use of a composition comprising an antibody or a pool of antibodies according to any of claims 55 to 57, for the preparation of a medicament against malaria.

59. A medicament for passive immunotherapy of malaria, comprising an antibody or a pool of antibodies according to any of claims 55 to 57.

60. The medicament of claim 60, further comprising antibodies directed against at least one antigen selected amongst LSA-1, LSA-3, LSA-5, SALSA, STARP, TRAP, PfEXP1, CS, MSP1, MSP2, MSP4, MSP5, AMA-1, SERP and GLURP.

61. A method for the in vitro diagnosis of malaria in an individual likely to be infected by P. falciparum, which comprises the bringing of a biological sample from said individual into contact with a protein according to claim 39, or an antigenic polypeptide of any of claims 40 to 46, or an antigenic composition according to anyone of claims 1 to 26, under conditions enabling the formation of antigen/antibody complexes between said antigenic peptide or polypeptide and the antibodies possibly present in the biological sample, and the in vitro detection of the antigen/antibody complexes possibly formed.

62. The method of claim 61, wherein the in vitro diagnosis is performed by an ELISA assay.

63. The method of claim 61 or claim 62, wherein the biological sample is further brought into contact with one or several antigenic peptides originating from other antigens selected amongst LSA-1, LSA-3, LSA-5, SALSA, STARP, TRAP, PfEXP1, CS, MSP-3-1, MSP-3-2, MSP-3-5, MSP-3-6, MSP1, MSP2, MSP4, MSP5, AMA-1, SERP and GLURP.

64. A kit for the in vitro diagnosis of malaria, comprising at least one peptide or polypeptide according to any of claims 39 to 46.

65. The kit of claim 64, wherein the antigenic peptide or polypeptide is bound to a support.

66. The kit of claim 64 or 65, further comprising reagents for enabling the formation of antigen/antibody complexes between said antigenic peptide or polypeptide and the antibodies possibly present in a biological sample, and reagents enabling the in vitro detection of the antigen/antibody complexes possibly formed.

67. A method for the in vitro diagnosis of malaria in an individual likely to be infected by P. falciparum, which comprises the bringing of a biological sample from said individual into contact with antibodies according to any of claims 54 to 57, under conditions enabling the formation of antigen/antibody complexes between said antibodies and the antigens specific for P. falciparum possibly present in the biological sample, and the in vitro detection of the antigen/antibody complexes possibly formed.

68. A kit for the in vitro diagnosis of malaria, comprising antibodies according to any of claims 54 to 57.

69. The kit of claim 68, further comprising reagents for enabling the formation of antigen/antibody complexes between said antibodies and antigens from the proteins of the MSP-3 family possibly present in a biological sample, and reagents enabling the in vitro detection of the antigen/antibody complexes possibly formed.

70. A recombinant nucleotide sequence comprising a sequence coding for a protein according to claim 39 or an antigenic polypeptide according to any of claims 40 to 46.

71. The recombinant nucleotide sequence according to claim 70, comprising a sequence encoding at least two MSP-3-b-like and/or MSP-3-c/d-like motifs, wherein at least one of said motifs is selected amongst the motifs of SEQ ID Nos: 19 to 24 and 27 to 30.

72. The recombinant nucleotide sequence according to claim 71, comprising a sequence encoding a fusion protein comprising several MSP-3-b-like motifs, wherein at least two of said motifs are selected amongst the motifs of SEQ ID Nos: 17 to 24.

73. The recombinant nucleotide sequence according to claim 71, comprising a sequence encoding a fusion protein comprising several MSP-3-b-like motifs, wherein at least two of said motifs are selected amongst the motifs of SEQ ID Nos: 25 to 30.

74. A recombinant cloning and/or expression vector, comprising a nucleotide sequence according to any of claims 33 to 38 or 70 to 73.

75. The recombinant cloning and/or expression vector of claim 74, wherein the nucleotide sequence is under the control of a promoter and regulatory elements homologous or heterologous vis-a-vis a host cell, for expression in the host cell.

76. Use of an expression vector according to claim 74 or 75, for the preparation of a medicament for genetic immunisation against Plasmodium falciparum.

77. A polynucleotide vaccine comprising a nucleotide sequence according to any of claims 70 to 73 or a gene of the gene family according to any of claims 27 to 33 or a gene according to any of claims 34 to 38.

78. A recombinant host cell, which is transformed by the vector of claim 77.

79. The host cell of claim 78, which is a bacterium, a yeast, an insect cell, or a mammalian cell.


SEQUENCE LISTING (30 sequences)

SEQ ID NO: 1 - DNA, 1065 bp, Plasmodium Falciparum
  misc_feature (1)..(1065): MSP 3.1
atgaaaagttttataaatattactctttcattatttttgttacatttatatatttatataaataatgttgctagtaaagaaattgtaaaaaaatataatcttaacttaagaaatgcaatattgaataataattctcaaatagaaaatgaagaaaatgtaaatactacaattactggtaatgattttagtggtggagaatttttgtggcctggttatacggaagaattaaaagctaaaaaagcttccgaagatgctgaaaaagctgctaatgatgctgaaaatgcttcaaaagaggcagaagaagctgctaaagaagcagtaaatttaaaggaatctgataaatcttatacaaaagcaaaagaagcatgtacagctgcttcaaaggcaaagaaagctgttgaaactgctttaaaggcaaaagatgatgctgaaaaatcttcaaaagctgatagtatttctacaaaaacaaaagaatatgctgaaaaagcaaaaaatgcttatgaaaaggcaaaaaatgcttatcaaaaagcaaaccaagctgttttaaaagcaaaagaagcttctagttatgattatattttaggttgggaatttggaggaggcgttccagaacacaaaaaagaagaaaatatgttatcacatttatatgtttcttcaaaggataaggaaaatatatctaaggaaaatgatgatgtattagatgagaaggaagaagaggcagaagaaacagaagaagaagaacttgaagaaaaaaatgaagaagaaacagaatcagaaataagtgaagatgaagaagaagaagaagaagaagaagaaaaggaagaagaaaatgacaaaaaaaaagaacaagaaaaagaacaaagtaatgaaaataatgatcaaaaaaaagatatggaagcacagaatttaatttctaaaaaccagaataataatgagaaaaacgtaaaagaagctgctgaaagcatcatgaaaactttagctggtttaatcaagggaaataatcaaatagattctaccttaaaagatttagtagaagaattatccaaatattttaaaaatcattaa

SEQ ID NO: 2 - protein, 354 aa, Plasmodium Falciuparum
  MISC_FEATURE (1)..(354): MSP 3.1
MKSFINITLSLFLLHLYIYINNVASKEIVKKYNLNLRNAILNNNSQIENEENVNTTITGNDFSGGEFLWPGYTEELKAKKASEDAEKAANDAENASKEAEEAAKEAVNLKESDKSYTKAKEACTAASKAKKAVETALKAKDDAEKSSKADSISTKTKEYAEKAKNAYEKAKNAYQKANQAVLKAKEASSYDYILGWEFGGGVPEHKKEENMLSHLYVSSKDKENISKENDDVLDEKEEEAEETEEEELEEKNEEETESEISEDEEEEEEEEEKEEENDKKKEQEKEQSNENNDQKKDMEAQNLISKNQNNNEKNVKEAAESIMKTLAGLIKGNNQIDSTLKDLVEELSKYFKNH

SEQ ID NO: 3 - DNA, 1116 bp, Plasmodium Falciparum
  misc_feature (1)..(1116): MSP 3.2
atgaataagatttataatattacttttcttttcattcttttaaacttatatataaatgaaaataactttatcagaaatgaacttataaacgaaaaaaaccataatttaagaaatggttcaatgtataataacgataaaatattaagtaaaaatgaagtagatactaatatagaaagtaacgaaaatagtattcacgaatctggacataagattgatggggaagaagttttaaaagctaatgtagatgatataacatacaaaaaaaaaaatgttgatgattcagaaattcctttttctggttatgatatacaagcaacatatcaatttccttctacatcaggaggaaataatgtaattccacttcctataaaacaaagtggagaaaatcaatatactgttacatctatatcaggtattcaaaagggagcaaatggtttaactggtgcaacagaaaatattacacaagttgtacaagcaaactctgaaacaaataaaaatcctacttctcatagtaatagtactacaacttctctgaataataatatacttggatgggaatttggaggaggtgctcctcaaaatggagctgcagaagataaaaagacagaatatttactagaacaaataaaaattccatcatgggatagaaataacatccccgatgagaatgaacaagtaatagaggaccctcaagaagataataaagatgaagatgaagatgaagaaacagaaacagaaaatttggaaacagaagatgataataatgaagagatagaagaaaatgaagaagatgacatagatgaagaaagtgtagaagaaaaggaagaagaggaagaaaaaaaggaagaagaagaaaaaaaggaagaaaaaaaagaagaaaaaaaaccagacaatgaaattacaaatgaagttaaagaggaacaaaaatatagttcaccaagtgatataaatgcccaaaatttaatttctaataagaataaaaagaatgatgaaacaaaaaagactgctgaaaatatagttaaaacattggttggattatttaatgaaaaaaatgagatagattctactataaataatttagtacaagaaatgatccatctatttagtaataattaa

SEQ ID NO: 4 - protein, 371 aa, Plasmodium Falciparum
  MISC_FEATURE (1)..(371): MSP 3.2
MNKIYNITFLFILLNLYINENNFIRNELINEKNHNLRNGSMYNNDKILSKNEVDTNIESNENSIHESGHKIDGEEVLKANVDDITYKKKNVDDSEIPFSGYDIQATYQFPSTSGGNNVIPLPIKQSGENQYTVTSISGIQKGANGLTGATENITQVVQANSETNKNPTSHSNSTTTSLNNNILGWEFGGGAPQNGAAEDKKTEYLLEQIKIPSWDRNNIPDENEQVIEDPQEDNKDEDEDEETETENLETEDDNNEEIEENEEDDIDEESVEEKEEEEEKKEEEEKKEEKKEEKKPDNEITNEVKEEQKYSSPSDINAQNLISNKNKKNDETKKTAENIVKTLVGLFNEKNEIDSTINNLVQEMIHLFSNN

SEQ ID NO: 5 - DNA, 1275 bp, Plasmodium Falciparum
  misc_feature (1)..(1275): MSP 3.3
atgaaaaaaatcgtgaatataatattttatatcttatatttatatatatataaaagaaacttagtacaaaacgaaaatgtaaataaatctaatctaagaaaaggattatctactaataattcagaaaatggaataaaaagtctaaaggatgaagatgaacatattaatattataggagatgatttttcagcattttcttatggtggttatcctatttatgaaactacaggaagtctaggaactggggttgaaagtgtaaaagctattgatggggaaagtggtacttcaatggattctaaacctaaagagaataaaattagtactgaaccaggagcagaccaggtgtctattggattggtcaacgaatctgatagtagtttagaaaatgataaaaaaaaaaaagaaaacgtaaaaaaagaaatgcttggtactgaaaaggaaggttctccagatagtcatgatagttctaaggaaaaattaaatcttaacgacaattccaaatggtctgattttcttaaaaatatcgtaacgtttggtggttttggtcctactgtggttcatgatgttagtgataccctttcagatatatctaaagatgaagttactcaaaaaacaactaaagatattggtagtactttacttgacttttttttaccattaccgactaaaaacactaatacatatgagaagaaaaatgaaaataaaaatgtatcaaatgtagattcaaaaacaaaatcaaatgaaaaaggaagacctcctacatattctcctattctggatgatgggatagaatttagtggtggtttatattttaatgagaaaaagtcgactgaagaaaataaacaaaaaaatgttttagaatcagtaaatttaacatcgtgggataaagaggatattgttaaggaaaatgaggatgttaaagatgaaaaggatgaagatgatgaagaagaagaagaaaaatacgaaaacgaaattataaagcaaccagaagacatattggatgaggaagaagtattagaagaagaaatattagaagaaaataaaaatgatacagtagatacaagtgatttagaaaagaaaaatataccagatttatcaaacgataataattattatagtttaatttataagaactataaggataatgataaatcagaaaaaactgcacaaacattaatcacagctctgataagtttattaaatggaaaaaatgaattagatgctaccataagaagattaaaacataggtttatggaattttttacatataattaa

SEQ ID NO: 6 - protein, 424 aa, Plasmodium Falciparum
  MISC_FEATURE (1)..(424): MSP 3.3
MKKIVNIIFYILYLYIYKRNLVQNENVNKSNLRKGLSTNNSENGIKSLKDEDEHINIIGDDFSAFSYGGYPIYETTGSLGTGVESVKAIDGESGTSMDSKPKENKISTEPGADQVSIGLVNESDSSLENDKKKKENVKKEMLGTEKEGSPDSHDSSKEKLNLNDNSKWSDFLKNIVTFGGFGPTVVHDVSDTLSDISKDEVTQKTTKDIGSTLLDFFLPLPTKNTNTYEKKNENKNVSNVDSKTKSNEKGRPPTYSPILDDGIEFSGGLYFNEKKSTEENKQKNVLESVNLTSWDKEDIVKENEDVKDEKDEDDEEEEEKYENEIIKQPEDILDEEEVLEEEILEENKNDTVDTSDLEKKNIPDLSNDNNYYSLIYKNYKDNDKSEKTAQTLITALISLLNGKNELDATIRRLKHRFMEFFTYN

SEQ ID NO: 7 - DNA, 2094 bp, Plasmodium Falciparum
  misc_feature (1)..(2094): MSP 3.4
atgaagaaaatatatagtattttcttttctttatttattttgaatcttcatatatatataaaaaatatcaaatgcaatgacctaataaattataatgattcgaatctaagaaacggattactaaataatagtttagatttaacaaatggattaaataacaaagataacagttttattgattctaaaattgaagaacatgaaaataaatcttaccaaaataaagataataatatctctatcgttggacaagatgtgcctattacatcggtatattcttctaaaattataaatgctaatgatttagaaggaaatagtattgacgatactaaaggtcttagtgttactaatagtggatttgatgatggtagtgcttttggtggtggactccctttttctggttattctcctctacaaggaaatcataataaatgtcctgatgaaaatttttgtaagggtattaaaaatgtcttatcctgtcctccaaaaaattctactggtagaaatggggattggattagtgtggctgttaaagaaagttcaactacaaataaaggtgttcttgttccccccagaagaacaaaattatgtctaagaaatattaacaaggtttggcatcgaatcaaagacgagaaaaattttaaagaagaatttgttaaagttgctttaggagaatcaaatgctttaatgaaacattataaagaaaaaaatctgaatgcccttacagctataaaatatggattttcagatatgggagatataataaagggaacagacctaattgactatcaaattactaaaaatataaatagggcattagataaaatattacgtaatgaaacaagtaatgacaaaattaaaaaacgtgtagactggtgggaagctaataaaagtgcattctgggatgcattcatgtgtggatataaagttcatatcggaaataaaccatgtccagaacatgataatatggacagaataccacaatatcttagatggtttagagaatggggaacatatgtttgcagcgaatataaaaataagtttgaggatgtaataaaattatgtaatatccaacaatttacaaaccaggatgattcacaactattagaaatatcaaaaaaggataaatgtaaagaagcattaaagcattatgaagaatgggttaatagaaggagacctgaatggaaaggccaatgtgataaatttgaaaaagaaaaaagtaaatatgaagatactaaaagtataactgctgaaaaatatttaaaagaaatatgttctgaatgtgattgtaaatataaagatttggataatacatttaaagaatttaaagataacgttacacttcttaaagcagtaattgataacaaaaaaaatcaagattctctaacaaccacttctttatcaacgtctattaatagtgttagggattctagtaatctagatcaacgagggaatataacaacatctcaaggaaattcacaccgtgcaactgttgtgcaacaagttgatcaaaccaacagattagataatgtaaactctgtaacgcaaagaggaaataataactacaacaataatttagagcgtggattgggttctggtgctcttcctggtacaaatattattactgaagaaaaatattctctagaattaataaaattaacatcaaaggatgaagaagatattataaagcataatgaggatgtgagagaagaaatagaagaacaacaagaagacatcgaggaagatgaagaagaattggaaaatgaaggagaagaaacaaaagaagaagatgatgaagaaaagaatgaaacaaatgatacggaagatacggacgatactgaagatacggaagatatagaagaggaaaataaggaaaaagaactcagtaatcaacaacaaagtgaaaaaaaaagtatttcaaaagttgacgaagattcatatcgaatactatcagtaagttataaggacaataatgaagtaaaaaatgttgctgaatctatagtgaaaaaactatttagtttatttaatgataataataatttggaaactatttttaagggtttgacagaagatatgacagatttatttcaaaaataa

SEQ ID NO: 8 - protein, 697 aa, Plasmodium Falciparum
  MISC_FEATURE (1)..(697): MSP 3.4
MKKIYSIFFSLFILNLHIYIKNIKCNDLINYNDSNLRNGLLNNSLDLTNGLNNKDNSFIDSKIEEHENKSYQNKDNNISIVGQDVPITSVYSSKIINANDLEGNSIDDTKGLSVTNSGFDDGSAFGGGLPFSGYSPLQGNHNKCPDENFCKGIKNVLSCPPKNSTGRNGDWISVAVKESSTTNKGVLVPPRRTKLCLRNINKVWHRIKDEKNFKEEFVKVALGESNALMKHYKEKNLNALTAIKYGFSDMGDIIKGTDLIDYQITKNINRALDKILRNETSNDKIKKRVDWWEANKSAFWDAFMCGYKVHIGNKPCPEHDNMDRIPQYLRWFREWGTYVCSEYKNKFEDVIKLCNIQQFTNQDDSQLLEISKKDKCKEALKHYEEWVNRRRPEWKGQCDKFEKEKSKYEDTKSITAEKYLKEICSECDCKYKDLDNTFKEFKDNVTLLKAVIDNKKNQDSLTTTSLSTSINSVRDSSNLDQRGNITTSQGNSHRATVVQQVDQTNRLDNVNSVTQRGNNNYNNNLERGLGSGALPGTNIITEEKYSLELIKLTSKDEEDIIKHNEDVREEIEEQQEDIEEDEEELENEGEETKEEDDEEKNETNDTEDTDDTEDTEDIEEENKEKELSNQQQSEKKSISKVDEDSYRILSVSYKDNNEVKNVAESIVKKLFSLFNDNNNLETIFKGLTEDMTDLFQK

SEQ ID NO: 9 - DNA, 2139 bp, Plasmodium Falciparum
  misc_feature (1)..(2139): MSP 3.5
atgaaatatatattaagtattagtctttttttaattcttttaaatttatataaatgtgctagtatatcatgtgaaatagattacacccctagtaccaatattactagcaatttaaattctaatttaagaagctgttcttctaatcttgttctttcttcaaatatagattctactaaattagaaggaacatatggtgaaaatttaaacgacagtgtatctttgacgaataatattaatgatcatacaactaacgaatcaaatatatcaaatgtatcaaatatatcaaatgaatcaaatatatcaaatgaatcaagtataactaaccagtcaaatttatctagcgaaacaaatatatctagcgaaacaaatatatctaacgaatcaagtgtaccaaatgaaagcagtgtcaatgaggtacctcaacttgaagtacctaaagatgcagtagaaaatcacacagaatccaaagatgttttattgaatgaaaaggaaaattttgcaaatggagtggaaacacatgttgatctaggatcccaagaacaatattttggatcttcctatgatatgaatatggacacagaaggaggaataaaaaagttcaaaaatgtgtttcagtcttattttaatcaatccaaaggaaatagtggtactgaaggtgatggatcaagtgtatttggatccatatttggtagcttattaacccctatagattcattgttagaaaaatttataggatctaacaatacaaattcggattctaatgtgaagaacacttctatgggaaatggacaaaataaatacgacaataatatatacttagatgaagaagatgctttgagtgatgcagaacattataatgatggtagtataagtttaggcgaagaagatgagttgagtgatgcagaacattataatgatggaagtataagtttagatgaagaagatgagttgagtgatgcagaacattataatgatggaggtatatgtttaggtgaagaagatgagttaagtgatgcagaacattataatgatggaggtataagtttagatgaagaagatgtgttgagtgatgcagaacattataatgatggagatataagtttagatgaagatgagttaagtgatacagaaaattattatgatggaggtataagtttagacgaatcagatgatttgagtgaccccgaaagtaaaacaaaagaagataactaccatttatattattgggatgatttctatcatgaatataaaccaacttatttaaattatcatatgcattatacactttatgaaccaaacaatttttatgatactactaatgaagaaacacacaatttttataatactactaatgaagaatcacacaatttttacaaccctactcatgaagaatcacacaatttttacaaccctactcatgaagaatcacacaatttttacaaccctactcatgaagaatcacacaatttttacaaccctactcatgaagaatcacacaatttttacaaccctactcatgaagaatcacacaatttttacaaccctactcatgaagaatcacacaatttttacaaccctactcatgaagaatcacacaatttttacaaccctactcatgaagaatcacacaatttttacaaccctactcatgaagaatcacacaatttttacaaccctactcatgaagaatcacacaatttttacaaccctactcatgaagaatcacacaatttttacacccctactcatgaagaatcacacaatttttacaaccctactcatgaagaatcacacaatttttacaaccctactcatgaagaatcacacaatttttacaaccctactcatgaagaatcacacaatttttacaaccctactcatgaagaatcacacaatttttacaaccctactcatgaagaatcacacaatttttacacccctactcatgatgaatttaatgttcctttaaattataaccatgactacgattacaattactttgaaaatgataattataatattcaaaatgttaaagacaatttggtaaaaaaagttaatgatttcatggaatcagataatttattagttaatacctttaaaggtatagctgggggtgttactagttttttcggatattaa

SEQ ID NO: 10 - protein, 712 aa, Plasmodium Falciparum
  MISC_FEATURE (1)..(712): MSP 3.5
MKYILSISLFLILLNLYKCASISCEIDYTPSTNITSNLNSNLRSCSSNLVLSSNIDSTKLEGTYGENLNDSVSLTNNINDHTTNESNISNVSNISNESNISNESSITNQSNLSSETNISSETNISNESSVPNESSVNEVPQLEVPKDAVENHTESKDVLLNEKENFANGVETHVDLGSQEQYFGSSYDMNMDTEGGIKKFKNVFQSYFNQSKGNSGTEGDGSSVFGSIFGSLLTPIDSLLEKFIGSNNTNSDSNVKNTSMGNGQNKYDNNIYLDEEDALSDAEHYNDGSISLGEEDELSDAEHYNDGSISLDEEDELSDAEHYNDGGICLGEEDELSDAEHYNDGGISLDEEDVLSDAEHYNDGDISLDEDELSDTENYYDGGISLDESDDLSDPESKTKEDNYHLYYWDDFYHEYKPTYLNYHMHYTLYEPNNFYDTTNEETHNFYNTTNEESHNFYNPTHEESHNFYNPTHEESHNFYNPTHEESHNFYNPTHEESHNFYNPTHEESHNFYNPTHEESHNFYNPTHEESHNFYNPTHEESHNFYNPTHEESHNFYNPTHEESHNFYNPTHEESHNFYTPTHEESHNFYNPTHEESHNFYNPTHEESHNFYNPTHEESHNFYNPTHEESHNFYNPTHEESHNFYTPTHDEFNVPLNYNHDYDYNYFENDNYNIQNVKDNLVKKVNDFMESDNLLVNTFKGIAGGVTSFFGY

SEQ ID NO: 11 - DNA, 1701 bp, Plasmodium Falciparum
  misc_feature (1)..(1701): MSP 3.6
atgttgaatatttttaatataattttcttgttgtttttaataaacatatatatatgtgaagccaatggaacactctctgaaaatattgaaagtgctgaagagatagatgctttaaaaacgaatttaagaaatggatatttaaataatacttattttaatgaagaaaacaataatttaaatatagaaaatgaaataaataatacaaattataatgaagtaacagaagaaactaaagaagaattatatgatataaatgaaaatattttccctgattatttttttcttgatatctttactgaaaataaagaacaaaaaaatgaagaagtaccaatgaaaatagaagtagtaaatgatggagaagaagtaaaaacagaatatgtatctgaaaaaaatgaggaagtagaaaataaatcggaaactgaaataggtgaagaattaactgaaaaagtagatgaaaaagtacctgaagaagtagctgaagaattagttgaaaaagtagatgaagaagtagctgaagaattagttgaaaaagtagatgaaaaagtagctgaagaagtagatcaaaaagtagatgaagaagtaactgaagaattaattgaaaaagtagatgaagaagtaactgaagaattaattgaaaaagtagatgaagaagttgctgaagaattaattgaaaaagtagatgaagaagttgctgaagaattaattgaaaaggtagctgatgaattaattgaaaaagtagatgaagaagttgctgaagaattaattgaaaaggtagctgatgaattagttgaaaaagtagctgaagaattagttgaaaaagtagatgaagaagtagctgaagaattagttgaaaaagtagatgaaaaagtagctgaagaagtagatcaaaaagtagatgaagaagtaactgaagaattaattgaaaaagtagatgaagaagtaactgaagaattaattgaaaaagtagatgaagaagttgctgaagaattaattgaaaaagtagatgaagaagttgctgaagaattaattgaaaaggtagctgatgaattagttgaaaaagtagctgaagaattagttgaaaaagtagatgaacaagtagctgaagaattagttgaaaaagtagatgaacaagtagctgaagaattagttgaaaaagtagatgaacaagtagttgaagaagtagctgaagaagtagctgaagaagtagttgaagaaggtgaaaaagtacctgaagaagtagctgaagaagtagctgaagaagtagctgaagaagtagctgaagaagtagctgaagaattagttgaaaaagtagatgaagaagtagctgaaaaagtagttgaagaagaaggtgaaaaagtacctgaagaagtagttgaagaagtagatgaagaagtagctgaaaaagtagttgaagaagaaggtgaaaaagtacttgaagaagtaattgaagaagtagttgaagaagtagccgaagaagtagctgaaaaagtagttgaagaacaaggtgaaaaagtaaacaaaaatgatttaaatgatgcatcttccgaggaaattaaggattctagtgattttaaagaatctcatgaggaattatttaaagttttcctggagttaattaataaaaacgatttagttaaagaaaatttaaaaaagattacaaacaatttaaatgaaatgcatttaagcactttatatccataa

SEQ ID NO: 12 - protein, 566 aa, Plasmodium Falciparum
  MISC_FEATURE (1)..(566): MSP 3.6
MLNIFNIIFLLFLINIYICEANGTLSENIESAEEIDALKTNLRNGYLNNTYFNEENNNLNIENEINNTNYNEVTEETKEELYDINENIFPDYFFLDIFTENKEQKNEEVPMKIEVVNDGEEVKTEYVSEKNEEVENKSETEIGEELTEKVDEKVPEEVAEELVEKVDEEVAEELVEKVDEKVAEEVDQKVDEEVTEELIEKVDEEVTEELIEKVDEEVAEELIEKVDEEVAEELIEKVADELIEKVDEEVAEELIEKVADELVEKVAEELVEKVDEEVAEELVEKVDEKVAEEVDQKVDEEVTEELIEKVDEEVTEELIEKVDEEVAEELIEKVDEEVAEELIEKVADELVEKVAEELVEKVDEQVAEELVEKVDEQVAEELVEKVDEQVVEEVAEEVAEEVVEEGEKVPEEVAEEVAEEVAEEVAEEVAEELVEKVDEEVAEKVVEEEGEKVPEEVVEEVDEEVAEKVVEEEGEKVLEEVIEEVVEEVAEEVAEKVVEEQGEKVNKNDLNDASSEEIKDSSDFKESHEELFKVFLELINKNDLVKENLKKITNNLNEMHLSTLYP

SEQ ID NO: 13 - DNA, 1218 bp, Plasmodium Falciparum
  misc_feature (1)..(1218): MSP 3.7
atgaataagtttttgaatattatattttacatttttctaatattaaatttctctttcttccaaagcaatgccacaagtaaggaaattcaaaaagatgaacaaaagaatttaagaaatggttcttcaataaataataacaaaaatatagaaaataaaaatgataatattgaaactcaatatgaagcttcagaatatatagaaaaacaaaatgacattttaaatatgtataatgatgaaaaagagaaaaataataataattcattagatacaaatgtaacaaaaaatactgtaattgataattcaaataaatttcaatcaattgaagacaataatgtatacaataaaggtatatttgtaggtactgggataaaattaaatgattcacaaactacatctgataattacaaaaatgaacgatatcaaatagacgatgaaaaattgaagtatggagggtcgtttgacacaattttttcaggttttgttaatttattaacaccatcaagtcctactcaaaacgatggatctacaggaagaaatgtaccacctcctagtgaacctaatgttgatacaccagatcctccaacagcacccgcacctgtaaaggtacctgaagatgcaaaattatcaagttctcctagacctgaaggaccaagagcaaacaatagaaatgaaaataatcaaaatacagatccatataaccactattttgcatgggaaattggaggtggtgctccaacgtataaacccgagaacaataagaacgataatattttgctagaacacgtaaaaattacctcgtgggataaagaagatataattaaagaaaatgaagacacaaaacgcgaagttcaagaaactgaagacactgacgaaactgaagatactgacgaaactgaagaaacagaagatatggaagatgaaaacgaaattgtggaagatcaattacaagaaaatgaagatgatgaggataatgtaaatttagaagatattaataaaaatactagaaatgatatatttgaagaacaaataaaattagattctacgcaagatgacaaagctcaaaaattaatttctaatgaatataaaaaaactgaagaaaaaaaatcattagaagatcatgtaaatctattatttaattttttacaaacaaataaccaactagatccttcactaaaagatttagaaaatgagttaactttttttttaaataactattga

SEQ ID NO: 14 - protein, 405 aa, Plasmodium Falciparum
  MISC_FEATURE (1)..(405): MSP 3.7
MNKFLNIIFYIFLILNFSFFQSNATSKEIQKDEQKNLRNGSSINNNKNIENKNDNIETQYEASEYIEKQNDILNMYNDEKEKNNNNSLDTNVTKNTVIDNSNKFQSIEDNNVYNKGIFVGTGIKLNDSQTTSDNYKNERYQIDDEKLKYGGSFDTIFSGFVNLLTPSSPTQNDGSTGRNVPPPSEPNVDTPDPPTAPAPVKVPEDAKLSSSPRPEGPRANNRNENNQNTDPYNHYFAWEIGGGAPTYKPENNKNDNILLEHVKITSWDKEDIIKENEDTKREVQETEDTDETEDTDETEETEDMEDENEIVEDQLQENEDDEDNVNLEDINKNTRNDIFEEQIKLDSTQDDKAQKLISNEYKKTEEKKSLEDHVNLLFNFLQTNNQLDPSLKDLENELTFFLNNY

SEQ ID NO: 15 - DNA, 2289 bp, Plasmodium Falciparum
  misc_feature (1)..(2289): MSP 3.8
atgatatatattttatctattgtattttatatattttttttacatattgatatatatgtaaacatttattctacatgttttgttgtaaatgaggggaaccctaatttaagaaataacataattaatgatgatgaactaaaggggaaagcatataataatactatagatgctaataaccaaaatatagaatataataaaaacttaaagcacaatgtaaactcatctcatatatctaaattttcggatattatggatcaagaagataaaggagataatgaaaattctcatgacataaaatttgaagaaaaaaaaaatattaataaatctttagacgctgaatccaattatggtattaatgaaattagtattactggtaatgatagtaatagtgataatagtaatcagaatatttttccagatggtagtgaattagctggaggtattcctcgttctatatatactattaaccttggttttaataaatgtcctactgaagagatttgtaaagactttagtaatcttccacaatgtcgaaagaatgtacatgaaagaaataattggttgggctcaagtgtaaaaaatttttcaagtgataataagggggttcttgttcctccaagaagacaatctttatgtttaagaattacattacaagattttcgtacgaaaaagaaaaaggaaggagattttgaaaaatttatttattcatatgcatcatctgaagctagaaaattaagaaccatacacaataataacttagaaaaagctcatcaagctataagatatagttttgcagatattggaaatattattagaggagatgacatgatggatacacctacgtcaaaagaaaccataacatatttagaaaaagtacttaaaatttataatgaaaataatgataaaccaaaagatgcaaaaaaatggtggacagaaaacaggcatcatgtttgggaagcaatgatgtgcggatatcagagtgcgcagaaagataaccaatgtacaggttatggtaacattgatgatataccacaatttttaaggtggttcagagagtggggaacatatgtctgtgaagaaagcgaaaaaaatatgaacacactaaaagctgtttgctttccgaaacagccaagaaccgaagcgaatcctgcattgactgtacatgaaaatgaaatgtgctcatcaactttaaaaaaatatgaagaatggtataataaaaggaaaactgaatggactgaacaatctattaaatataacaatgacaaaattaattatacagatataaaaacattatctccttctgaatatttaatagaaaaatgtcctgaatgtaaatgtaccaaaaaaaatttgcaagatgtatttgaacttacatttgatggaaaagctttattagaaaagctaaaaaaagaagaatcacctgtgagtaatagtgtgaatgccttacctgaaccaggtcaaattacattacctgatccttcattaaaacaaacaacacaacaggaaaatcaacctgttgtagaaacacctgttaccacagctgttattaatgaacatcaaggacaaacagaaccgaataaaggtgacaacaataatgaaagagaaaatcatgaaagtaatgttggtagcatccaagaagtaaaccaaggtagcgtgagcgaagaatcacattctaaaactatagatccttctaagattgacgaccgtttggaattaagtagtgggtcatcatctcttgaacaacactctaaggaagatgtaaaaaagggatgtgctttagaattggtacctttatctttatcggatattgaacagatagctaatgaaagcgaagatgtactggaagagatagaagaagaaattaatacagatggggaaatagaatatataacagaagaagaaataaaagaagatatagaagaagaaacagaagaagatatagaagaagaaacagaagaagaaacagaagaagaaacagaagaagaagcagatgaagaaacagtaaaagaaatagaagacaaaccagaacaagaaattaaaaataaatcgctagaagaaaaacaaatagataaaaatacagataccagtgaaaagaaaggatttaataattcagaaaaagatgaaaaagctcgaaatttaatttctaaaaattataaaaattataatgaactagataaaaacgttcatactttagtaaattcaattattagtttattagaagaaggtaatggaagtgattctaccttgaatagtttatcaaaagatattacaaatttatttaaaaattaa

SEQ ID NO: 16 - protein, 762 aa, Plasmodium Falciparum
  MISC_FEATURE (1)..(762): MSP 3.8
MIYILSIVFYIFFLHIDIYVNIYSTCFVVNEGNPNLRNNIINDDELKGKAYNNTIDANNQNIEYNKNLKHNVNSSHISKFSDIMDQEDKGDNENSHDIKFEEKKNINKSLDAESNYGINEISITGNDSNSDNSNQNIFPDGSELAGGIPRSIYTINLGFNKCPTEEICKDFSNLPQCRKNVHERNNWLGSSVKNFSSDNKGVLVPPRRQSLCLRITLQDFRTKKKKEGDFEKFIYSYASSEARKLRTIHNNNLEKAHQAIRYSFADIGNIIRGDDMMDTPTSKETITYLEKVLKIYNENNDKPKDAKKWWTENRHHVWEAMMCGYQSAQKDNQCTGYGNIDDIPQFLRWFREWGTYVCEESEKNMNTLKAVCFPKQPRTEANPALTVHENEMCSSTLKKYEEWYNKRKTEWTEQSIKYNNDKINYTDIKTLSPSEYLIEKCPECKCTKKNLQDVFELTFDGKALLEKLKKEESPVSNSVNALPEPGQITLPDPSLKQTTQQENQPVVETPVTTAVINEHQGQTEPNKGDNNNERENHESNVGSIQEVNQGSVSEESHSKTIDPSKIDDRLELSSGSSSLEQHSKEDVKKGCALELVPLSLSDIEQIANESEDVLEEIEEEINTDGEIEYITEEEIKEDIEEETEEDIEEETEEETEEETEEEADEETVKEIEDKPEQEIKNKSLEEKQIDKNTDTSEKKGFNNSEKDEKARNLISKNYKNYNELDKNVHTLVNSIISLLEEGNGSDSTLNSLSKDITNLFKN

SEQ ID NO: 17 - protein, 11 aa, Plasmodium Falciparum
  MISC_FEATURE (1)..(11): MSP 3-1 MSP3b-like motif
ILGWEFGGGVP

SEQ ID NO: 18 - protein, 11 aa, Plasmodium Falciparum
  MISC_FEATURE (1)..(11): MSP 3-2 MSP3b-like motif
ILGWEFGGGAP

SEQ ID NO: 19 - protein, 14 aa, Plasmodium Falciparum
  MISC_FEATURE (1)..(14): MSP 3-3 MSP3b-like motif
ILDDGIEFSGGLYF

SEQ ID NO: 20 - protein, 11 aa, Plasmodium Falciparum
  MISC_FEATURE (1)..(11): MSP 3-4 MSP3b-like motif
LERGLGSGALP

SEQ ID NO: 21 - protein, 11 aa, Plasmodium Falciparum
  MISC_FEATURE (1)..(11): MSP 3-4 MSP3b-like motif
DDGSAFGGGLP

SEQ ID NO: 22 - protein, 11 aa, Plasmodium Falciparum
  MISC_FEATURE (1)..(11): MSP 3-7 MSP3b-like motif
YFAWEIGGGAP

SEQ ID NO: 23 - protein, 11 aa, Plasmodium Falciparum
  MISC_FEATURE (1)..(11): MSP 3-8 MSP3b-like motif
RLELSSGSSLE

SEQ ID NO: 24 - protein, 11 aa, Plasmodium Falciparum
  MISC_FEATURE (1)..(11): MSP 3-8 MSP3b-like motif
PDGSELAGGIP

SEQ ID NO: 25 - protein, 20 aa, Plasmodium Falciparum
  MISC_FEATURE (1)..(20): MSP3-1 MSP3c/d-like motif
LSHLYVSSKDKENISKENDD

SEQ ID NO: 26 - protein, 20 aa, Plasmodium Falciparum
  MISC_FEATURE (1)..(20): MSP 3-2 MSP3c/d-like motif
LEQIKIPSWDRNNIPDENEQ

SEQ ID NO: 27 - protein, 20 aa, Plasmodium Falciparum
  MISC_FEATURE (1)..(20): MSP 3-3 MSP3c/d-like motif
LESVNLTSWDKEDIVKENED

SEQ ID NO: 28 - protein, 20 aa, Plasmodium Falciparum
  MISC_FEATURE (1)..(20): MSP 3-4 MSP3c/d-like motif
LELIKLTSKDEEDIIKHNED

SEQ ID NO: 29 - protein, 20 aa, Plasmodium Falciparum
  MISC_FEATURE (1)..(20): MSP 3-7 MSP3c/d-like motif
LEHVKITSWDKEDIIKENED

SEQ ID NO: 30 - protein, 20 aa, Plasmodium Falciparum
  MISC_FEATURE (1)..(20): MSP 3-8 MSP3c/d-like motif
LELVPLSLSDIEQIANESED